(19) United States
(12) Patent Application Publication
(10) Pub. No.: US 2019/0319197 A1
(43) Pub. Date: Oct. 17, 2019

(54) ORGANIC COMPOUND, APPLICATIONS THEREOF, ORGANIC MIXTURE, AND ORGANIC ELECTRONIC DEVICE

(71) Applicant: GUANGZHOU CHINARAY OPTOELECTRONIC MATERIALS LTD., Guangzhou, Guangdong (CN)

(72) Inventors: Junyou PAN, Guangzhou, Guangdong (CN); Hong HUANG, Guangzhou, Guangdong (CN)

(21) Appl. No.: 16/463,643

(22) PCT Filed: Nov. 23, 2017
(86) PCT No.: PCT/CN2017/112711

§ 371 Date: May 23, 2019

(30) Foreign Application Priority Data
Nov. 23, 2016 (CN) 201611047599.X

(51) Int. Cl. H01L 51/00; C07D 471/04; C09K 11/06; C07D 209/86; C07D 251/24

(52) U.S. Cl. CPC H01L 51/0072; C07D 471/04; C09K 11/06; C07D 209/86; C07D 251/24; H01L 51/5016; H01L 51/0054; H01L 51/0056; H01L 2251/552; C09K 2211/1018; H01L 51/0067

(57) ABSTRACT

An organic compound, applications thereof, an organic mixture, and an organic electronic device. The structure of the organic compound is represented by general formula (1), and definitions of substituent groups in the general formula (1) are the same as those in the specifications.

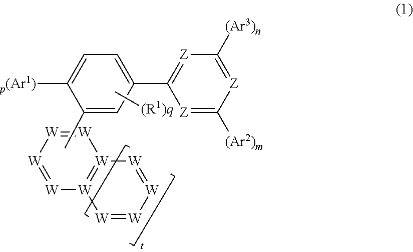

The present disclosure is the National Stage of International Application No. PCT/CN2017/112711, filed Nov. 23, 2017, entitled “ORGANIC COMPOUND, APPLICATIONS THEREOF, ORGANIC MIXTURE, AND ORGANIC ELECTRONIC DEVICE”, which claims priority to Chinese Patent Application No. 201611047599.X entitled “Organic compound, mixture and application thereof”, filed on Nov. 23, 2016, which is incorporated by reference herein for all purposes.

### TECHNICAL FILED

The present disclosure relates to the technical field of organic optoelectronic materials, and in particular to an organic compound and an application thereof, an organic mixture, and an organic electronic device.

### BACKGROUND

The diversity and synthesis of organic electroluminescent materials have established a solid foundation for the implement of large-area new display devices. In order to improve the luminous efficiency of organic light-emitting diodes, light-emitting material systems based on fluorescence and phosphorescence has been developed. The organic light-emitting diodes using the fluorescent materials have high reliability, however, their internal electroluminescence quantum efficiency is limited to 25% under electrical excitation, which is because the exciton has a branching ratio between the singlet excited state and the triplet excited state of 1:3. The organic light-emitting diodes using phosphorescent materials have achieved almost 100% internal electroluminescence quantum efficiency. However, a stability of a phosphorescent OLED (Organic Light-Emitting Diode) needs to be improved. The stability of the OLED relates to its emitter itself, a host material is also one of key factors.

Regarding red and green phosphorescent light-emitting devices, single host materials are generally used in order to simplify the manufacturing process of devices. However, the single host materials may cause different carrier transport rates, which causes serious roll-off of the device efficiency under high luminance, resulting in a shortening of the device lifetime. In addition, if two host materials are used for co-evaporation, some problems caused by the single host can be reduced. However, the material evaporation process is complicated, which is not conducive to a mass production of the device.

### SUMMARY

In accordance with various embodiments of the present disclosure, an organic compound and an application thereof, an organic mixture, and an organic electronic device are provided, solving one or more of problems involved in the related art.

An organic compound for an electronic device has a general formula (1) as following:

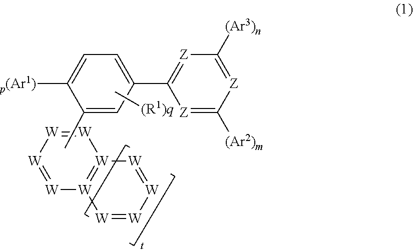

Wherein,

Z is selected from N or CR⁷, and at least one Z is N;

W is selected from N or CR⁷, and two linked Ws are not N simultaneously;

Ar¹ to Ar₃ are independently selected from the group consisting of an aromatic ring system containing 5 to 30 ring atoms, a heteroaromatic ring system containing 5 to 30 ring atoms, and a non-aromatic ring system containing 5 to 30 ring atoms; Ar¹ to Ar³ have a group R⁸ on rings thereof;

R¹ represents H, D, F, CN, alkenyl, alkynyl, nitrile, amino, nitro, acyl, alkoxy, carbonyl, sulfonyl, an alkyl containing 1 to 30 carbon atoms, a cycloalkyl containing 3 to 30 carbon atoms, an aromatic hydrocarbyl containing 5 to 60 ring atoms, or an aromatic heterocyclic group containing 5 to 60 ring atoms;

R⁷ and R⁸ are independently selected from the group consisting of hydrogen, deuterium, a substituted or unsubstituted alkyl containing 1 to 10 carbon atoms, a substituted or unsubstituted aromatic ring system containing 5 to 12 ring atoms, and a substituted and unsubstituted heteroaromatic ring system containing 5 to 12 ring atoms;

n, m, p, and q are independently selected from 1, 2, or 3; t is 0 or 1.

An organic mixture is provided, including an organic compound H2 and the aforementioned organic compound H1; min((LUMO(H1)-HOMO(H2)), (LUMO(H2)-HOMO(H1)))≤min(E_{T}(H1), E_{T}(H2))+0.1 eV. LUMO(H1), HOMO(H1), and E_{T}(H1) are energy levels of a lowest unoccupied molecular orbital, a highest occupied molecular orbital, and a triplet excited state of the organic compound H1, respectively. LUMO(H2), HOMO(H2), and E_{T}(H2) are energy levels of a lowest unoccupied molecular orbital, a highest occupied molecular orbital, and a triplet excited state of the organic compound H2, respectively.

A formulation comprising an organic solvent and the aforementioned organic compound is also provided.

An application of the aforementioned organic compound in an organic electronic device is also provided.

An organic electronic device including the aforementioned organic compound is also provided.

A method of manufacturing the aforementioned organic electronic device is further provided, including the following steps:

Grinding and mixing the organic compound H1 and the organic compound H2; And evaporating the ground and mixed organic compound H1 and the organic compound H2 in an organic source, and forming a functional layer of the organic electronic device.

A method of manufacturing the aforementioned organic electronic device is further provided, including the following steps:

Disposing the organic compound H1 and the organic compound H2 in two sources under vacuum, respectively; Evaporating the organic compound H1 and the organic compound H2, and forming a functional layer of the organic electronic device.

Details of one or more embodiments of the present disclosure are set forth in the accompanying drawings and description below. Other features, objects, and advantages of the present disclosure will be apparent from the description, accompanying drawings, and claims.

### BRIEF DESCRIPTION OF THE DRAWINGS

To illustrate the technical solutions according to the embodiments of the present disclosure or in the prior art more clearly, the accompanying drawings for describing the embodiments or the prior art are introduced briefly in the following. Apparently, the accompanying drawings in the following description are only some embodiments of the present disclosure, and persons of ordinary skill in the art can derive other drawings from the accompanying drawings without creative efforts.

FIG. 1 is a schematic view of heterojunction structures of an organic semiconductor material A and an organic semiconductor material B according to an embodiment.

### DETAILED DESCRIPTION OF THE EMBODIMENTS

In order to make the objects, technical solutions, and advantages of the present disclosure more clearly, the present disclosure will be further described in detail below with reference to the accompanying drawings and embodiments. It is understood that the specific examples described herein are merely illustrative of the disclosure and are not intended to limit the disclosure.

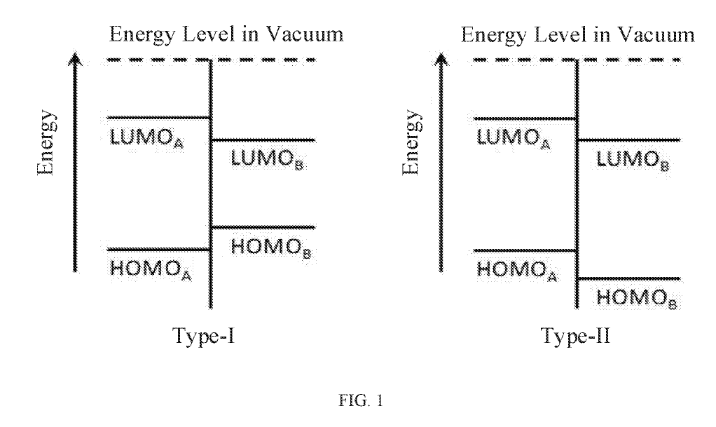

Formulations, printing inks, and inks described herein have the same meaning and are interchangeable in use. Host materials, matrix materials, Host material, and Matrix material described herein have the same meaning and are interchangeable in use. Metal organic clathrate, metal organic complexes, and organometallic complexes described herein have the same meaning and are interchangeable in use. In addition, in this context, a heterojunction refers to an interface region formed by two different semiconductors contacting each other, which may be divided into type-I and type-II according to the alignment of the conduction band (LUMO) and valence band (HOMO) of the two materials in the heterojunction, as shown in FIG. 1. A basic characteristic of the type-II heterojunction is a spatial separation of both electrons and holes in the vicinity of the interface, and a localization in a plurality of self-consistent quantum wells. Due to overlapping of a plurality of wave functions near the interface, it leads to a reduction of a plurality of optical matrix elements, which extends a radiation lifetime and reduces an exciton binding energy. In this context, (HOMO−1) is defined as an energy level of the second highest occupied molecular orbital, (HOMO−2) is an energy level of the third highest occupied molecular orbital, and so on. (LUMO+1) is defined as an energy level of the second lowest unoccupied molecular orbital, (LUMO+2) is an energy level of the third lowest occupied molecular orbital, and so on.

An organic compound for an electronic device according to an embodiment is provided, the organic compound has a general formula (1) as following:

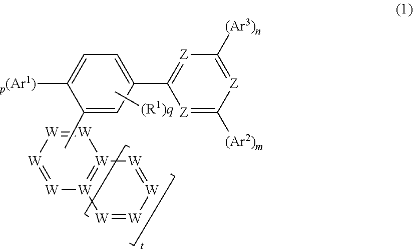

Wherein,

Z is selected from N or CR⁷, and at least one Z is selected from N atom;

W is selected from N or CR⁷, and two linked Ws are not N atoms simultaneously;

Ar¹ to Ar³ are independently selected from the group consisting of an aromatic ring system containing 5 to 30 ring atoms, a heteroaromatic ring system containing 5 to 30 ring atoms, and a non-aromatic ring system containing 5 to 30 ring atoms; hydrogens on rings of Ar¹ to Ar³ may be optionally substituted by a group R⁸;

R¹ represents H, D, F, CN, alkenyl, alkynyl, nitrile, amino, nitro, acyl, alkoxy, carbonyl, sulfonyl, an alkyl containing 1 to 30 carbon atoms, a cycloalkyl containing 3 to 30 carbon atoms, an aromatic hydrocarbyl containing 5 to 60 ring atoms, or an aromatic heterocyclic group containing 5 to 60 ring atoms;

R⁷ and R⁸ are independently selected from the group consisting of hydrogen, deuterium, a substituted or unsubstituted alkyl containing 1 to 10 carbon atoms, a substituted or unsubstituted aromatic ring system containing 5 to 12 ring atoms, and a substituted or unsubstituted heteroaromatic ring system containing 5 to 12 ring atoms;

n, m, p, and q are independently selected from 1, 2, or 3; and t is 0 or 1.

R¹ may be linked to any carbon atom on the ring, and there may be any number of carbon atoms substituted by R.

It should be noted that the hydrogen on rings of Ar¹ to Ar³ may be substituted by one or more groups R⁸, and the group R⁸ may be the same or different in multiple occurrences. In one of the embodiments, n, m, p, and q are 1. In one of the embodiments, t is 0.

In one of the embodiments, t is 1.

In one of the embodiments, W is selected from CR⁷. In addition, in one of the embodiments, at least one W is selected from CH. In one of the embodiments, all of the Ws are selected from CH.

In one of the embodiments, R⁷ is one or more selected from the group consisting of H, D, a linear alkyl group containing 1 to 20 carbon atoms, an linear alkoxy group containing 1 to 20 carbon atoms, a linear thioalkoxy group containing 1 to 20 carbon atoms, a branched or a cyclic alkyl containing 3 to 20 carbon atoms, a branched or a cyclic alkoxy containing 3 to 20 carbon atoms, a branched or a cyclic thioalkoxy group containing 3 to 20 carbon atoms, a branched or a cyclic silyl group containing 3 to 20 carbon atoms, a substituted keto group containing 1 to 20 carbon atoms, an alkoxy carbonyl group containing 2 to 20 carbon atoms, an aryloxy carbonyl group containing 7 to 20 carbon atoms, a cyano group (—CN), a carbamoyl group (—C(═O)NH₂), a haloformyl group (—C(═O)—X, and X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a crosslinkable group, a substituted or unsubstituted aromatic ring system containing 5 to 40 ring atoms or a substituted or unsubstituted heteroaromatic ring system containing 5 to 40 ring atoms, and an aryloxy group containing 5 to 40 ring atoms or a heteroaryloxy group containing 5 to 40 ring atoms; R⁷ forms a monocyclic or polycyclic aliphatic or aromatic ring with a ring bonded to the group, or R¹ form a monocyclic or polycyclic aliphatic or aromatic ring with each other at multiple occurrences.

In one of the embodiments, R⁸ is the same or different in each occurrence, and is selected from the group consisting of-H, —F, —Cl, Br, I, -D, —CN, —NO₂, —CF₃, B(OR⁹), Si(R⁹)₃, a linear alkyl, an alkane ether group, an alkane thioether group containing 1 to 10 carbon atoms, a branched alkyl containing 1 to 10 carbon atoms, a cycloalkyl containing 1 to 10 carbon atoms, and an alkane ether group containing 3 to 10 carbon atoms.

In one of the embodiments, R⁹ is selected from the group consisting of H, D, an aliphatic alkyl containing 1 to 10 carbon atoms, an aromatic hydrocarbon group containing 1 to 10 carbon atoms, and a substituted or unsubstituted aromatic ring group containing 5 to 10 ring atoms or a substituted or unsubstituted heteroaryl containing 5 to 10 ring atoms.

In one of the embodiments, Ar¹ to Ar³ are the same or different in each occurrence, and are selected from an aromatic ring containing 5 to 30 ring atoms or a heteroaromatic ring containing 5 to 30 ring atoms.

In addition, in one of the embodiments, the aromatic ring system contains 5 to 25 ring atoms in the ring system. The heteroaromatic ring system contains 5 to 25 ring atoms and at least one heteroatom in the ring system, provided that the total number of carbon atoms and heteroatoms is at least 5.

In one of the embodiments, the aromatic ring system contains 5 to 20 ring atoms in the ring system, and the heteroaromatic ring system contains 5 to 20 ring atoms and at least one heteroatom in the ring system. In one of the embodiments, the aromatic ring system contains 5 to 16 ring atoms in the ring system, and the heteroaromatic ring system contains 5 to 16 ring atoms and at least one heteroatom in the ring system.

In one of the embodiments, the heteroatom is one or more selected from the group consisting of Si, N, P, O, S, and Ge. In one of the embodiments, the heteroatom is one or more selected from the group consisting of Si, N, P, O, and S.

The aromatic group refers to a hydrocarbyl containing at least one aromatic ring, and the aromatic ring system refers to a ring system including a monocyclic group and a polycyclic group. The heteroaromatic group may also refer to a hydrocarbyl (containing heteroatoms) containing at least one heteroaromatic ring, and the heteroaromatic ring system may also refer to a ring system including a monocyclic group and a polycyclic group. Such polycyclic rings may have two or more rings, in which two carbon atoms are shared by two adjacent rings, i.e., a fused ring. At least one ring of such polycyclic rings is aromatic or heteroaromatic. In one of the embodiments, the aromatic or heteroaromatic ring system includes not only aromatic or heteroaromatic systems, but also systems in which a plurality of aryl or heteroaryl, but also a plurality of aryl or heteroaryl may also be interrupted by short non-aromatic units (<10% non-H atoms, in one embodiment less than 5% of non-H atoms, such as C, N, or O atoms). Therefore, systems such as 9, 9′-spirobifluorene, 9, 9-diarylfluorene, triarylamine, diaryl ether, and the like are also considered to be the aromatic ring system for the purpose of the present disclosure.

In one of the embodiments, the aromatic group is selected from the group consisting of benzene, naphthalene, anthracene, phenanthrene, perylene, naphthacene, pyrene, benzopyrene, triphenylene, acenaphthene, fluorene, and derivatives thereof.

In one of the embodiments, the heteroaromatic group is selected from the group consisting of furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, cinnoline, quinoxaline, phenanthridine, perimidine, quinazoline, quinazolinone, and derivatives thereof.

In one of the embodiments, at least one of Ar¹ to Ar³ includes a non-aromatic ring system containing 2 to 20 carbon atoms which is unsubstituted or substituted with R¹⁰.

In one of the embodiments, the non-aromatic ring system contains 1 to 10 carbon atoms in the ring system. In one embodiment, the non-aromatic ring system contains 1 to 6 carbon atoms in the ring system. The non-aromatic ring system includes not only saturated ring systems but also partially unsaturated ring systems, which may be unsubstituted or mono- or polysubstituted by group R¹¹. The group R¹¹ may be the same or different in each occurrence and may also contain one or more heteroatoms. The heteroatom is one or more selected from the group consisting of Si, N, P, O, S, and Ge. In one of the embodiments, the heteroatom is one or more selected from the group consisting of Si, N, P, O, and S. These may be, for example, cyclohexyl- or piperidine-like systems, but also can be cyclooctadiene-like cyclic systems. The term also applies to a fused non-aromatic ring system.

In one of the embodiments, Ar¹ to Ar³ are independently selected from one of the following groups:

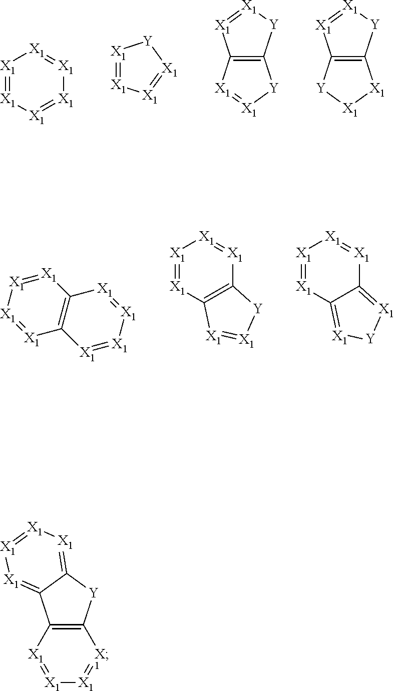

X₁ is selected from CR¹⁰ or N;

Y is selected from the group consisting of CR¹¹R¹², SiR¹³R¹⁴, NR¹⁵, C(═O), S, and O:

R¹⁰, R¹¹, R¹², R¹³, R¹⁴, and R¹⁵ are one or more selected from the group consisting of H, D, a linear alkyl containing 1 to 20 carbon atoms, an linear alkoxy containing 1 to 20 carbon atoms, a linear thioalkoxy containing 1 to 20 carbon atoms, a branched or a cyclic alkyl containing 3 to 20 carbon atoms, a branched or a cyclic alkoxy containing 3 to 20 carbon atoms, a branched or a cyclic thioalkoxy group containing 3 to 20 carbon atoms, a branched or a cyclic silyl group containing 3 to 20 carbon atoms, a substituted keto group containing 1 to 20 carbon atoms, an alkoxy carbonyl group containing 2 to 20 carbon atoms, an aryloxy carbonyl group containing 7 to 20 carbon atoms, a cyano group (—CN), a carbamoyl group (—C(═O)NH₂), a haloformyl group (—C(═O)—X, and X represents a halogen atom), a formyl group (—C(═O)—H), an isocyano group, an isocyanate group, a thiocyanate group, an isothiocyanate group, a hydroxyl group, a nitro group, a CF; group, Cl, Br, F, a crosslinkable group, a substituted or unsubstituted aromatic ring system containing 5 to 40 ring atoms or a substituted or unsubstituted heteroaromatic ring system containing 5 to 40 ring atoms, and an aryloxy group containing 5 to 40 ring atoms or a heteroaryloxy group containing 5 to 40 ring atoms. At least one of R¹⁰, R¹¹, R¹², R¹³, R¹⁴, and R¹⁵ forms a monocyclic or polycyclic aliphatic or aromatic ring system with a ring bonded to the group, or at least two of R¹⁰, R¹¹, R¹², R¹³, R¹⁴, and R¹⁵ form a monocyclic or polycyclic aliphatic or aromatic ring with each other. Ar₁ to Ar³ may be the same or different in multiple occurrences.

In one of the embodiments, Ar¹ to Ar³ contain one or more of the following structural formulas. Any one of the structural formulas may be substituted with one or more groups R¹⁰.

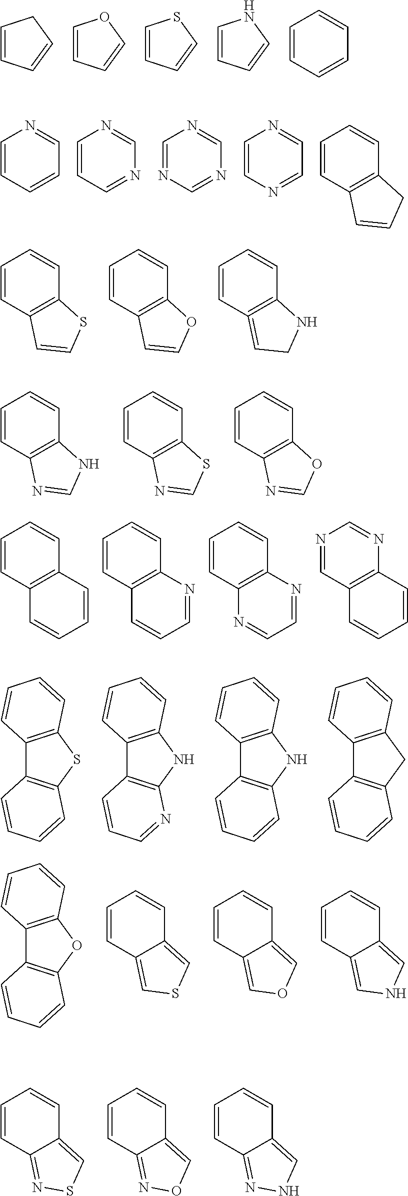

In one of the embodiments, Ar¹ to Ar³ may be the same or different in multiple occurrences and contain one of the following structural groups.

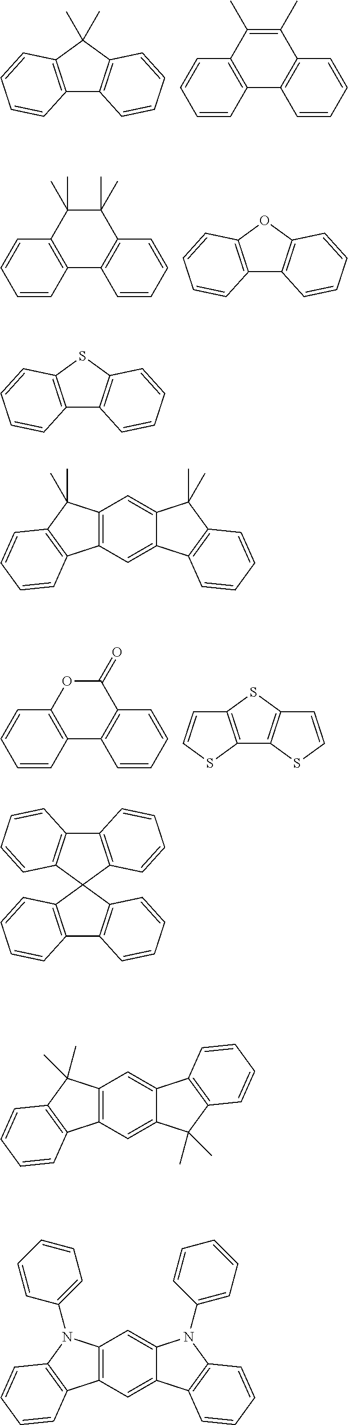

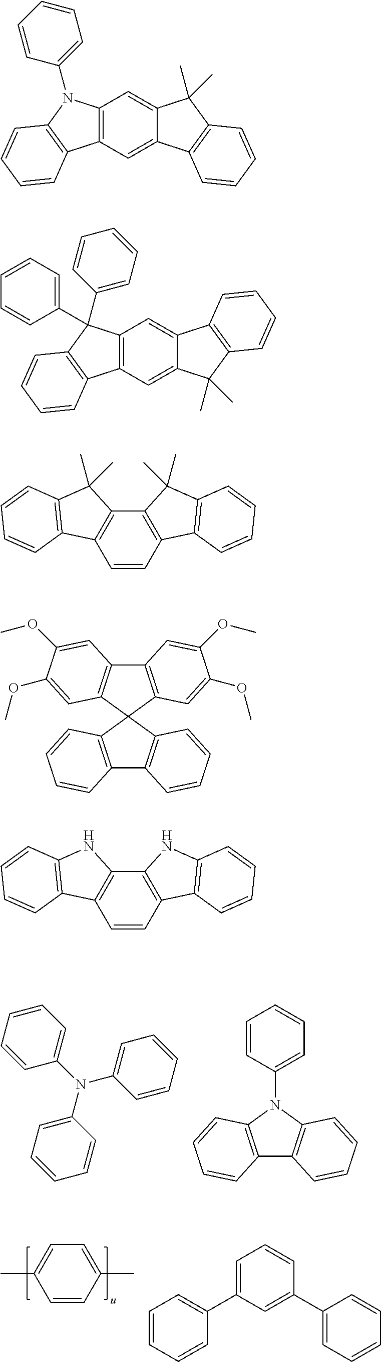

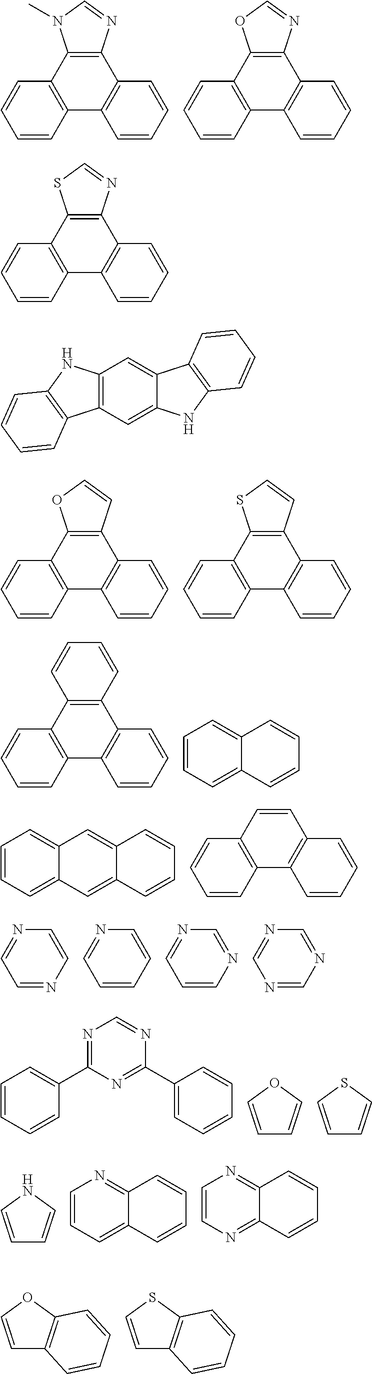

u is seleted from 1, 2, 3, or 4.

In one of the embodiments, Ar¹, Ar², and Ar³ are independently selected from the group consisting of an aromatic ring system containing 5 to 30 ring atoms.

In one of the embodiments, Ar¹ is independently selected from the group consisting of:

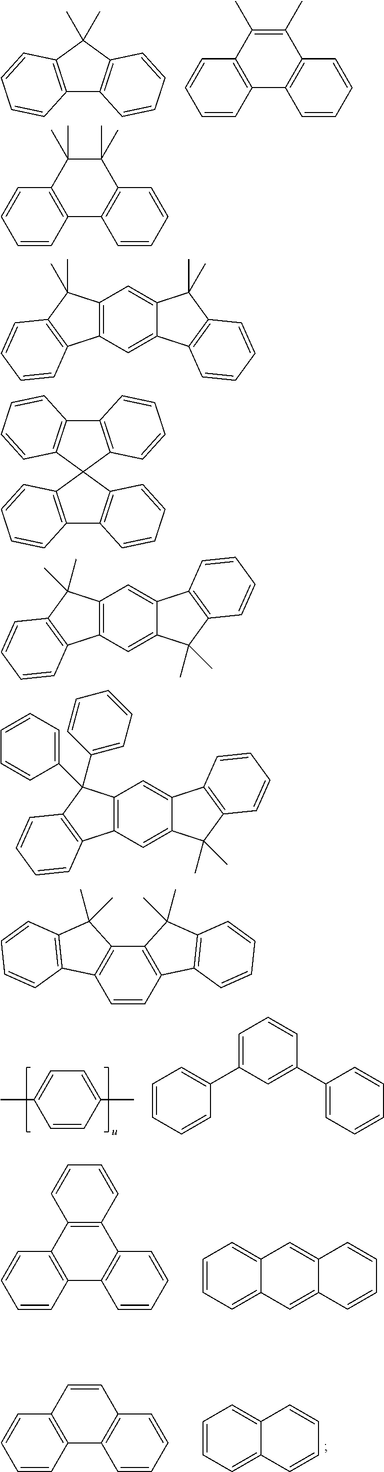

wherein u is selected from 1, 2, 3, or 4.

In one of the embodiments, Ar¹ is selected from the group consisting of:

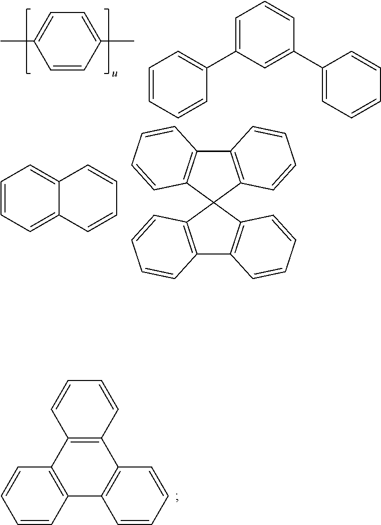

and

Ar² and Ar³ are selected from the group consisting of:

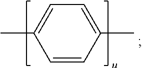

wherein u is selected from 1, 2, 3, or 4.

In one of the embodiments, all of Zs are selected from N, and a linking group of the benzene at an ortho position can be linked at different positions.

In addition, in one of the embodiments, the organic compound is a compound represented by one of the following general formulas (2) to (3):

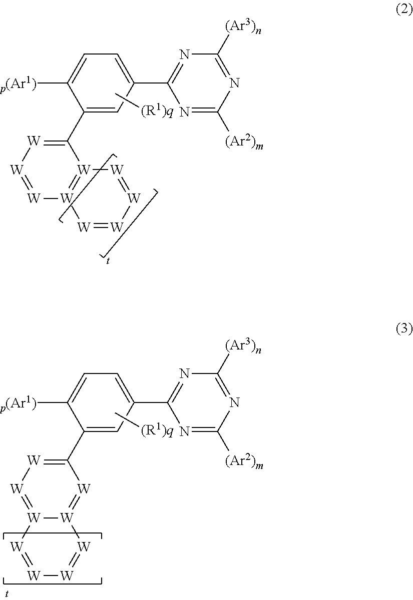

In one of the embodiments, the organic compound has a general formula (1b) as following.

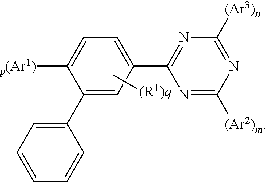

In one of the embodiments, the compound has a higher triplet excited state energy level T₁, for example, T₁≥2.2 eV. In one of the embodiments, T₁≥2.4 eV. In one of the embodiments, T₁≥2.5 eV. In one of the embodiments, T₁≥2.6 eV In one of the embodiments, T₁≥2.8 eV.

Typically, the triplet excited state energy level T₁ of the organic compound depends on the substructure having the largest conjugated system in the compound. In general, T₁ decreases as the conjugated system increases. In one of the embodiments, the structure of the organic compound is represented by the general formula (1a).

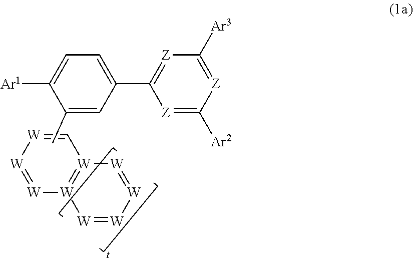

In one of the embodiments, when the substituent of the general formula (1a) is removed, ring atoms of the general formula (1a) are less than or equal to 90. In one of the embodiments, ring atoms are less than or equal to 80. In one of the embodiments, ring atoms are less than or equal to 70. In one of the embodiments, ring atoms are less than or equal to 60.

In one of the embodiments, the general formula (1a) has a higher triplet excited state energy level T₁, for example, T₁≥2.2 eV. In one of the embodiments, T₁≥2.4 eV In one of the embodiments, T₁≥2.5 eV. In one of the embodiments, T₁≥2.6 eV. In one of the embodiments, T₁≥2.8 eV.

In one of the embodiments, the organic compound is at least partially deuterated. In one of the embodiments, 10% of H is deuterated. In one of the embodiments, 20% of H is deuterated. In one of the embodiments, 30% of H is deuterated. In one of the embodiments, 40% of H is deuterated.

In one of the embodiments, the organic compound is capable of achieving thermally activated delayed fluorescence effect. When ΔE(S1-TI) of the organic compound is sufficiently small, the triplet excitons of the organic compound can be converted to singlet excitons through reverse intersystem crossing, thereby achieving high efficient light emission and improving the stability of the material. In general, this type of material is obtained by linking an electron-donating group (Donor) to an electron-deficiency group or an electron-accepting group (Acceptor). In other words, the material has a distinct D-A structure.

In one of the embodiments, at least one Ar contains the electron-donating group in multiple occurrences, in which Ar is Ar¹, Ar², or Ar³. In one of the embodiments, at least one Ar contains the electron-accepting group. In one of the embodiments, at least one Ar contains the electron-donating group, and at least one Ar contains the electron-accepting group.

In one of the embodiments, the electron-donating group is selected from at least one of the group consisting of:

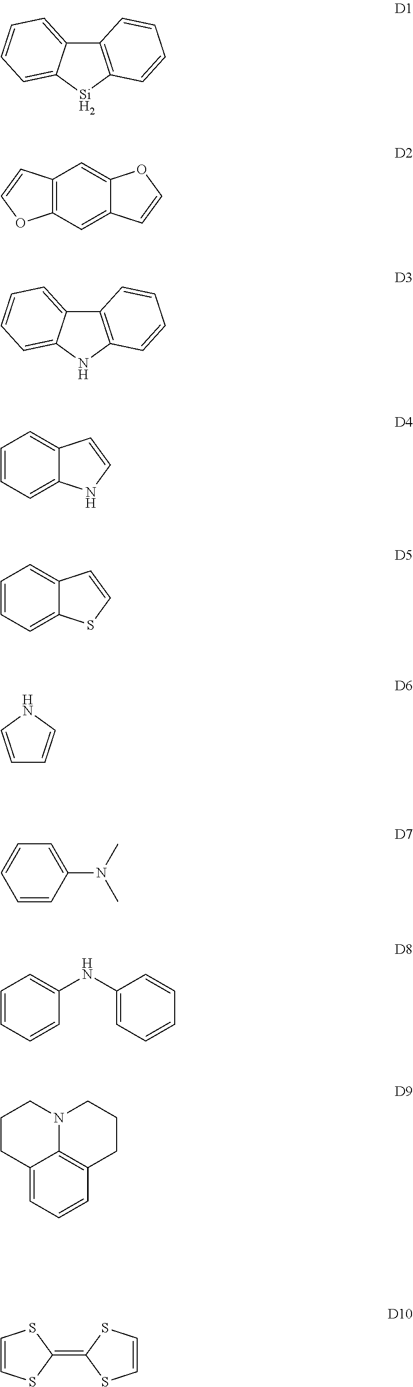

In one of the embodiments, the electron-accepting group is selected from F, a cyano group, or any one of the following groups.

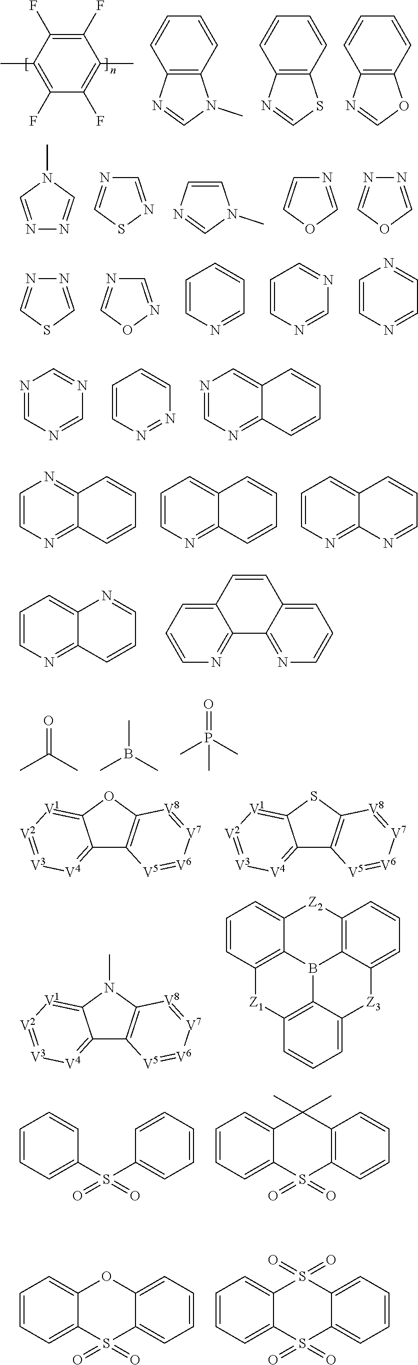

n is selected from 1, 2, or 3. V¹ to V⁸ are independently selected from CR¹⁶ or N, and at least one of V¹ to V⁸ is N. R¹⁶ is selected from the group consisting of hydrogen, alkyl, alkoxy, amino, alkene, alkyne, aralkyl, heteroalkyl, aryl, and heteroaryl, Z₁ to Z₃ are selected from the group consisting of a single bond, 2 C(R¹⁶)₂, O, and S.

In one of the embodiments, the organic compound is selected from one of the compounds represented by the following structures. These structures can be substituted at all possible substitution positions.

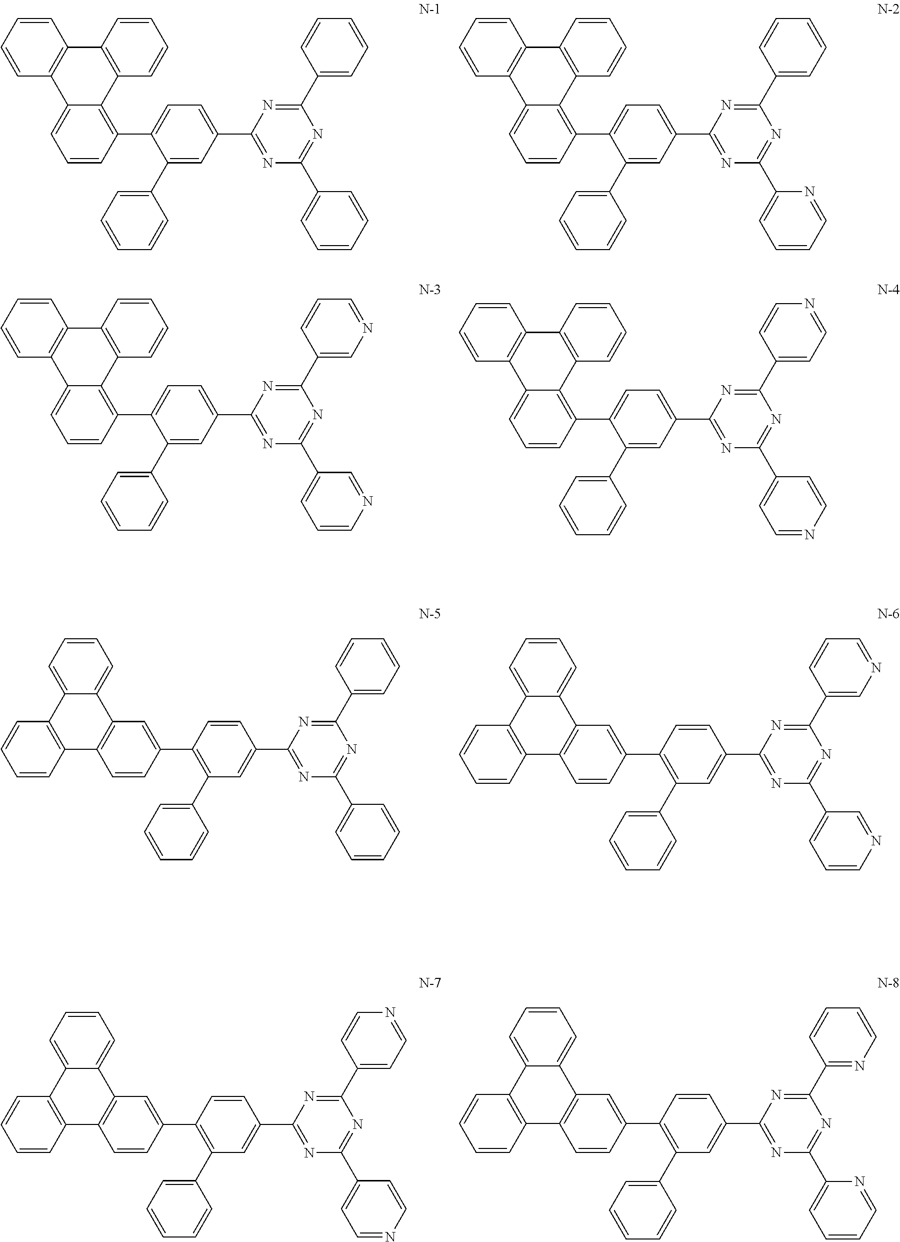

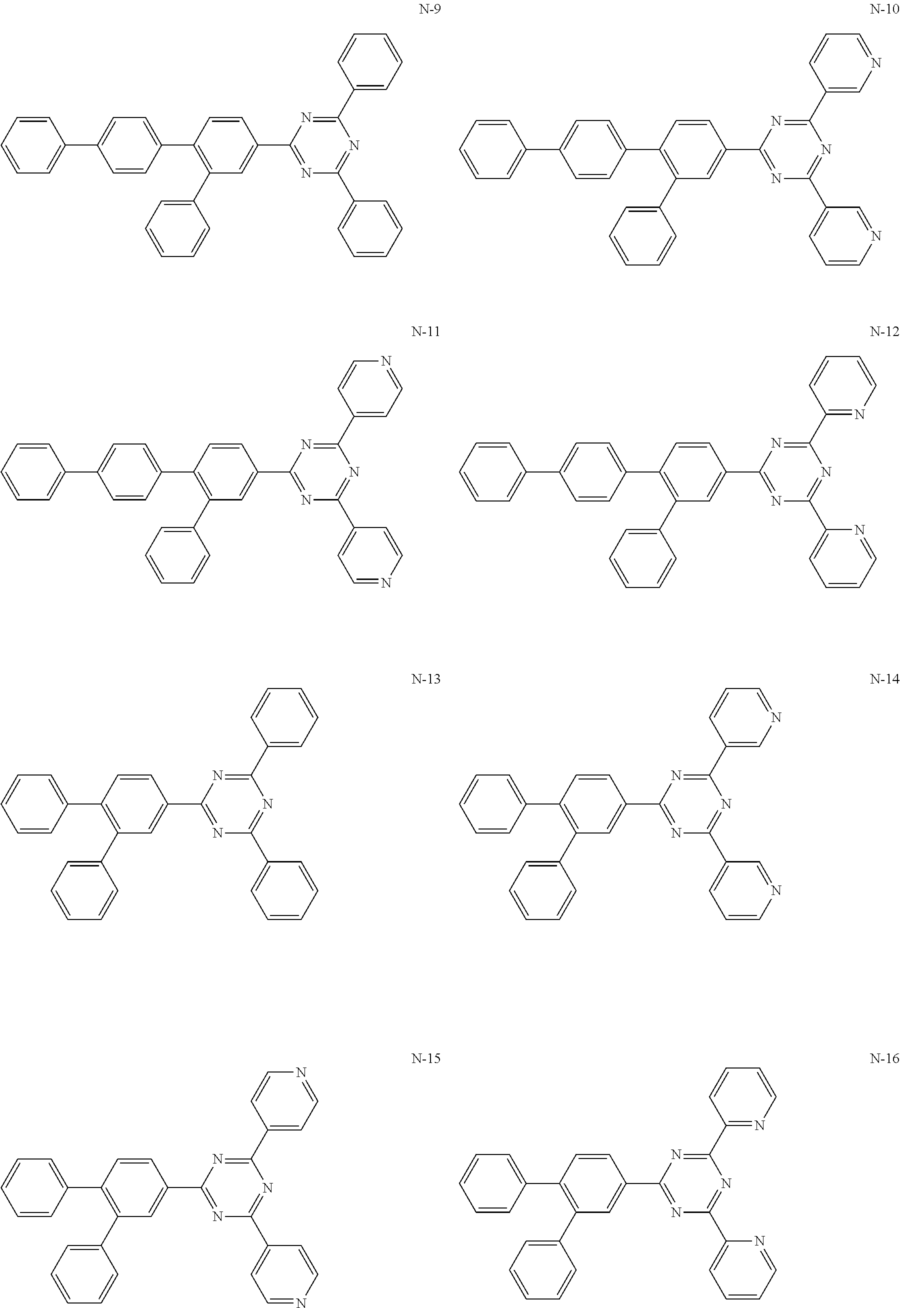

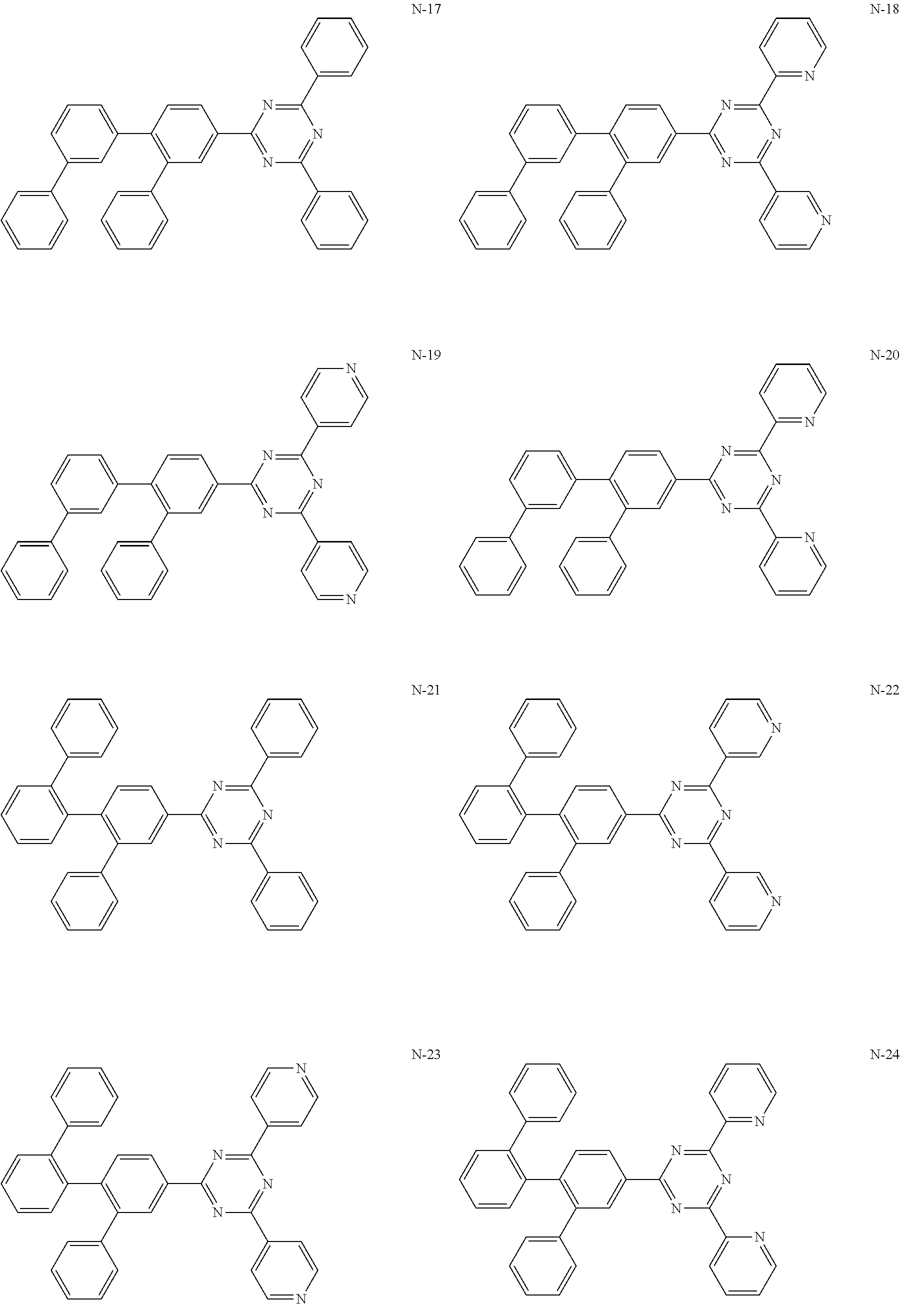

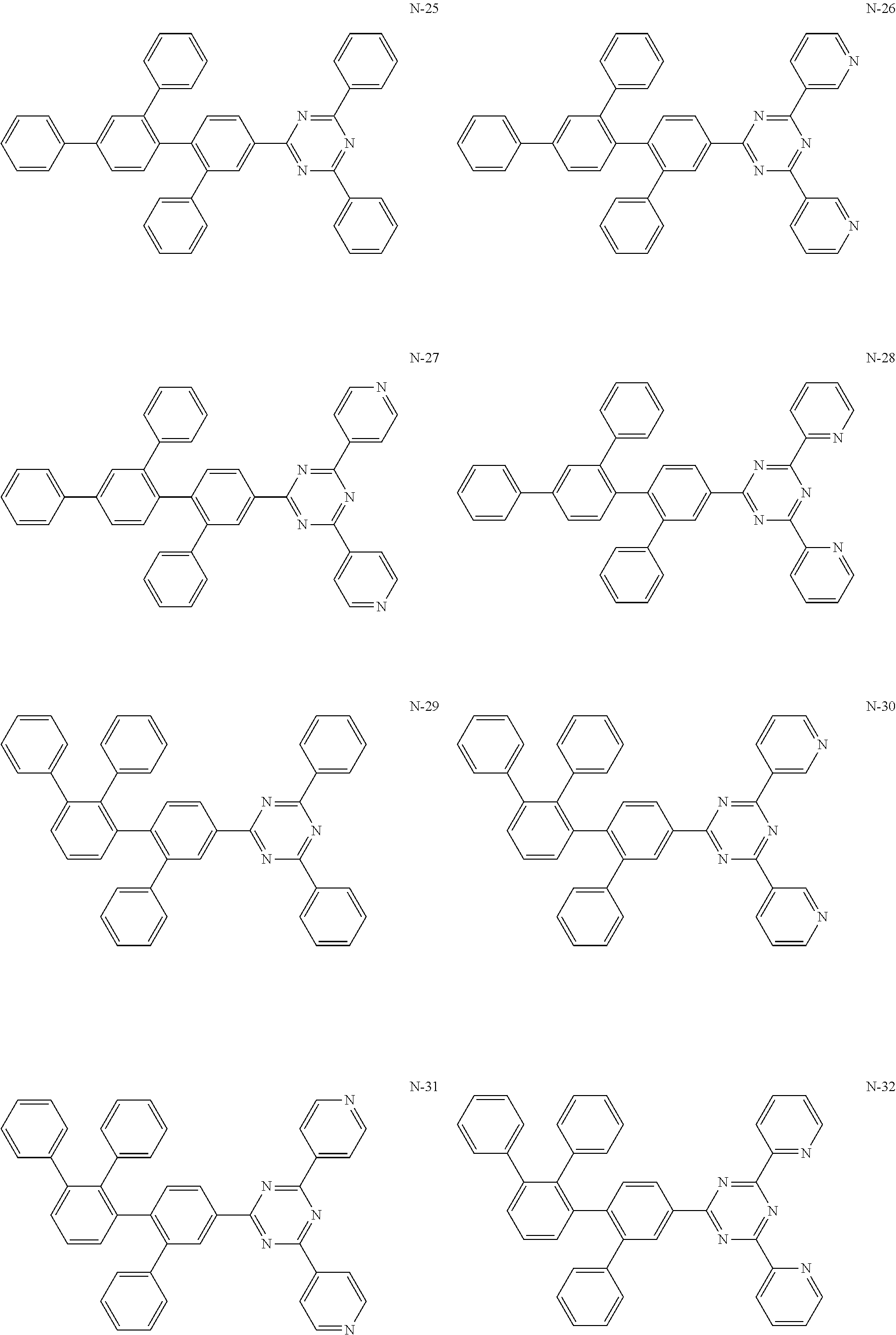

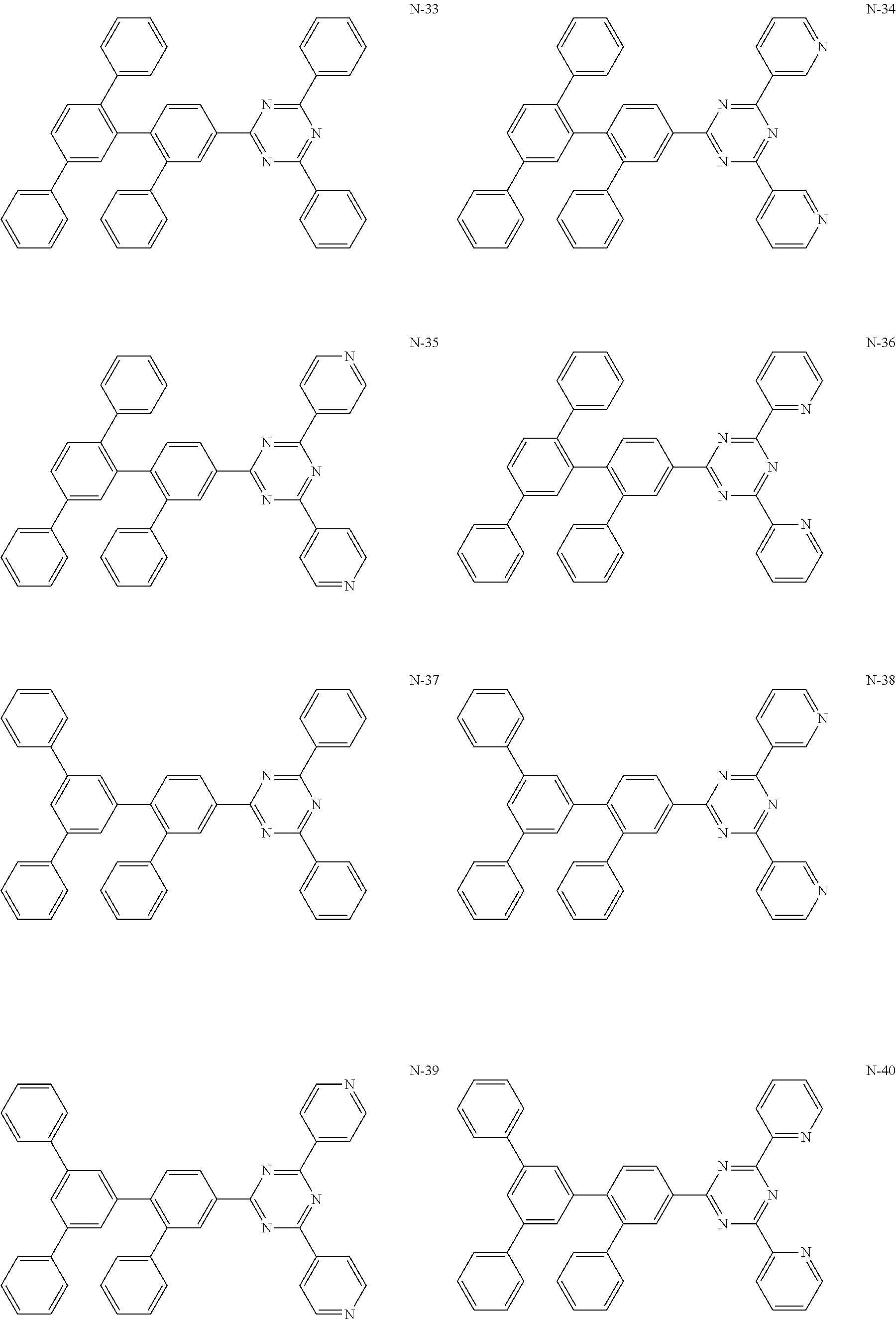

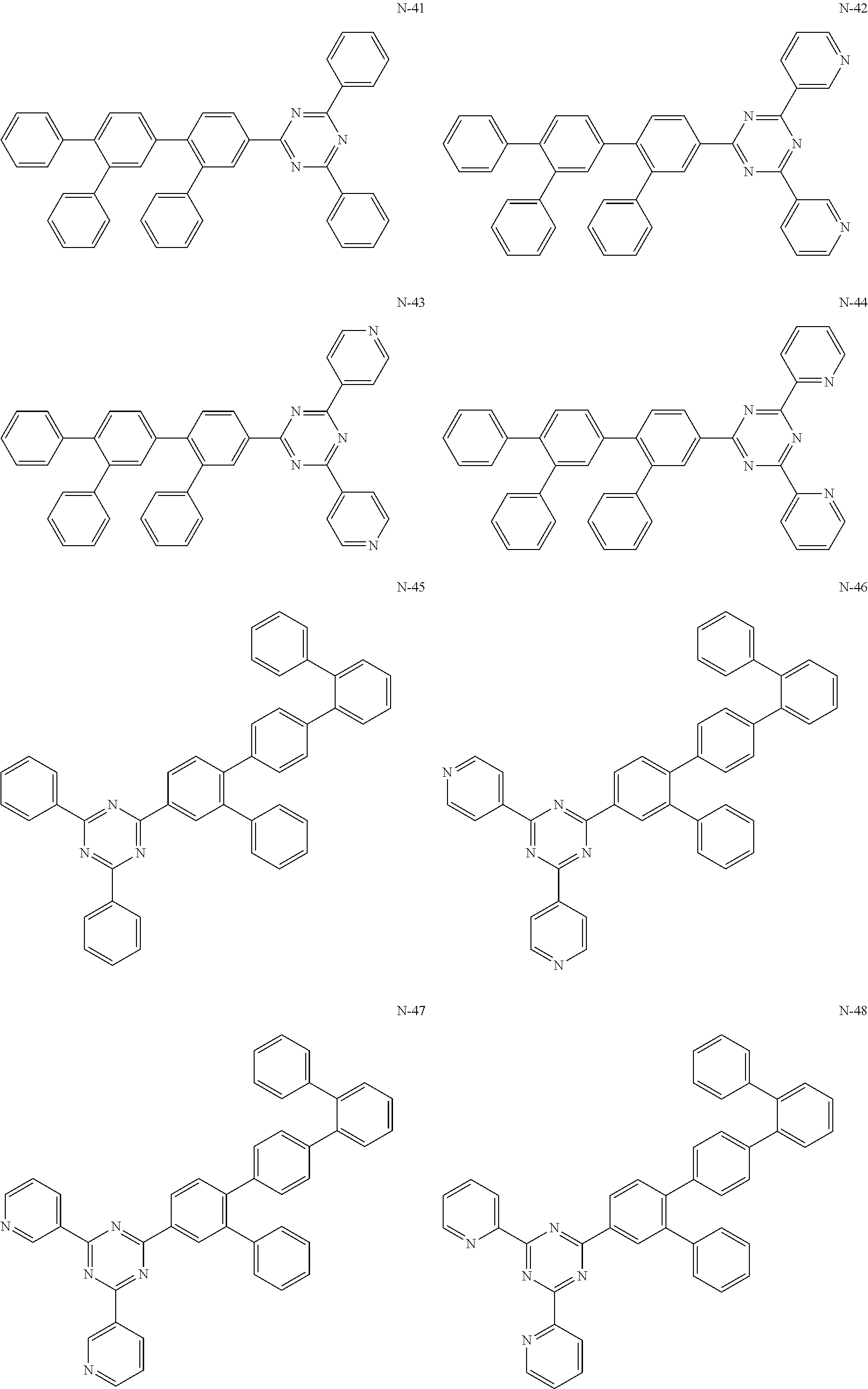

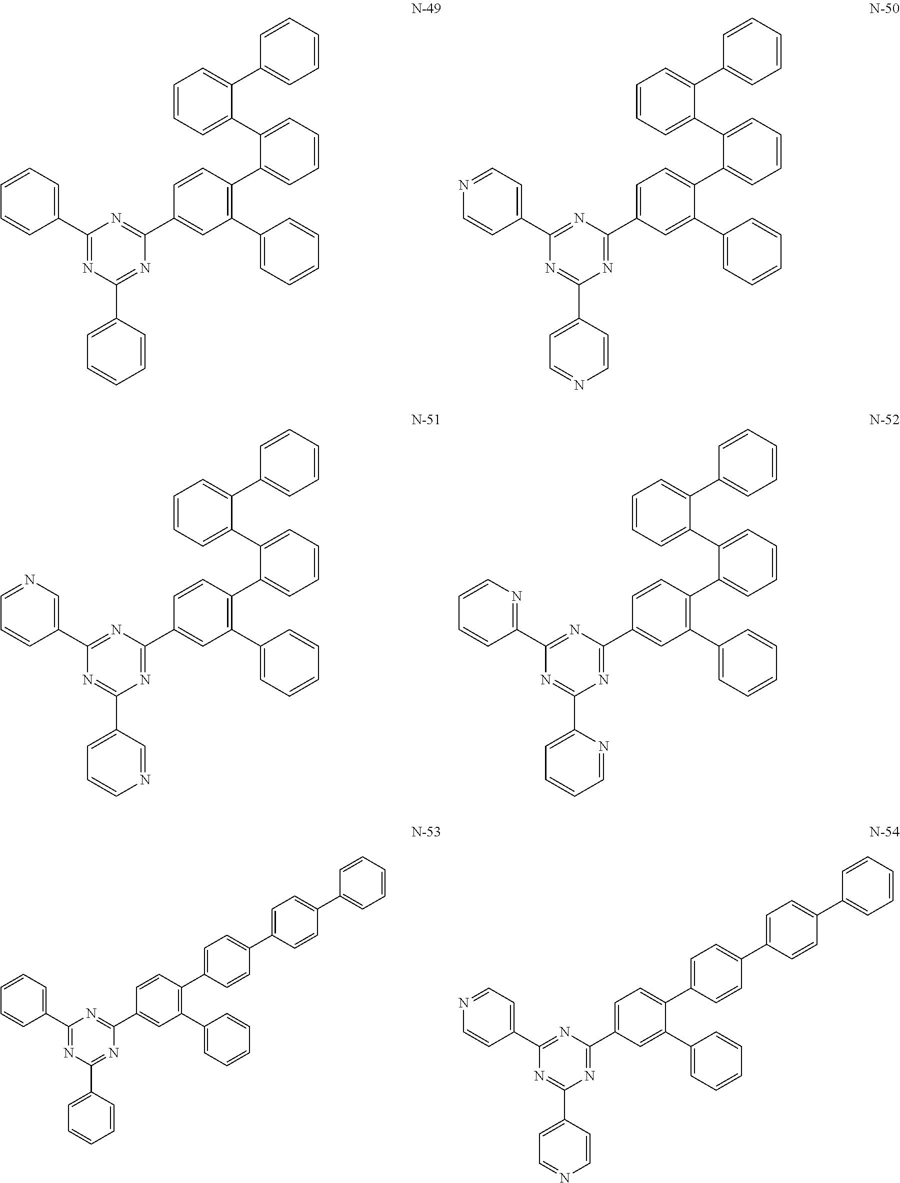 [SMILES: c1ccc(-c2nc(-c3ccccc3)nc(-c3ccc(-c4ccccc4-c4ccccc4-c4ccccc4)c(-c4ccccc4)c3)n2)cc1 ; c1ccc(-c2ccccc2-c2ccccc2-c2ccc(-c3nc(-c4ccncc4)nc(-c4ccncc4)n3)cc2-c2ccccc2)cc1 ; c1ccc(-c2ccccc2-c2ccccc2-c2ccc(-c3nc(-c4cccnc4)nc(-c4cccnc4)n3)cc2-c2ccccc2)cc1 ; c1ccc(-c2ccccc2-c2ccccc2-c2ccc(-c3nc(-c4ccccn4)nc(-c4ccccn4)n3)cc2-c2ccccc2)cc1 ; c1ccc(-c2ccc(-c3ccc(-c4ccc(-c5nc(-c6ccccc6)nc(-c6ccccc6)n5)cc4-c4ccccc4)cc3)cc2)cc1 ; c1ccc(-c2ccc(-c3ccc(-c4ccc(-c5nc(-c6ccncc6)nc(-c6ccncc6)n5)cc4-c4ccccc4)cc3)cc2)cc1]

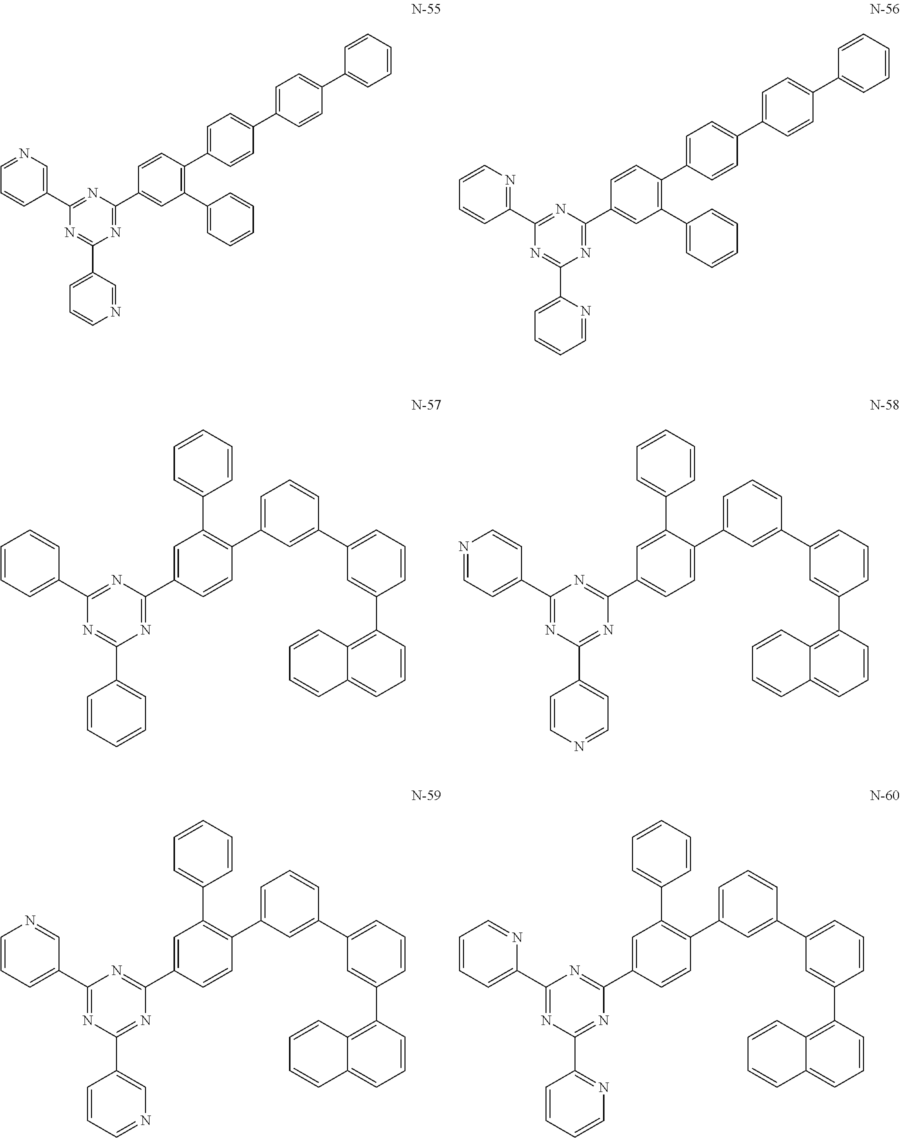

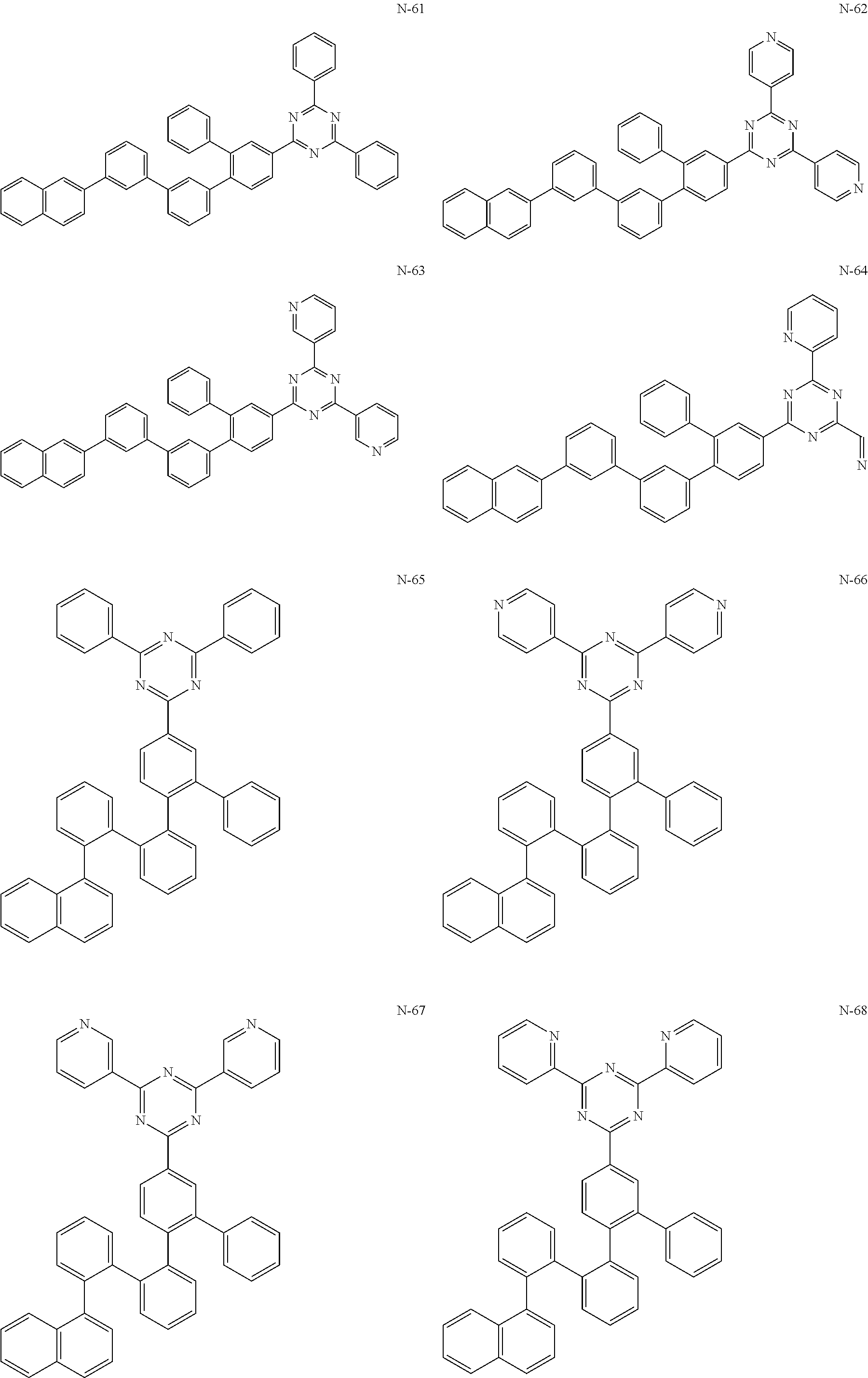

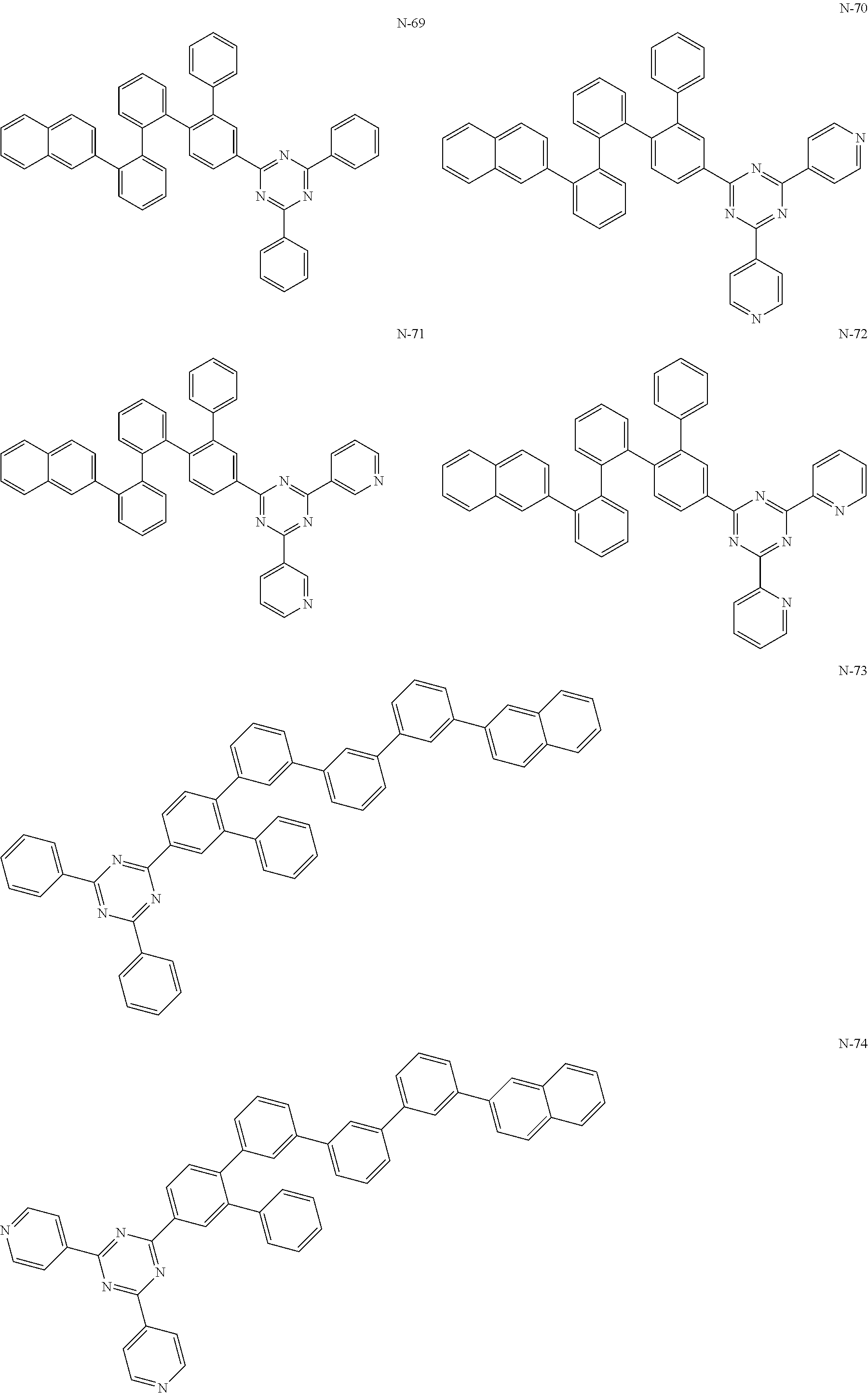

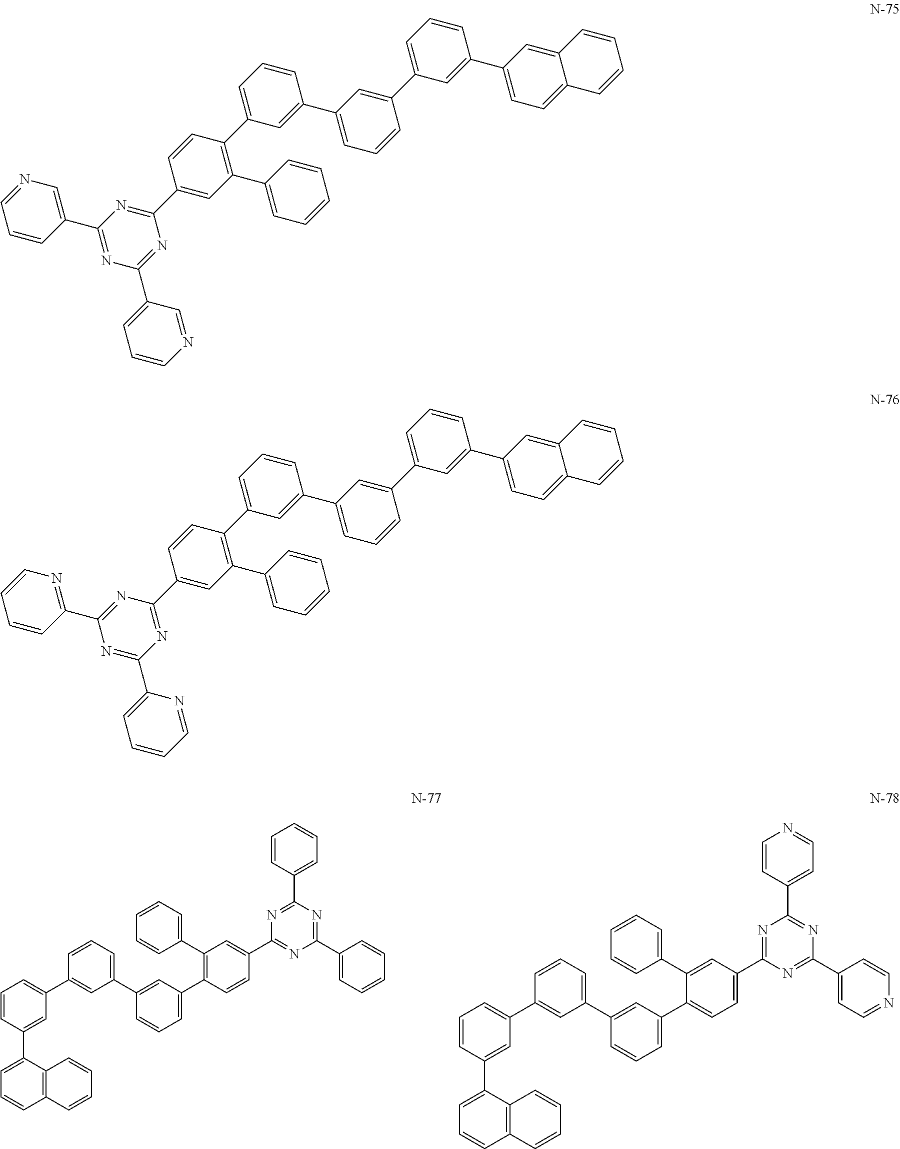 [SMILES: c1ccc(-c2cc(-c3nc(-c4cccnc4)nc(-c4cccnc4)n3)ccc2-c2cccc(-c3cccc(-c4cccc(-c5ccc6ccccc6c5)c4)c3)c2)cc1 ; c1ccc(-c2cc(-c3nc(-c4ccccn4)nc(-c4ccccn4)n3)ccc2-c2cccc(-c3cccc(-c4cccc(-c5ccc6ccccc6c5)c4)c3)c2)cc1 ; c1ccc(-c2nc(-c3ccccc3)nc(-c3ccc(-c4cccc(-c5cccc(-c6cccc(-c7cccc8ccccc78)c6)c5)c4)c(-c4ccccc4)c3)n2)cc1 ; c1ccc(-c2cc(-c3nc(-c4ccncc4)nc(-c4ccncc4)n3)ccc2-c2cccc(-c3cccc(-c4cccc(-c5cccc6ccccc56)c4)c3)c2)cc1]

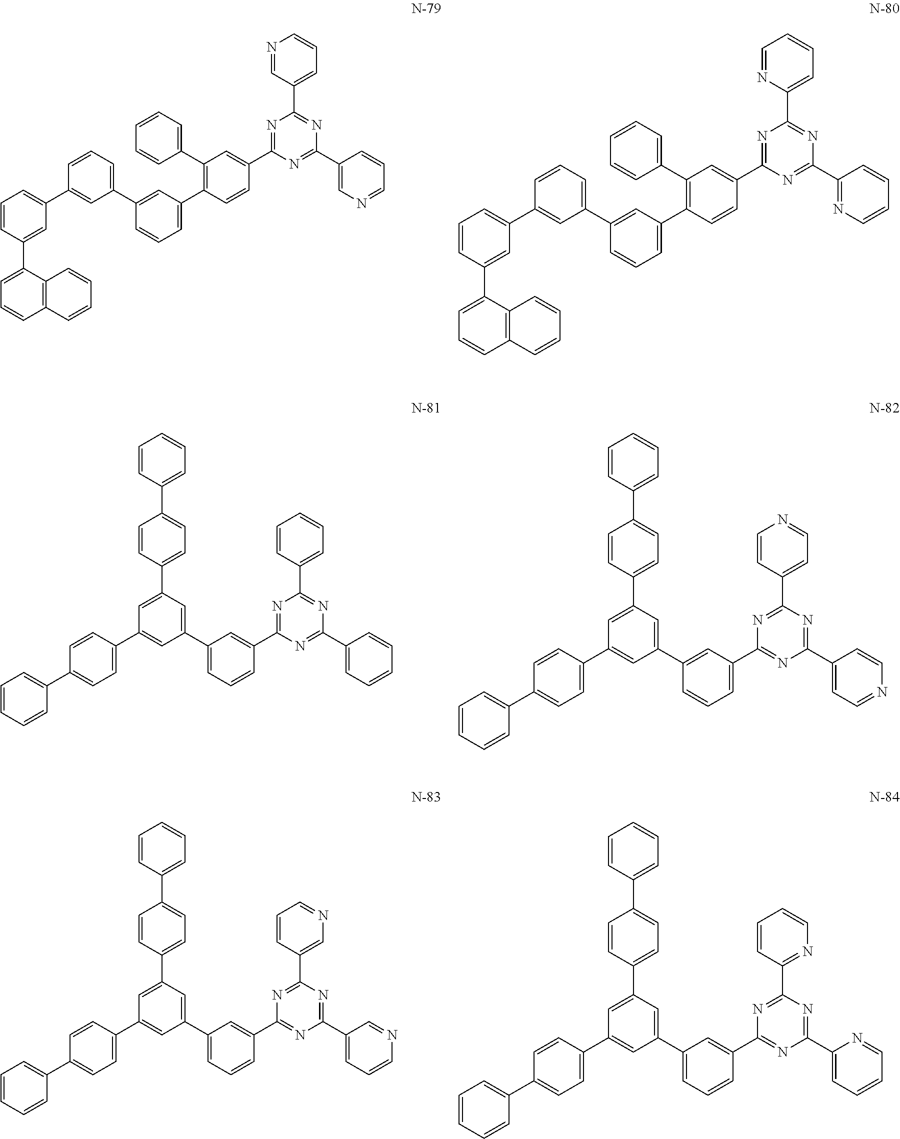

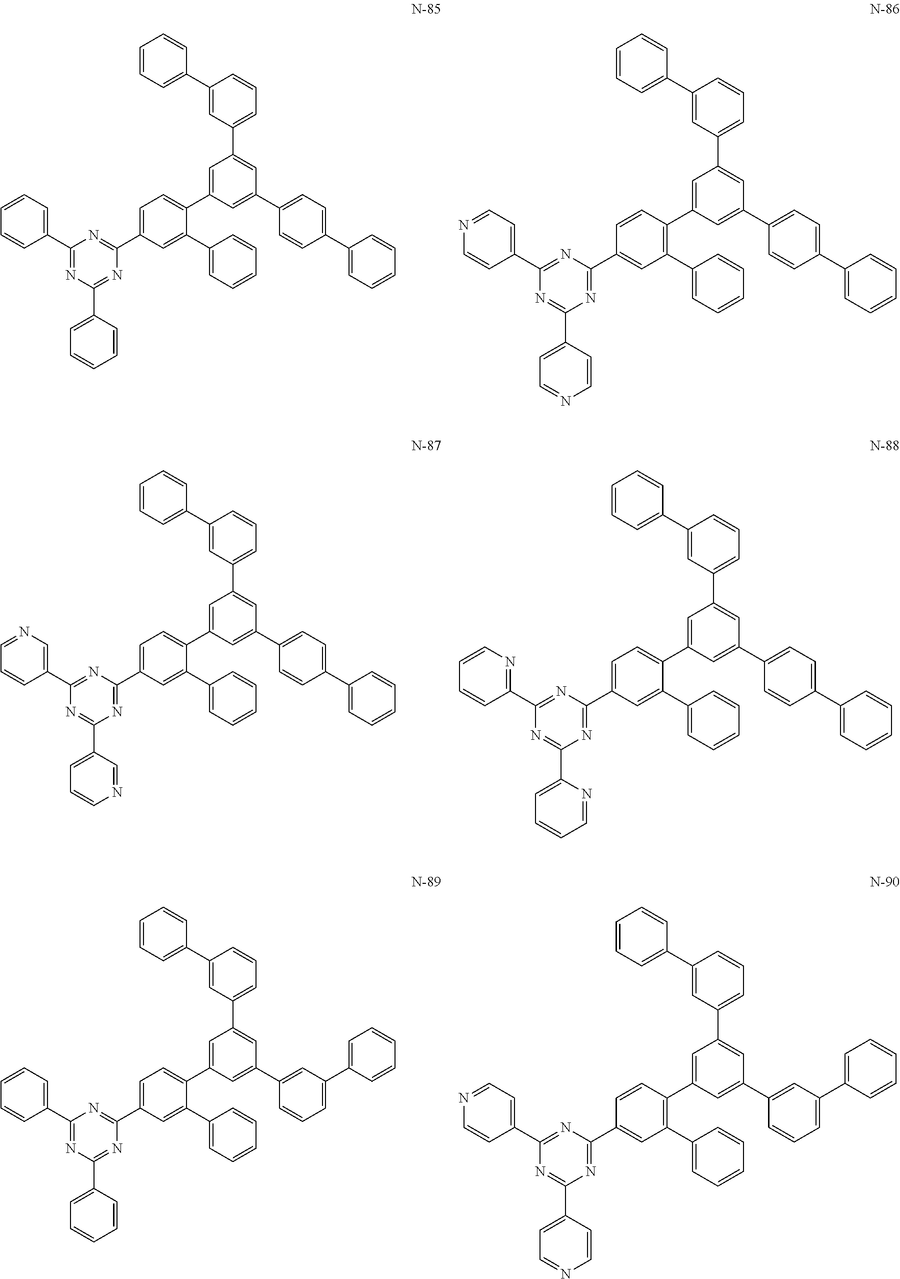

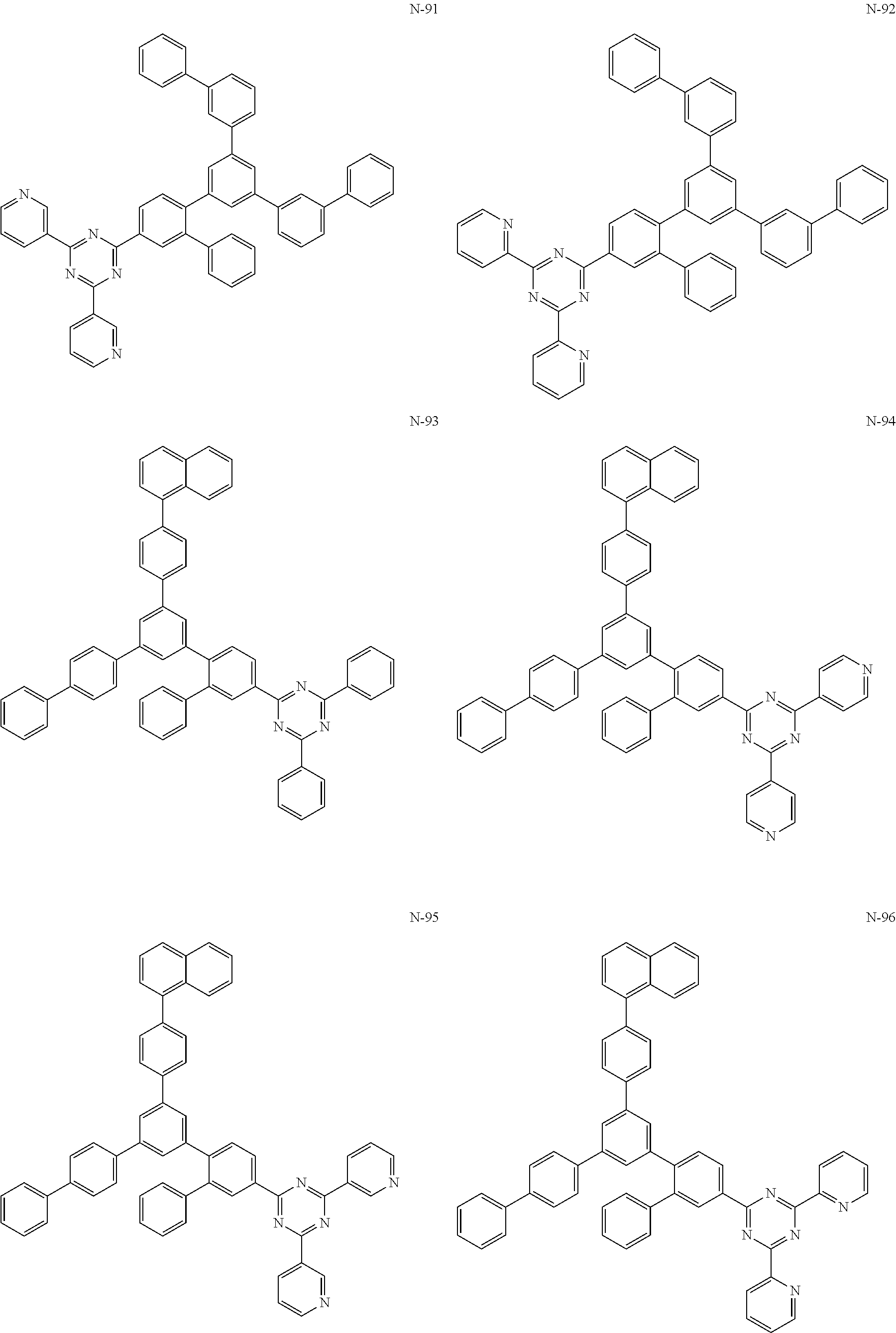

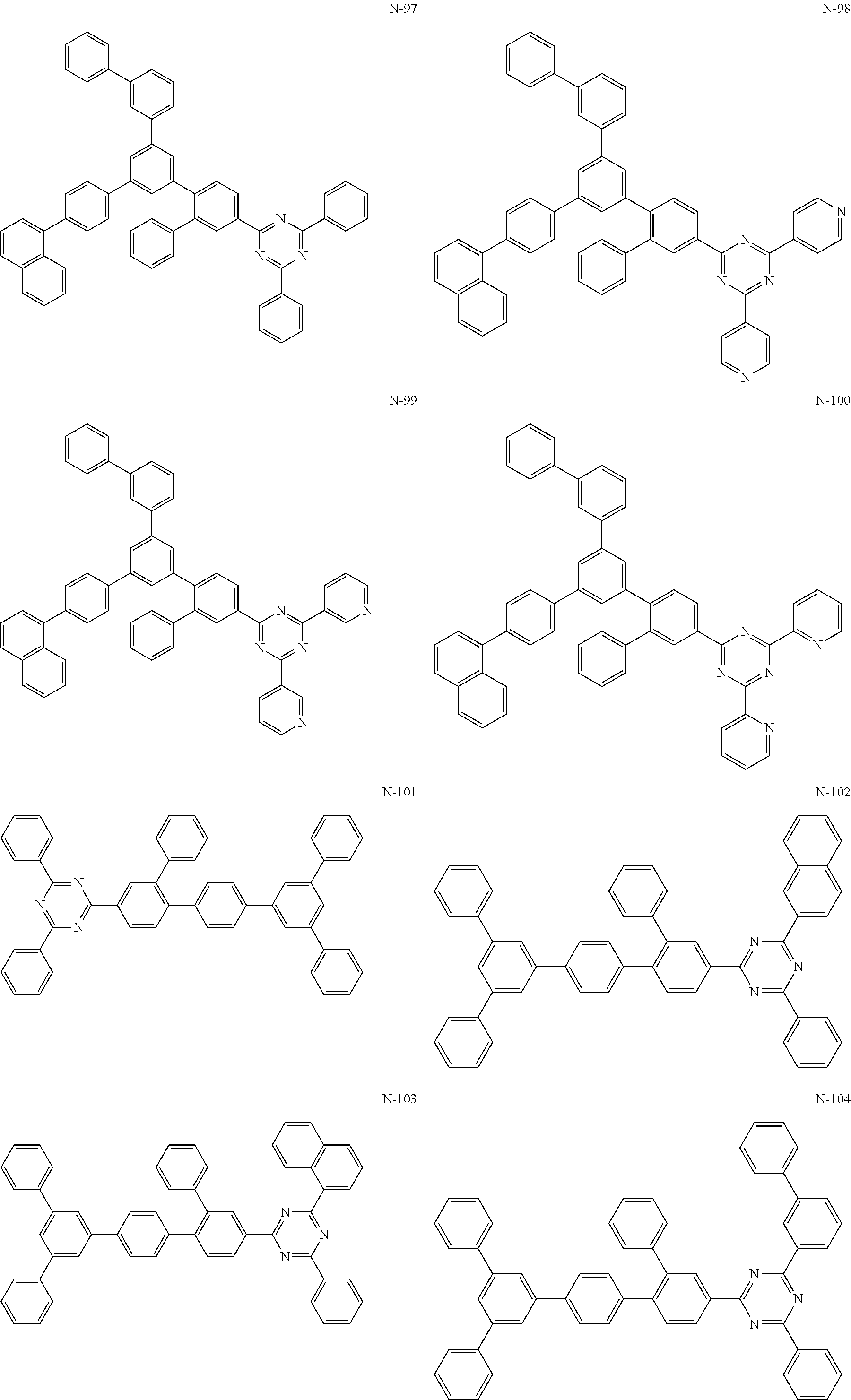

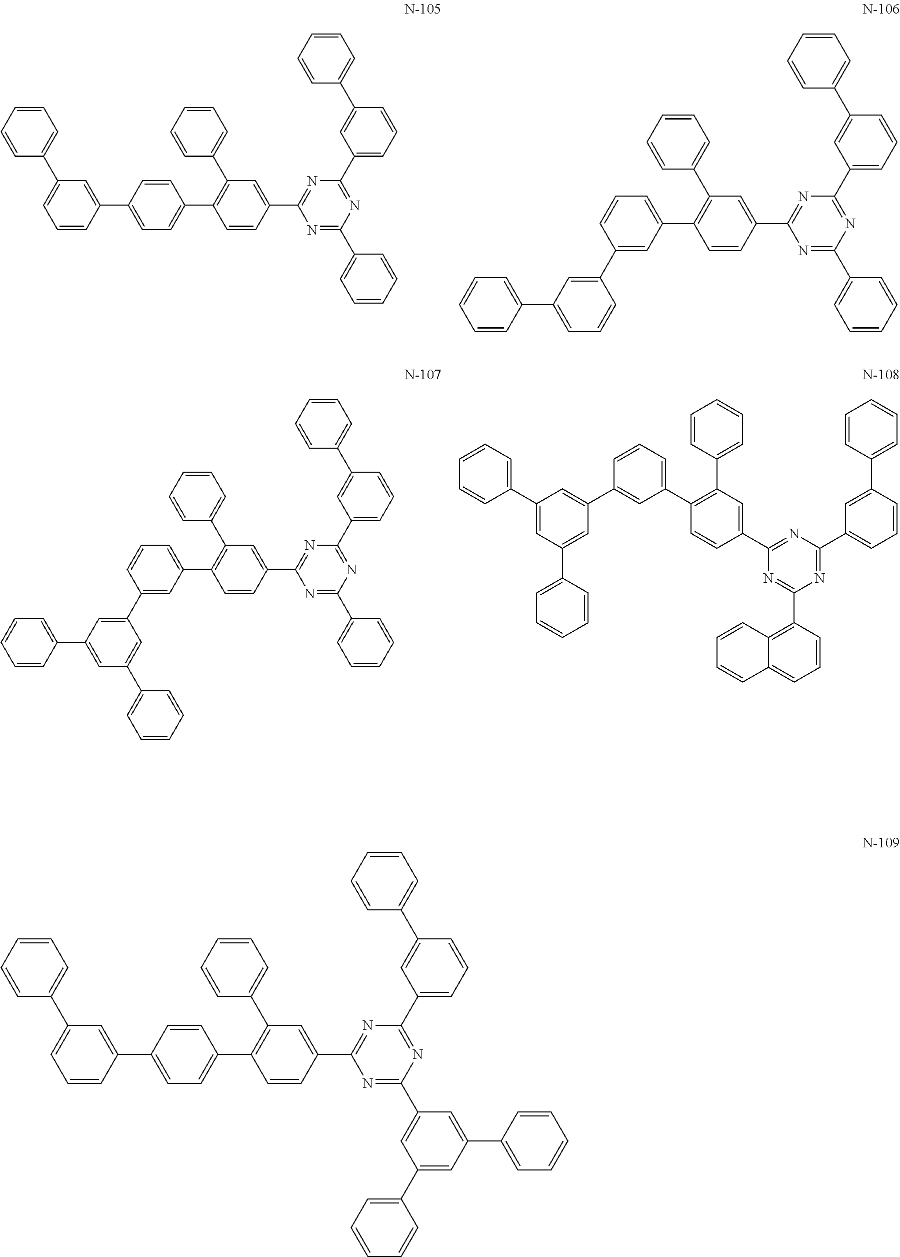

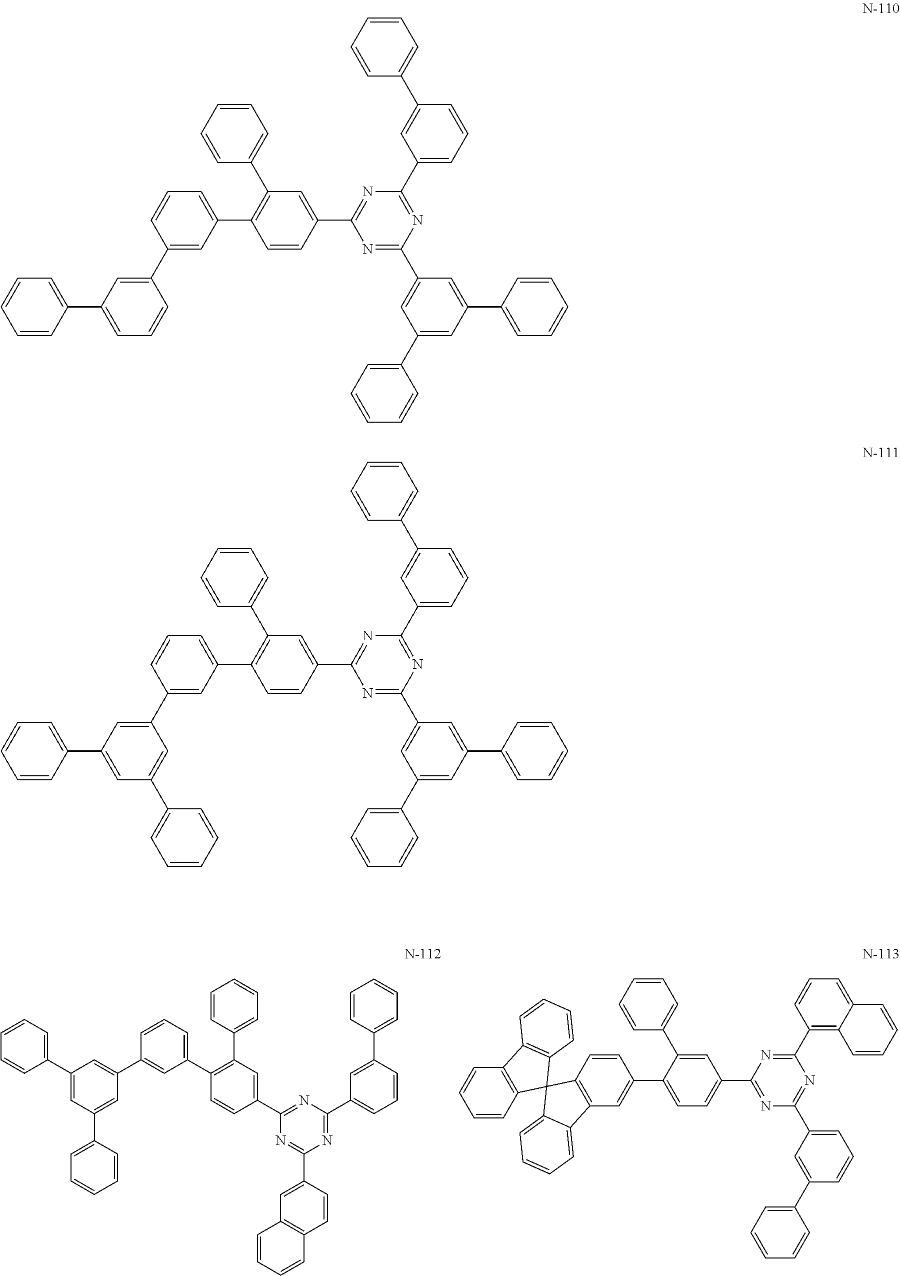

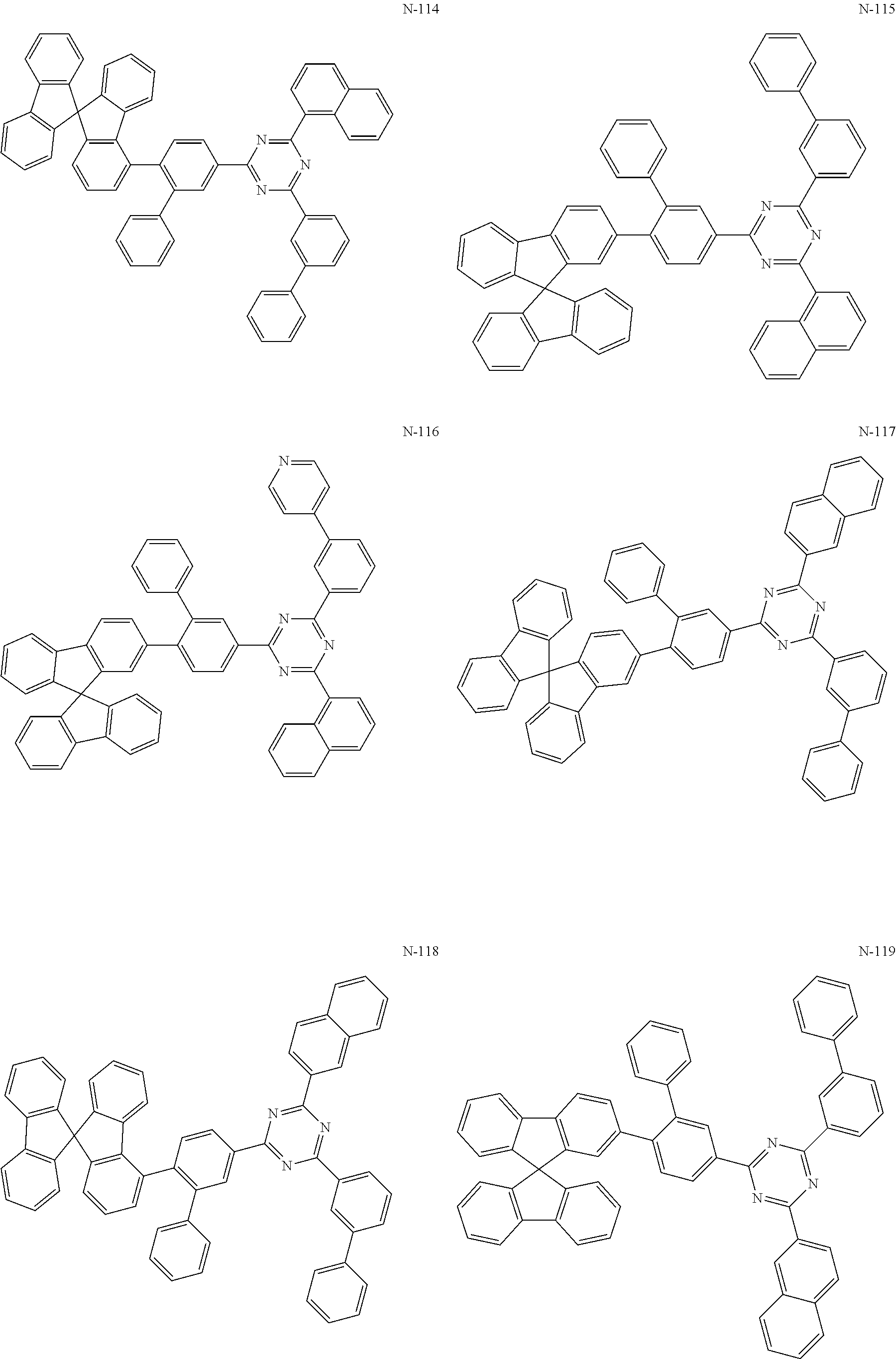

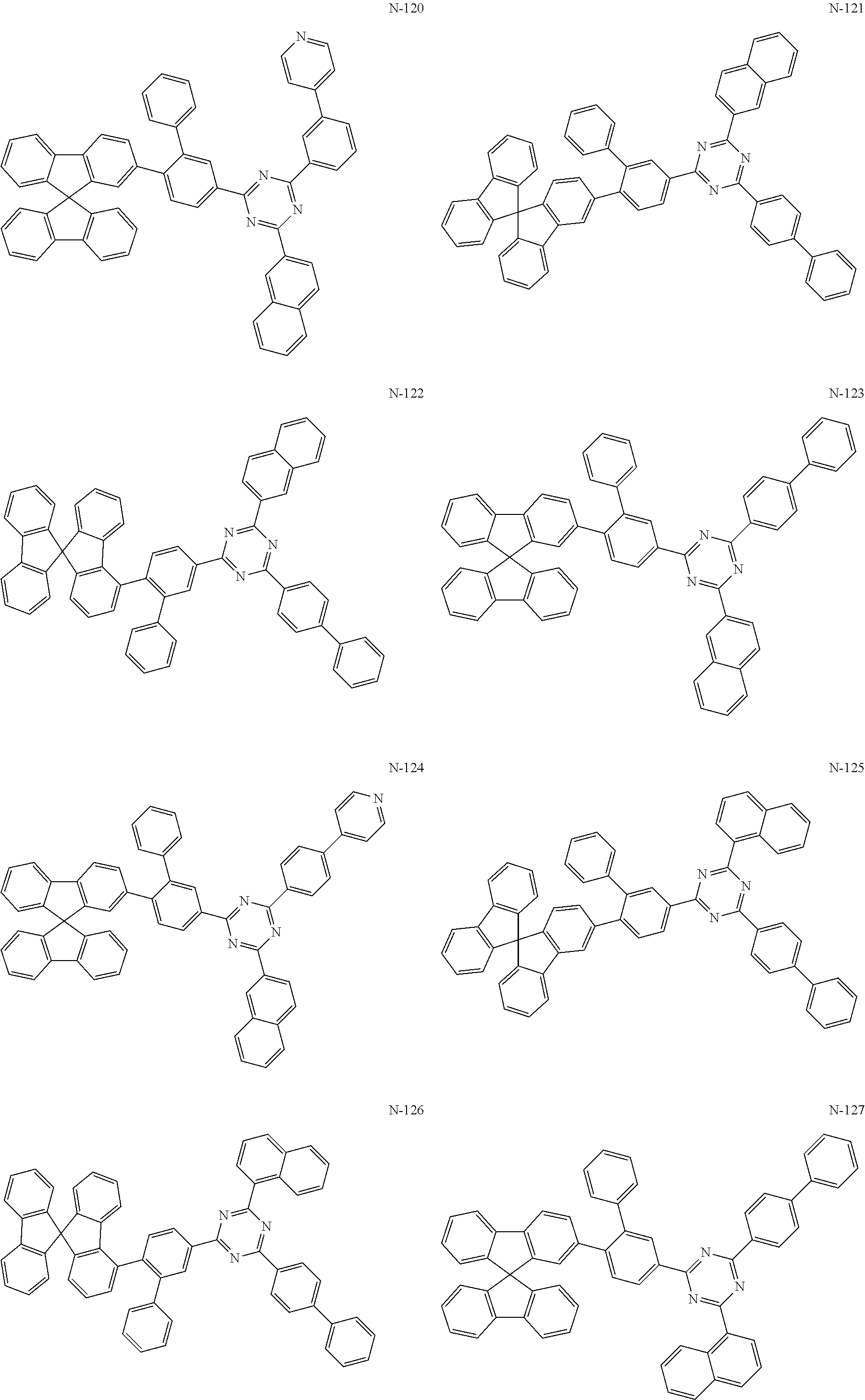

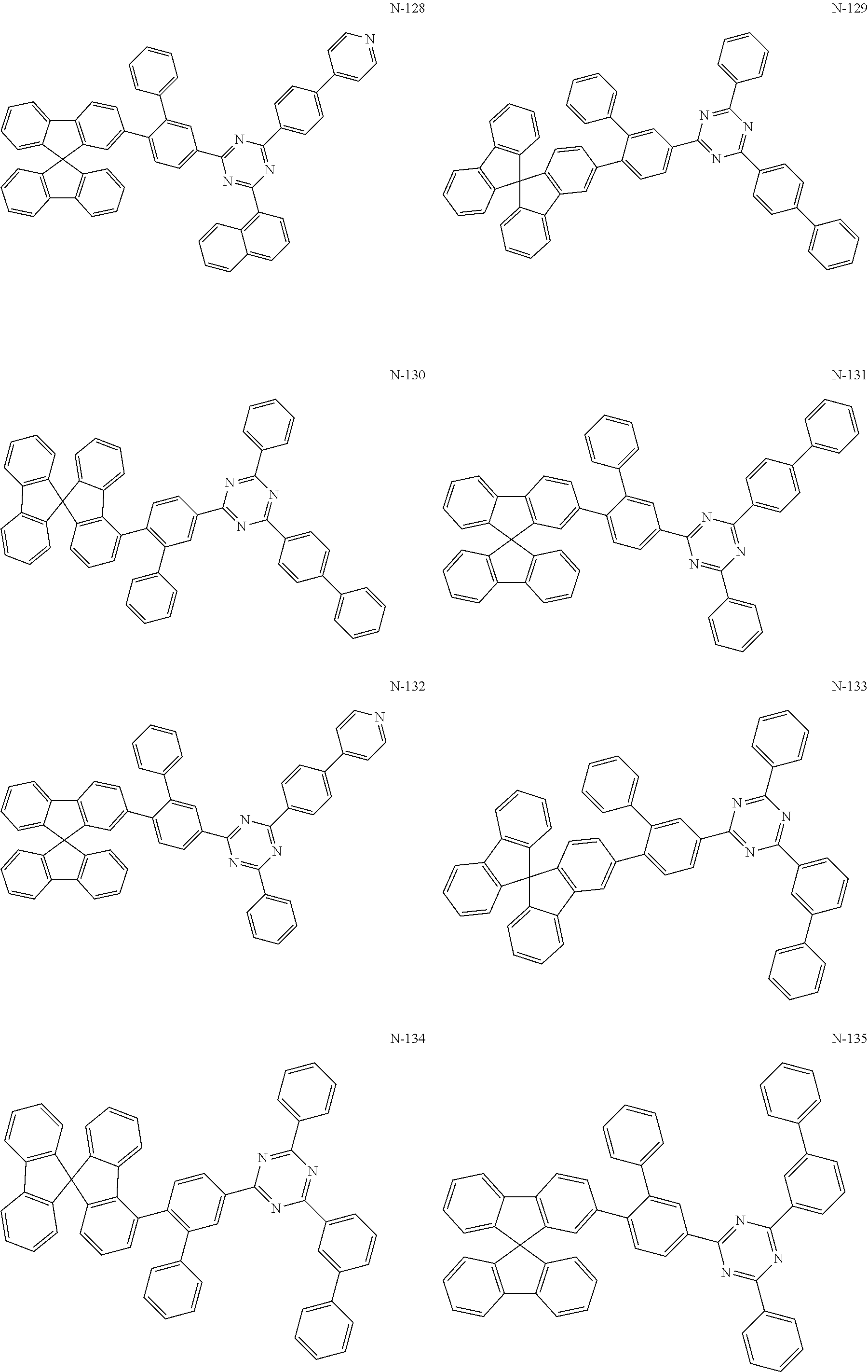

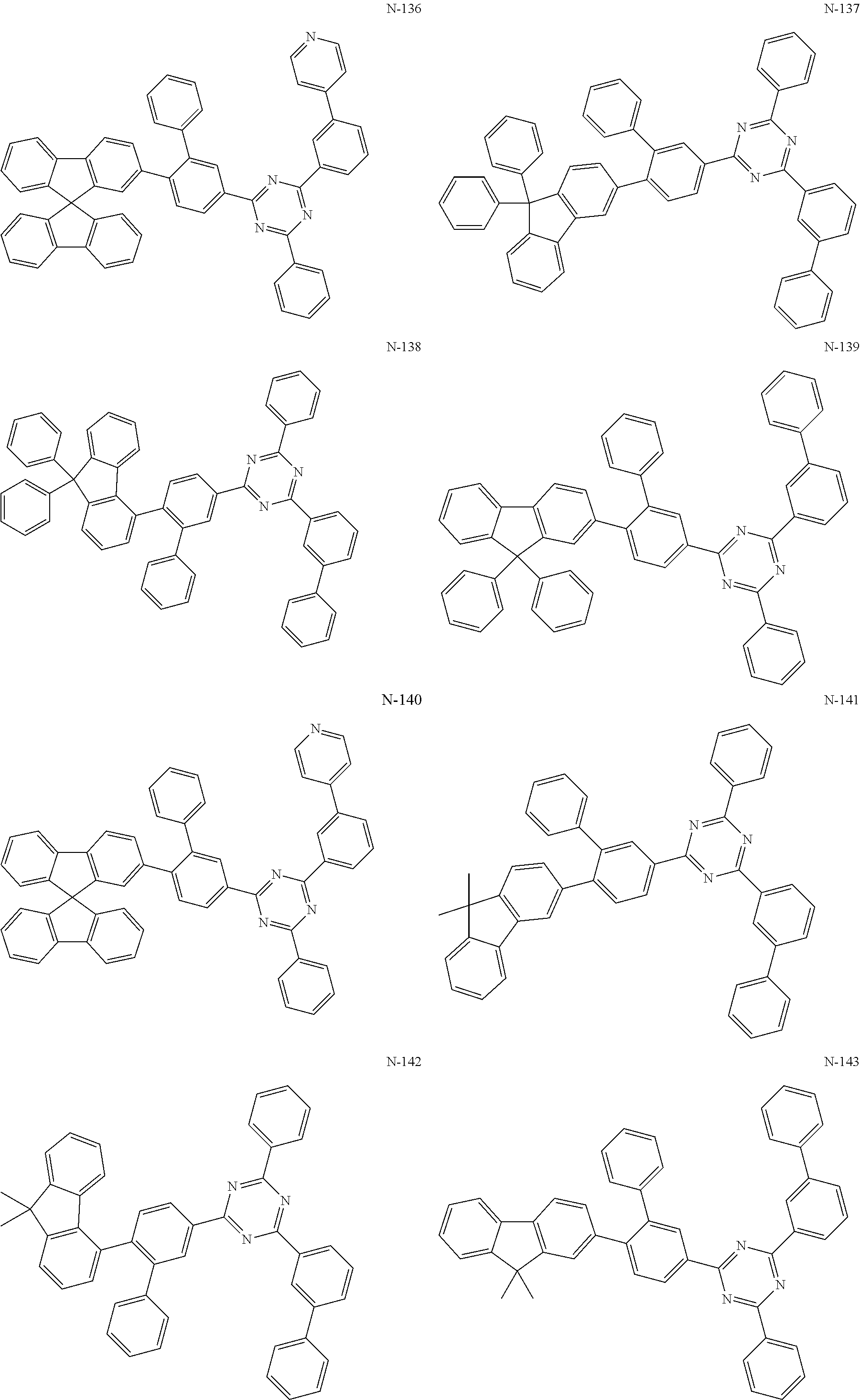

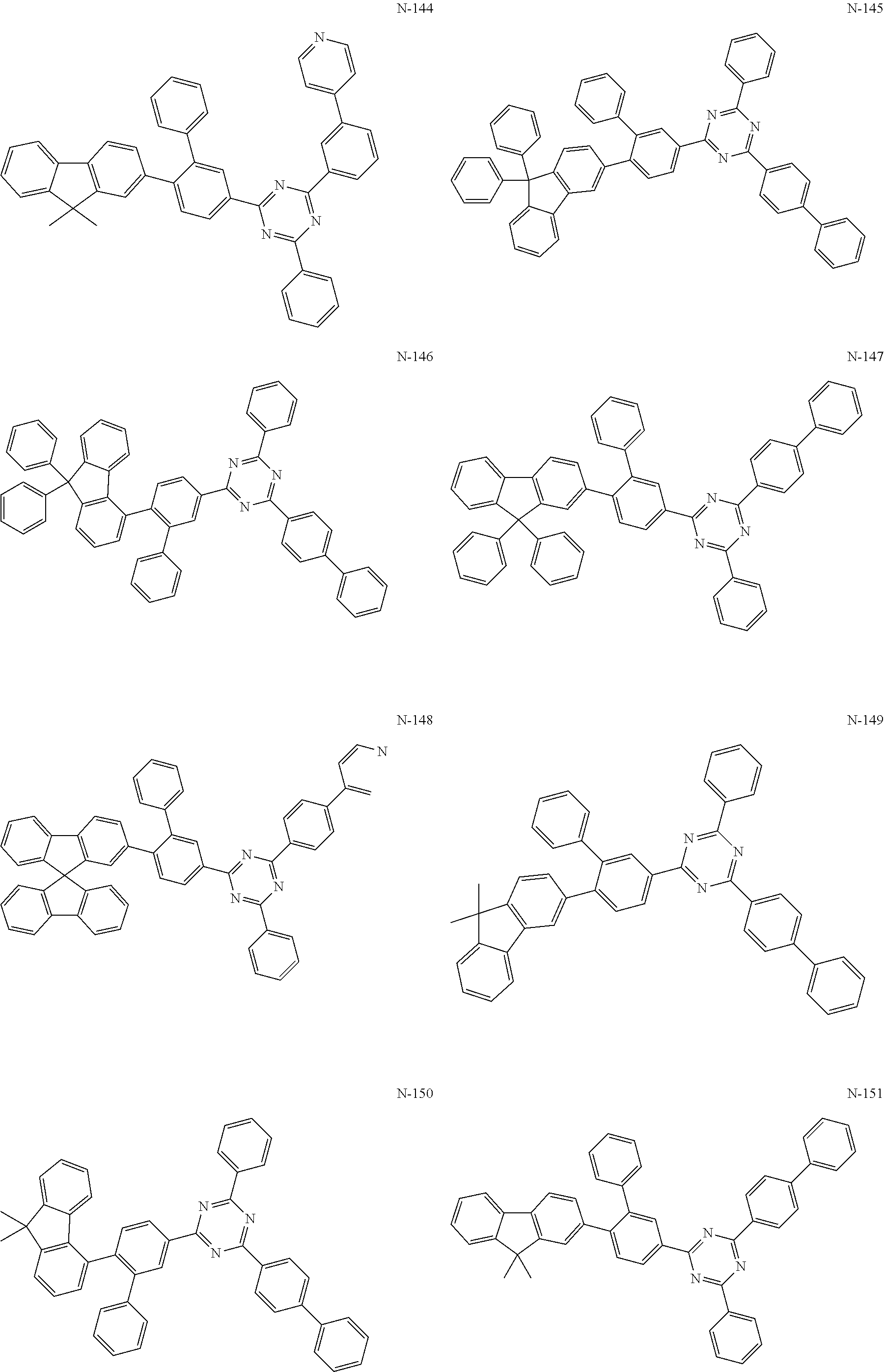

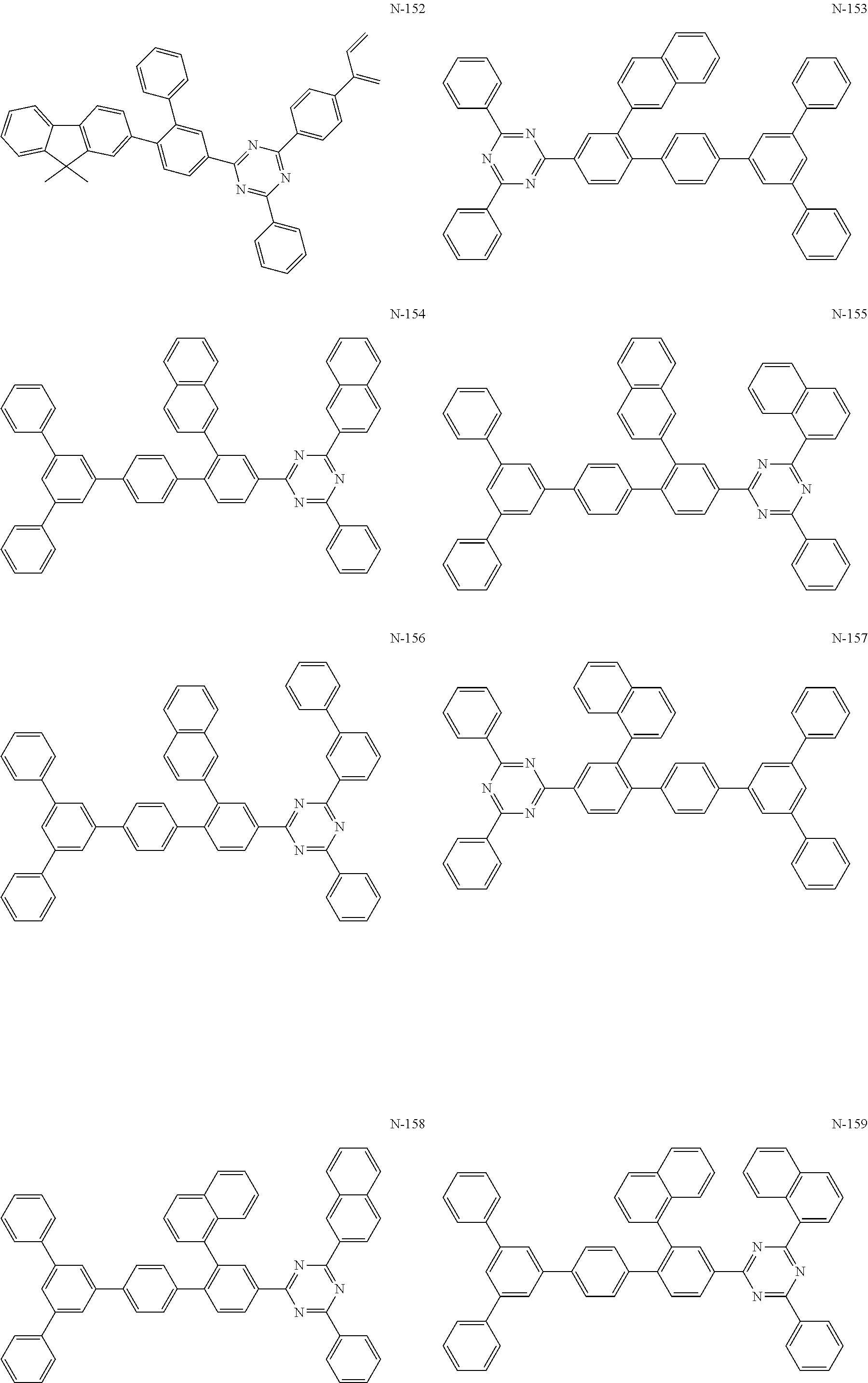

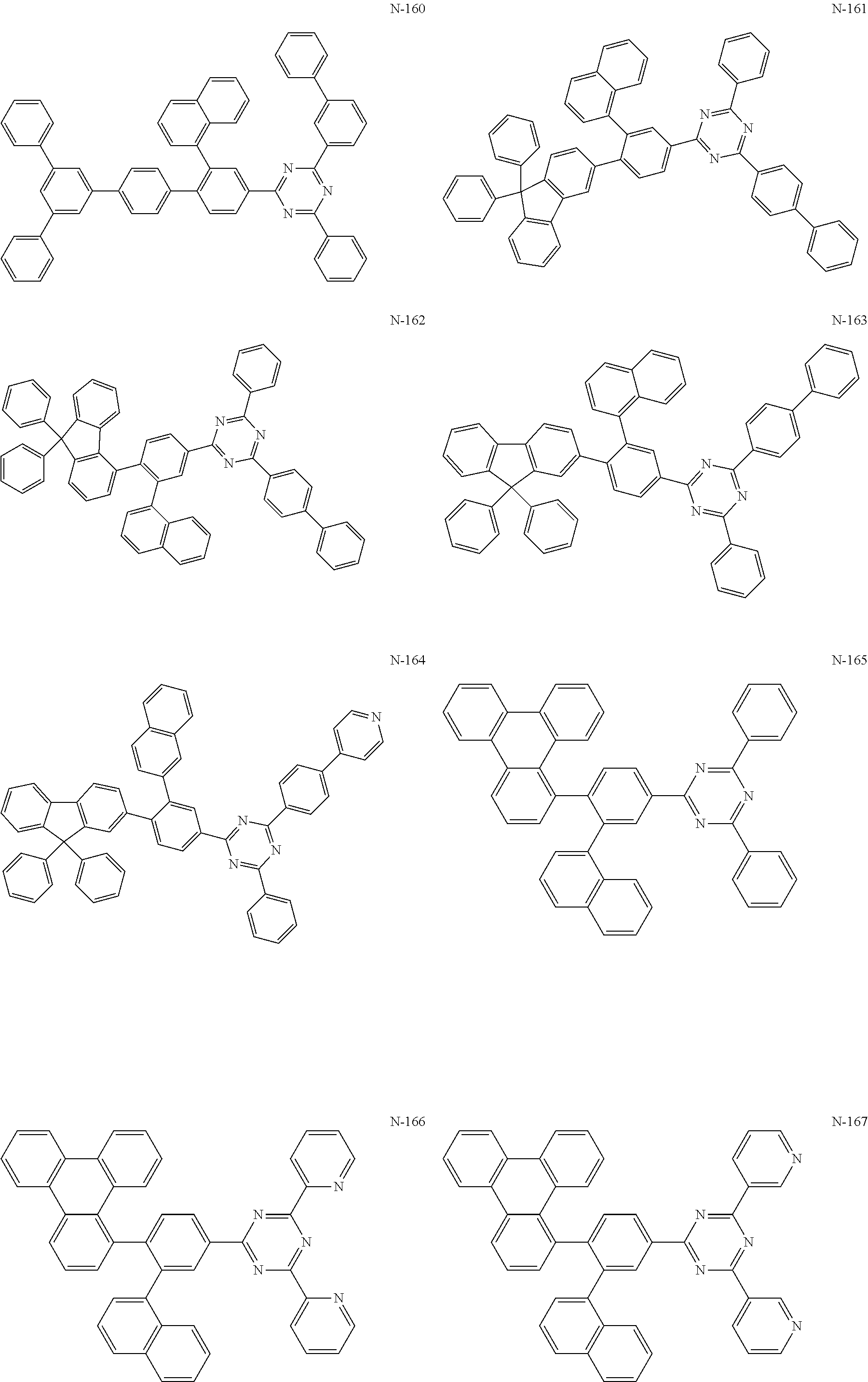

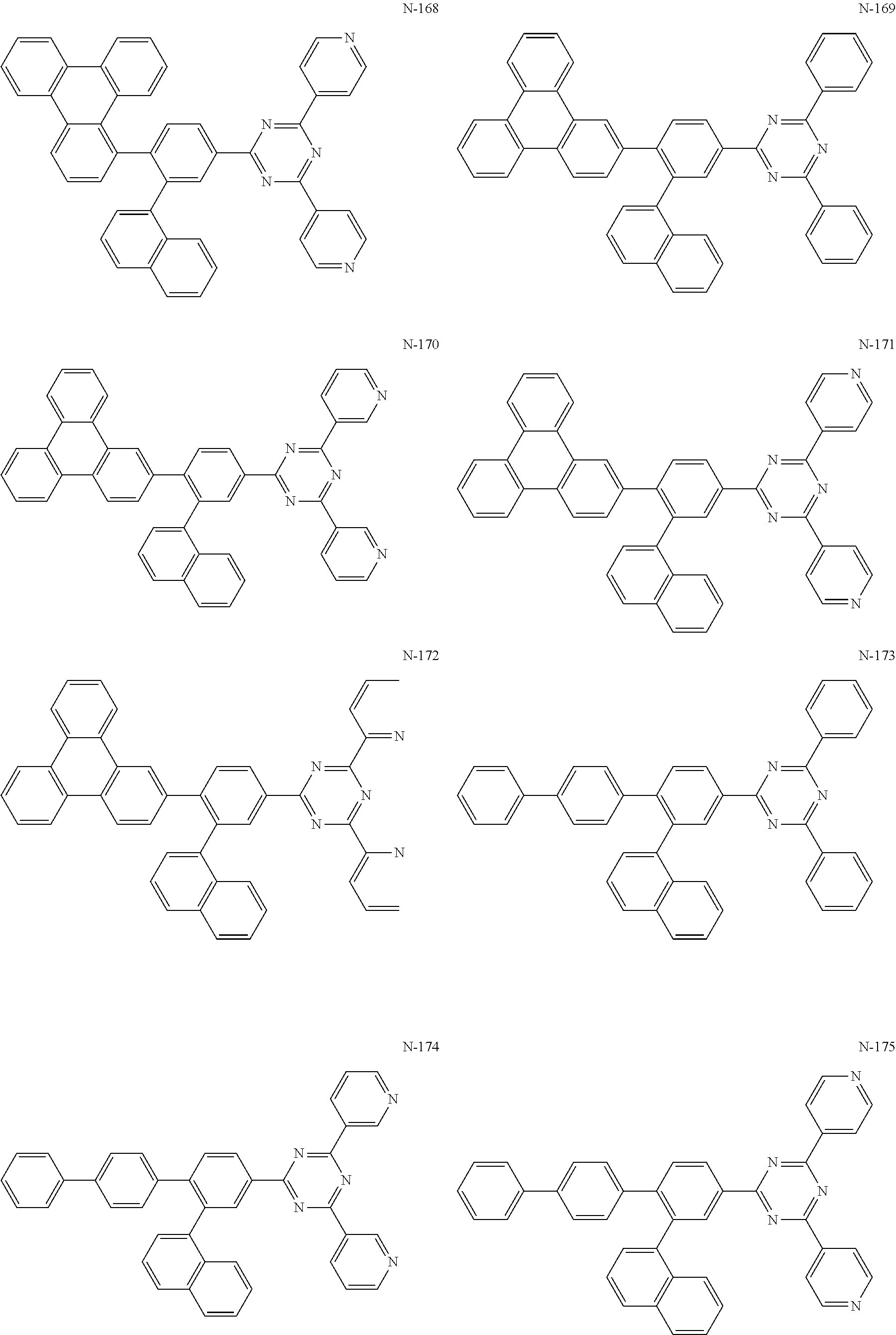

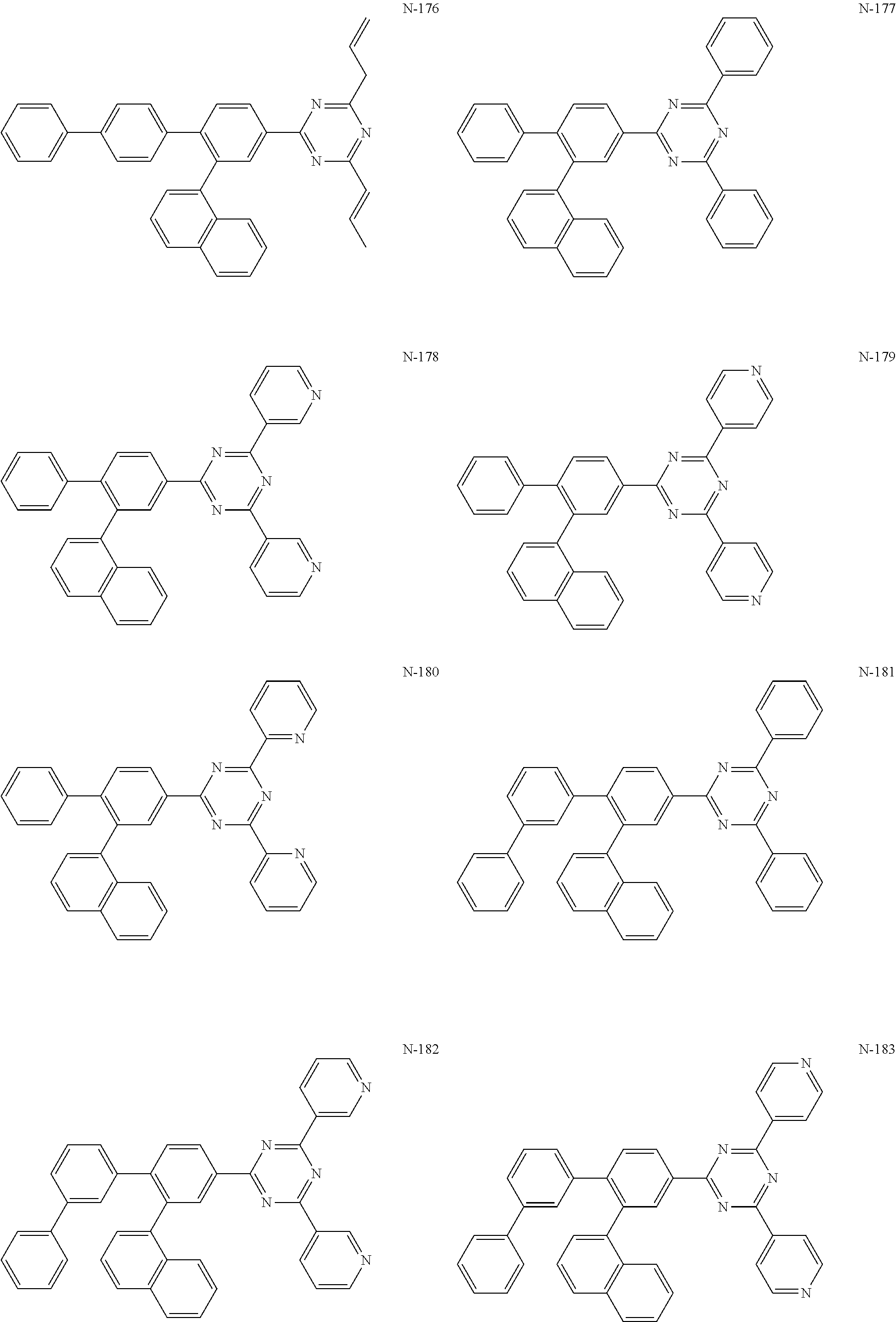

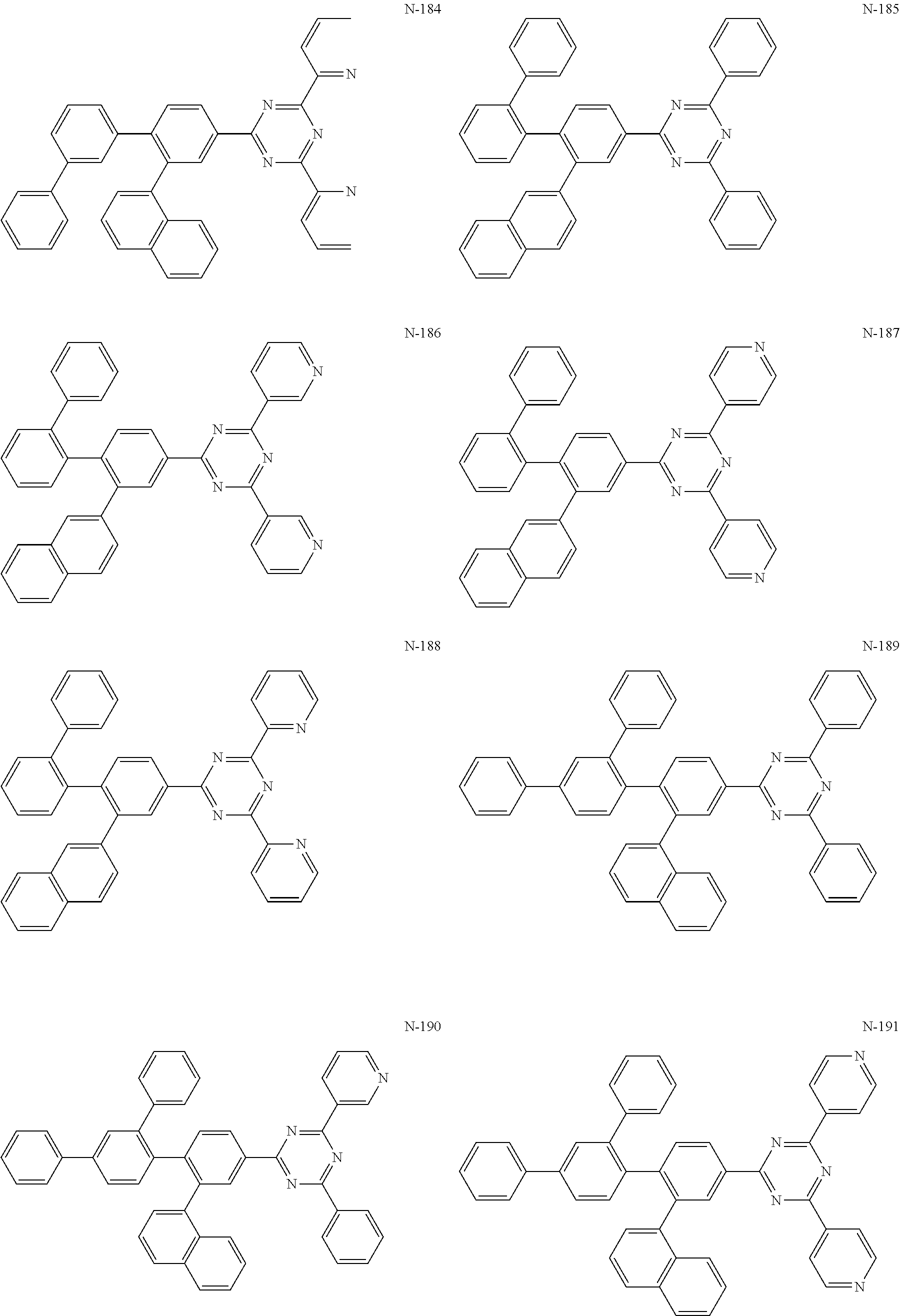

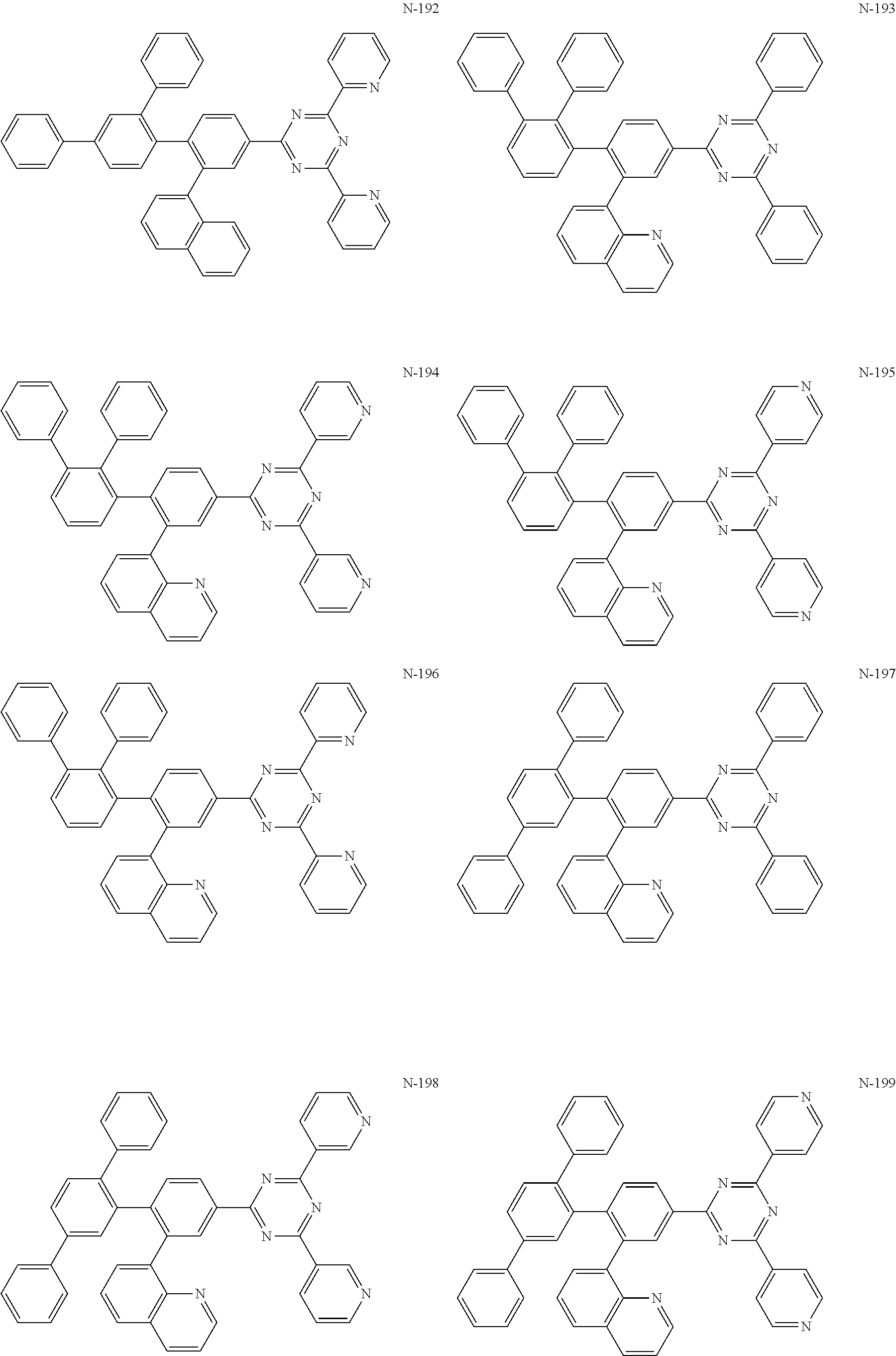

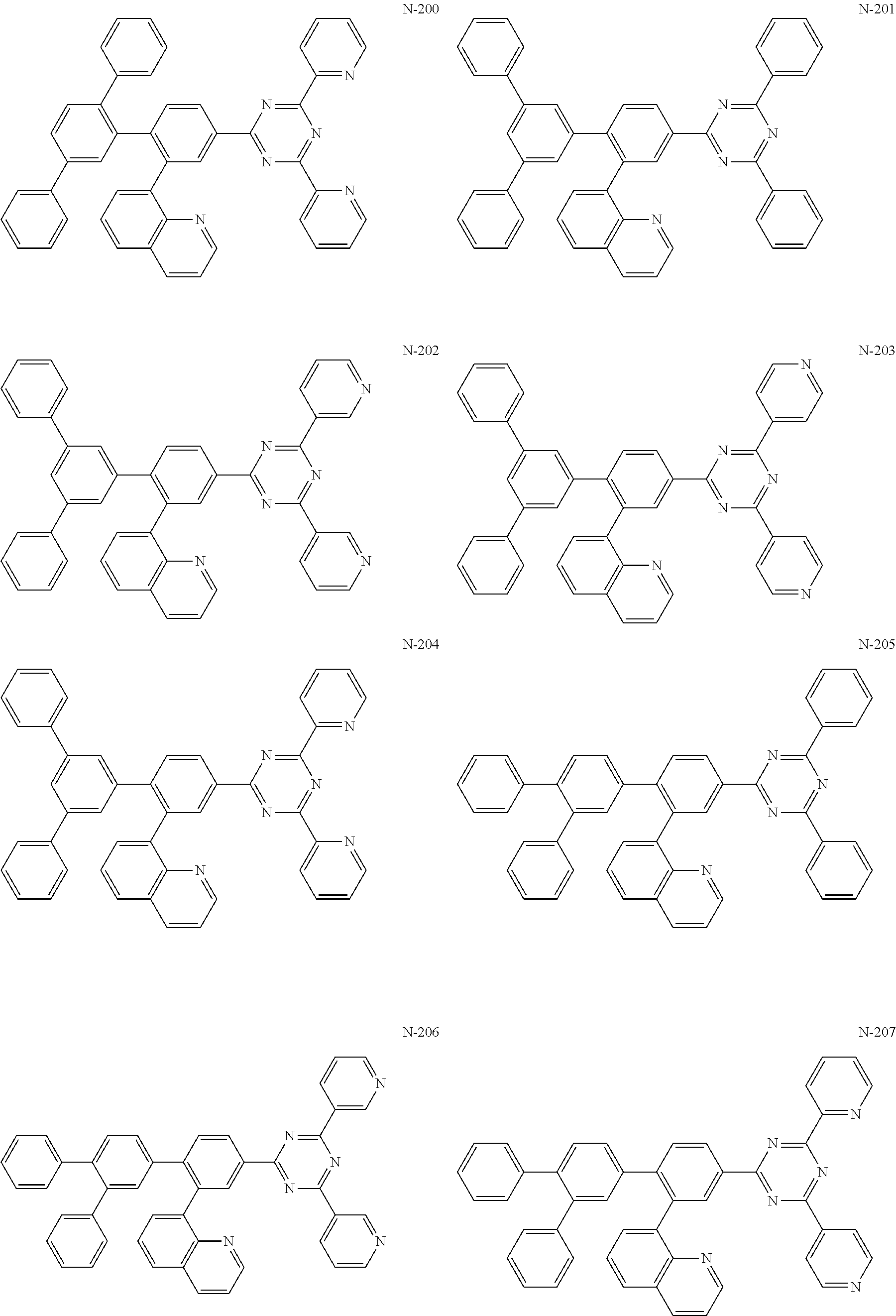

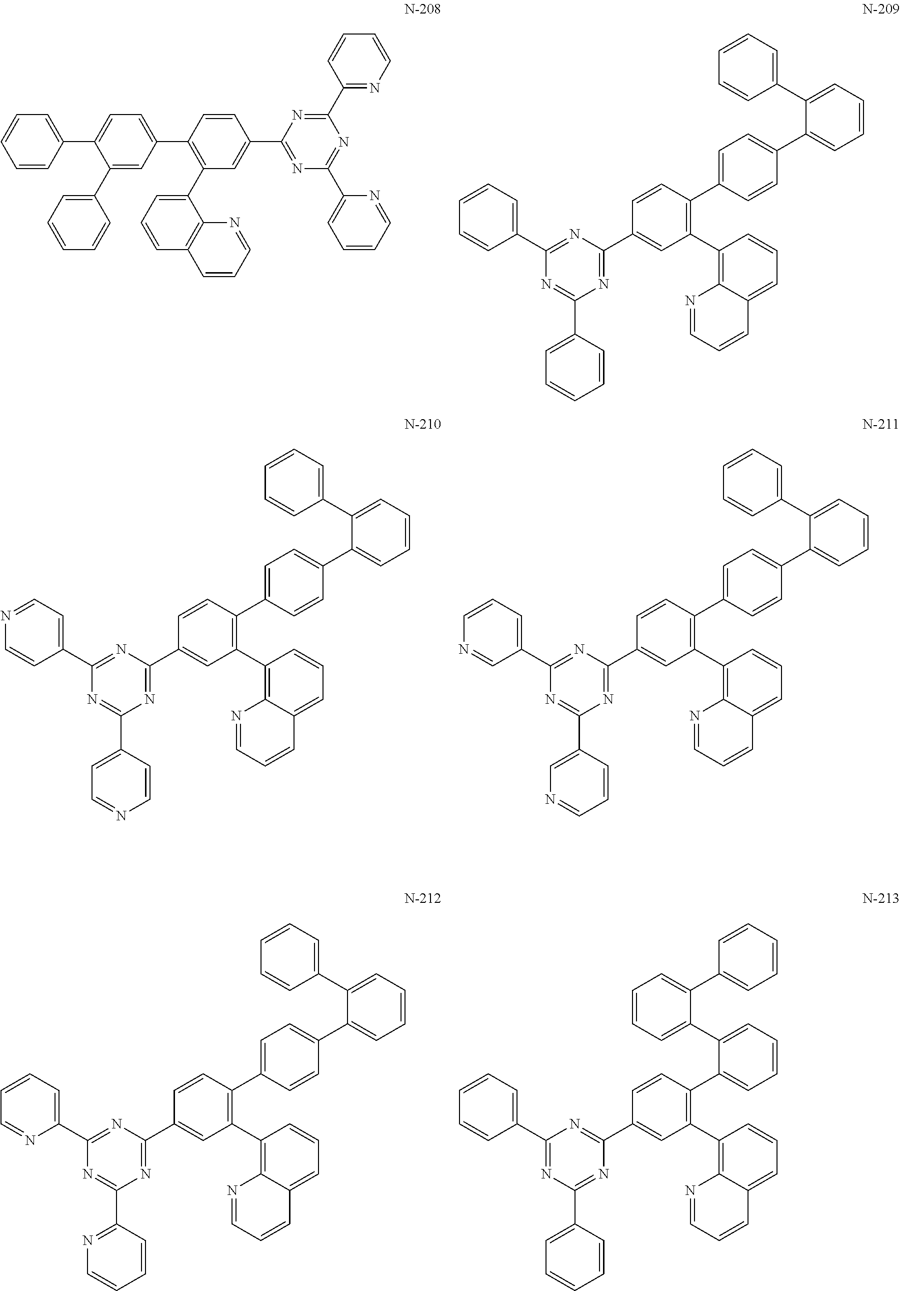

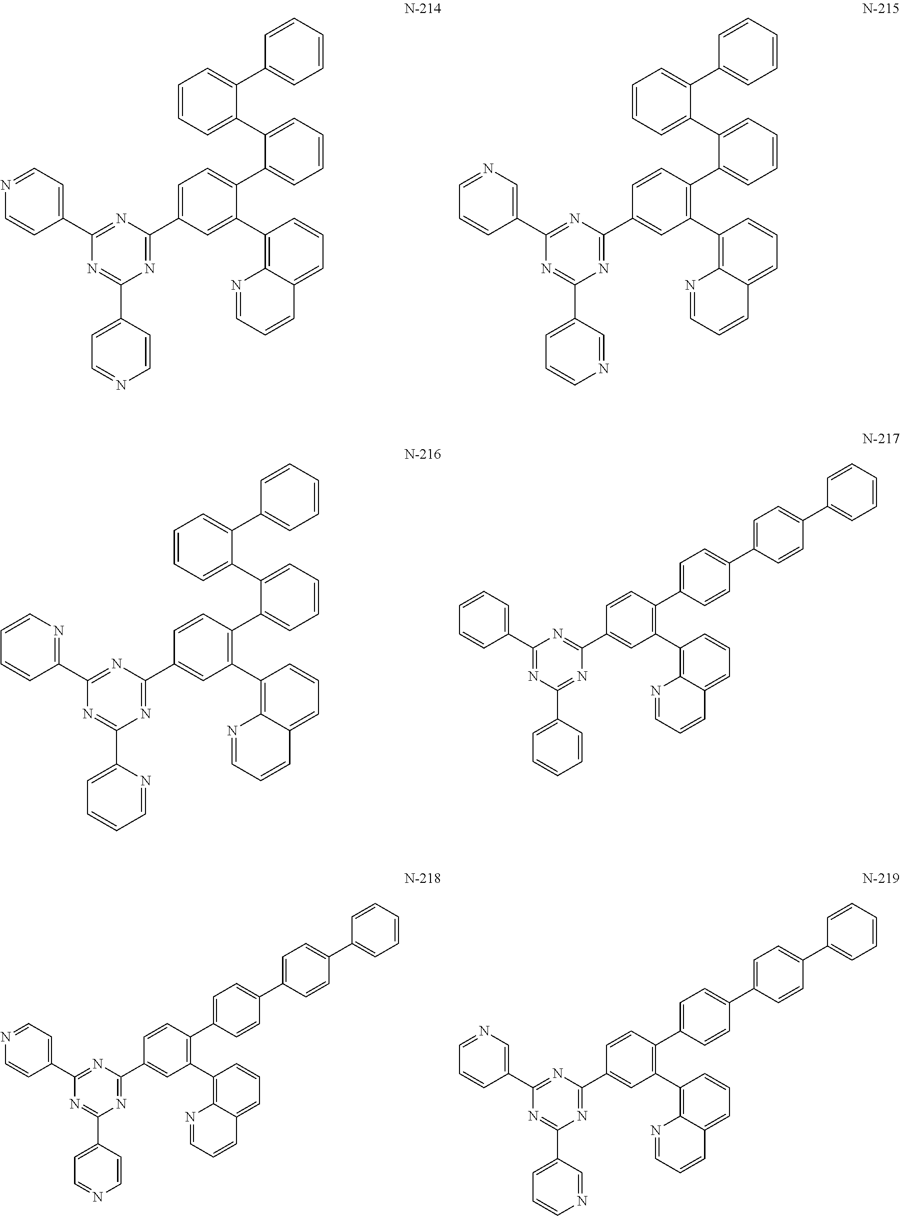

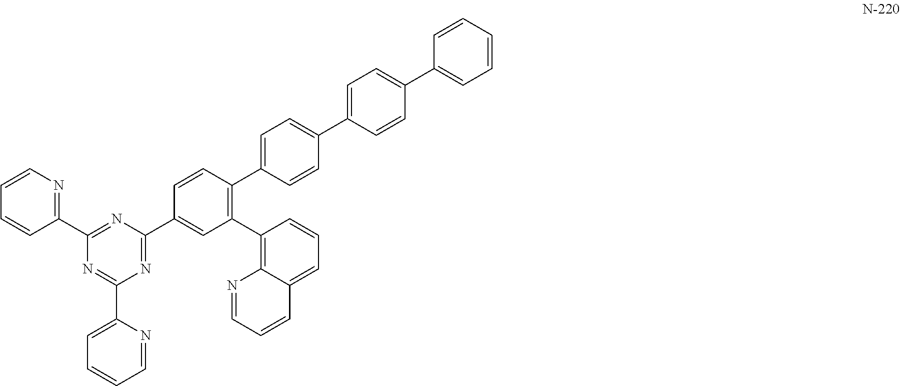

In one of the embodiments, the organic compound is used for an evaporated OLED device, and thus the organic compound has a molecular weight less than or equal to 1000 g/mol. In one of the embodiments, the organic compound has a molecular weight less than or equal to 900 g/mol. In one of the embodiments, the organic compound has a molecular weight less than or equal to 850 g/mol. In one of the embodiments, the organic compound has a molecular weight less than or equal to 800 g/mol. In one of the embodiments, the organic compound has a molecular weight less than or equal to 750 g/mol.

The aforementioned organic compound can be used in an organic mixture. The aforementioned organic compound can also be used in a formulation. The aforementioned organic compound can also be used in an organic electronic device. The aforementioned organic compound can also be used in an electronic device.

A polymer according to an embodiment has at least one repeating unit comprising a structure represented by general formula (1). In one of the embodiments, the polymer is a non-conjugated polymer, in which the structural unit represented by the general formula (1) is on the side chain. In another embodiment, the polymer is a conjugated polymer.

An organic mixture according to an embodiment includes the aforementioned organic compound which used as a first compound H1, and the organic mixture further includes a second compound H2. min((LUMO(H1)-HOMO(H2), (LUMO(H2)-HOMO(H1)))≤min(E_{T}(H1), E_{T}(H2))+0.1 eV. LUMO(H1), HOMO(H1), and E_{T}(H1) are energy levels of the lowest unoccupied molecular orbital, the highest occupied molecular orbital, and the triplet excited state of the organic compound H1, respectively. LUMO(H2), HOMO(H2), and E_{T}(H2) are energy levels of the lowest unoccupied molecular orbital, the highest occupied molecular orbital, and the triplet excited state of the organic compound H2, respectively. It should be noted that the organic compound H1 and the organic compound H2 in the organic mixture may be of various types.

In one of the embodiments, the organic compound H1 and the organic compound H2 form a type-II heterojunction structure.

In one of the embodiments, min((LUMO(H1)-HOMO(H2)), (LUMO(H2)-HOMO(H1)))≤min(E_{T}(H1), E_{T}(H2)).

In one of the embodiments, min((LUMO(H1)-HOMO(H2)), (LUMO(H2)-HOMO(H1)))≤min(E_{T}(H1), E_{T}(H2))−0.05 eV.

In one of the embodiments, min((LUMO(H1)-HOMO(H2)), (LUMO(H2)-HOMO(H1)))≤min(E_{T}(H1), E_{T}(H2))−0.1 eV.

In one of the embodiments, min((LUMO(H1)-HOMO(H2)), (LUMO(H2)-HOMO(H1)))≤min(E_{T}(H1), E_{T}(H2))−0.15 eV.

In one of the embodiments, min((LUMO(H1)-HOMO(H2)), (LUMO(H2)-HOMO(H1)))≤min(E_{T}(H1), E_{T}(H2))−0.2 eV.

In the present disclosure, triplet excited state energy level Er, HOMO, and LUMO play a key role in the energy level structure of the organic material. The determination of these energy levels is introduced as follows.

HOMO and LUMO energy levels can be measured by optoelectronic effects, such as XPS (X-ray photoelectron spectroscopy) and UPS (UV photoelectron spectroscopy), or by cyclic voltammetry (hereinafter referred to as CV). Quantum chemical methods, such as density functional theory (hereinafter referred to as DFT), have also become effective methods for calculating molecular orbital energy levels.

The triplet excited state energy level Er of an organic material can be measured by a low-temperature time-resolved spectroscopy or by quantum simulation calculation (for example, by Time-dependent DFT), such as by commercial software Gaussian 03W (Gaussian Inc.). Detailed simulation methods can be found in WO2011141110 or as described below in the examples.

It should be noted that the absolute values of HOMO, LUMO, and E_{T} depend on the measurement method or calculation method used, and even for the same method but different evaluation method, for example, different HOMO/LUMO value can be provided at the starting point and peak point on a CV curve. Therefore, a reasonable and meaningful comparison should be carried out using the same measurement method and the same evaluation method. As described in the embodiments of the present disclosure, the values of HOMO, LUMO, and E_{T} are simulations based on Time-dependent DFT, without affecting the application of other measurement or calculation methods.

In the aforementioned organic mixture, the excited state of the system may preferentially occupy the exciplex states with the lowest energy or may facilitate energy transfer from the triplet excited states of H1 and H2 to the exciplex states, thereby increasing the density of the exciplex states. The organic mixture includes the first organic compound H1 and the second organic compound H2 which are capable of forming the exciplex. Both the organic compound H1 and the organic compound H2 are used as host materials, and H1 and H2 have a type-II semiconductor heterojunction structure, which has better stability and simplifies the subsequent evaporation process.

The aforementioned organic compound or organic mixture can be used as an electrophosphorescent host material or a co-host material thereof. By cooperating with a suitable guest material, the luminous efficiency and lifetime of the organic compound or the organic mixture as an electroluminescent device can be improved. The aforementioned organic mixture can also be used as a fluorescent co-host material or a light-emitting material, and by cooperating with a suitable fluorescent host material or guest material, the luminous efficiency and lifetime of the organic compound or the organic mixture as the electroluminescent device can be improved. In this way, a solution for a light-emitting device with low manufacturing cost, high efficiency, and long lifetime is provided.

In one of the embodiments, the organic mixture may be used as a phosphorescent host material.

In one of the embodiments, min((LUMO(H1)-HOMO(H2)), (LUMO(H2)-HOMO(H1))) is less than or equal to the energy level of the triplet excited state of H1, and min((LUMO(H1)-HOMO(H2)), (LUMO(H2)-HOMO (H1))) is less than or equal to the energy level of the triplet excited state of H2. The energy of the exciplex formed by the organic compound H1 and the organic compound H2 is determined by min((LUMO(H1)-HOMO(H2)), (LUMO(H2)-HOMO(H1))).

In one of the embodiments, at least one of the organic compound H1 and the organic compound H2 has a ((HOMO-(HOMO-1)) greater than or equal to 0.2 eV. In one of the embodiments, at least one of the organic compound H1 and the organic compound H2 has a ((HOMO-(HOMO-1)) greater than or equal to 0.25 eV. In one of the embodiments, at least one of the organic compound H1 and the organic compound H2 has a ((HOMO-(HOMO-1)) greater than or equal to 0.3 eV. In one of the embodiments, at least one of the organic compound H1 and the organic compound H2 has a ((HOMO-(HOMO-1)) greater than or equal to 0.35 eV. In one of the embodiments, at least one of the organic compound H1 and the organic compound H2 has a ((HOMO-(HOMO-1)) greater than or equal to 0.4 eV. In one of the embodiments, at least one of the organic compound H1 and the organic compound H2 has a ((HOMO-(HOMO-1)) greater than or equal to 0.45 eV.

In one of the embodiments, the organic compound H2 contains the electron-donating group. Thus, the organic compound H1 and the organic compound H2 are prone to form a type-II semiconductor heterojunction.

In one of the embodiments, the organic compound H2 has a ((HOMO-(HOMO-1)) greater than or equal to 0.2 eV. In one of the embodiments, the organic compound H2 has a ((HOMO-(HOMO-1)) greater than or equal to 0.25 eV In one of the embodiments, the organic compound H2 has a ((HOMO-(HOMO-1)) greater than or equal to 0.3 eV. In one of the embodiments, the organic compound H2 has a ((HOMO-(HOMO-1)) greater than or equal to 0.35 eV. In one of the embodiments, the organic compound H2 has a ((HOMO-(HOMO-1)) greater than or equal to 0.4 eV. In one of the embodiments, the organic compound H2 has a ((HOMO-(HOMO-1)) greater than or equal to 0.45 eV.

In one of the embodiments, the organic compound H2 is a compound represented by one of e following general formulas (4) to (7):

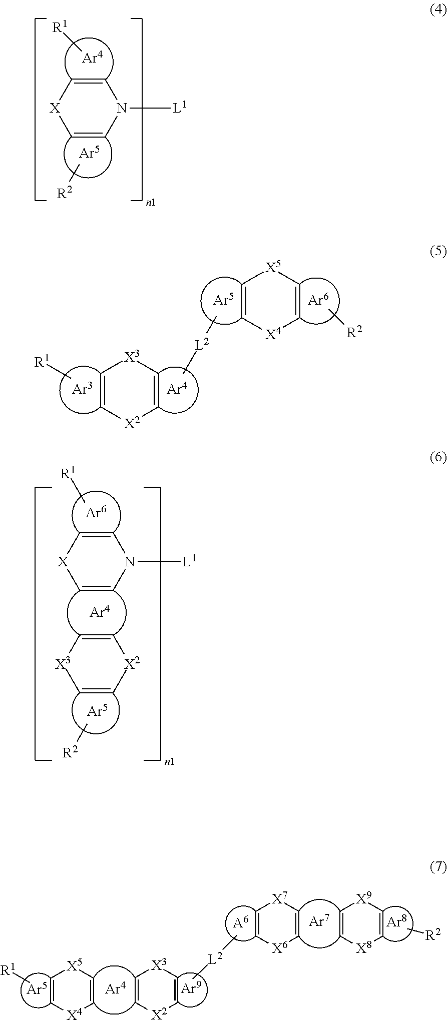

Wherein, L¹ is selected from the group consisting of an aromatic group containing 5 to 60 ring atoms and a heteroaromatic group containing 5 to 60 ring atoms. L² is selected from the group consisting of a single bond, an aromatic group containing 5 to 30 ring atoms, and a heteroaromatic group containing 5 to 30 ring atoms. A linking position of L² is any one of carbon atoms on a ring. Ar⁴, Ar⁵, Ar⁶, Ar⁷, Ar⁸, and Ar⁹ are independently selected from the group consisting of an aromatic group containing 5 to 30 ring atoms and a heteroaromatic group containing 5 to 30 ring atoms. X is selected from the group consisting of a signal bond, N(R), C(R)₂, Si(R)₂, O, C═N(R), C═C(R)₂, P(R), P(═O)R, S, S═O, and SO₂. X², X³, X⁴, X⁵, X⁶, X⁷, X⁸, and X⁹ are independently selected from the group consisting of a single bond, N(R), C(R)₂, Si(R)₂, O, C═N(R), C═C(R)₂, P(R), P(═O)R, S, S═O, and SO₂. However, X² and X³ are not both single bonds simultaneously, X⁴ and X⁵ are not both single bonds simultaneously, X⁶ and X⁷ are not both single bonds simultaneously, and X⁸ and X⁹ are not both single bonds simultaneously. R¹, R², and R are independently selected from the group consisting of H, D, F, CN, alkenyl, alkynyl, nitrile, amino, nitro, acyl, alkoxy, carbonyl, sulfonyl, an alkyl containing 1 to 30 carbon atoms, a cycloalkyl containing 3 to 30 carbon atoms, an aromatic hydrocarbyl containing 5 to 60 ring atoms, or an aromatic heterocyclic group containing 5 to 60 ring atoms. Linking positions of R¹ and R² are any one or more of carbon atoms on a fused ring. n1 is selected from 1, 2, 3, or 4.

In addition, in one of the embodiments, the organic compound H2 is a compound represented by one of the following general formulas (8) to (11):

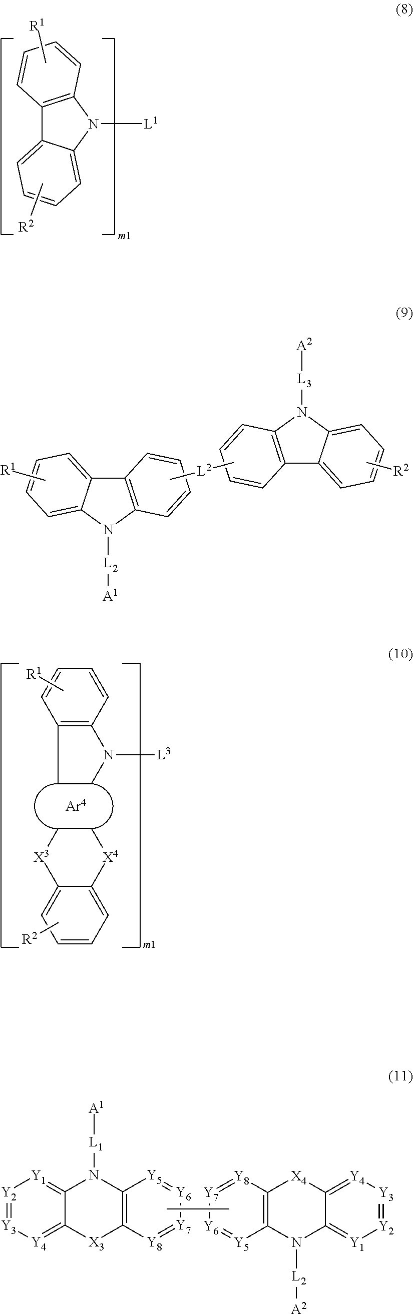

L³ is selected from the group consisting of an aromatic group containing 5 to 60 ring atoms and a heteroaromatic group containing 5 to 60 ring atoms. A¹ and A² are independently selected from the group consisting of an aromatic group containing 5 to 30 ring atoms and a heteroaromatic group containing 5 to 30 ring atoms. Y¹ to Y⁸ are independently selected from N or CR, and adjacent Y¹ to Y⁸ are not both N simultaneously. m1 is selected from 1, 2, 3, or 4.

In one of the embodiments, the organic compound H2 represented by the general formulas (4) to (11) is selected from the group consisting of compounds represented by the following structures.

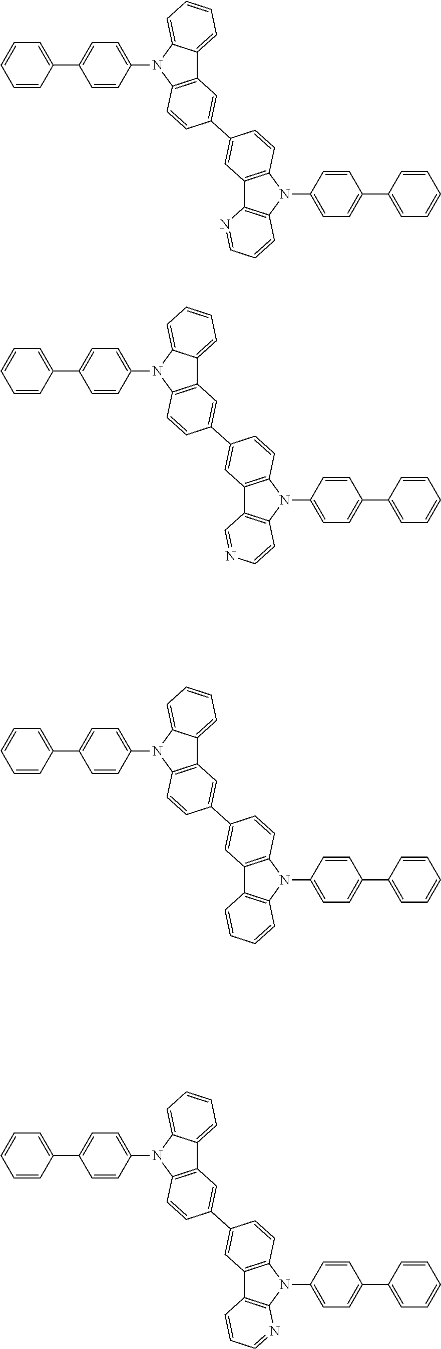

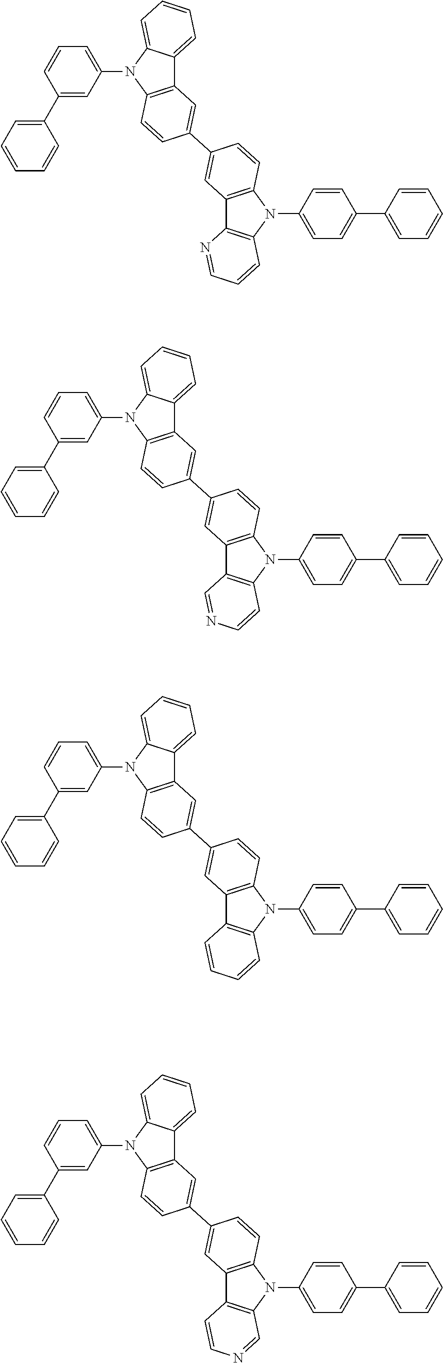

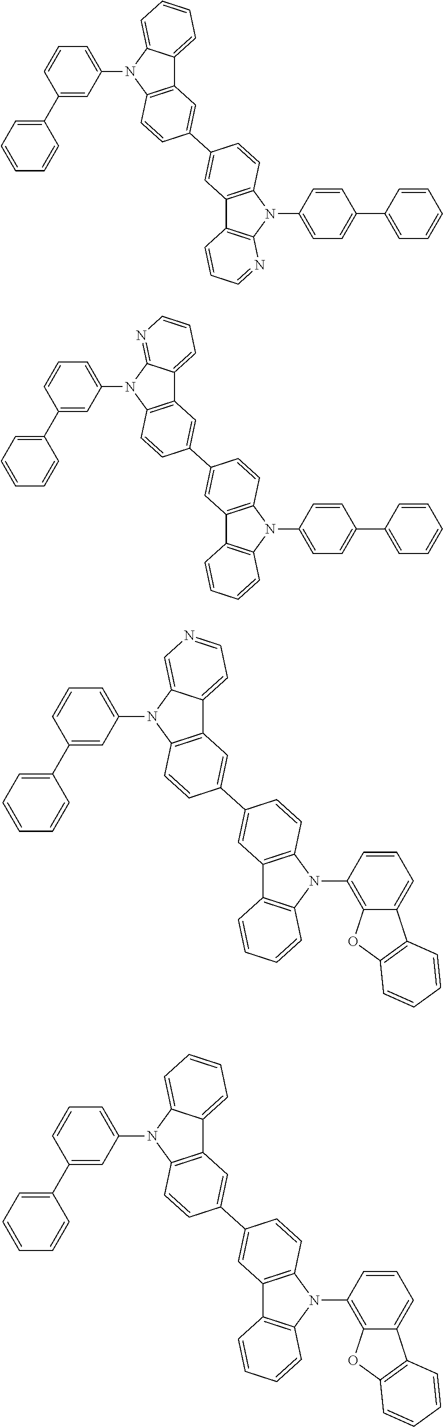

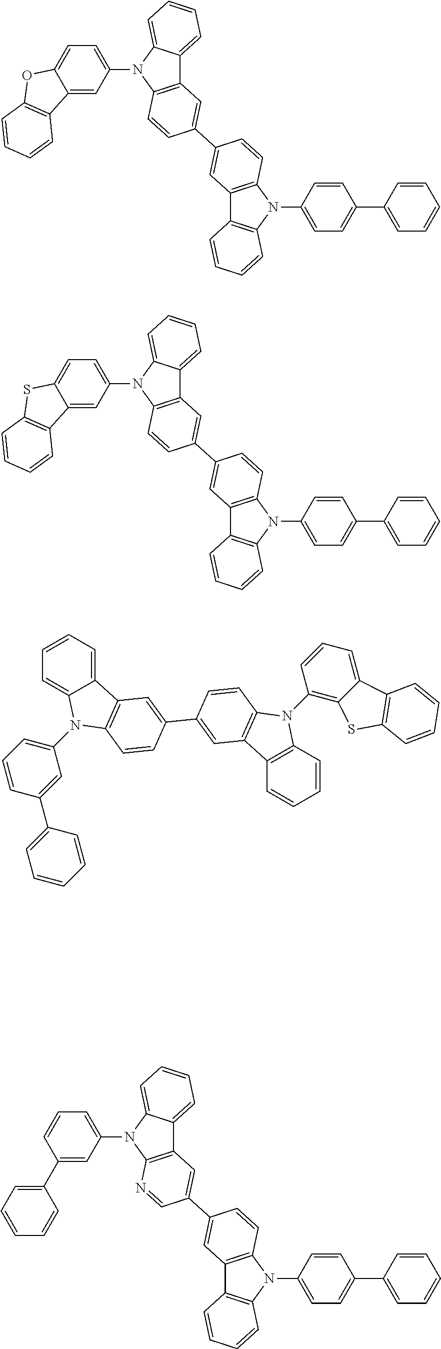

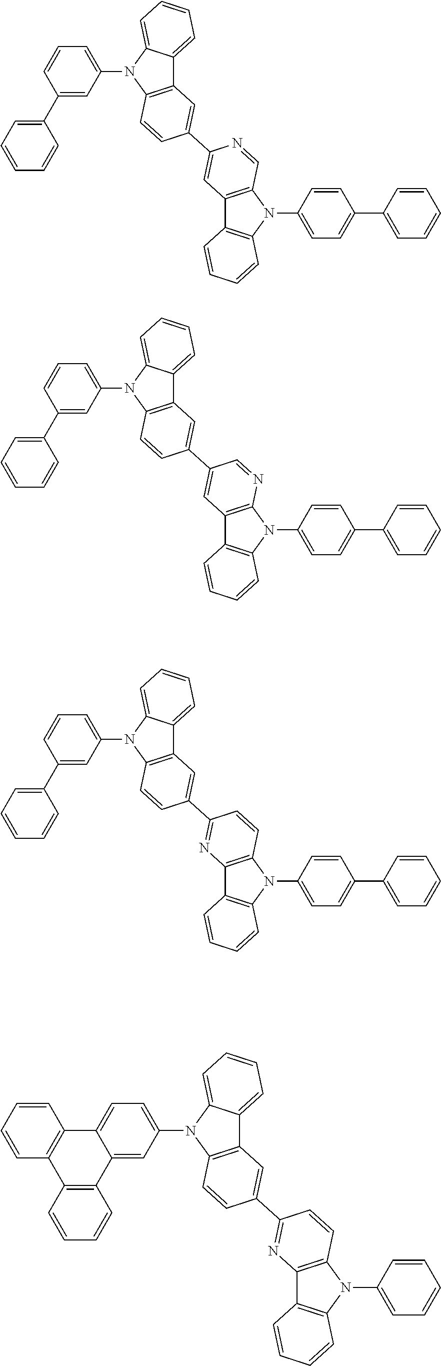

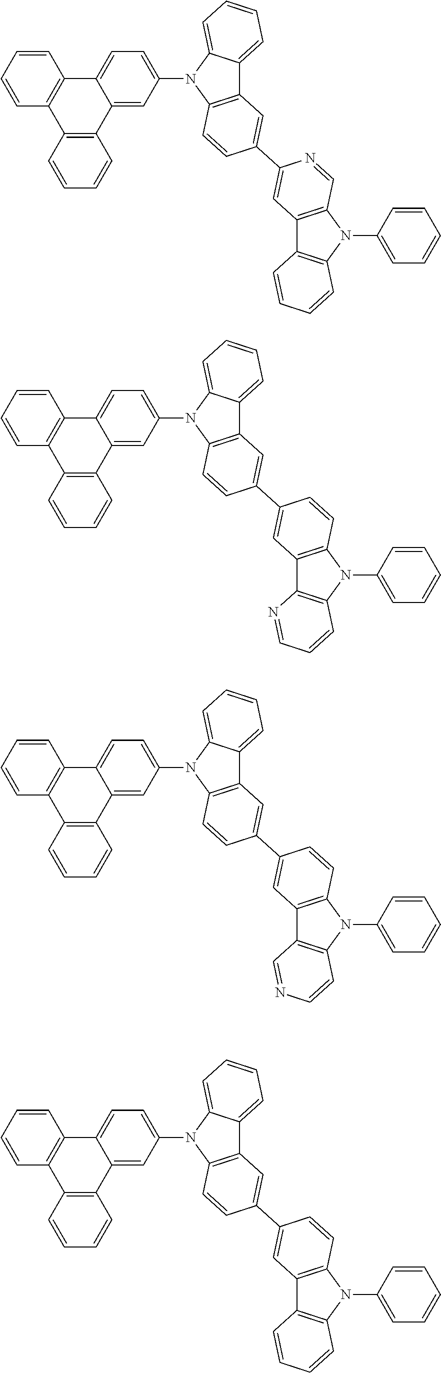

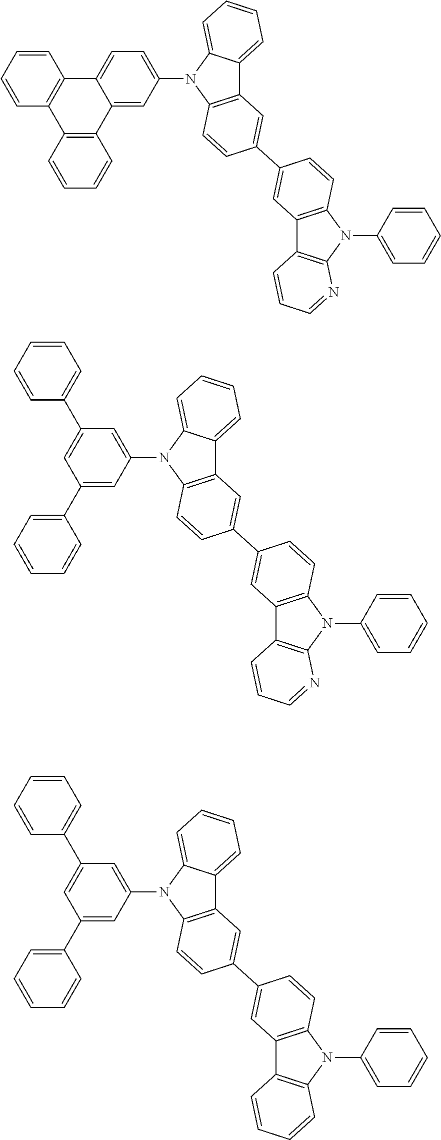

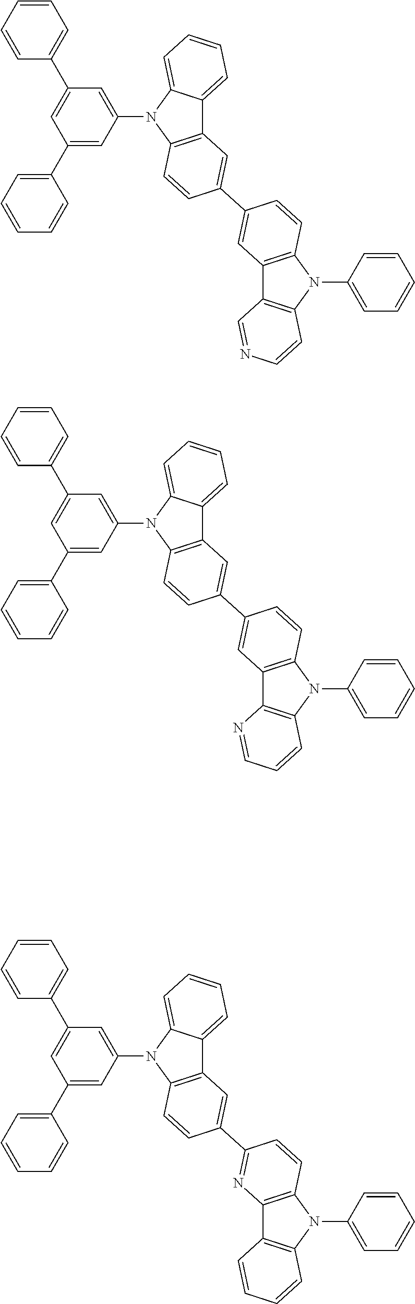

In one of the embodiments, the organic mixture is used as the host material of the light-emitting layer in the electroluminescent device, and at this time, min((LUMO(H1)-HOMO(H2)), (LUMO(H2)-HOMO(H1))) is less than or equal to the energy level of the triplet excited state of H1, and min((LUMO(H1)-HOMO(H2)), (LUMO(H2)-HOMO(H1))) is less than or equal to the energy level of the triplet excited state of H2.

When the light-emitting layer is formed using a single material that is electron-biased or hole-biased, excitons may be formed relatively more at interfaces of the light-emitting layer, and an electron transport layer and a hole transport layer. Therefore, the excitons of the light-emitting layer may interact with the interface charges of the electron transport layer or the hole transport layer, thereby causing a serious roll-off of the device efficiency under high luminance and a shortening of the lifetime. In order to solve the problem, the organic compound H1 and the organic compound H2 are mixed and introduced into the light-emitting layer to balance mobolities of holes and electrons in the light-emitting layer, so that the light-emitting region can emit light in the middle of the light-emitting layer, thereby improving the device efficiency while improving the device lifetime.

In one of the embodiments, a mass ratio of the organic compound H1 to the organic compound H2 ranges from (2:8) to (8:2). In one of the embodiments, the mass ratio of the organic compound H1 to the organic compound H2 ranges from (3:7) to (7:3). In one of the embodiments, the mass ratio of the organic compound H1 to the organic compound H2 ranges from (4:6) to (6:4). In one of the embodiments, the mass ratio of the organic compound H1 to the organic compound H2 ranges from (4.5:5.5) to (5.5:4.5). In one of the embodiments, the mass ratio of the organic compound H1 to the organic compound H2 is (5:5).

In one of the embodiments, the organic compound is a small molecule material and the aforementioned organic mixture is also a small molecule organic mixture.

The term “small molecule” as defined herein refers to a molecule that is not a polymer, oligomer, dendrimer, or blend. In particular, there are no repeating structures in the small molecules. The small molecule has a molecular weight less than or equal to 4000 g/mol. In one embodiment, the molecular weight is less than or equal to 3000 g/mol. In another embodiment, the molecular weight is less than or equal to 2000 g/mol. In yet another embodiment, the molecular weight is less than or equal to 1500 g/mol.

The polymer includes a homopolymer, a copolymer, and a block copolymer. In addition, in the present disclosure, the polymer also includes a dendrimer. Regarding the synthesis and application of the dendrimer, please refer to [Dendrimers and Dendrons, Wiley-VCH Verlag GmbH & Co. KGaA, 2002, Ed. George R. Newkome, Charles N. Moorefield, Fritz Vogtle.].

The conjugated polymer is a polymer whose backbone is predominantly composed of sp² hybrid orbital of carbon (C) atoms. Some known examples are: polyacetylene and poly(phenylene vinylene), on the backbone of which the C atom can also be substituted by other non-C atoms, and which is still considered to be a conjugated polymer when the sp² hybridization on the backbone is interrupted by some natural defects. In addition, the conjugated polymer in the present disclosure also includes aryl amine, aryl phosphine and other heteroarmotics, organometallic complexes, and the like on its backbone.

In one of the embodiments, in order to improve the material evaporating efficiency, improve the material utilization, and simplify the material evaporating process, a difference between the molecular weight of the organic compound H1 and the molecular weight of the organic compound H2 is less than or equal to 100 g/mol. In one of the embodiments, the difference between the molecular weight of the organic compound H1 and the molecular weight of the organic compound H2 is less than or equal to 90 g/mol. In one of the embodiments, the difference between the molecular weight of the organic compound H1 and the molecular weight of the organic compound H2 is less than or equal to 70 g/mol. In one of the embodiments, the difference between the molecular weight of the organic compound H1 and the molecular weight of the organic compound H2 is less than or equal to 60 g/mol. In one of the embodiments, the difference between the molecular weight of the organic compound H1 and the molecular weight of the organic compound H2 is less than or equal to 50 g/mol. In one of the embodiments, the difference between the molecular weight of the organic compound H1 and the molecular weight of the organic compound H2 is less than or equal to 20 g/mol.

In one of the embodiments, a difference between a sublimation temperature of the organic compound H1 and a sublimation temperature of the organic compound H2 is less than or equal to 40 K. In one of the embodiments, the difference between the sublimation temperature of the organic compound H1 and the sublimation temperature of the organic compound H2 is less than or equal to 30 K. In one of the embodiments, the difference between the sublimation temperature of the organic compound H1 and the sublimation temperature of the organic compound H2 is less than or equal to 25 K. In one of the embodiments, the difference between the sublimation temperature of the organic compound H1 and the sublimation temperature of the organic compound H2 is less than or equal to 20 K. In one of the embodiments, the difference between the sublimation temperature of the organic compound H1 and the sublimation temperature of the organic compound H2 is less than or equal to 18 K. In one of the embodiments, the difference between the sublimation temperature of the organic compound H1 and the sublimation temperature of the organic compound H2 is less than or equal to 15 K.

It should be noted that, the solubility of the organic small molecule compound is ensured by the substituent R on the units of the general formulas (1) to (11) and optionally on units which are present additionally, and by adjusting the linking position between the core structure and the substituents. These substituents may also promote the solubility if other substituents are present. The structural units of the general formulas (1) to (11) are suitable for various functions in the organic small molecule compounds depending on the type of substitution. Therefore, they can be used as the main backbone of the small molecule compound.

In one of the embodiments, according to the general formulas (4) to (13), the organic compound H2 is selected from the group consisting of compounds represented by the following structures. These structures can be substituted at all possible substitution positions.

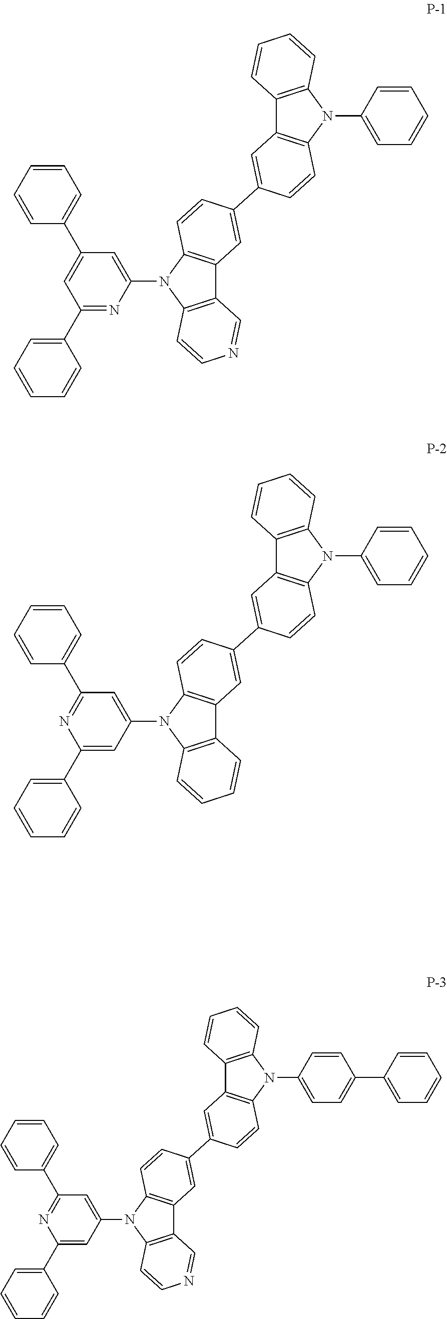

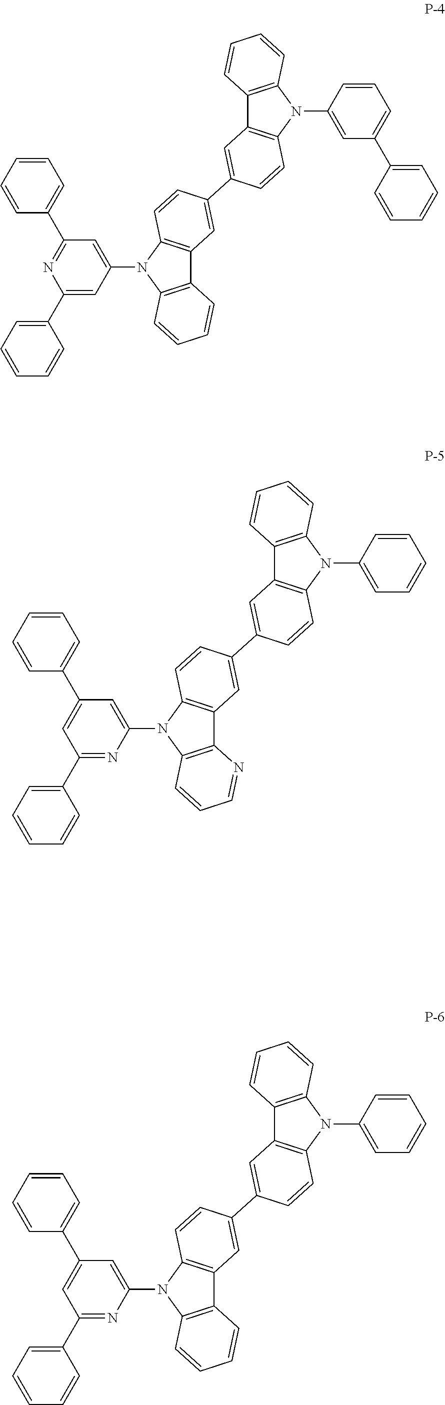

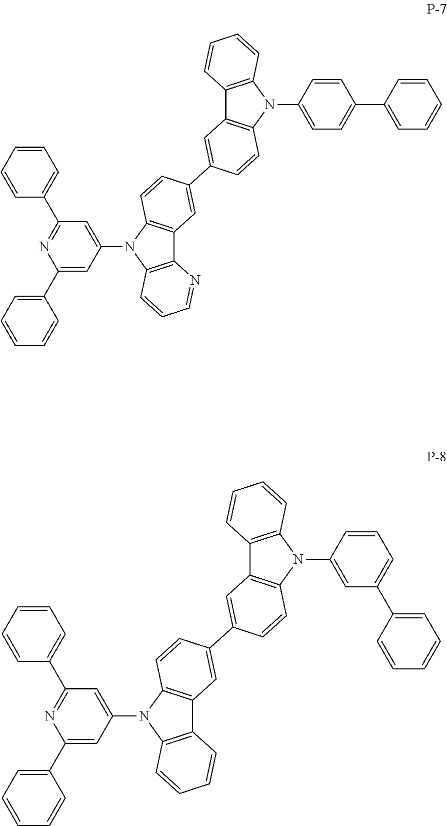

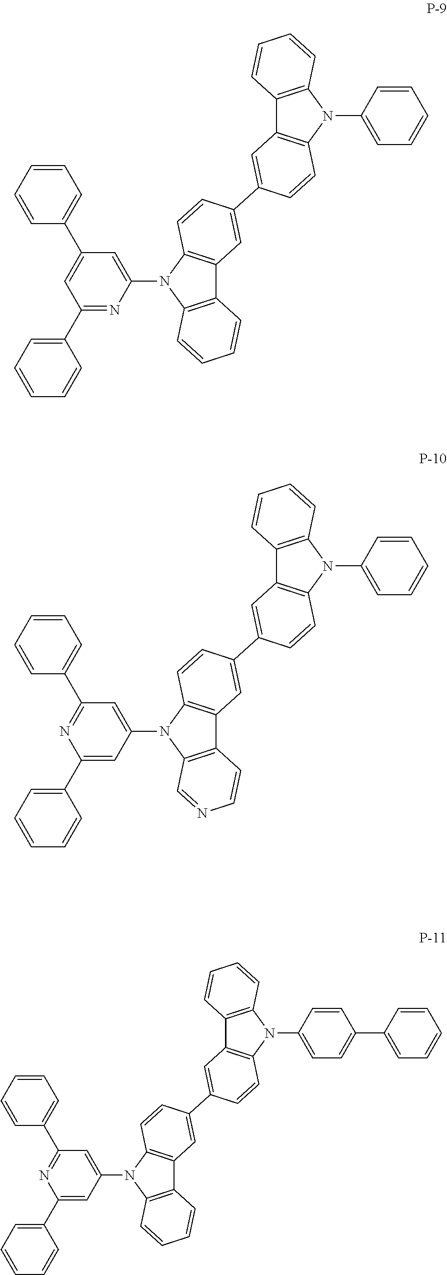

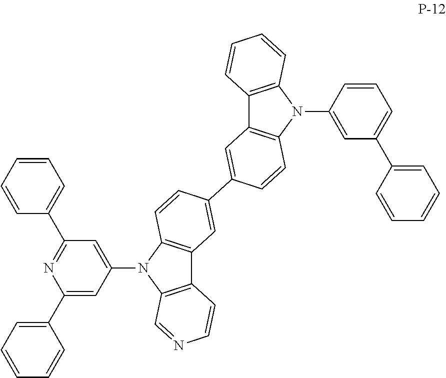

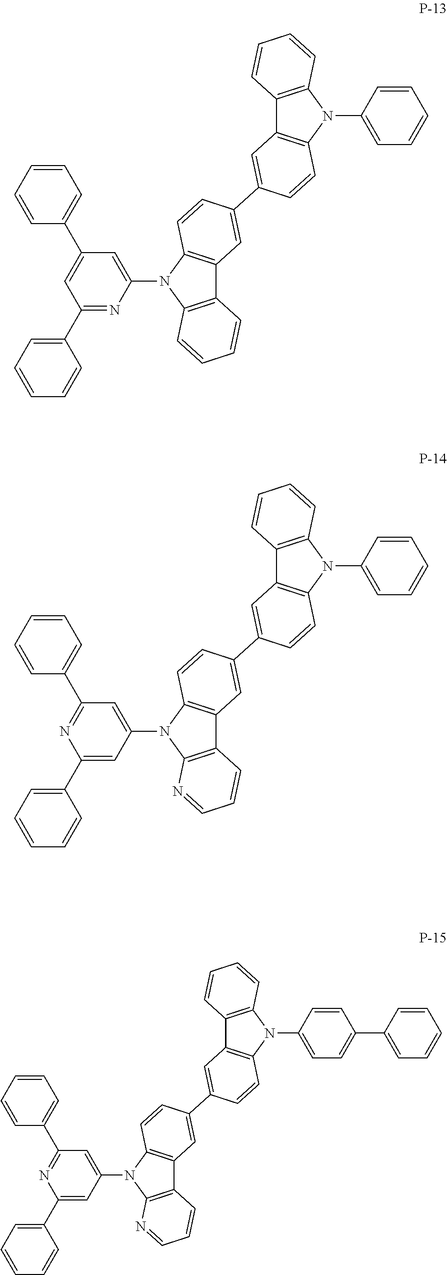

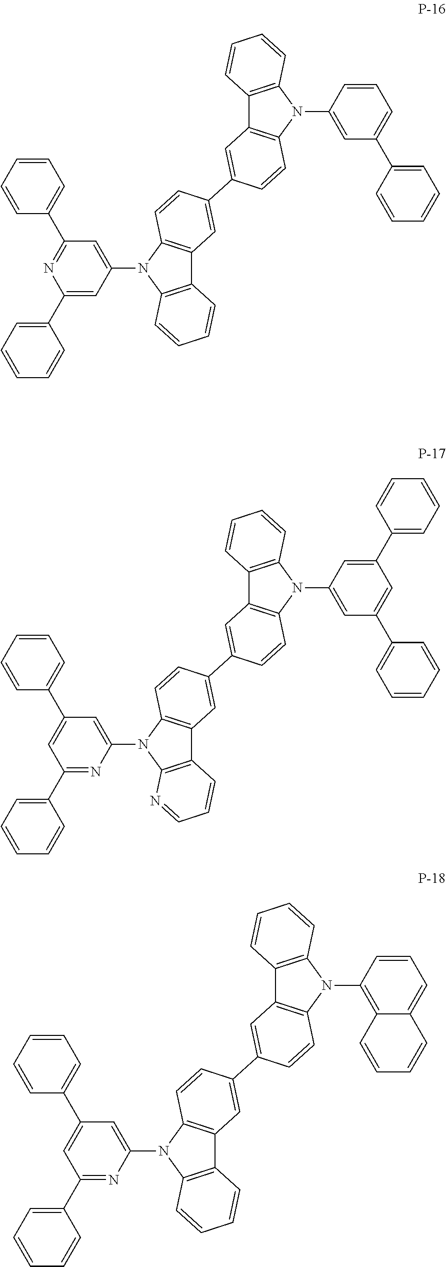

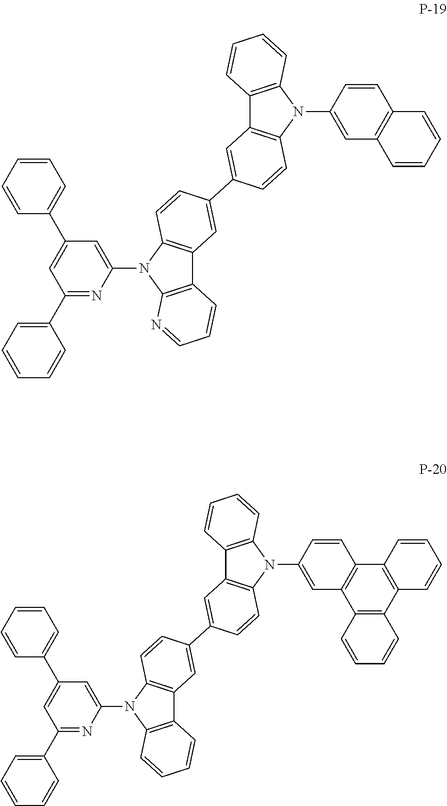

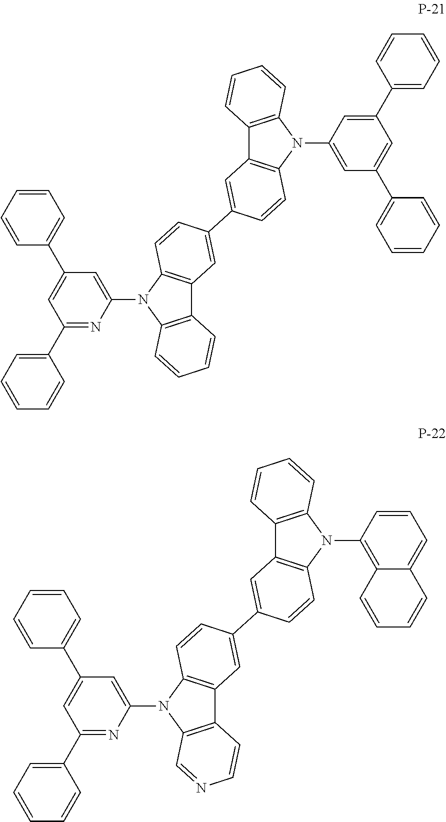

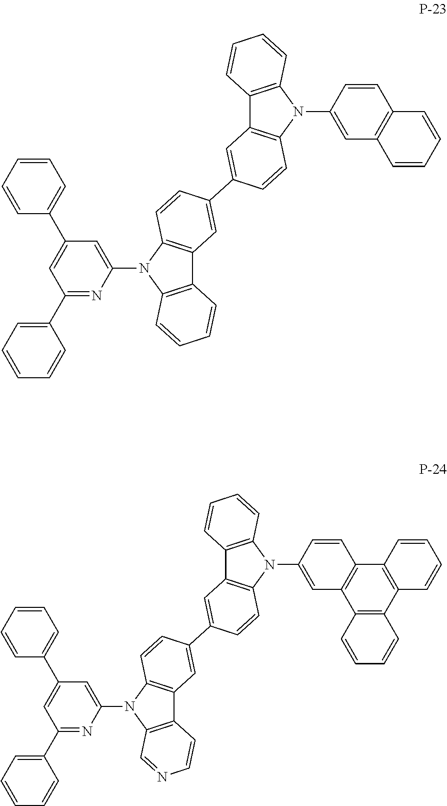

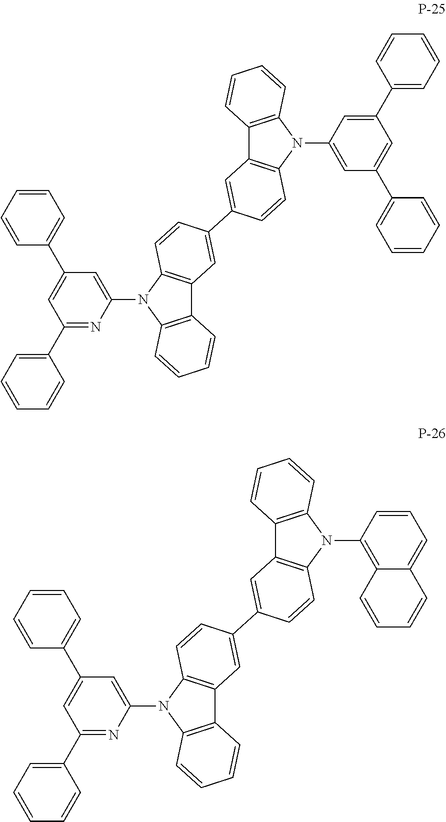

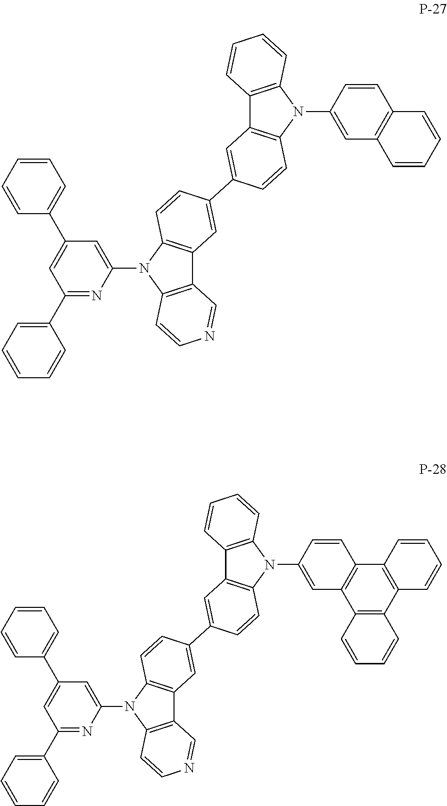

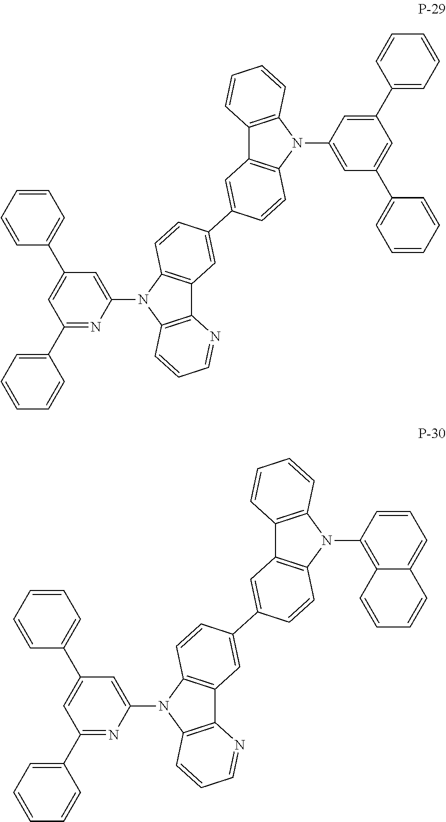

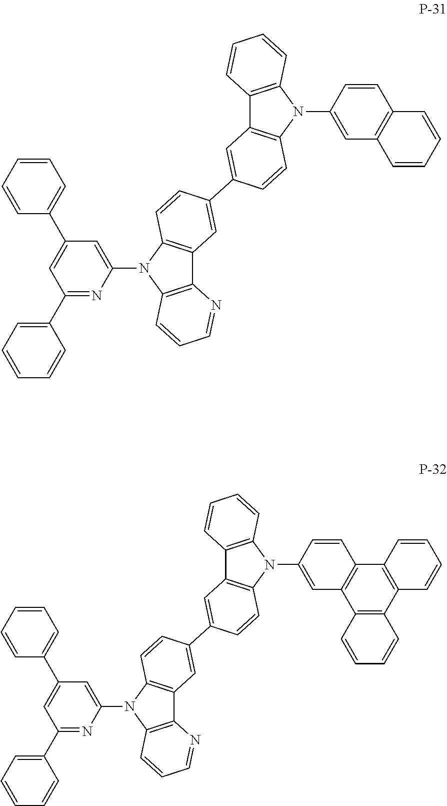

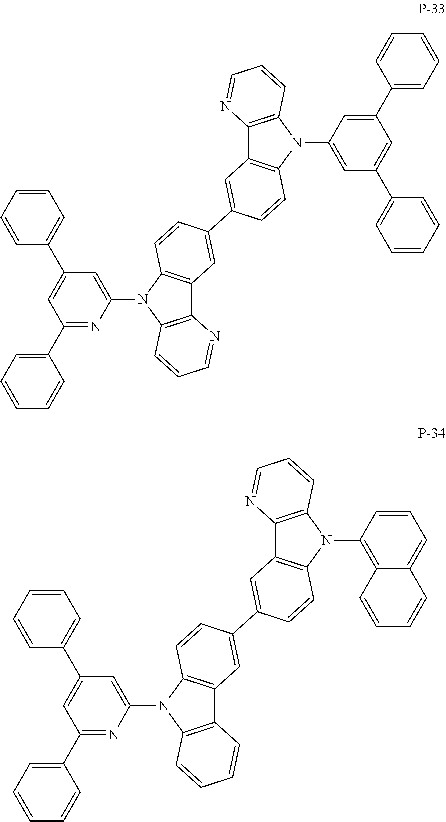

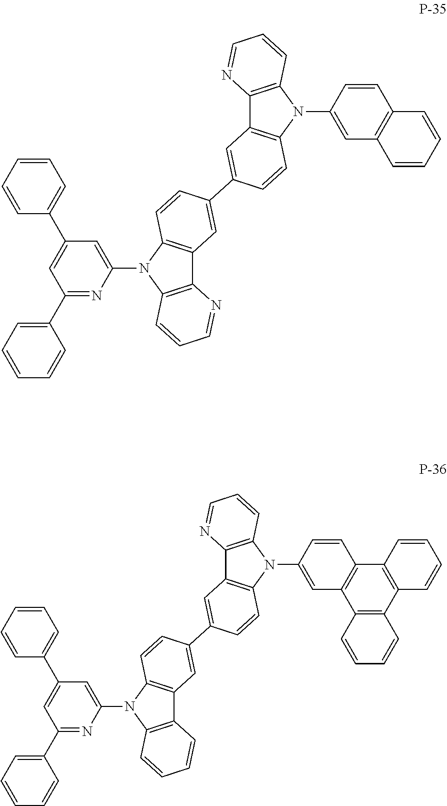

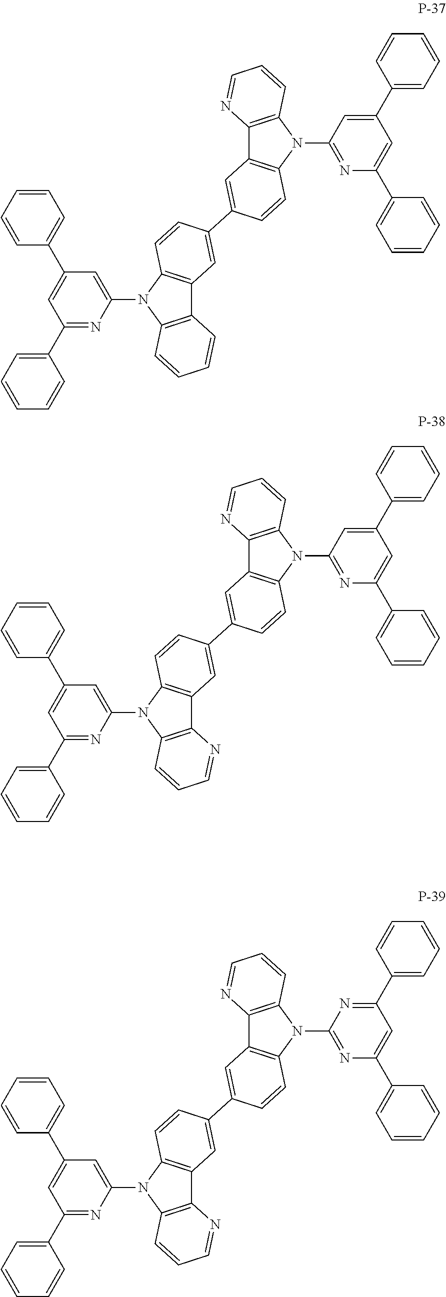

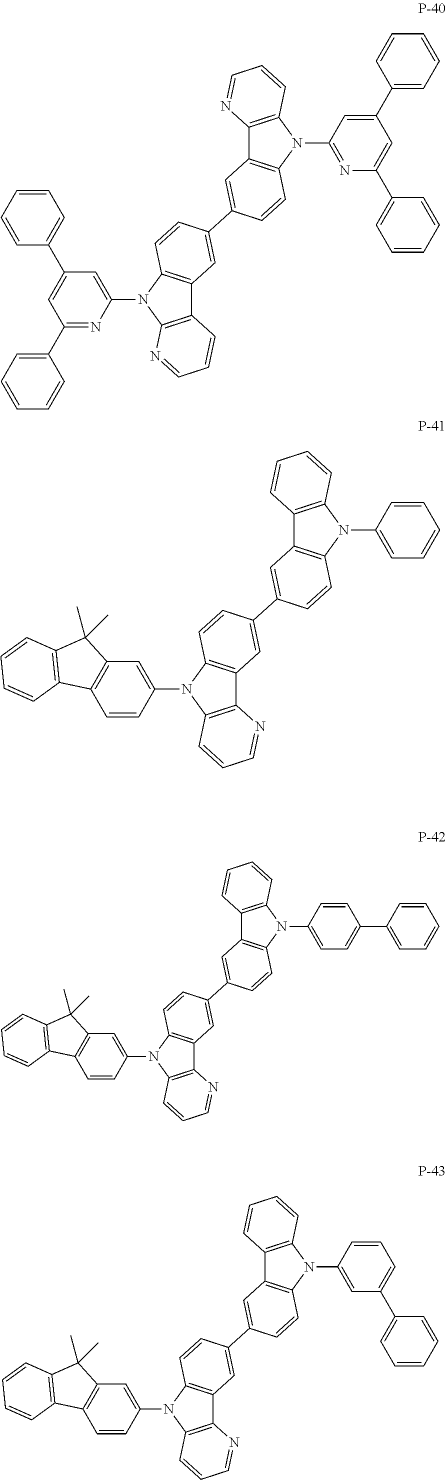

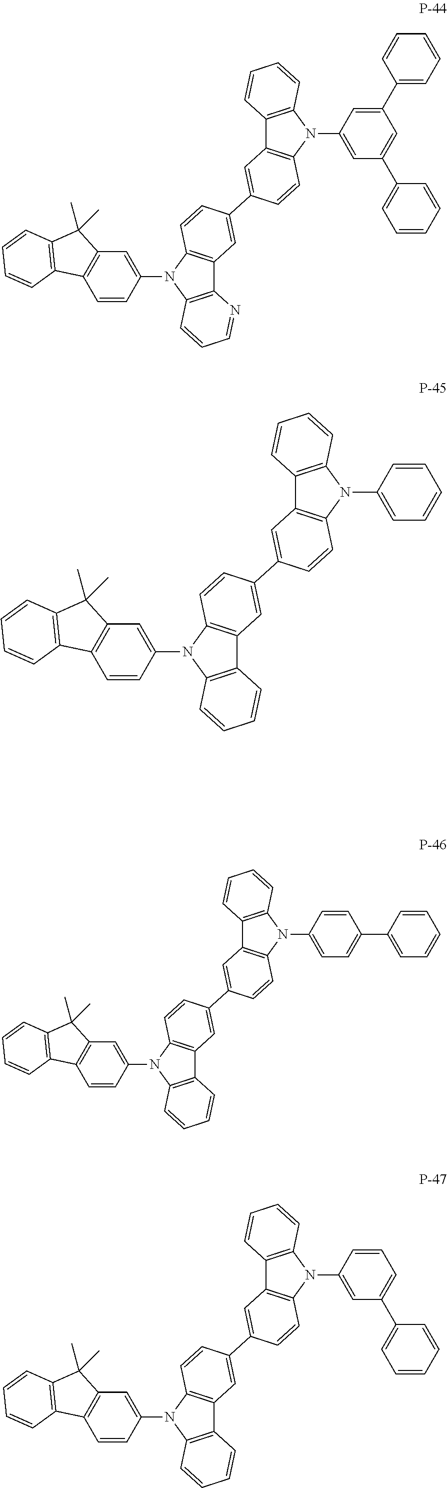

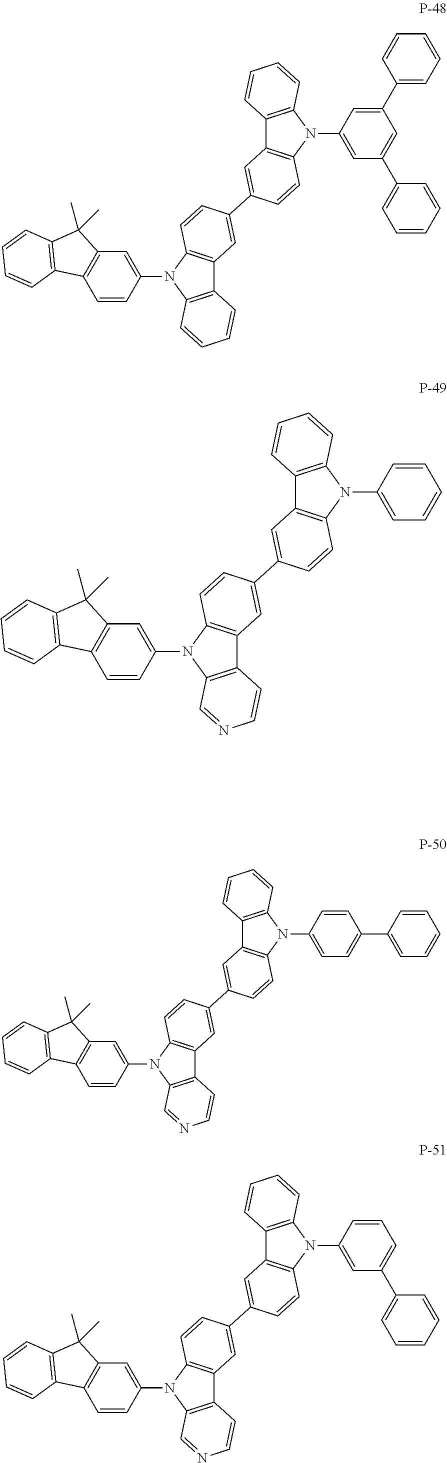

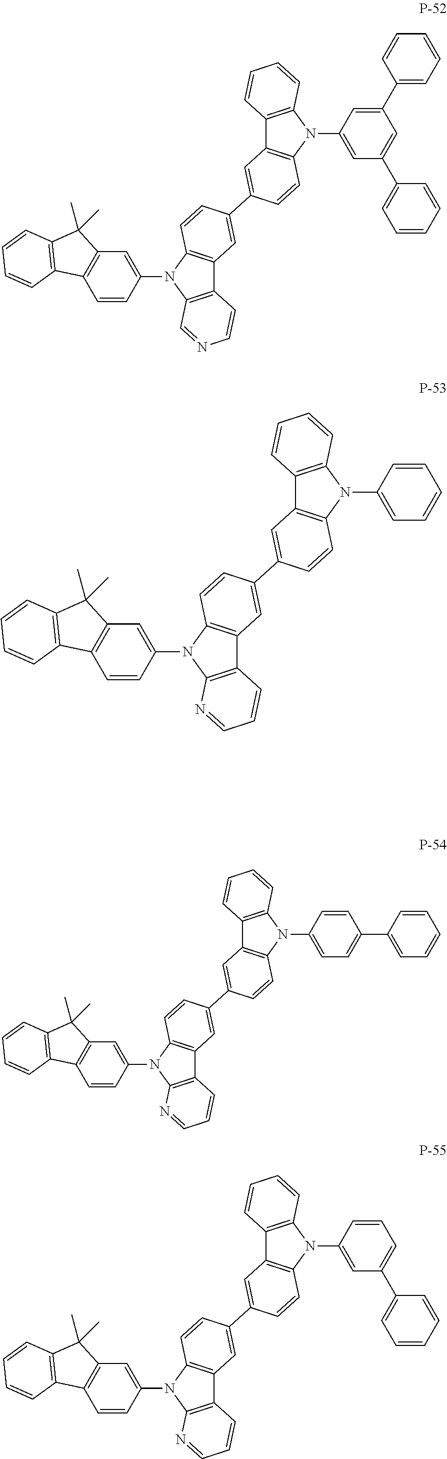

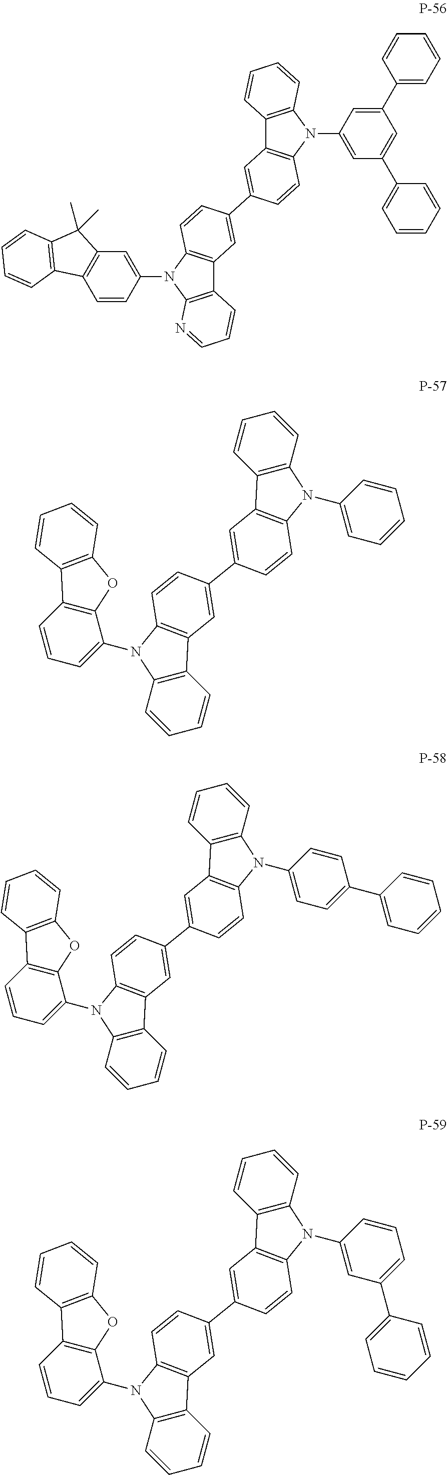

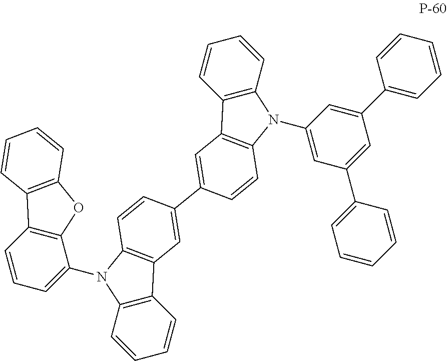

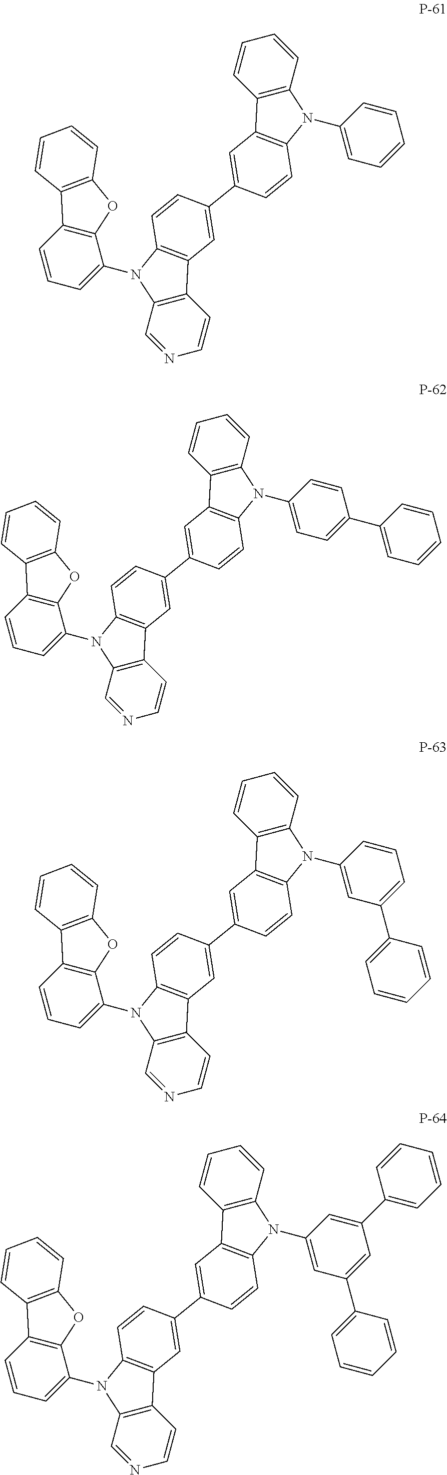

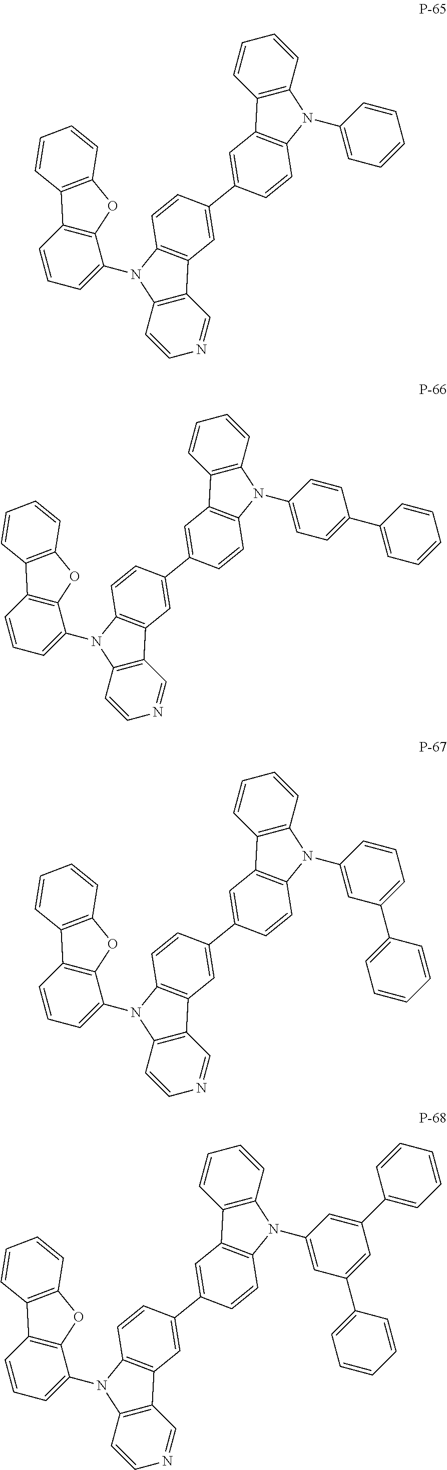

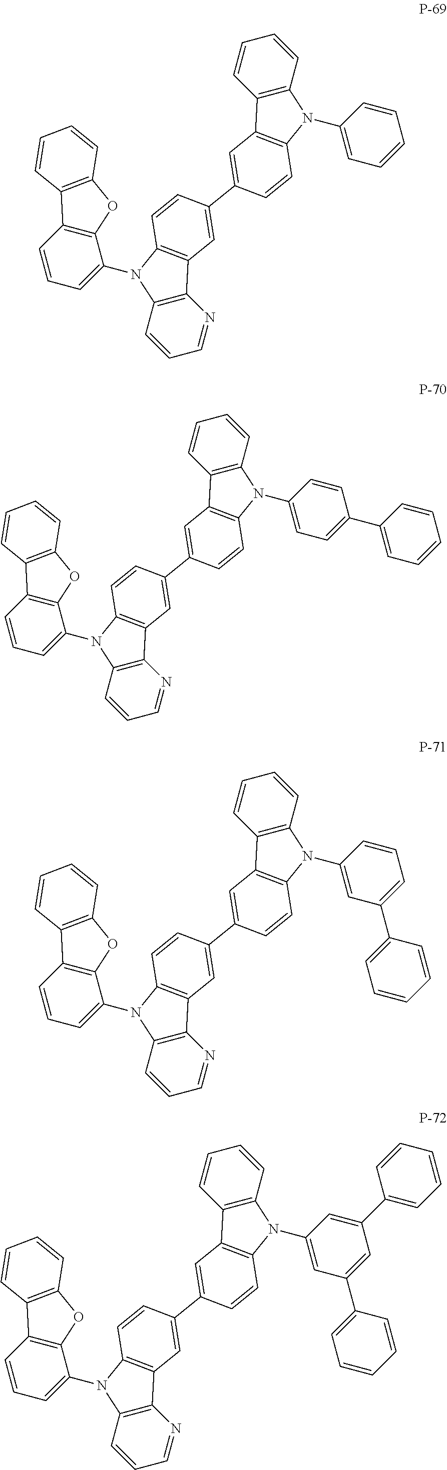

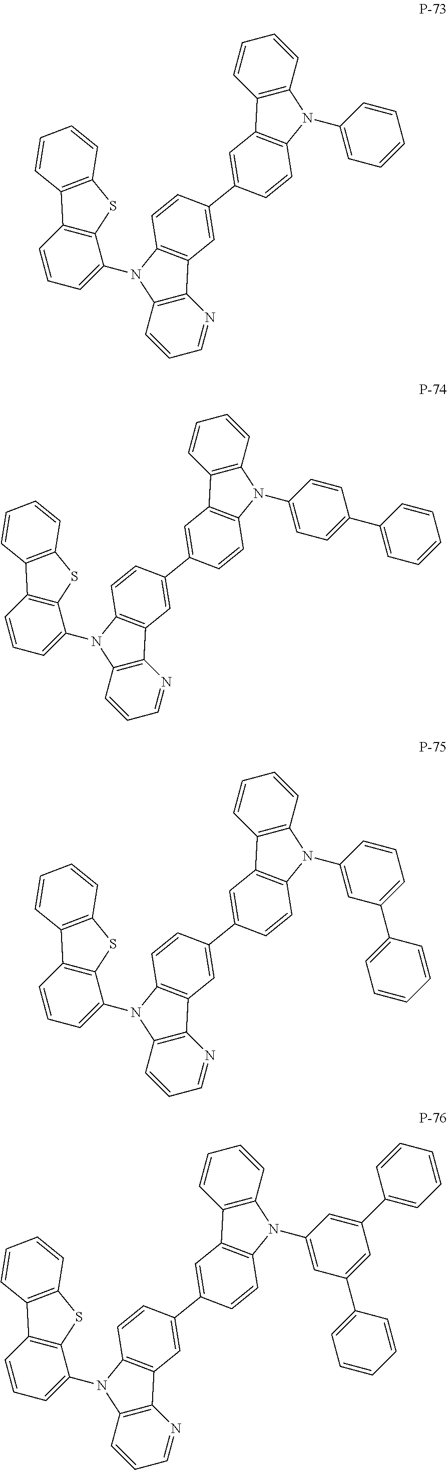

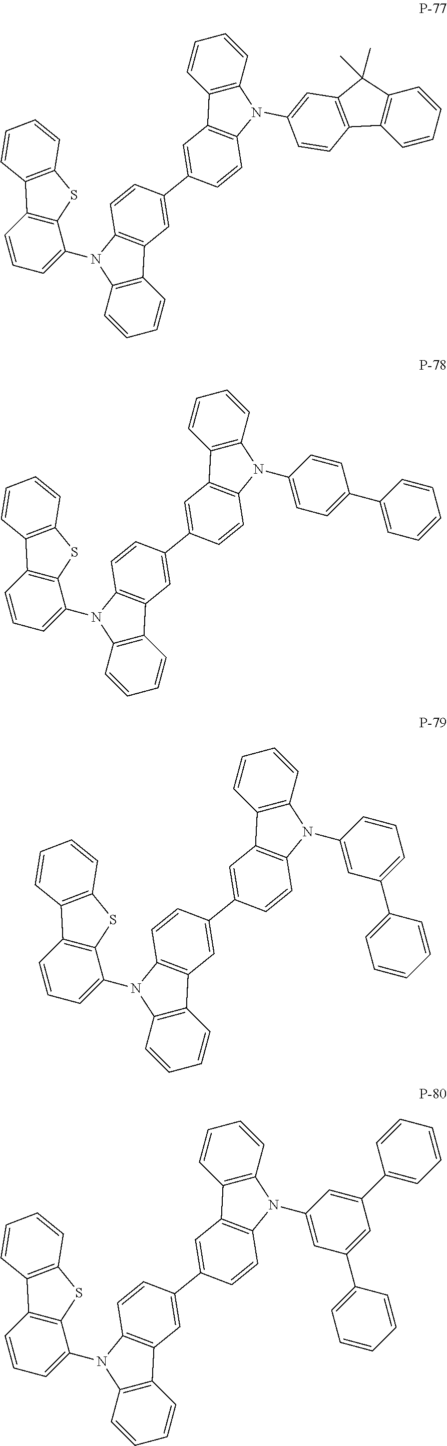

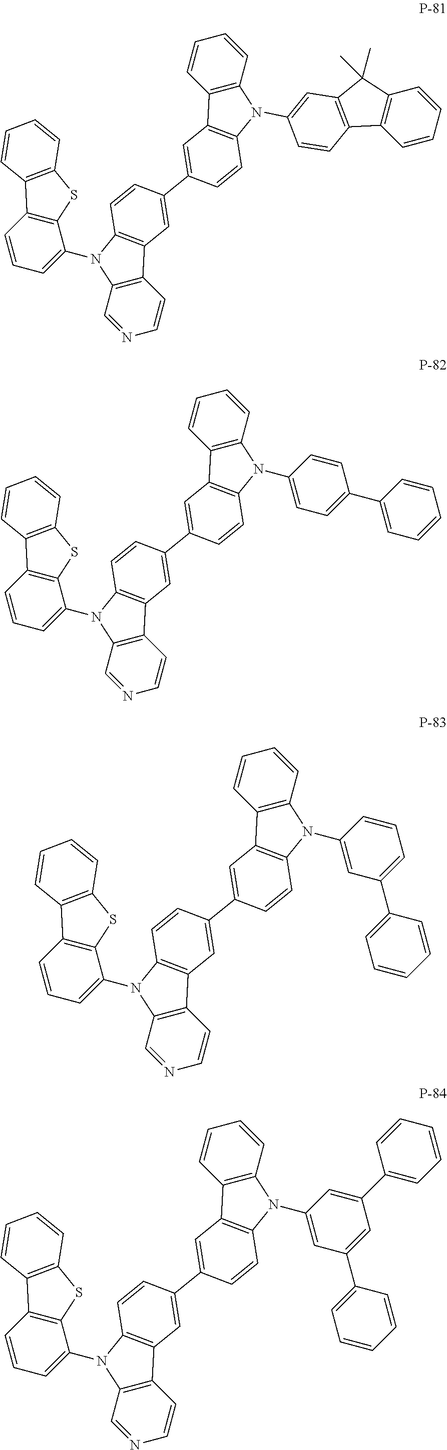

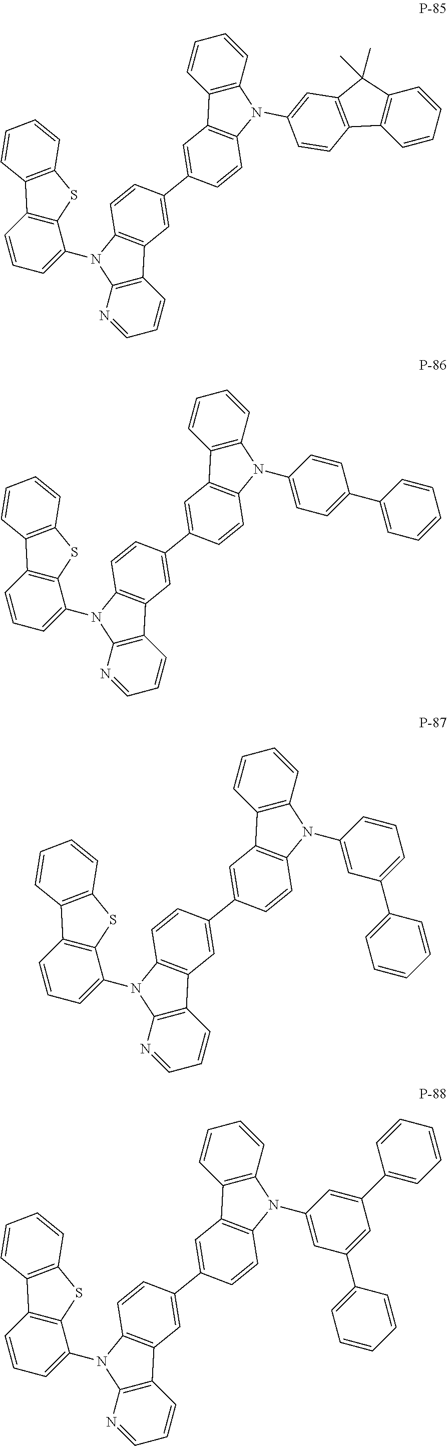

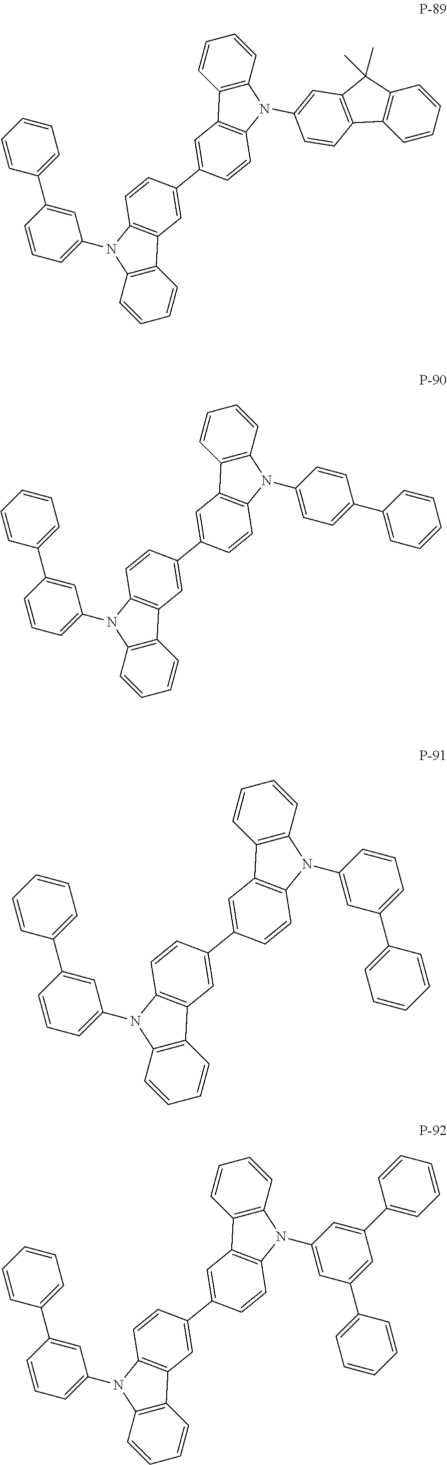

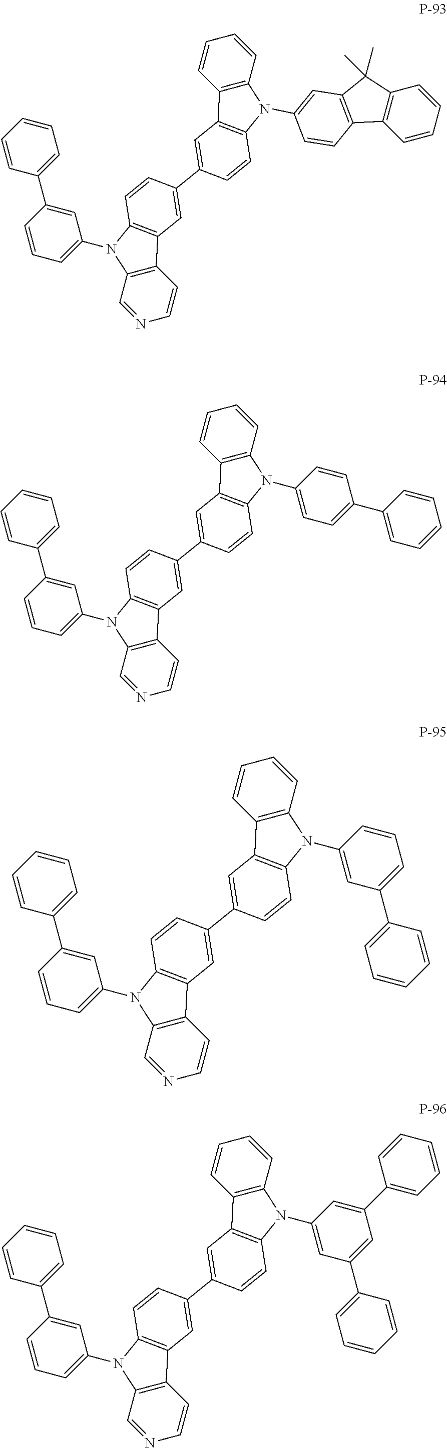

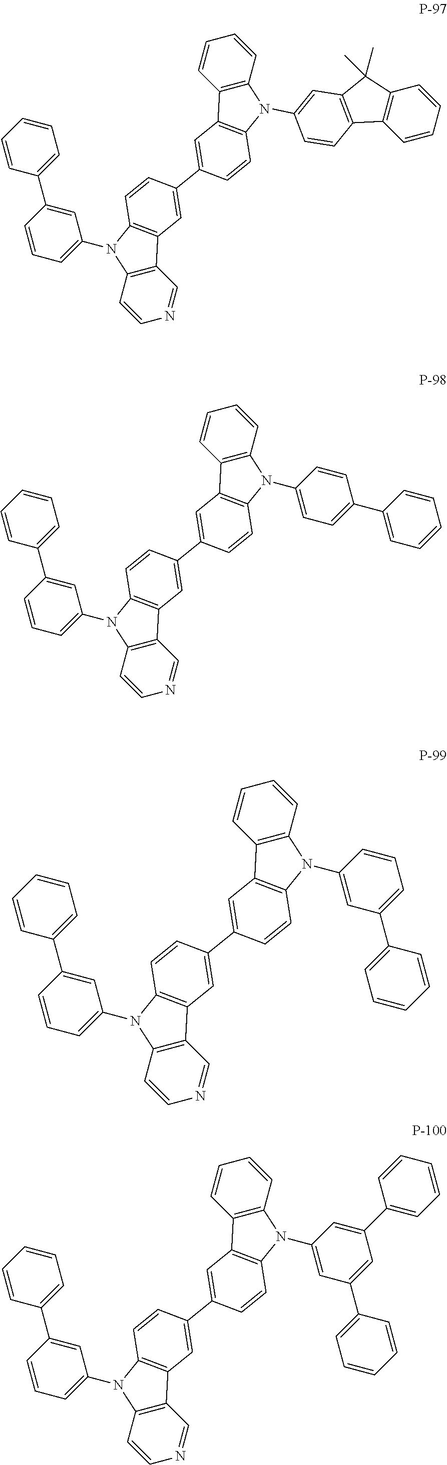

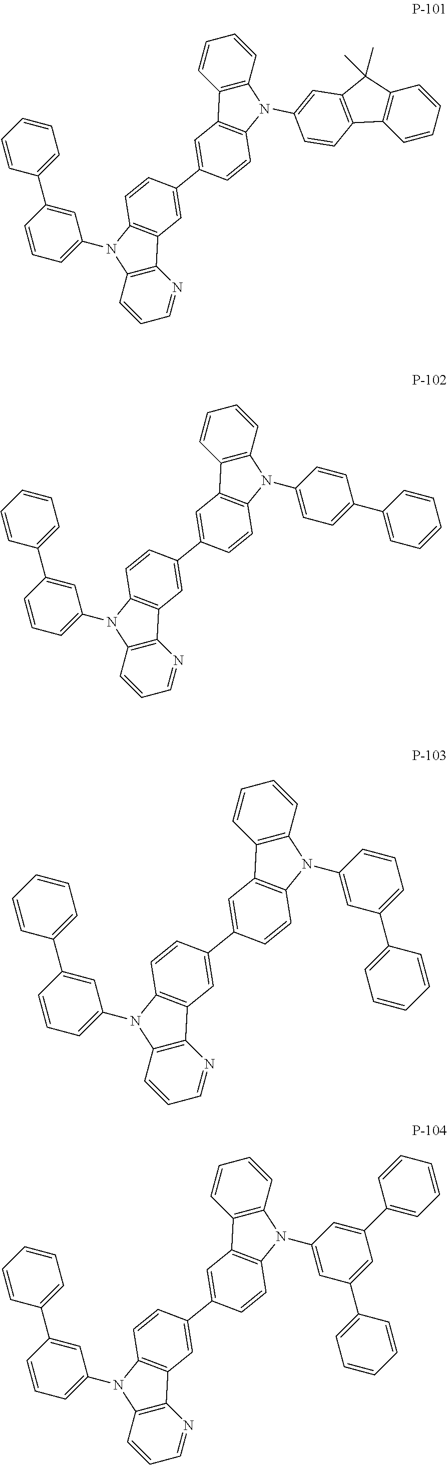

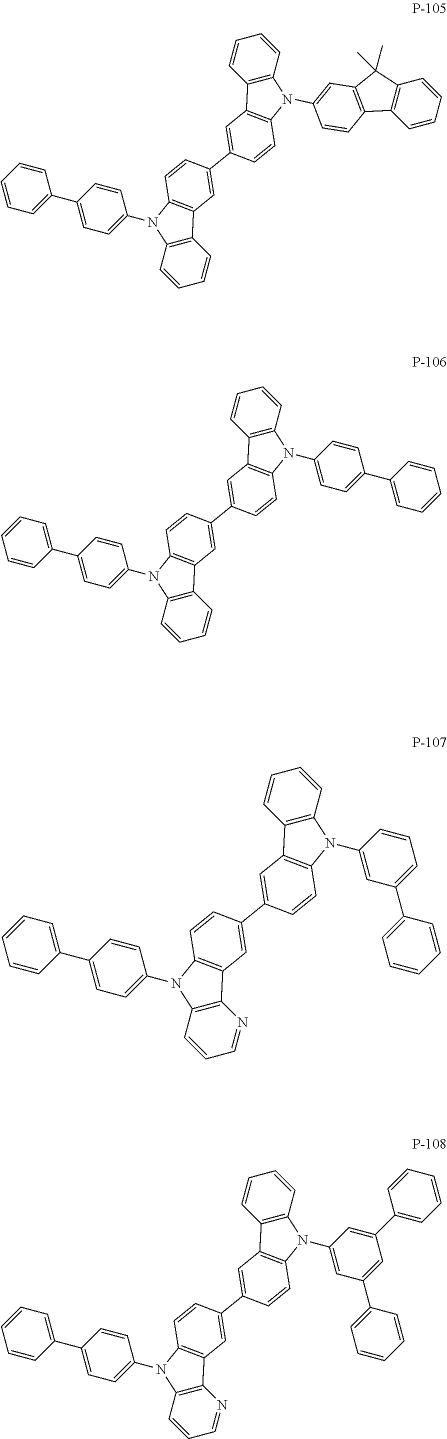

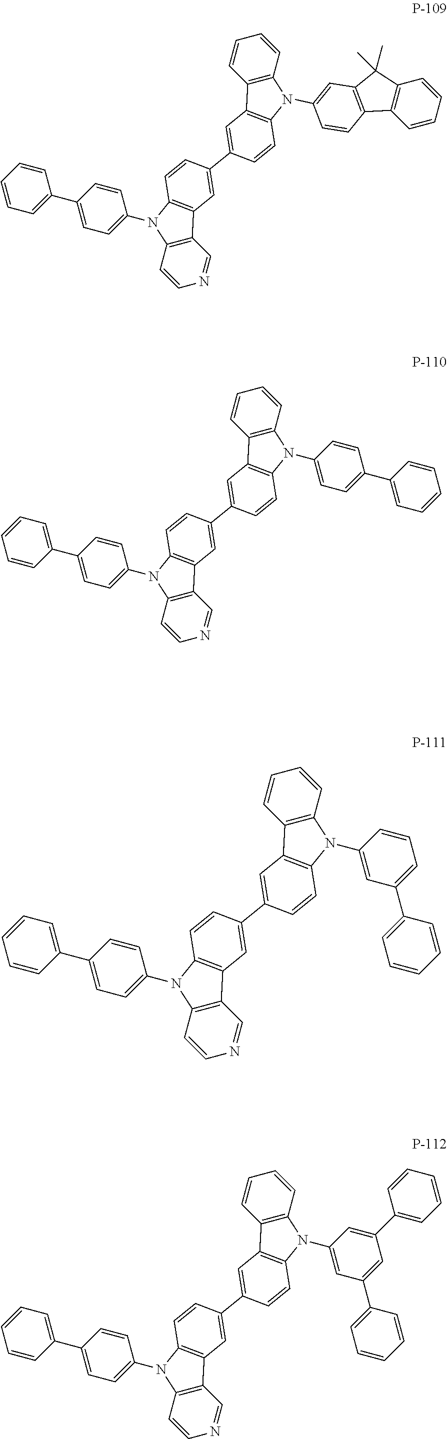

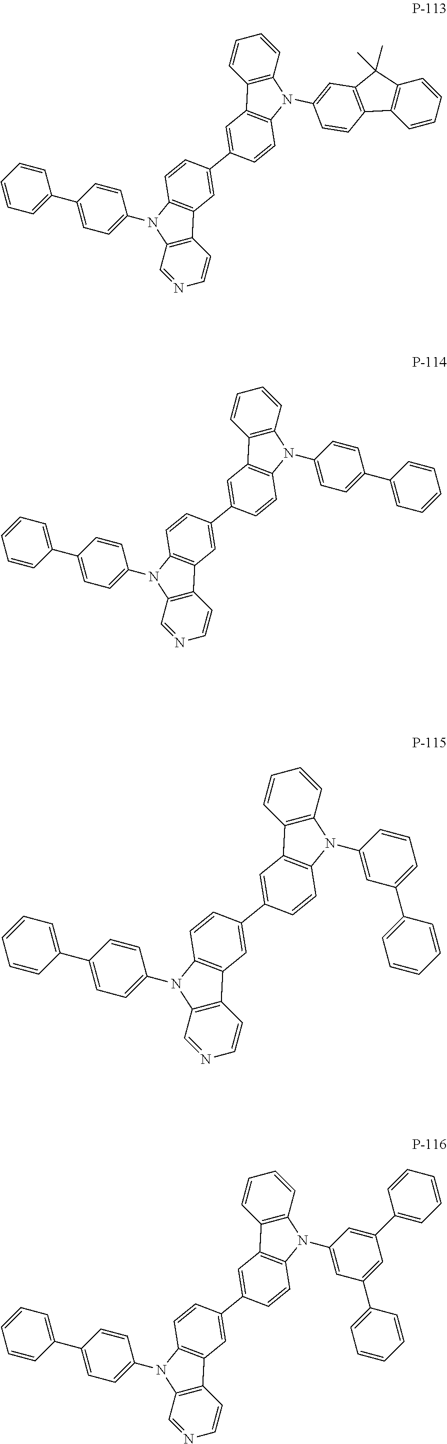

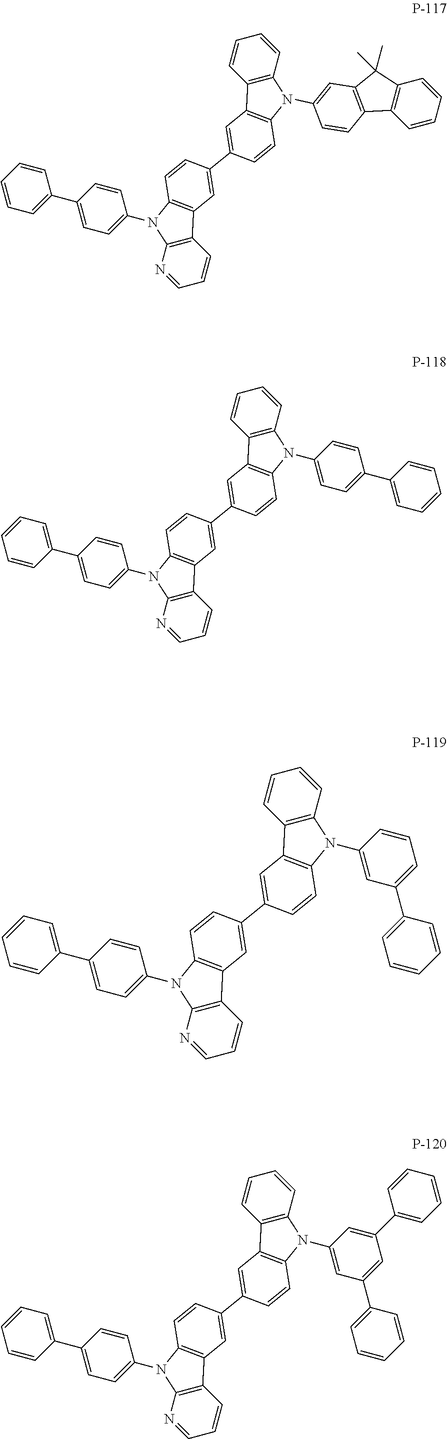

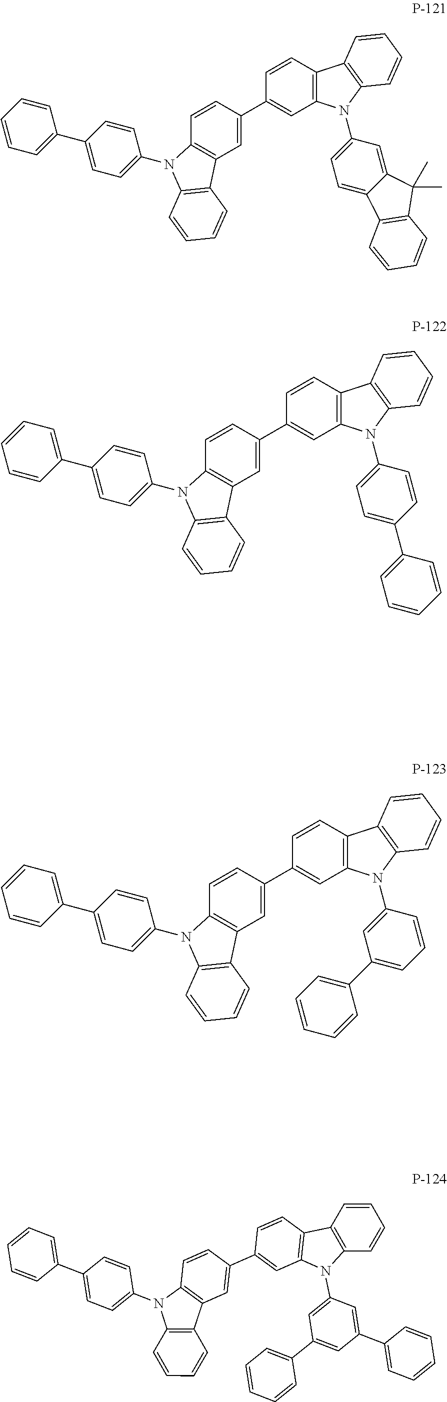

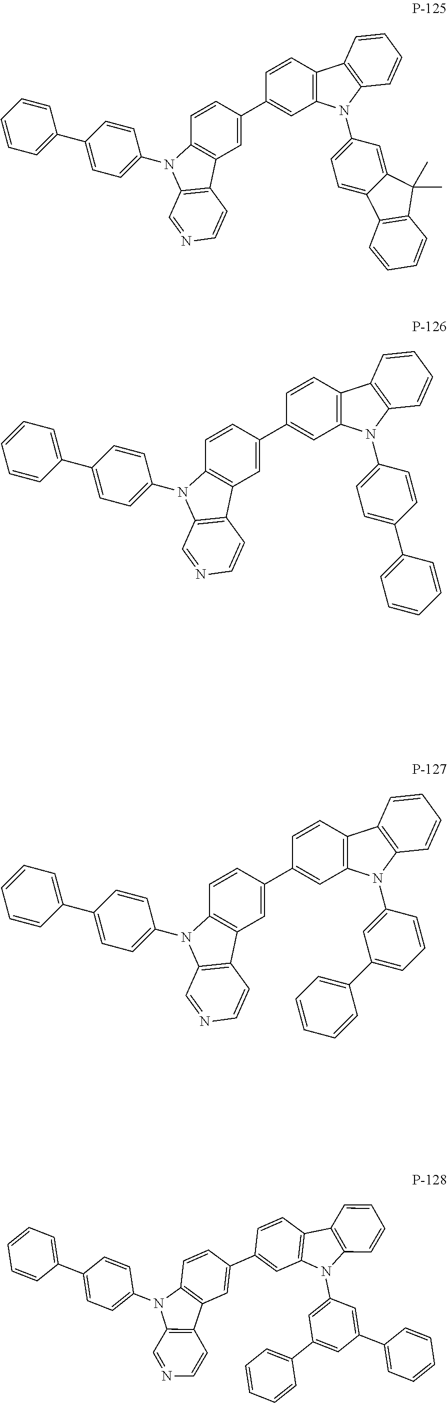

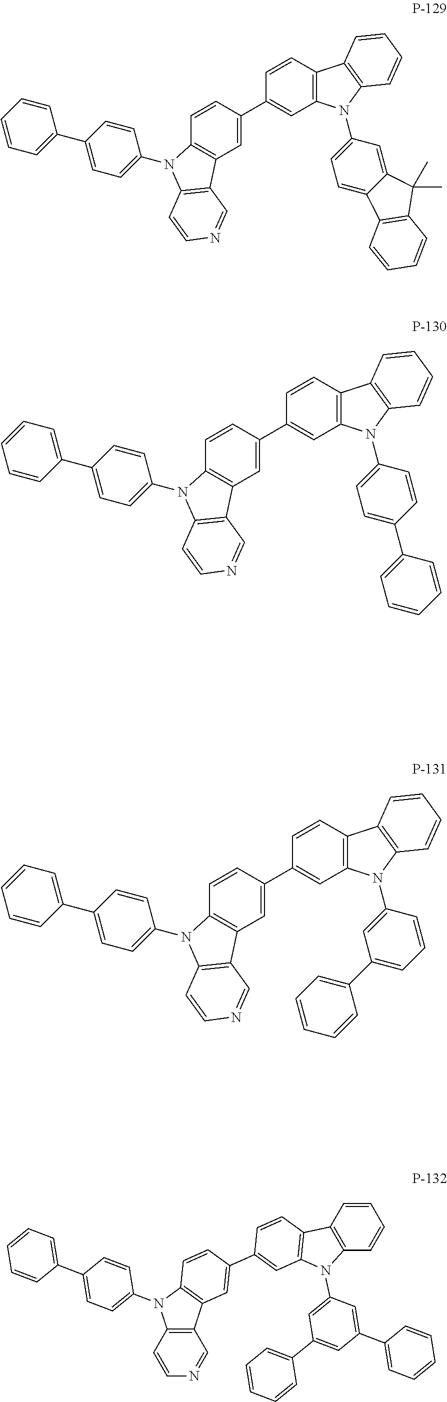

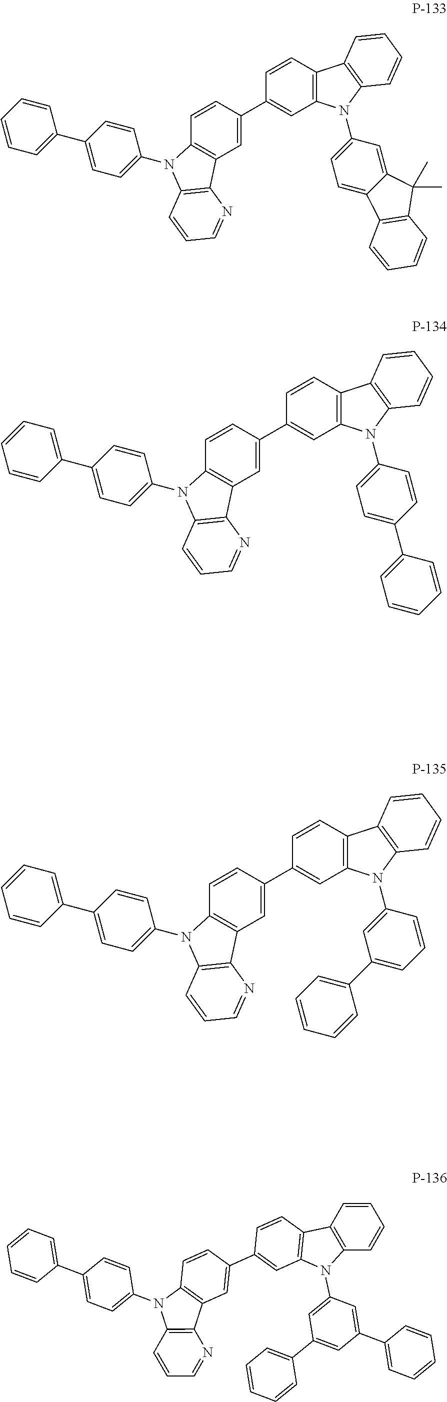

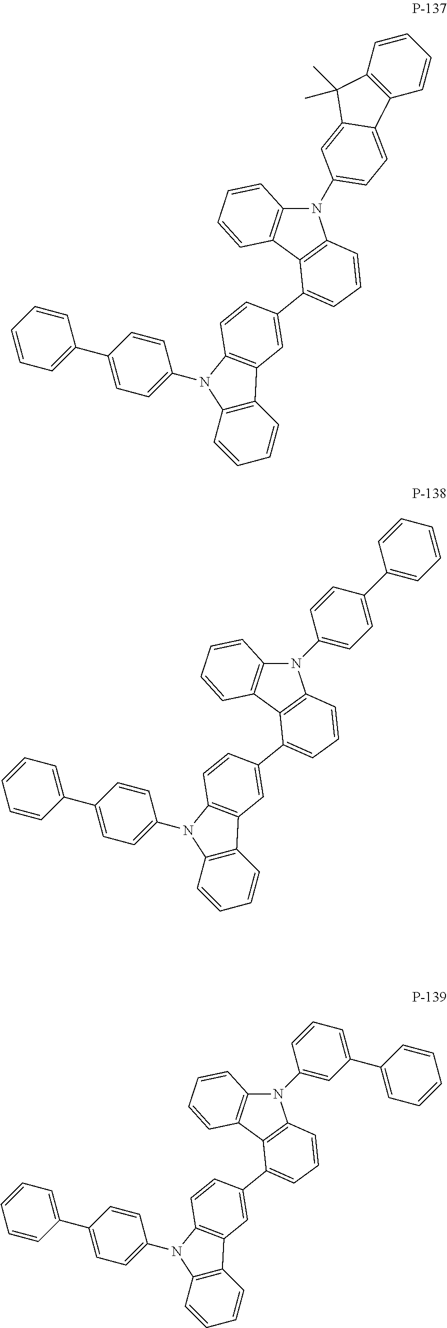

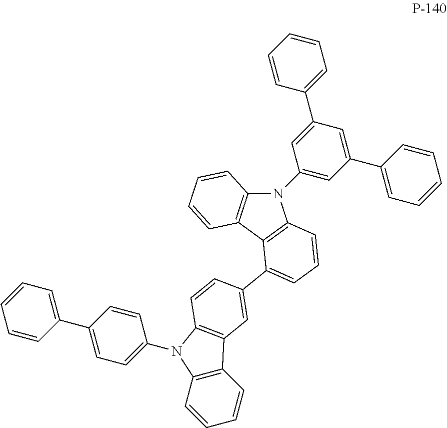

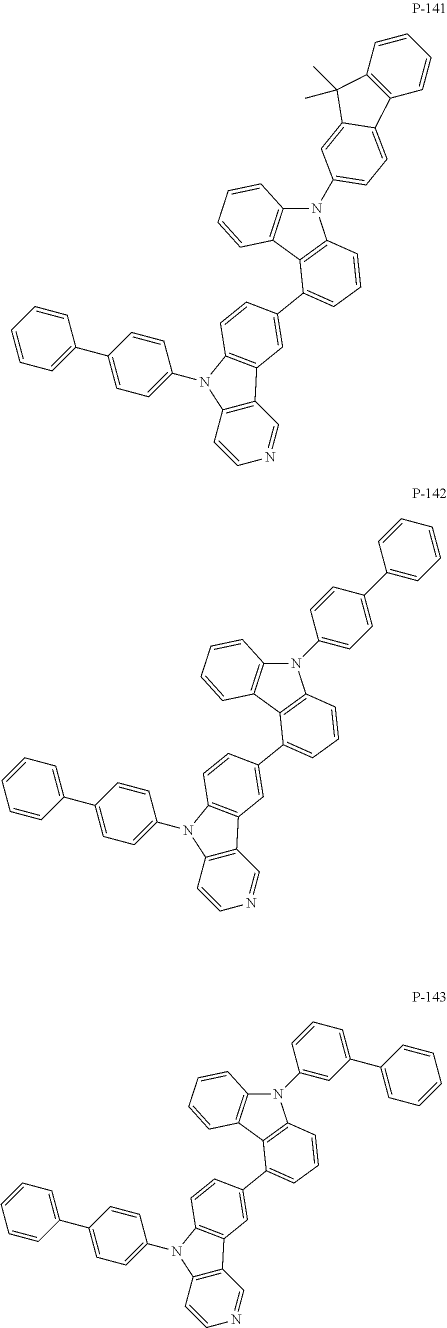

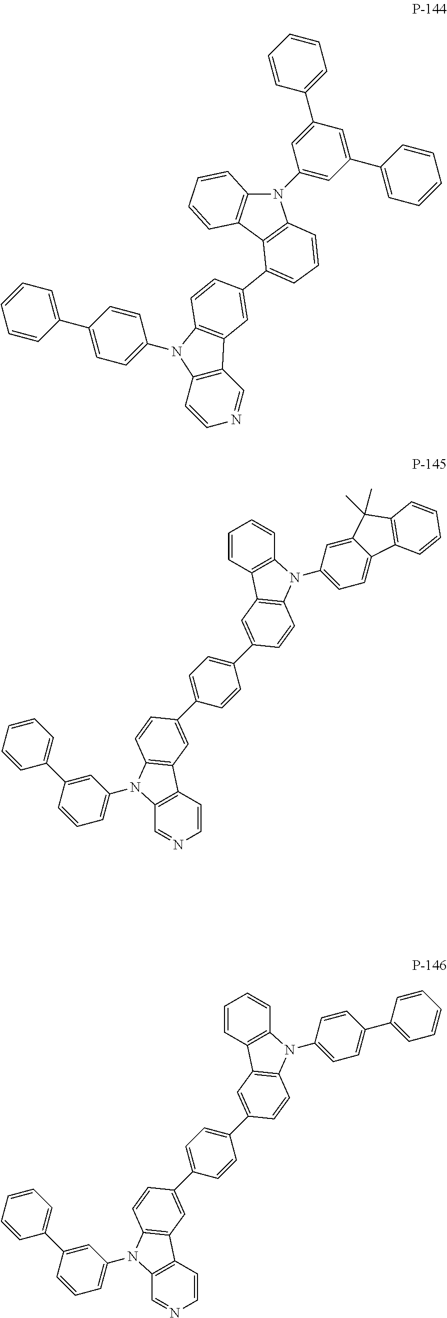

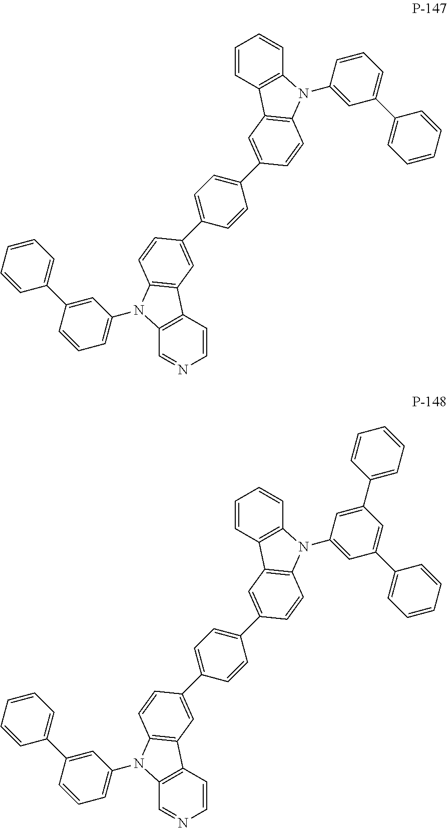

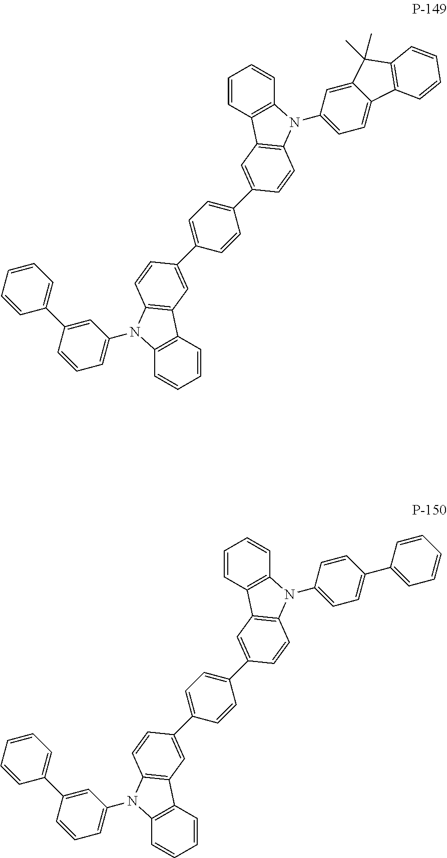

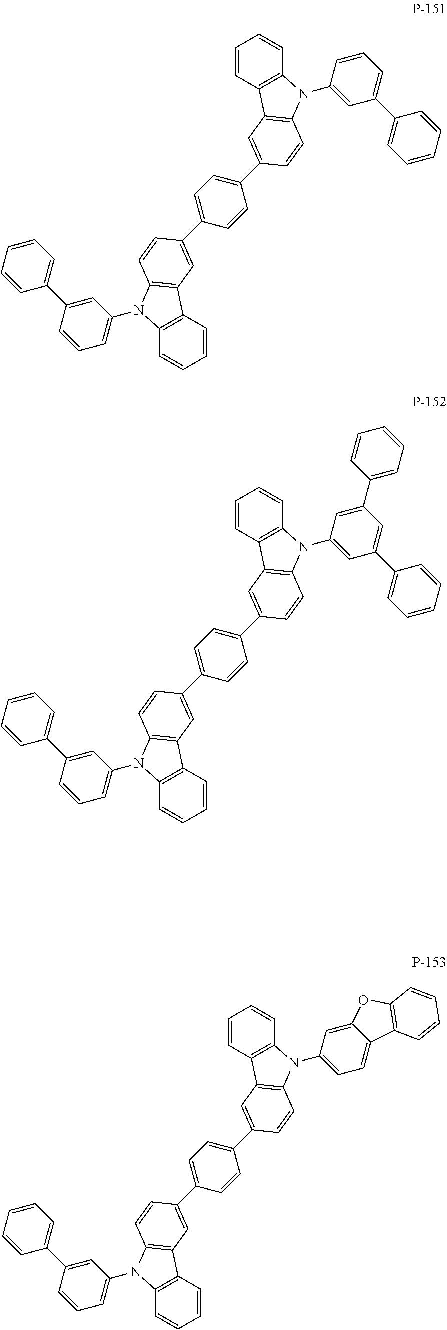

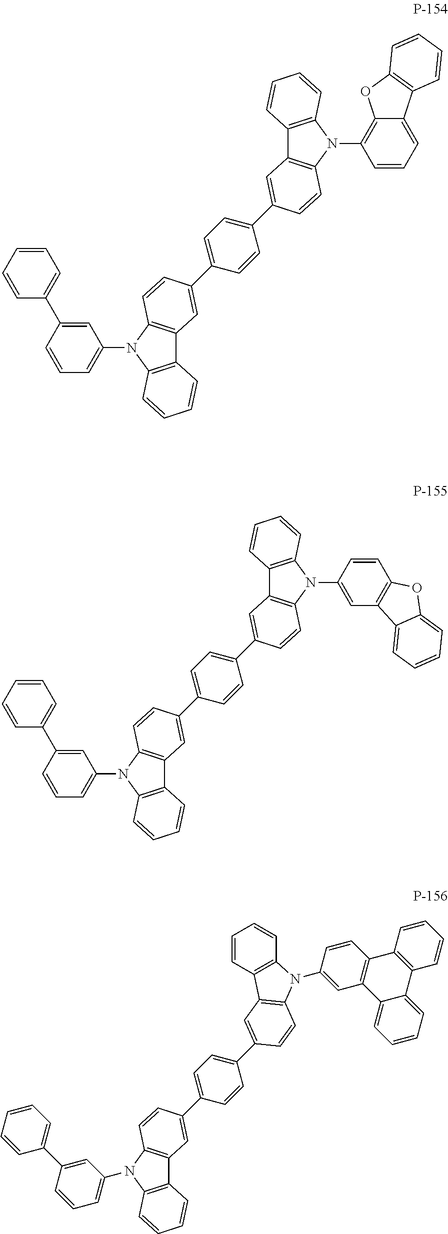

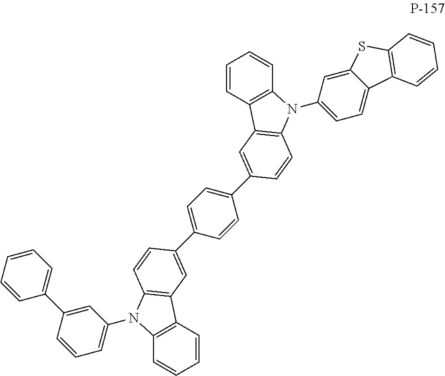

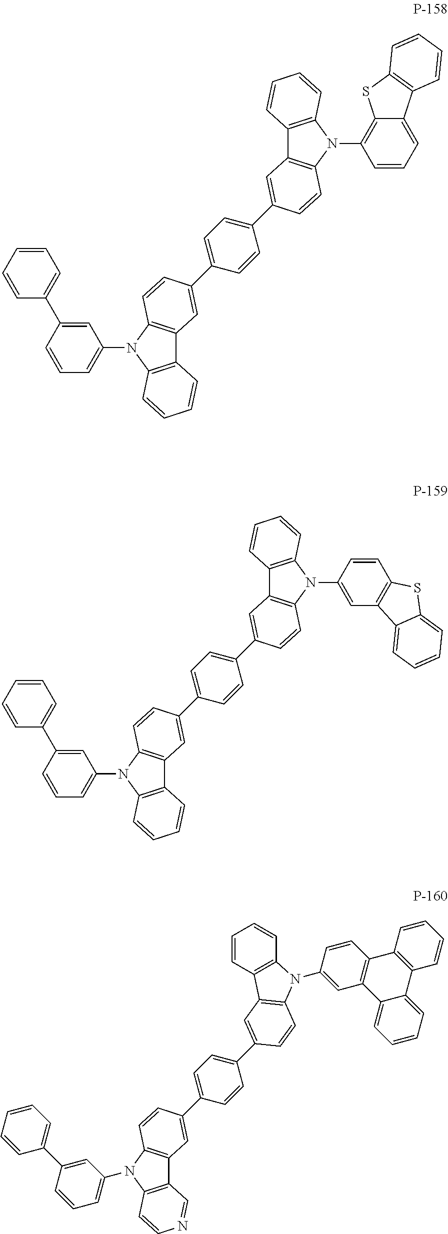

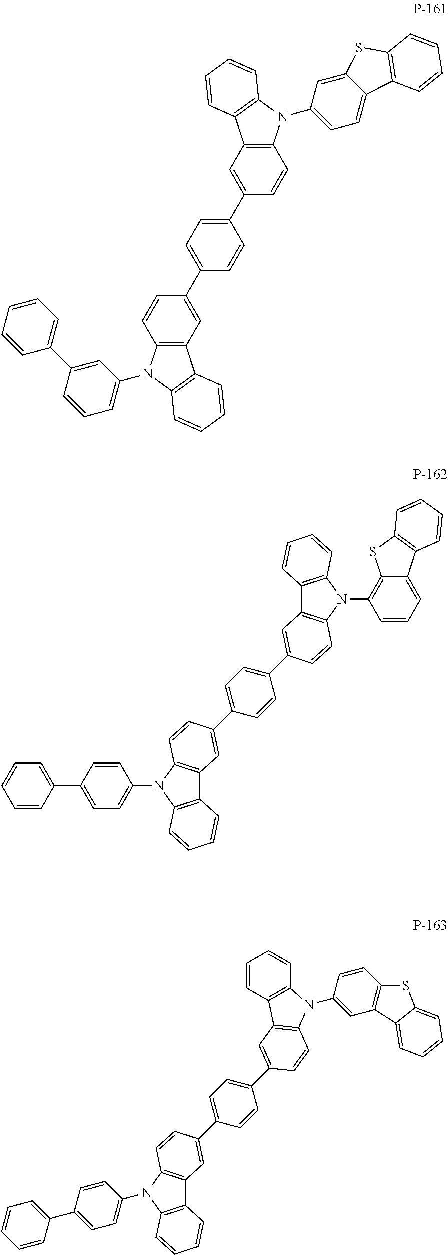

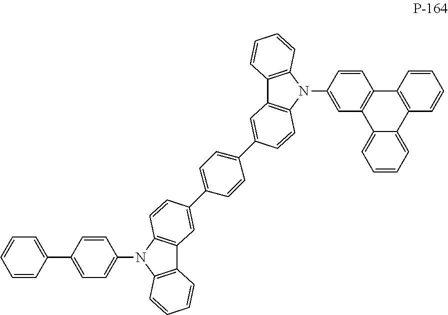

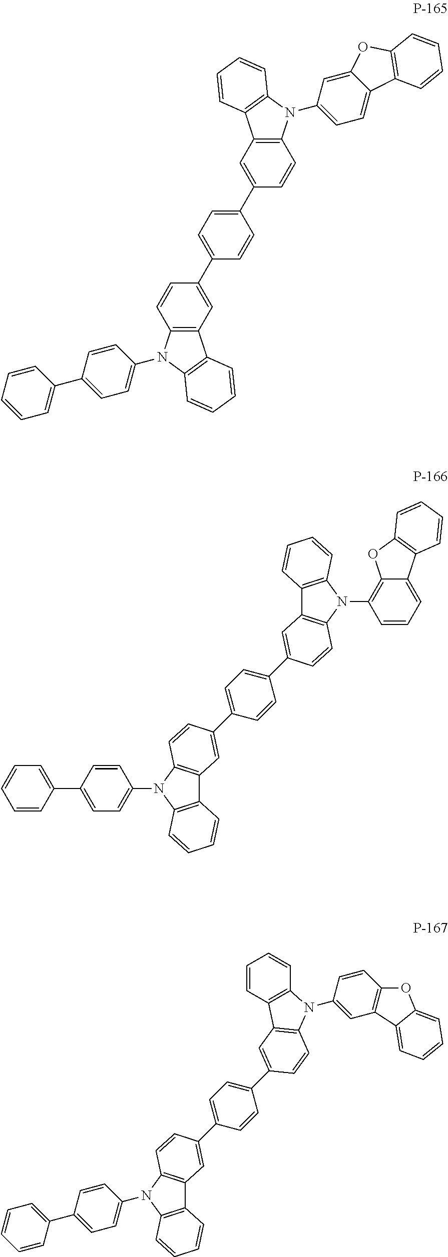

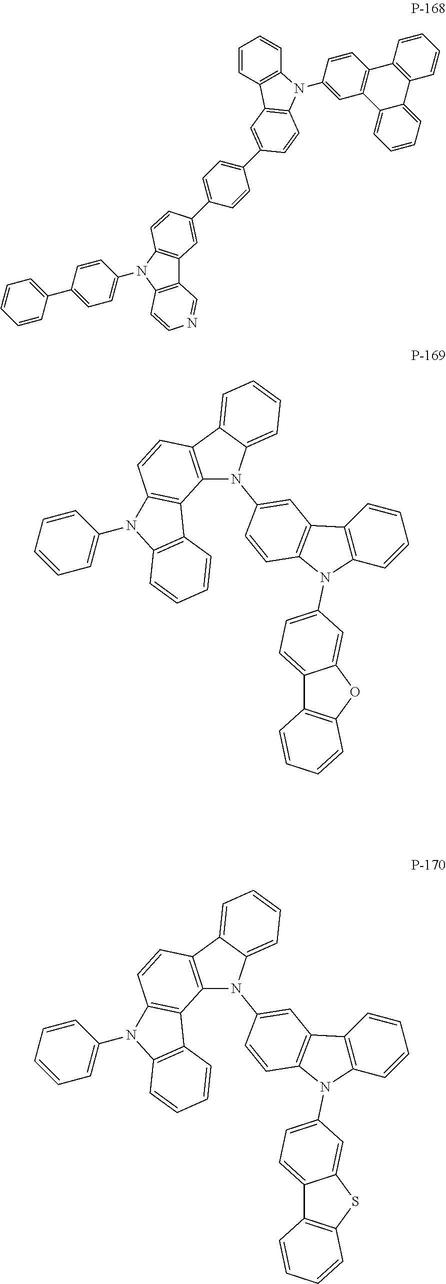

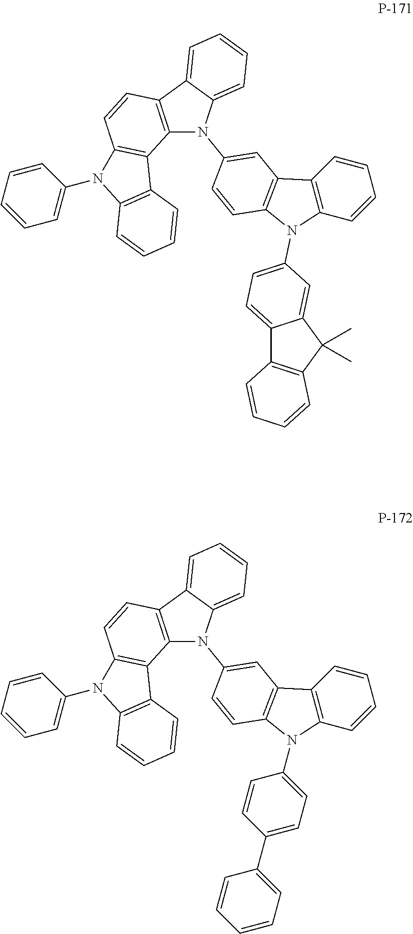

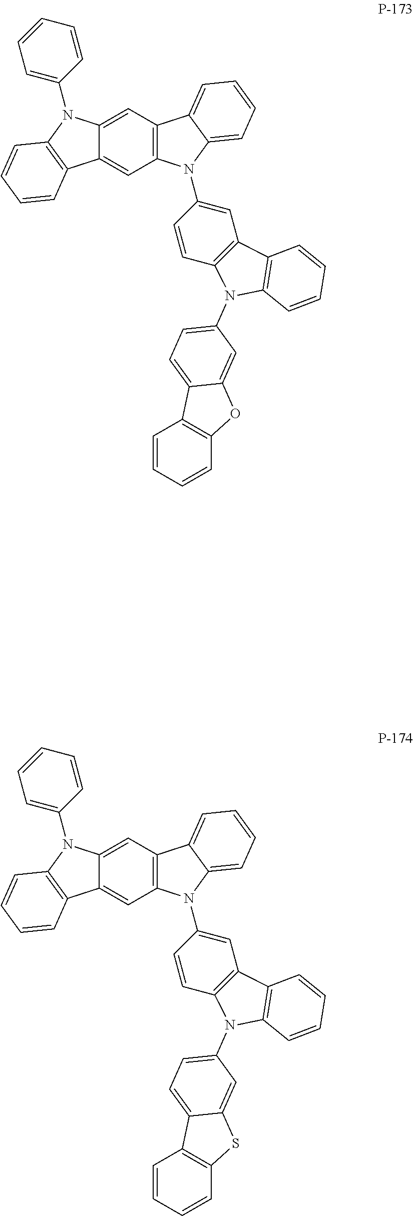

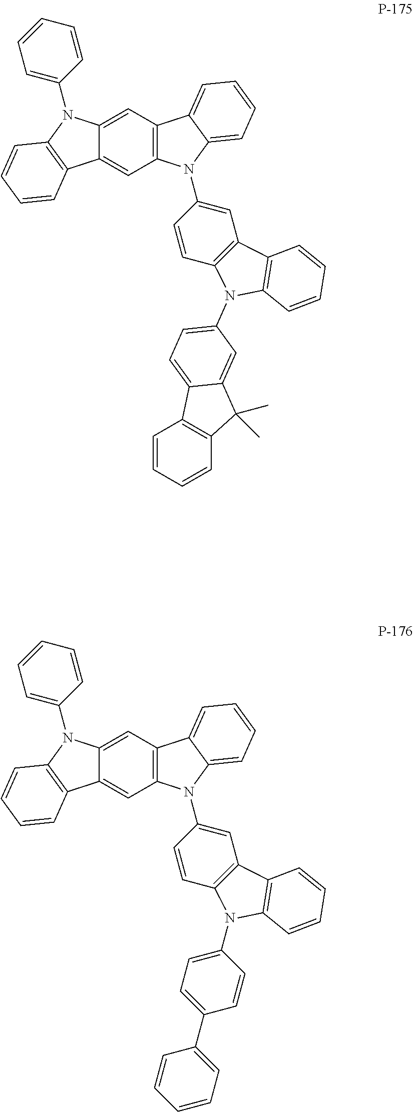

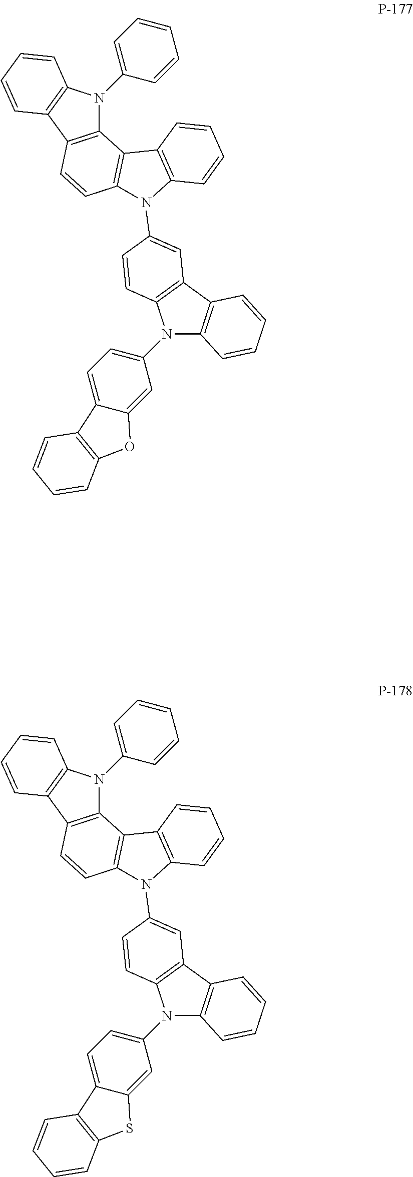

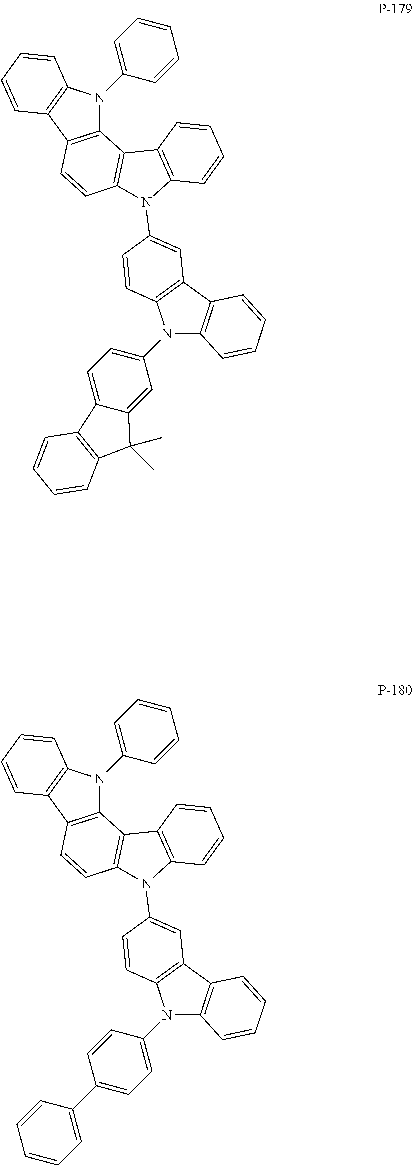

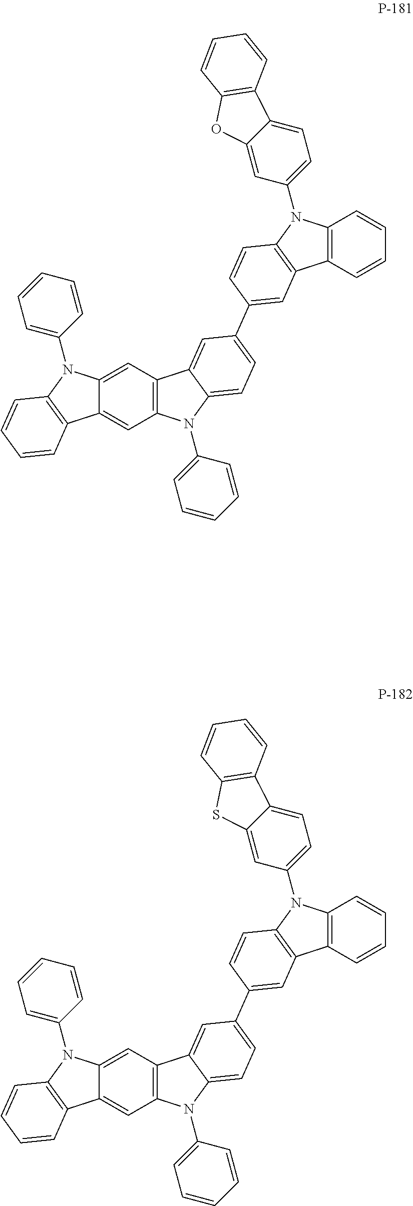

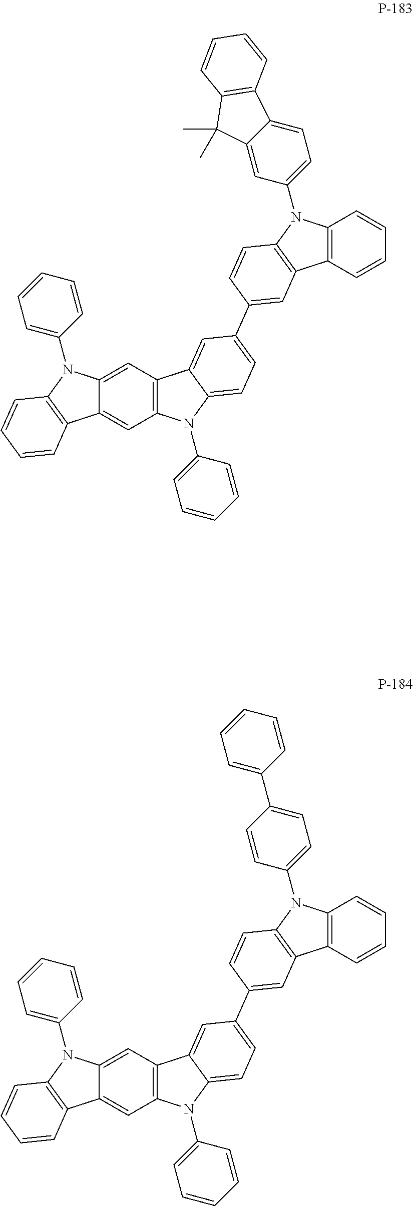

An organic mixture according to another embodiment includes the aforementioned organic compound or the aforementioned organic mixture including the organic compound H1 and the organic compound H2, and an organic functional material. The organic functional material includes a hole (also called an electron hole) injection or a hole transport material (HIM/HTM), a hole blocking material (HBM), an electron injection or transport material (EIM/ETM), an electron blocking material (EBM), an organic host material (Host), a singlet emitter (fluorescent emitter), a triplet emitter (phosphorescent emitter), or a thermally activated delayed fluorescent material (TADF material), especially a light-emitting organometallic complex. Various organic functional materials are described in detail, for example, in WO2010135519A1, US20090134784A1, and WO2011110277A1, the entire contents of which are hereby incorporated herein by reference. The organic functional material may be a small molecule material or a polymer material.

In one of the embodiments, the organic functional material is a phosphorescent emitter. In this case, the organic compound or the aforementioned organic mixture including the organic compound H1 and the organic compound H2 is used as a host material, and the weight percentage of the phosphorescent emitter in the mixture is less than or equal to 30 wt %. In one of the embodiments, the weight percentage of the phosphorescent emitter in the mixture is less than or equal to 25 wt %. In one of the embodiments, the weight percentage of the phosphorescent emitter in the mixture is less than or equal to 20 wt %.

In one of the embodiments, the organic mixture includes the fluorescent host material and the aforementioned organic mixture including the organic compound H1 and the organic compound H2. In this case, the aforementioned organic mixture including the organic compound H1 and the organic compound H2 is used as the fluorescent material, and the weight percentage of the organic mixture including the organic compound H1 and the organic compound H2 is less than or equal to 15 wt %. In one of the embodiments, the weight percentage of the organic mixture including the organic compound H1 and the organic compound H2 is less than or equal to 10 wt %. In one of the embodiments, the weight percentage of the organic mixture including the organic compound H1 and the organic compound H2 is less than or equal to 8 wt %.

In one of the embodiments, the organic mixture includes the phosphorescent emitter, the host material, and the organic mixture including the organic compound H1 and the organic compound H2. In this case, the organic mixture including the organic compound H1 and the organic compound H2 is used as an auxiliary light-emitting material, and a weight ratio of the organic mixture including the organic compound H1 and the organic compound H2 to the phosphorescent emitter ranges from 1:2 to 2:1. In one of the embodiments, the T₁ of the organic mixture is higher than that of the phosphorescent emitter.

In one of the embodiments, the organic mixture includes the TADF material, and includes the organic compound. In this case, the organic compound is used as the host material of TADF, and a weight percentage of the TADF material in the mixture ≤15 wt %/o. In one of the embodiments, the weight percentage of the TADF material in the mixture ≤10 wt %. In one of the embodiments, the weight percentage of the TADF material in the mixture ≤8 wt %.

The host material, the phosphorescent material, the fluorescent host material, the fluorescent material, and the TADF material are described in further detail below (but are not limited thereto).

1. Host Material (Triplet Host)

Examples of a triplet host material are not particularly limited, and any metal complex or organic compound may be used as the host material as long as its triplet energy is higher than that of the emitter, particularly the triplet emitter or the phosphorescent emitter. Examples of metal complexes that can be used as triplet hosts includes, but are not limited to, the general structure as follows:

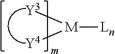

M is a metal; (Y³-Y⁴) is a bidentate ligand, Y³ and Y⁴ are independently selected from C, N, O, P, or S; L is an auxiliary ligand; m is an integer with the value from 1 to the maximum coordination number of the metal; and m+n is the maximum number of coordination of the metal.

In one embodiment, the metal complex which can be used as the triplet host has the following formula:

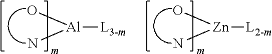

(O—N) is a bidentate ligand in which the metal is coordinated to O and N atoms.

In one of the embodiments, M is selected from Ir or Pt.

Examples of the organic compounds which can be used as the triplet host are selected from the group consisting of compounds containing cyclic aryl, such as benzene, biphenyl, triphenyl, benzo, and fluorene; and compounds containing heterocyclic aryl, such as dibenzothiophene, dibenzofuran, dibenzoselenophen, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, oxazole, bibenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furopyridine, benzothienopyridine, thienopyridine, benzoselenophenopyridine, and selenophenobenzodipyridine; and groups containing 2 to 10 ring structures, which may be the same or different types of cyclic aryl or heterocyclic aryl and are linked to each other directly or by at least one of the following groups, such as oxygen atom, nitrogen atom, sulfur atom, silicon atom, phosphorus atom, boron atom, chain structural unit, and aliphatic ring group. Each Ar may be further substituted, and the substituents may be selected from the group consisting of hydrogen, alkyl, alkoxy, amino, alkenyl, alkynyl, aralkyl, heteroalkyl, aryl, and heteroaryl.

In one of the embodiments, the triplet host material is selected from compounds including at least one of the following groups:

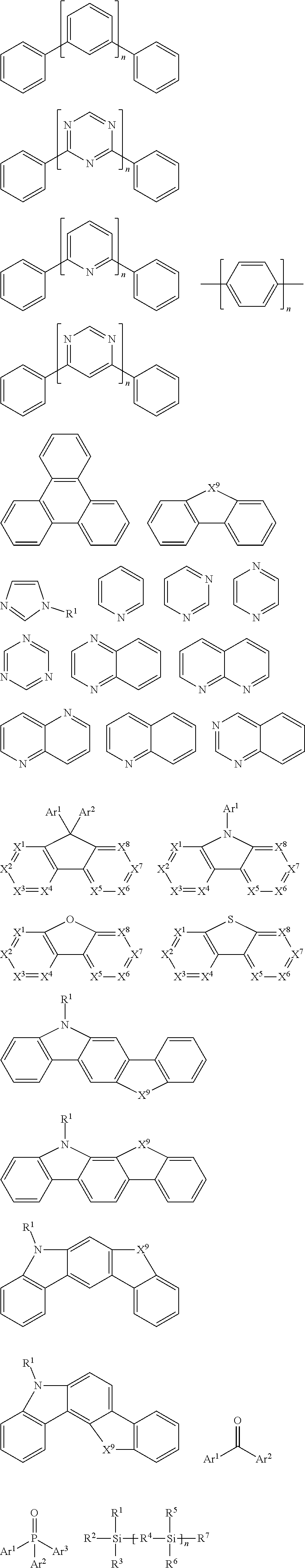

where R¹ to R⁷ are independently selected from the group consisting of hydrogen, alkyl, alkoxy, amino, alkene, alkyne, aralkyl, heteroalkyl, aryl, and heteroaryl, which have the same meaning as the aforementioned Ar¹ and Ar² when R¹ to R⁷ are selected from aryl or heteroaryl. n is selected from 1, 2, 3, 4, 5, 6, 7, 8, 9, 10, 11, 12, 13, 14, 15, 16, 17, 18, 19, or 20. X¹ to X⁸ are selected from CH or N; and X⁹ is selected from CR¹R² or NR¹.

Examples of suitable triplet host materials are listed in the table below.

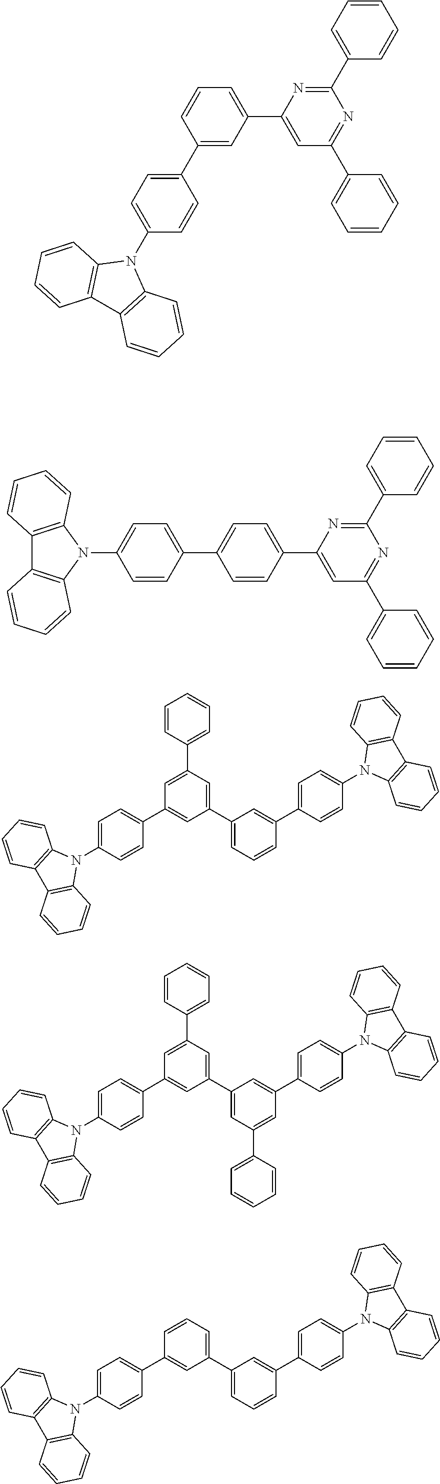

2. Phosphorescent Material

The phosphorescent material is also called the triplet emitter. In one embodiment, the triplet emitter is a metal complex having a general formula M(L)n. M is a metal atom. L may be the same or different at each occurrence, and is an organic ligand which is bonded or coordinated to the metal atom M at one or more positions. n is an integer greater than 1. In one embodiment, n is selected from 1, 2, 3, 4, 5, or 6. In one of the embodiments, these metal complexes are attached to a polymer at one or more positions. In one embodiment, these metal complexes are attached to the polymer particularly by organic ligands.

In one of the embodiments, the metal atom M is selected from the group consisting of a transition metal element, a lanthanide element, and an actinide element. In one of the embodiments, M is selected from the group consisting of Ir, Pt, Pd, Au, Rh, Ru, Os, Sm, Eu, Gd, Tb, Dy, Re, Cu, and Ag. In one of the embodiments, M is selected from the group consisting of Os, Ir, Ru, Rh, Re, Pd, and Pt.

In one of the embodiments, the triplet emitter includes a chelating ligand, i.e., a ligand, and is coordinated to the metal by at least two binding sites. In one of the embodiments, the triplet emitter includes two or three identical or different bidentate or multidentate ligands. The chelating ligand is beneficial for improve the stability of the metal complexes.

Examples of the organic ligand are selected from the group consisting of phenylpyridine derivatives, 7, 8-benzoquinoline derivatives, 2(2-thienyl) pyridine derivatives, 2(1-naphthyl) pyridine derivatives, and 2-phenylquinoline derivatives. All of these organic ligands may be substituted, for example, by fluoromethyl or trifluoromethyl. The auxiliary ligand may be selected from acetylacetonate or picric acid.

In one embodiment, the metal complex which can be used as the triplet emitters has the following form:

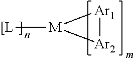

M is a metal and is selected from the group consisting of a transition metal element, a lanthanide element, and an actinide element.

Ar¹ is a cyclic group, which may be the same or different at each occurrence, and Ar¹ contains at least one donor atom, that is, an atom having a lone pair of electrons, such as nitrogen or phosphorus, through which the cyclic group is coordinated to a metal. Ar² is a cyclic group, which may be the same or different at each occurrence, and Ar² contains at least one C atom, through which the cyclic group is linked to the metal. Ar¹ and Ar² are covalently bonded together, and each of Ar¹ and Ar³ can have one or more substituents, which may also be linked together by the substituents. L may be the same or different at each occurrence, and L is an auxiliary ligand. In one embodiment, L is a bidentate chelating ligand. In another embodiment, L is a monoanionic bidentate chelating ligand. m is selected from 1, 2, or 3. n is selected from 0, 1, or 2. In one of the embodiments, L is a bidentate chelating ligand. In one of the embodiments, L is a monoanionic bidentate chelating ligand. In one of the embodiments, m is 2 or 3. In one of the embodiments, m is 3. In one of the embodiments, n is 0 or 1. In one of the embodiments, n is 0.

Examples of materials for some triplet emitters and applications thereof can be found in the following patent documents and literatures: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728, WO 2010086089, WO 2010099852, WO 2010102709, US 20070087219 A1, US 20090061681 A1, US 20010053462 A1, Baldo, Thompson et al. Nature 403, (2000), 750-753, US 20090061681 A1, US 20090061681 A1, Adachi et al. Appl. Phys. Lett. 78 (2001), 1622-1624, J. Kido et al. Appl. Phys. Lett. 65 (1994), 2124, Kido et al. Chem. Lett. 657, 1990, US 2007/0252517 A1, Johnson et al., JACS 105, 1983, 1795, Wrighton, JACS 96, 1974, 998, Ma et al., Synth. Metals 94, 1998, 245, U.S. Pat. Nos. 6,824,895, 7,029,766, 6,835,469, 6,830,828, US 20010053462 A1, WO 2007095118 A1, US 2012004407A1, WO 2012007088A1, WO2012007087A1, WO 2012007086A1, US 2008027220A1, WO 2011157339A1, CN 102282150A, WO 2009118087A1. The entire contents of the above listed patent documents and literatures are hereby incorporated by reference.

Examples of some suitable triplet emitters are listed in the table below.

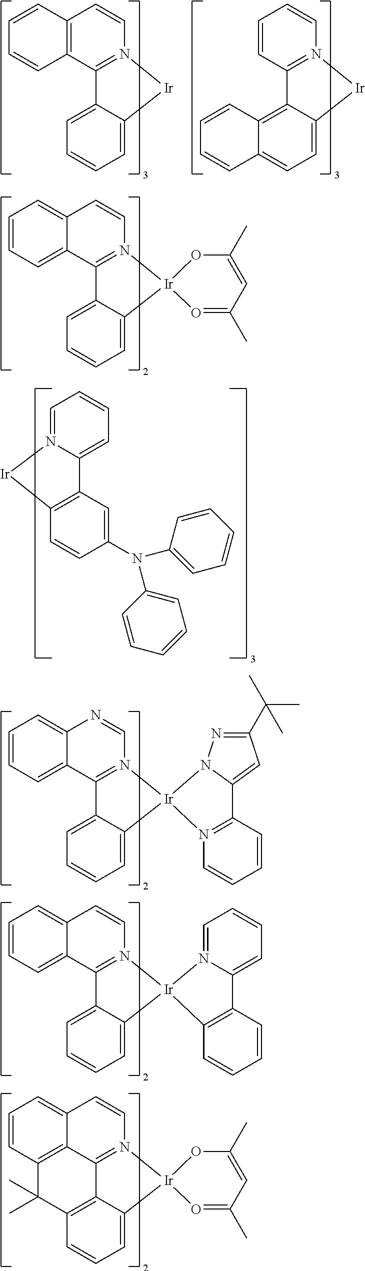

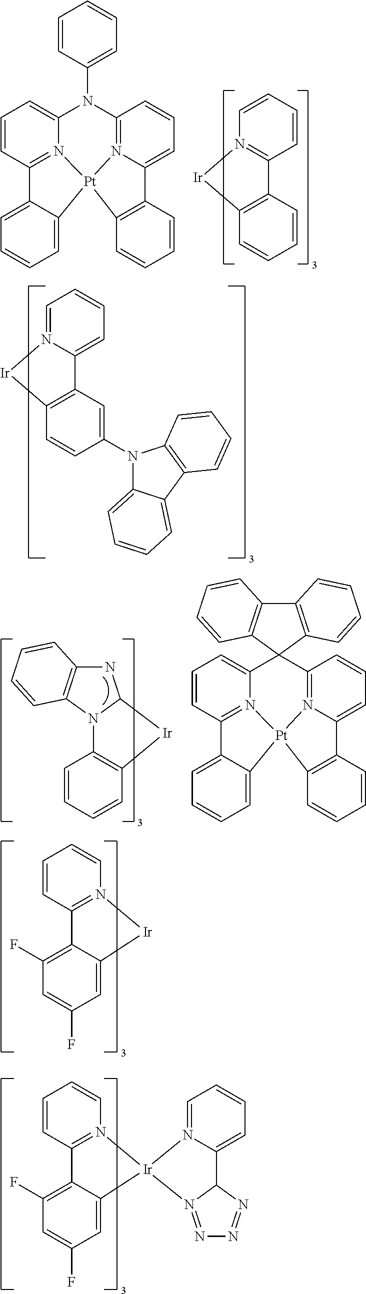

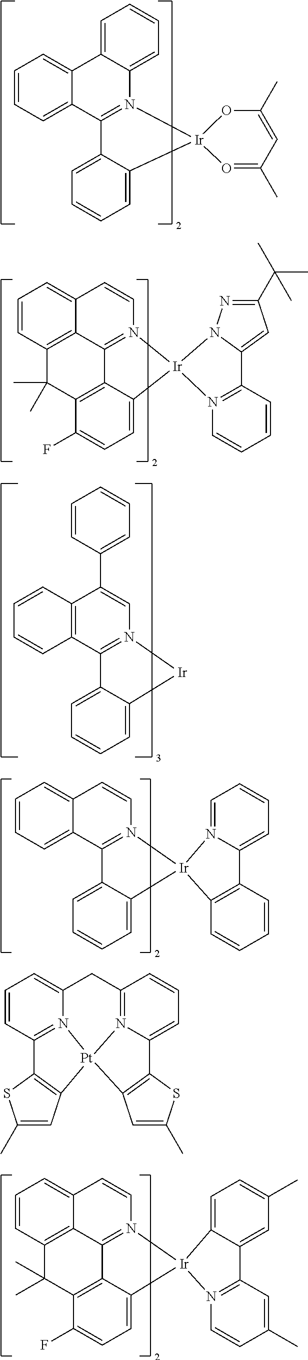

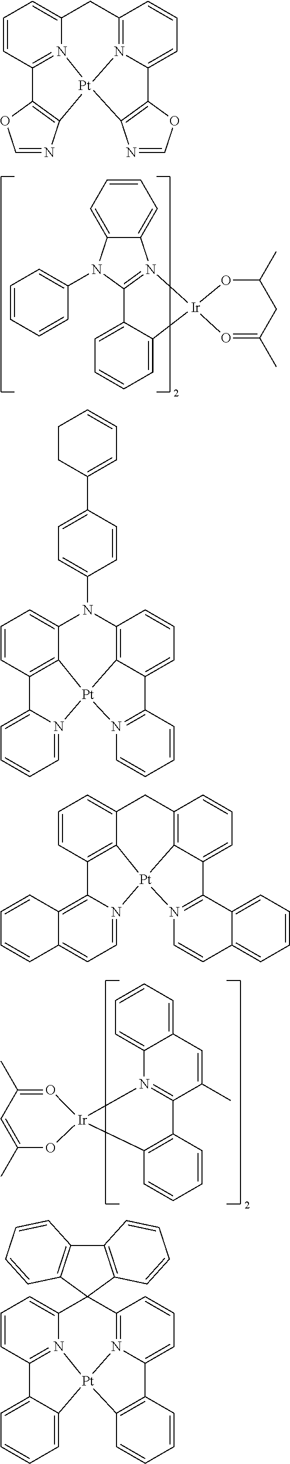

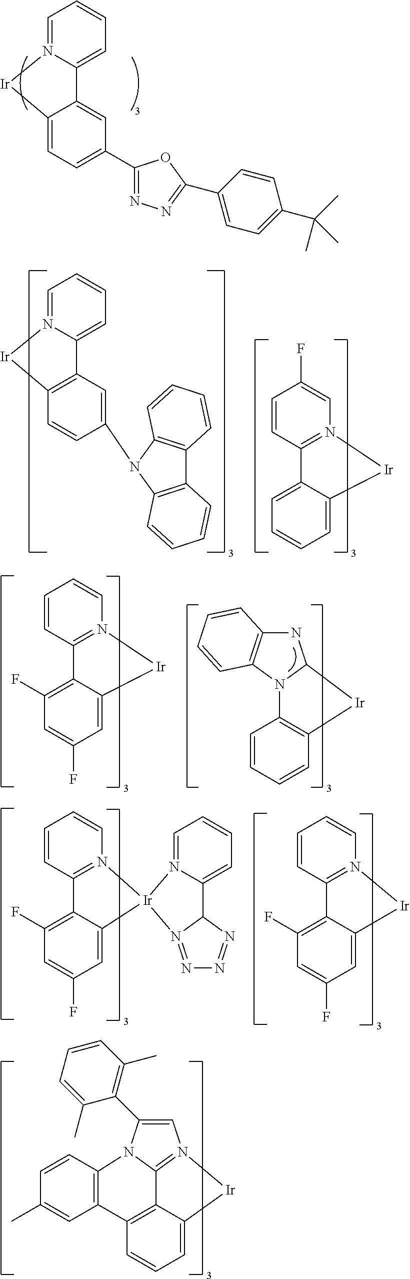

3. Singlet Host Material (Singlet Host):

Examples of the singlet host materials are not particularly limited, and any organic compound can be used as the host as long as its singlet energy is greater than that of the emitter, particularly the singlet emitter or the fluorescent emitter.

Examples of organic compounds which can be used as the singlet host materials are selected from the group consisting of: compounds containing cyclic aryl groups, such as benzene, biphenyl, triphenyl, benzo, naphthalene, anthracene, phenalene, phenanthrene, fluorene, pyrene, chrysene, perylene, azulene; and aromatic heterocyclic compounds such as dibenzothiophene, dibenzofuran, dibenzoselenophen, furan, thiophene, benzofuran, benzothiophene, benzoselenophene, carbazole, indolocarbazole, indolopyridine, pyrrolodipyridine, pyrazole, imidazole, triazole, isoxazole, thiazole, oxadiazole, oxatriazole, dioxazole, thiadiazole, pyridine, pyridazine, pyrimidine, pyrazine, triazine, oxazine, oxathiazin, oxadiazine, indole, benzimidazole, indazole, indolizine, benzoxazole, benzoisoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and groups containing 2 to 10 ring structures, which may be the same or different types of cyclic aryl or aromatic heterocyclic groups, and are linked to each other directly or through at least one of the following groups, such as an oxygen atom, a nitrogen atom, a sulfur atom, a silicon atom, a phosphorus atom, a boron atom, a chain structure unit, and an aliphatic ring group.

In one embodiment, the singlet host material may be selected from compounds containing at least one of the following groups.

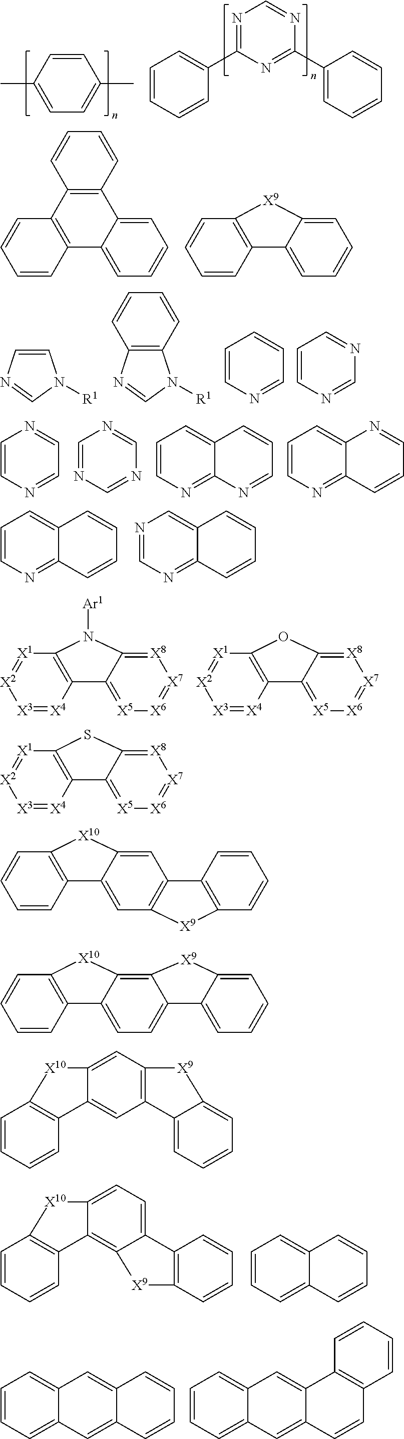

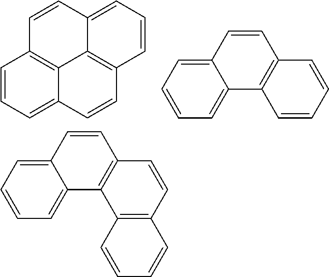

R¹ is selected from the group consisting of hydrogen, alkyl, alkoxy, amino, alkene, alkyne, aralkyl, heteroalkyl, aryl, and heteroaryl. Ar¹ is aryl or heteroaryl, and has the same meaning as Ar¹ defined in the aforementioned HTM. n is selected from 1, 2, 3, 4, 5, 6, 7, 8, 9, 10, 11, 12, 13, 14, 15, 16, 17, 18, 19, or 20. X¹ to X⁸ are selected from CH or N. X⁹ is selected from CR¹R² or NR¹.

Some examples of anthracene-based singlet host materials are listed in the table below.

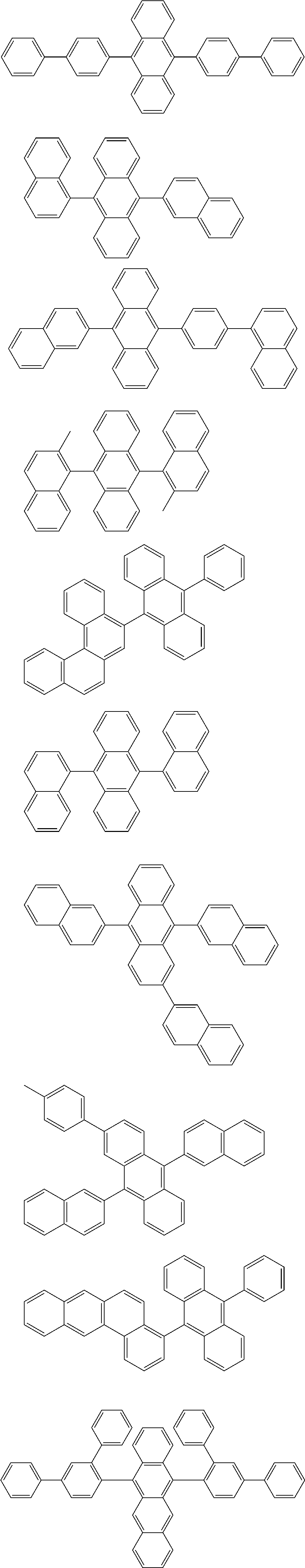

4. Fluorescent Emitter (Singlet Emitter)

The singlet emitters tend to have longer conjugated n-electron systems. To date, there have been many examples, such as styrylamine and its derivatives disclosed in JP2913116B and WO2001021729A1, and indenofluorene and its derivatives disclosed in WO2008/006449 and WO2007/140847.

In one of the embodiments, the singlet emitter is selected from the group consisting of monostyrylamines, distyrylamines, tristyrylamines, tetrastyrylamines, styrylphosphines, styryl ethers, and arylamines.

The monostyrylamine refers to a compound which includes one unsubstituted or substituted styryl group and at least one amine, in which the amine may be an aromatic amine. The distyrylamine refers to a compound which includes two unsubstituted or substituted styryl groups and at least one amine, in which the amine may be the aromatic amine. The tristyrylamine refers to a compound which includes three unsubstituted or substituted styryl groups and at least one amine, in which the amine may be the aromatic amine. The tetrastyrylamine refers to a compound which includes four unsubstituted or substituted styryl groups and at least one amine, in which the amine may be the aromatic amine.

In one of the embodiments, styrene is stilbene, which may be further substituted. The corresponding phosphines and ethers are defined similarly to amines. Aryl amine or aromatic amine refers to a compound containing three unsubstituted or substituted aromatic ring or heterocyclic systems directly attached to nitrogen. In one embodiment, at least one of these aromatic ring or heterocyclic systems is selected from fused ring systems. In another embodiment, at least one of these aromatic ring or heterocyclic systems has at least 14 aryl ring atoms. In one of the embodiments, the aromatic ring or the heterocyclic system may be selected from the group consisting of aromatic anthramine, aromatic anthradiamine, aromatic pyrene amine, aromatic pyrene diamine, aromatic chrysene amine, and aromatic chrysene diamine. The aromatic anthramine refers to a compound in which one diarylamino group is directly attached to anthracene, for example, at position 9. The aromatic anthradiamine refers to a compound in which two diarylamino groups are directly attached to anthracene, for example, at positions 9, 10. The aromatic pyrene amine, the aromatic pyrene diamine, the aromatic chrysene amine, and the aromatic chrysene diamine are similarly defined, and the diarylarylamino group is, for example, attached to position 1 or 1 and 6 of pyrene.

Examples of singlet emitters based on vinylamine and aromatic amine are also examples and can be found in the following patent documents: WO 2006/000388, WO 2006/058737, WO 2006/000389, WO 2007/065549, WO 2007/115610, U.S. Pat. No. 7,250,532 B2, DE 102005058557 A1, CN 1583691 A, JP 08053397 A, U.S. Pat. No. 6,251,531 B1, US 2006/210830 A, EP 1957 606 A1, and US 2008/0113101 A1. The patent documents listed above are specially incorporated herein by reference in their entirety.

Examples of singlet emitters based on distyrylbenzene and derivatives thereof may be found in, for example, U.S. Pat. No. 5,121,029.

In one of the embodiments, the singlet emitters are selected from the group consisting of: indenofluorene-amine and indenofluorene-diamine, such as those disclosed in WO 2006/122630; benzoindenofluorene-amine and benzoindenofluorene-diamine, such as those disclosed in WO 2008/006449; and dibenzoindenofluorene-amine and dibenzoindenofluorene-diamine, such as those disclosed in WO2007/140847.

Other materials useful as singlet emitters include polycyclic aryl compounds, especially one selected from the derivatives of the following compounds: anthracenes such as 9, 10-di-naphthylanthracene, naphthalene, tetraphenyl, phenanthrene, perylene such as 2, 5, 8, 11-tetra-t-butylatedylene, indenoperylene, phenylenes such as 4, 4′-(bis(9-ethyl-3-carbazovinylene)-1, 1′-biphenyl, periflanthene, decacyclene, coronene, fluorene, spirobifluorene, arylpyren (e.g., US20060222886), arylenevinylene (e.g., U.S. Pat. Nos. 5,121,029, 5,130,603), cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds (US 2007/0092753 A1), bis (azinyl) methene compounds, carbostyryl compounds, oxazone, benzoxazole, benzothiazole, benzimidazole, and diketopyrrolopyrrole. Some singlet emitter materials may be found in the following patent documents: US20070252517A1, U.S. Pat. Nos. 4,769,292, 6,020,078, US2007/0252517A1, and US2007/0252517A1. The patent documents listed above are specially incorporated herein by reference in their entirety.

Examples of suitable singlet emitters are listed in the table below.

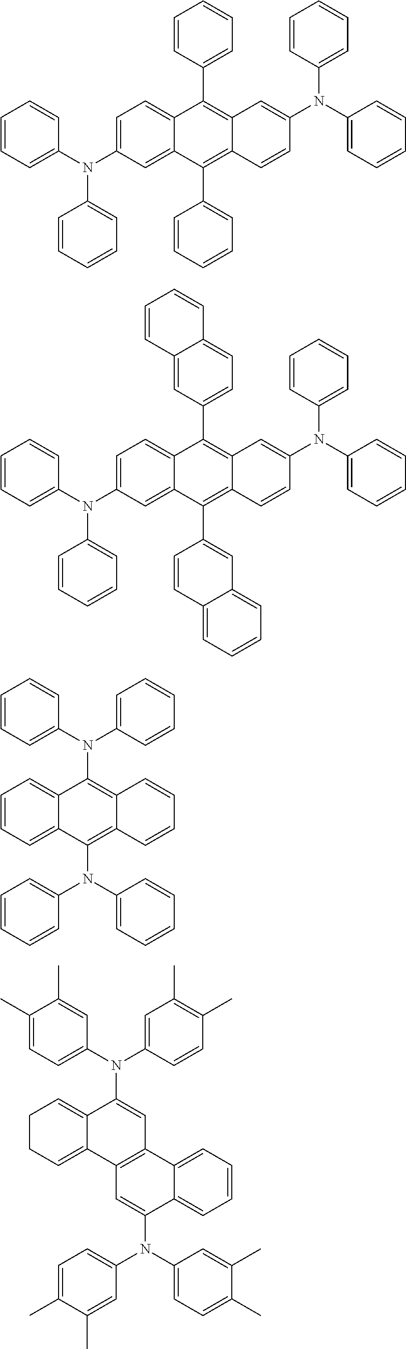

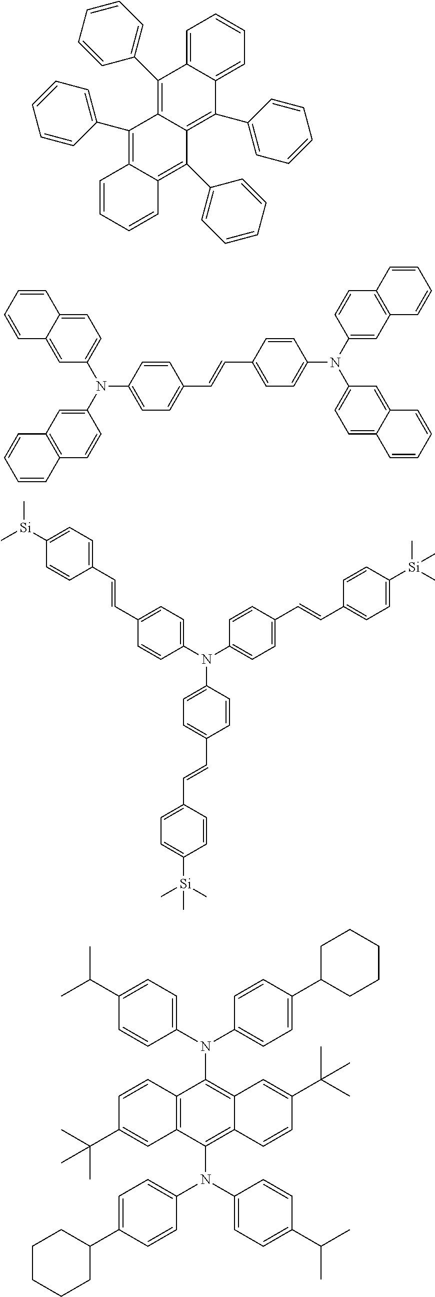

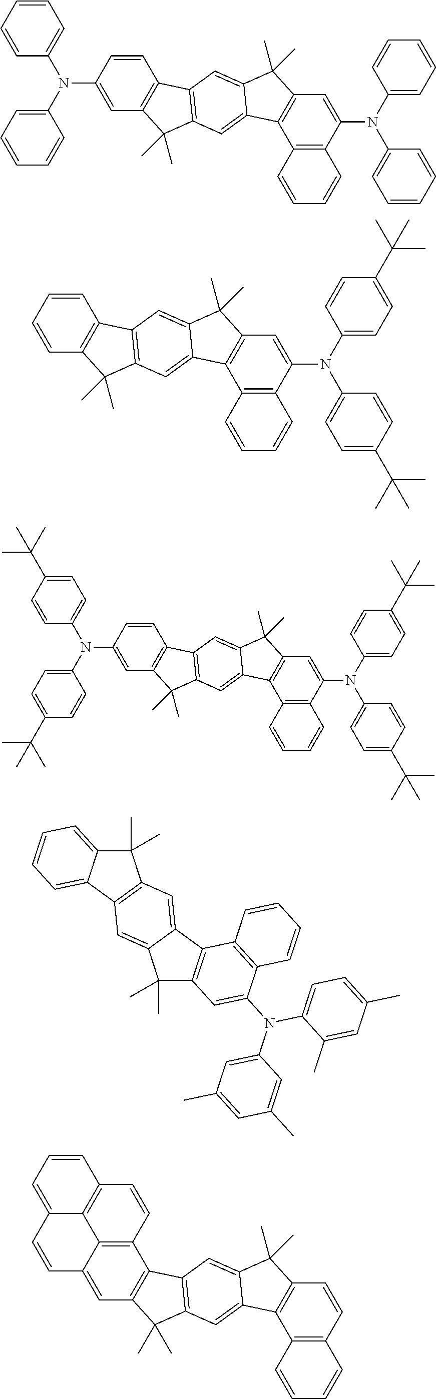

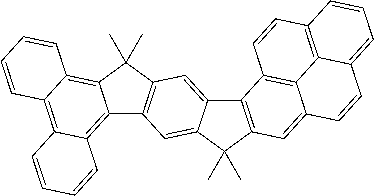 [SMILES: CC1(C)c2cc3c(cc2-c2c1cc1ccc4cccc5ccc2c1c45)C(C)(C)c1c-3c2ccccc2c2ccccc12]

5. TADF Material

Conventional organic fluorescent materials can only emit light by using 25% singlet excitons formed by electrical excitation, and the internal quantum efficiency of the device is low (up to 25%). The phosphorescent material enhances the intersystem crossing due to the strong spin-orbit coupling of the center of the heavy atom, the singlet excitons and triplet excitons formed by electrical excitation can be effectively utilized to emit light, so that the internal quantum efficiency of the device reaches 100%. However, the problems of expensive phosphorescent materials, poor material stability, and severe roll-off of device efficiency limit their application in OLEDs. The thermally activated delayed fluorescent material is a third generation organic luminescent material developed after the organic fluorescent materials and the organic phosphorescent materials. Such materials generally have a small singlet-triplet excited state energy level difference (ΔEst), the triplet excitons can be converted into the singlet excitons by reverse intersystem crossing to emit light. This can make full use of the singlet excitons and triplet excitons formed under electrical excitation, and the internal quantum efficiency of the device can reach 100%. In addition, the material has a controllable structure, stable property, and cheap price, no precious metal is required, and the applications in the OLED field is promising.

The TADF material needs to have a smaller singlet-triplet excited state energy level difference. In one embodiment, ΔEst<0.3 eV. In another embodiment, ΔEst<0.2 eV. In yet another embodiment, ΔEst<0.1 eV. In one of the embodiments, the TADF material has a relatively small ΔEst. In another embodiment, TADF has a better fluorescence quantum efficiency. Some TADF materials can be found in the following patent documents: CN103483332(A), TW201309696(A), TW201309778(A), TW201343874(A), TW201350558(A), US20120217869(A1), WO2013133359(A1), WO2013154064(A1), Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013, 3766, Adachi, et. al. J. Mater. Chem. C., 1, 2013, 4599, Adachi, et. al. J. Phys. Chem. A., 117, 2013, 5607. The entire contents of the above-listed patents and literature documents are hereby incorporated by reference.

Some examples of the suitable TADF light-emitting materials are listed in the following table.

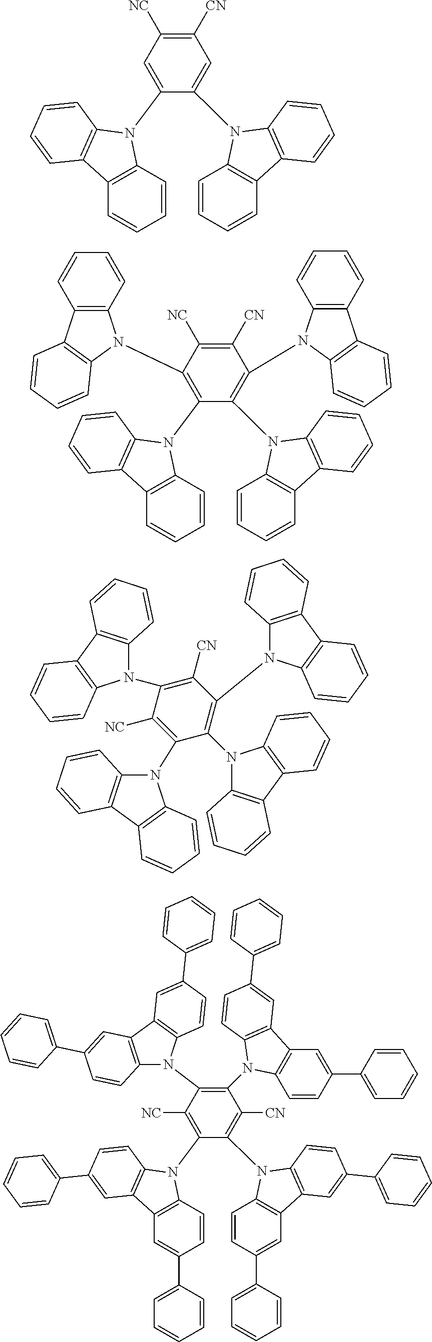

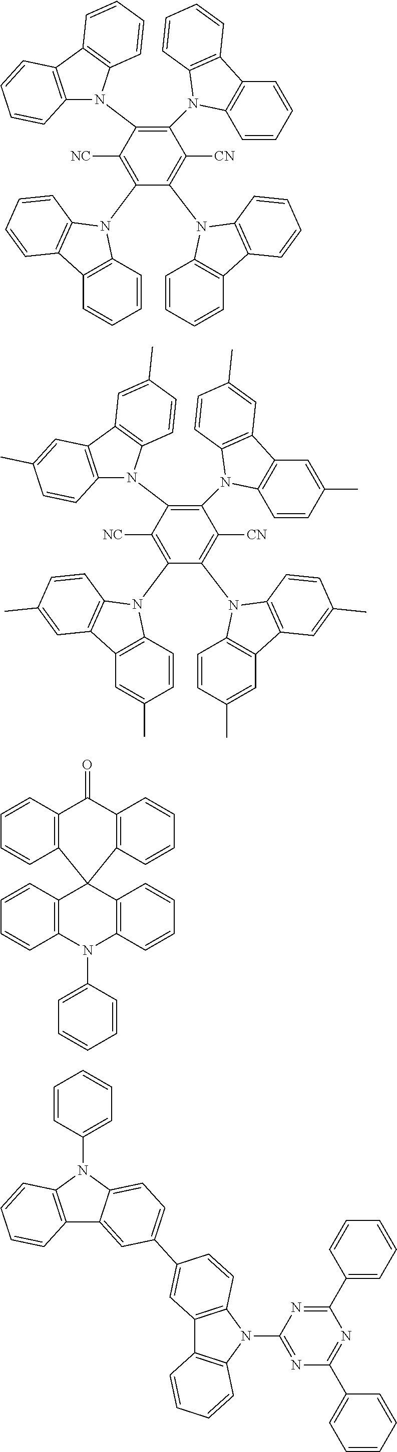

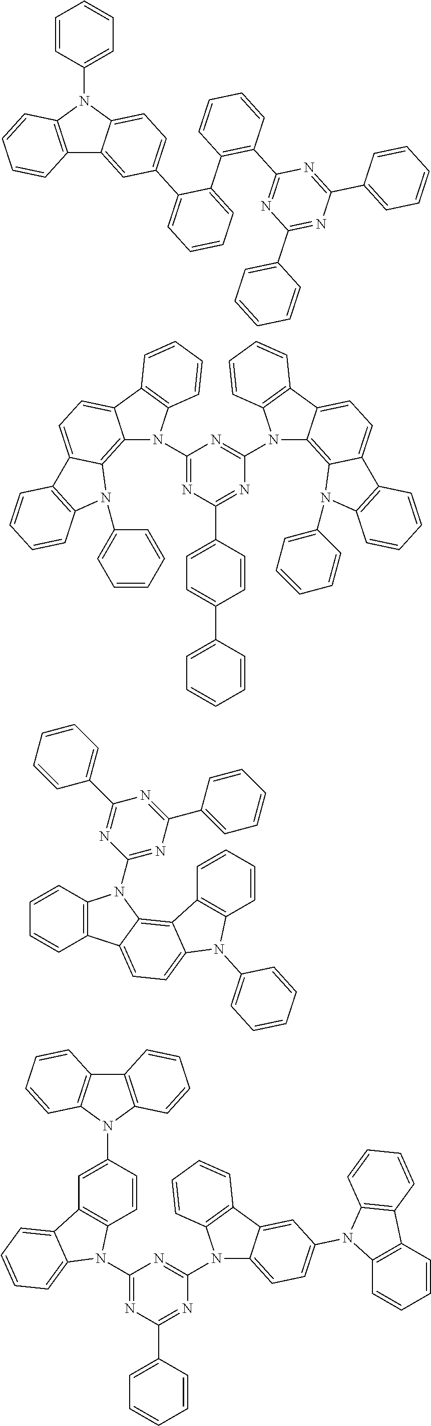

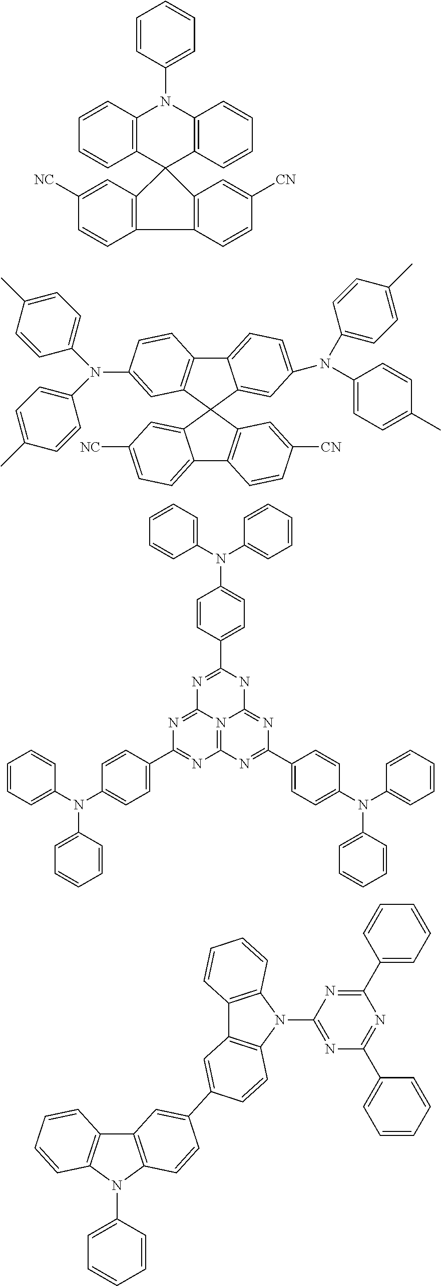

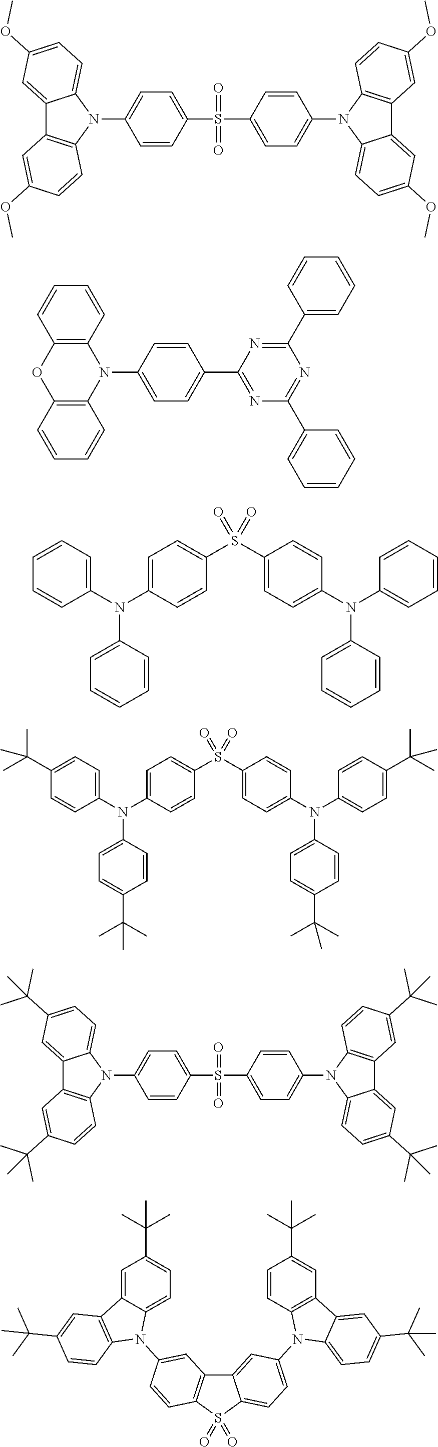

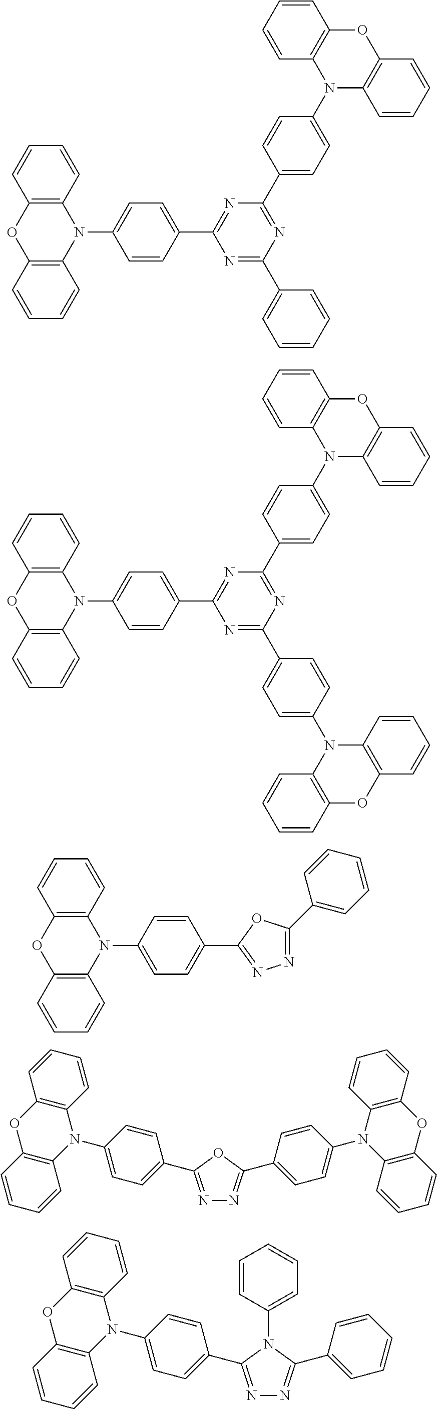

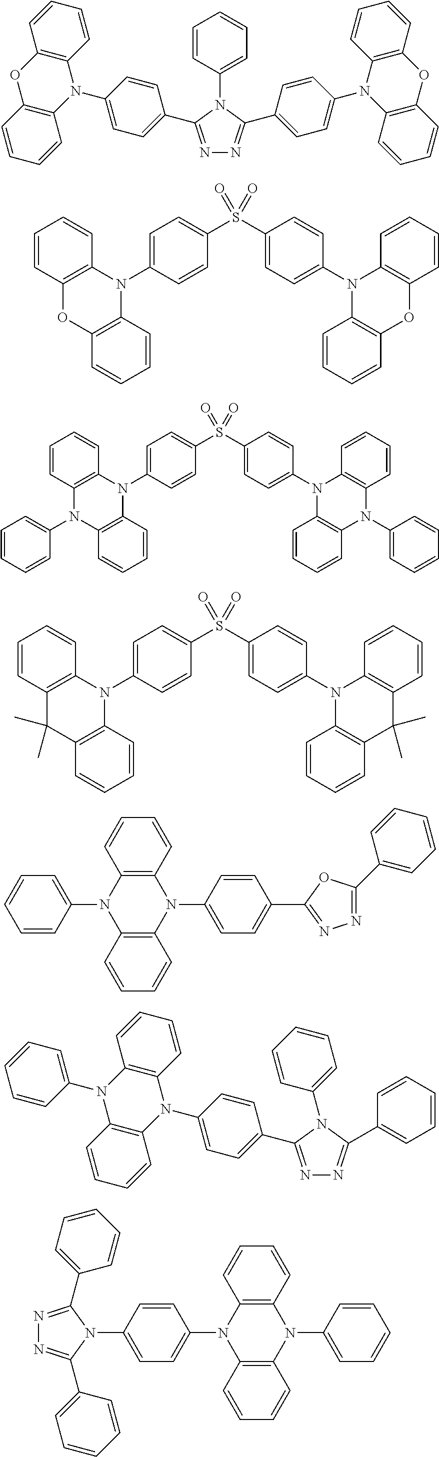

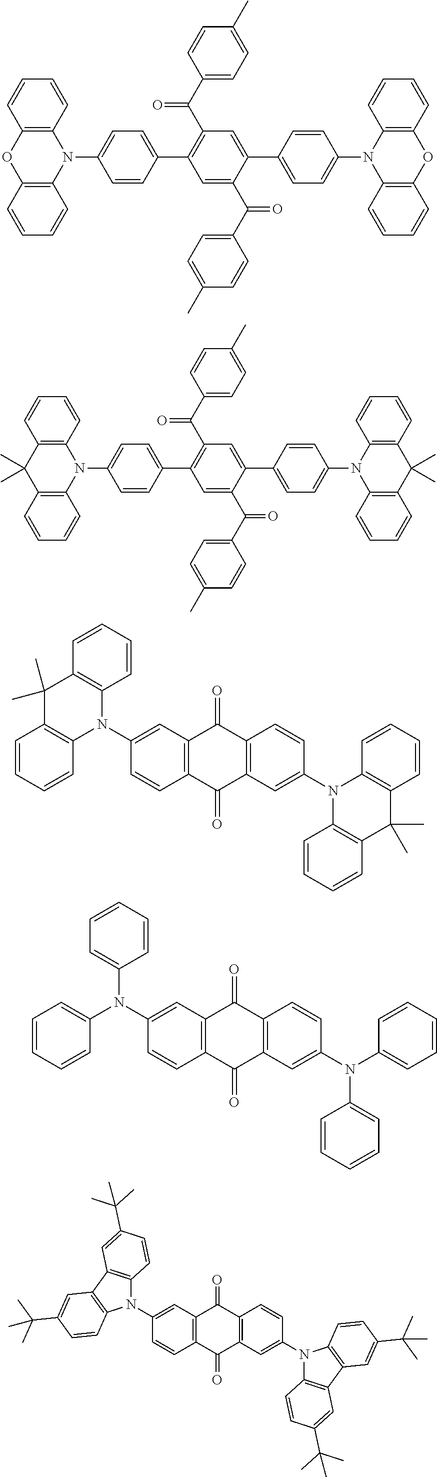

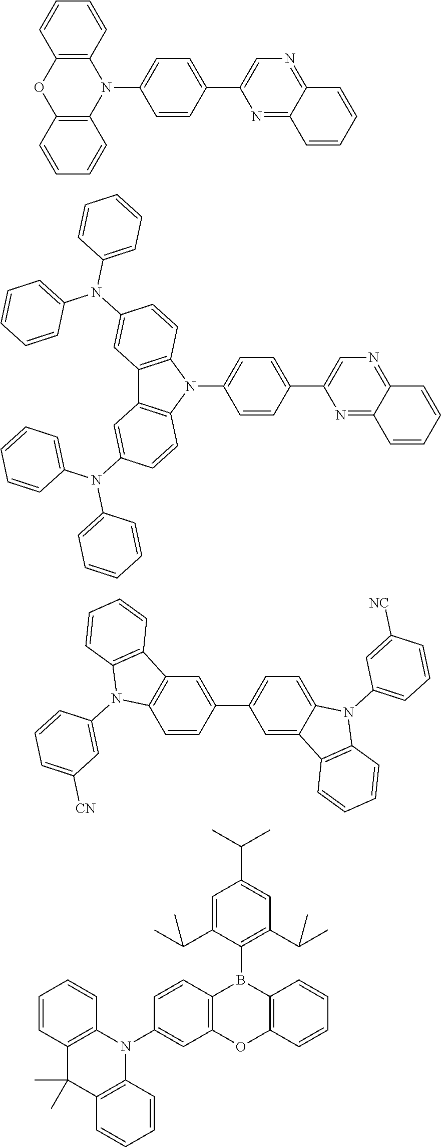

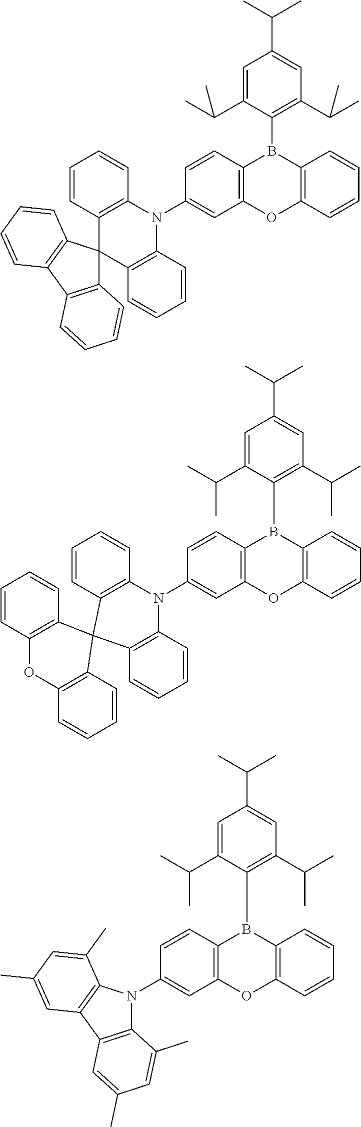

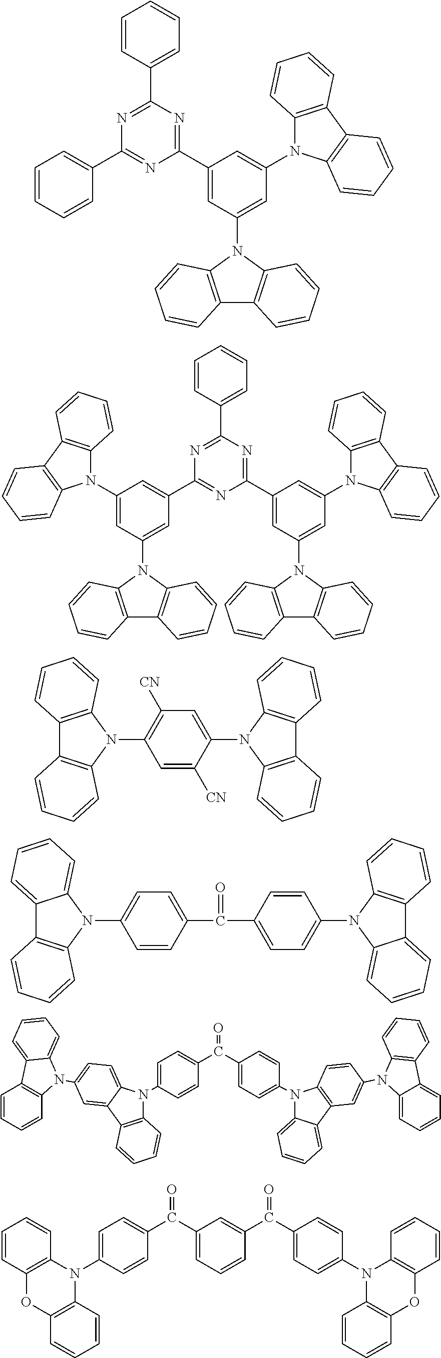

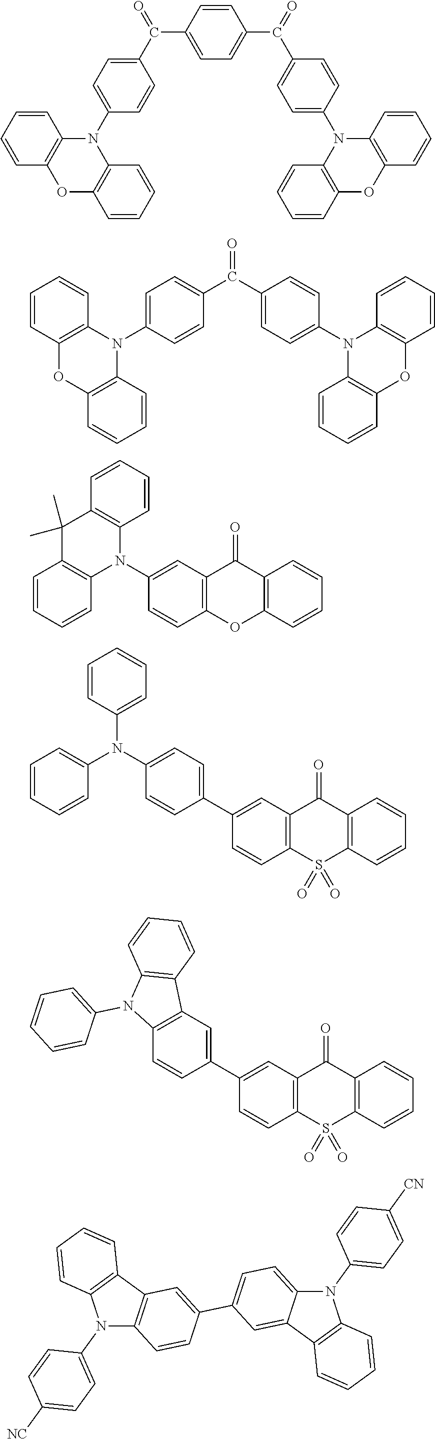

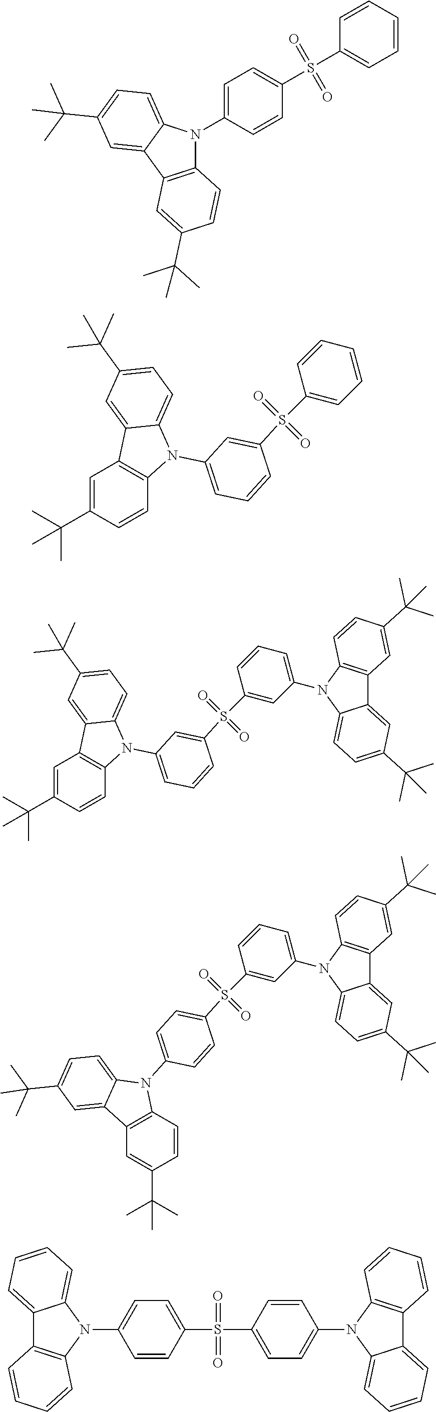

In one of the embodiments, the aforementioned organic compound is used for printing OLEDs, which has a molecular weight greater than or equal to 700 mol/kg. In one of the embodiments, the molecular weight of the organic compound is greater than or equal to 800 mol/kg. In one of the embodiments, the molecular weight of the organic compound is greater than or equal to 900 mol/kg. In one of the embodiments, the molecular weight of the organic compound is greater than or equal to 1000 mol/kg. In one of the embodiments, the molecular weight of the organic compound is greater than or equal to 1100 mol/kg.

In one of the embodiments, the aforementioned organic compound or organic mixture has a solubility in toluene greater than or equal to 10 mg/ml at 25° C. In one of the embodiments, the solubility in toluene is greater than or equal to 15 mg/ml. In one of the embodiments, the solubility in toluene is greater than or equal to 20 mg/ml.

An application of the aforementioned organic mixture in an organic electronic device is provided.

An application of the aforementioned organic mixture in an electronic device is provided.

A formulation according to an embodiment includes the aforementioned organic compound and an organic solvent. In the present embodiment, the formulation is an ink. Thus, the viscosity and surface tension of the ink are important parameters when the formulation is used in a printing process. Suitable surface tension parameters of the ink are suitable for a particular substrate and a particular printing method.

In one embodiment, the surface tension of the ink at an operating temperature or at 25° C. is in a range of 19 dyne/cm to 50 dyne/cm. In one of the embodiments, the surface tension of the ink an operating temperature or at 25° C. is in a range of 22 dyne/cm to 35 dyne/cm. In one of the embodiments, the surface tension of the ink an operating temperature or at 25° C. is in a range of 25 dyne/cm to 33 dyne/cm.

In one embodiment, the viscosity of the ink at an operating temperature or at 25° C. is in a range of 1 cps to 100 cps. In one of the embodiments, the viscosity of the ink at an operating temperature or at 25° C. is in a range of 1 cps to 50 cps. In one of the embodiments, the viscosity of the ink at an operating temperature or at 25° C. is in a range of 1.5 cps to 20 cps. In one of the embodiments, the viscosity of the ink at an operating temperature or at 25° C. is in a range of 4.0 cps to 20 cps. Therefore, the formulation is more convenient for inkjet printing.

The viscosity can be adjusted by various methods, such as by a proper solvent selection and the concentration of functional materials in the ink. The ink including a metal organic complex or a polymer can facilitate the adjustment of the printing ink in an appropriate range according to the used printing method. The organic functional material contained in the formulation has a weight ratio of 0.3 wt % to 30 wt %. In one of the embodiments, the weight ratio of the organic functional material contained in the formulation ranges from 0.5 wt % to 20 wt %. In one of the embodiments, the weight ratio of the organic functional material contained in the formulation ranges from 0.5 wt % to 15 wt %. In one of the embodiments, the weight ratio of the organic functional material contained in the formulation ranges from 0.5 wt % to 10 wt %. In one of the embodiments, the weight ratio of the organic functional material contained in the formulation ranges from 1 wt % to 5 wt %.

In one embodiment, the organic solvent includes a first solvent selected from solvents based on aromatic and/or heteroaromatic. In addition, the first solvent may be an aliphatic chain/cycle-substituted aromatic solvent, an aromatic ketone solvent, or an aromatic ether solvent.

Examples of the first solvent include, but are not limited to, solvents based on aromatic or heteroaromatic: such as p-diisopropylbenzene, amylbenzene, tetrahydronaphthalene, phenylcyclohexane, chloronaphthalene, 1, 4-dimethylnaphthalene, 3-isopropylbiphenyl, p-methylisopropylbenzene, diamylbenzene, triamylbenzene, pentyltoluene, o-xylene, m-xylene, p-xylene, o-diethylbenzene, m-diethylbenzene, p-diethylbenzene, 1, 2, 3, 4-tetramethylbenzene, 1, 2, 3, 5-tetramethylbenzene, 1, 2, 4, 5-tetramethylbenzene, butylbenzene, dodecylbenzene, dihexylbenzene, dibutylbenzene, p-diisopropylbenzene, 1-methoxynaphthalene, phenylcyclohexane, dimethylnaphthalene, 3-isopropylbiphenyl, p-methylisopropylbenzene, 1-methylnaphthalene, 1, 2, 4-trichlorobenzene, 1, 3-dipropoxybenzene, 4, 4-difluorodiphenylmethane, 1, 2-dimethoxy-4-(1-propenyl)benzene, diphenylmethane, 2-phenylpyridine, 3-phenylpyridine, N-methyldiphenylamine, 4-isopropylbiphenyl, α, α-dichlorodiphenylmethane, 4-(3-phenylpropyl)pyridine, benzyl benzoate, 1, 1-bis(3, 4-dimethylphenyl)ethane, 2-isopropylnaphthalene, dibenzyl ether, and the like; solvents based on ketone, such as 1-tetralone, 2-tetralone, 2-(phenylepoxy)tetralone, 6-(methoxy)-tetralone, acetophenone, propiophenone, benzophenone, and derivatives thereof, such as 4-methylacetophenone, 3-methylacetophenone, 2-methylacetophenone, 4-methylpropiophenone, 3-methylpropiophenone, 2-methylpropiophenone, isophorone, 2, 6, 8-trimethyl-4-nonanone, fenchone, 2-nonanone, 3-nonanone, 5-nonanone, 2-decanone, 2,5-hexanedione, phorone, and di-n-amyl ketone; aromatic ether solvents, such as 3-phenoxytoluene, butoxybenzene, benzyl butylbenzene, p-anisaldehyde dimethyl acetal, tetrahydro-2-phenoxy-2H-pyran, 1, 2-dimethoxy-4-(1-propenyl)benzene, 1, 4-benzodioxane, 1, 3-dipropylbenzene, 2, 5-dimethoxytoluene, 4-ethylphenetole, 1, 2, 4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1, 2-dimethoxybenzene, 1-methoxynaphthalene, diphenyl ether, 2-phenoxymethyl ether, 2-phenoxytetrahydrofuran, ethyl-2-naphthyl ether, amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether; and ester solvents, such as alkyl octoate, alkyl sebacate, alkyl stearate, alkyl benzoate, alkyl phenylacetate, alkyl cinnamate, alkyl oxalate, alkyl maleate, alkyl lactone, alkyl oleate, and the like.

In addition, the first solvent may also be one or more selected from the group consisting of: aliphatic ketone, such as 2-nonanone, 3-nonanone, 5-nonanone, 2-decanone, 2,5-hexanedione, 2,6,8-trimethyl-4-nonanone, phorone, di-n-amyl ketone, and the like; and aliphatic ether, such as pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, and the like.

In one embodiment, the organic solvent further includes a second solvent, which is one or more selected from the group consisting of methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxahexane, acetone, methyl ethyl ketone, 1, 2-dichloroethane, 3-phenoxytoluene, 1, 1, 1-trichloroethane, 1, 1, 2, 2-tetrachloroethane, ethyl acetate, butyl acetate, dimethylformamide, dimethylacetamide, dimethyl sulfoxide, tetrahydronaphthalene, decalin, and indene.

In one embodiment, the formulation may be a solution or a suspension, which is determined based on the compatibility between the organic compound and the organic solvent.

In one embodiment, the organic compound in the formulation has a weight percentage of 0.01 wt % to 20 wt %. In one of the embodiments, the weight percentage of the organic compound in the formulation ranges from 0.1 wt % to 15 wt %. In one of the embodiments, the weight percentage of the organic compound in the formulation ranges from 0.2 wt % to 10 wt %. In one of the embodiments, the weight percentage of the organic compound in the formulation ranges from 0.25 wt % to 5 wt %.

In one embodiment, the aforementioned formulation is used in the manufacturing of an organic electronic device. In particular, the aforementioned formulation is used as a coating or printing ink in the manufacturing of the organic electronic device, particularly the manufacturing method is that by printing or coating.

Suitable printing or coating techniques include, but are not limited to, inkjet printing, nozzle printing, typography printing, screen printing, dip coating, spin coating, blade coating, roller printing, torsion printing, lithography, flexographic printing, rotary printing, spraying, brushing or pad printing, or slit-type extrusion coating and so on. In one embodiment, the printing or coating techniques are gravure printing, nozzle printing, and inkjet printing. The formulation may further include a component, which is one or more selected from the group consisting of a surface active compound, a lubricant, a wetting agent, a dispersing agent, a hydrophobic agent, and a binder, for adjusting the viscosity and film forming properties, and improving adhesion. Detailed information relevant to printing technology and related requirements for related solutions, such as solvent, concentration, viscosity, and the like, may be referred to Handbook of Print Media: Technologies and Production Methods, Helmut Kipphan, ISBN 3-540-67326-1.

A formulation according to another embodiment includes the aforementioned organic mixture and the organic solvent. The content, structure, and the like of each component in the formulation are as described in the previous embodiment, and will not be described in detail herein.

In one embodiment, an application of the aforementioned organic compound or organic mixture in an organic electronic device is provided. The organic electronic device may be selected from the group consisting of an organic light-emitting diode (OLED), an organic photovoltage cell (OPV), an organic light-emitting electrochemical cell (OLEEC), an organic field-effect transistor (OFET), an organic light-emitting field-effect transistor, an organic laser, an organic spin electronic device, an organic sensor, and an organic plasmon emitting diode. In one embodiment, the organic electronic device is an OLED. In addition, the organic compound or mixture is used for a light-emitting layer of the OLED device.

An organic electronic device according to an embodiment includes at least one organic compound or organic mixture as described above. The organic electronic device may include a cathode, an anode, and a functional layer located between the cathode and the anode, and the functional layer includes the aforementioned organic compound or mixture. Specifically, the organic electronic device includes at least a cathode, an anode, and one functional layer located between the cathode and the anode, and the functional layer includes at least one organic compound or mixture as described above. The functional layer is one or more selected from the group consisting of a hole injection layer, a hole transport layer, a hole blocking layer, an electron injection layer, an electron transport layer, an electron blocking layer, and a light-emitting layer.

The organic electronic device may be selected from the group consisting of an organic light-emitting diode (OLED), an organic photovoltage cell (OPV), an organic light-emitting electrochemical cell (OLEEC), an organic field-effect transistor (OFET), an organic light-emitting field-effect transistor, an organic laser, an organic spin electronic device, an organic sensor, and an organic plasmon emitting diode. In one embodiment, the organic electronic device is an organic electroluminescent device such as an OLED, an OLEEC, and an organic light-emitting field-effect transistor. In addition, the organic light-emitting diode may be an evaporated organic light-emitting diode or a printed organic light-emitting diode.

In an embodiment, the light-emitting layer of the organic electroluminescent device includes the aforementioned organic compound or the organic mixture, or includes one of the aforementioned organic compounds or mixtures and a phosphorescent emitter, or includes one of the aforementioned organic compounds or mixtures and a host material, or includes one of the aforementioned organic compounds or mixtures, a phosphorescent emitter, and a host material.

In one of the embodiments, the electron transport layer of the electroluminescent device includes the aforementioned organic compound.

In an embodiment, the organic electroluminescent device includes a substrate, an anode, a light-emitting layer, and a cathode, which are sequentially laminated. There is at least one light-emitting layer.

The substrate may be opaque or transparent. The transparent substrate can be used to manufacture a transparent light-emitting device, which may be referred to Bulovic et al., Nature 1996, 380, p 29 and Gu et al., Appl. Phys. Lett. 1996, 68, p 2606. The substrate may be rigid or elastic. The substrate may also be plastic, metal, semiconductor wafer, or glass. In one embodiment, the substrate has a smooth surface. The substrates without surface defects are a particularly desirable selection. In an embodiment, the substrate is flexible and may be selected from a polymer film or a plastic having a glass transition temperature T_{g} greater than 150° C. The flexible substrate may be poly(ethylene terephthalate) (PET) or polyethylene glycol (2, 6-naphthalene) (PEN). In one of the embodiments, the glass transition temperature T_{g} of the substrate is greater than 200° C. In one of the embodiments, the glass transition temperature T_{g} of the substrate is greater than 250° C. In one of the embodiments, the glass transition temperature T_{g} of the substrate is greater than 300° C.

The anode may include a conductive metal, a metal oxide, or a conductive polymer. The anode can easily inject holes into the hole injection layer (HIL), or the hole transport layer (HTL), or the light-emitting layer. In one embodiment, the absolute value of the difference between the work function of the anode and the HOMO energy level or the valence band energy level of the emitter in the light-emitting layer or of the p-type semiconductor material as the HIL or HTL or the electron blocking layer (EBL) is less than 0.5 eV, further less than 0.3 eV, and still further less than 0.2 eV. Examples of the anode materials include, but are not limited to, Al, Cu, Au, Ag, Mg, Fe, Co, Ni, Mn, Pd, Pt, ITO, aluminum-doped zinc oxide (AZO), and the like. The anode material can also be other materials. The anode material can be deposited using any suitable technique, such as a suitable physical vapor deposition process, including radio frequency magnetron sputtering, vacuum thermal evaporation, electron beam (e-beam), and the like. In other embodiments, the anode is patterned. A patterned ITO conductive substrate is commercially available and can be used to fabricate the organic electronic device according to the present embodiment.

The cathode may include a conductive metal or metal oxide. The cathode can easily inject electrons into the EIL or ETL or directly into the light-emitting layer. In one embodiment, the absolute value of the difference between the work function of the cathode and the LUMO energy level or the conduction band energy level of the emitter in the light-emitting layer or of the n-type semiconductor material as the electron injection layer (EIL) or the electron transport layer (ETL) or the hole blocking layer (HBL) is less than 0.5 eV, further less than 0.3 eV, and still further less than 0.2 eV. All the materials that can be used as the cathode of the OLED can serve as the cathode material of the organic electronic device of the present embodiment. Examples of the cathode material include, but are not limited to, Al, Au, Ag, Ca, Ba, Mg, LiF/Al, MgAg alloy, BaFi/Al, Cu, Fe, Co, Ni, Mn, Pd, Pt, ITO, and the like. The cathode material can be deposited using any suitable technique, such as a suitable physical vapor deposition process, including radio frequency magnetron sputtering, vacuum thermal evaporation, and electron beam (e-beam), and the like.

The OLED may further include other functional layers such as a hole injection layer (HIL), a hole transport layer (HTL), an electron blocking layer (EBL), an electron injection layer (EIL), an electron transport layer (ETL), or a hole blocking layer (HBL). Suitable materials for these functional layers are described in detail above and in WO2010135519A1, US20090134784A1, and WO2011110277A1, the entire contents of these three patent documents are hereby incorporated by reference.

In one of the embodiments, the electron transport layer (ETL) or the hole blocking layer (HBL) of the electroluminescent device includes the aforementioned organic compound or polymer. In one of the embodiments, the light-emitting layer of the electroluminescent device is manufactured by using the aforementioned formulation.

In an embodiment, the light-emitting layer is separately manufactured by the following two methods:

(1) the mixture including the organic compound H1 and the organic compound H2 is deposited as one source. The light-emitting layer may be manufactured by a printing method using the aforementioned formulation, or by vacuum evaporation of the mixture as one source.

(2) the organic compound H1 and the organic compound H2 are evaporated as two separate sources.

In one of the embodiments, the compounds represented by the general formula (1) and the general formula (2) have a small molecular weight difference and a corresponding small sublimation temperature. In order to further simplify the material evaporation process and reduce the production cost of the OLED display device, the two host materials, that is, the organic compound H1 and the organic compound H2 may be uniformly mixed in a certain proportion, and then are evaporated by one evaporation heat source when manufacturing the light-emitting layer by using the organic mixture.

In an embodiment, the organic electroluminescent device has a light emission wavelength between 300 nm and 1000 nm. In one of the embodiments, the light emission wavelength of the organic electroluminescent device is between 350 nm and 900 nm. In one of the embodiments, the light emission wavelength of the organic electroluminescent device is between 400 nm and 800 nm.

In an embodiment, the aforementioned organic electronic device can be used in an electronic device. The electronic device is selected from display device, a lighting device, a light source, or a sensor. The organic electronic device may be an organic electroluminescent device.

An electronic device includes the aforementioned organic electronic device.

Synthesis of the Organic Compound H2

### Example 1

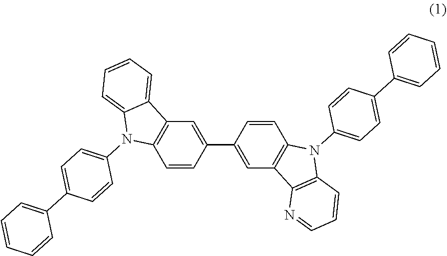 [SMILES: c1ccc(-c2ccc(-n3c4ccccc4c4cc(-c5ccc6c(c5)c5ncccc5n6-c5ccc(-c6ccccc6)cc5)ccc43)cc2)cc1]

### 5-([1, 1′-diphenyl]-4-yl)-8-(9-([1, 1′-diphenyl]-4-yl)-9H-carbazole-3-yl)-5H-pyridine [3, 2-b] indole

In a 250 ml three-necked flask, 3.63 g, 10 mmol of(9-([1, 1′-diphenyl]-4-yl)-9H-carbazole-3-yl)boronic acid, 3.98 g, 10 mmol of 5-([1, 1′-diphenyl]-4-yl)-8-bromo-5H-pyridine [3, 2-b]indole, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and methanol to give 5.8 g of product 5-([1, 1′-diphenyl]-4-yl)-8-(9-([1, 1′-diphenyl]-4-yl)-9H-carbazole-3-yl)-5H-pyridine [3, 2-b] indole, MS (ASAP)=637.4.

### Example 2

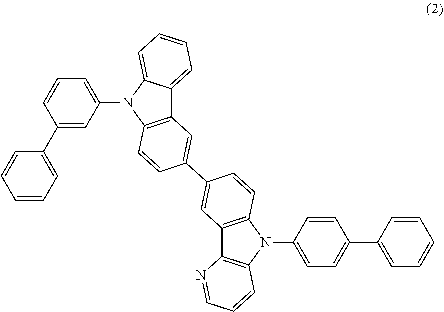 [SMILES: c1ccc(-c2ccc(-n3c4ccc(-c5ccc6c(c5)c5ccccc5n6-c5cccc(-c6ccccc6)c5)cc4c4ncccc43)cc2)cc1]

### 8-(9-([1, 1′-diphenyl]-3-yl)-9H-carbazole-3-yl)-5-([1, 1′-diphenyl]-4-yl)-5H-pyridine [3, 2-b] indole

In a 250 ml three-necked flask, 3.63 g, 10 mmol of (9-([1, 1′-diphenyl]-3-yl)-9H-carbazole-3-yl)boronic acid, 3.98 g, 10 mmol of 5-([1, 1′-diphenyl]-4-yl)-8-bromo-5H-pyridine [3, 2-b]indole, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and methanol to give 5.4 g of product 8-(9-([1, 1′-diphenyl]-3-yl)-9H-carbazole-3-yl)-5-([1, 1′-diphenyl]-4-yl)-5H-pyridine [3, 2-b] indole, MS (ASAP)=637.8.

### Example 3

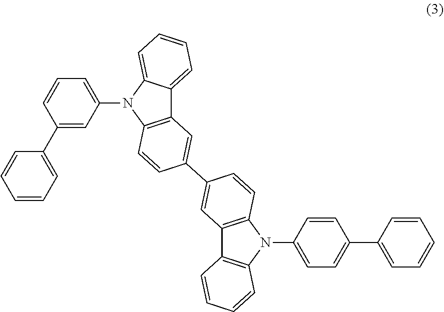 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### 6-(9-([1, 1′-diphenyl]-3-yl)-9H-carbazole-3-yl)-9-([1, 1′-diphenyl]-4-yl)-9H-pyridine [2, 3-b] indole

In a 250 ml three-necked flask, 3.63 g, 10 mmol of(9-([1, 1′-diphenyl]-3-yl)-9H-carbazole-3-yl)boronic acid, 3.98 g, 10 mmol of 9-([1, 1′-diphenyl]-4-yl)-3-bromo-9H-carbazole, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and methanol to give 5.4 g of product 6-(9-([1, 1′-diphenyl]-3-yl)-9H-carbazole-3-yl)-9-([1, 1′-diphenyl]-4-yl)-9H-pyridine [2, 3-b] indole, MS (ASAP)=636.6.

### Example 4

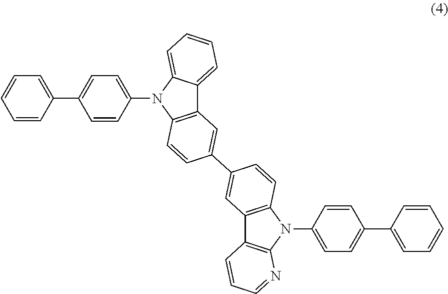 [SMILES: c1ccc(-c2ccc(-n3c4ccccc4c4cc(-c5ccc6c(c5)c5cccnc5n6-c5ccc(-c6ccccc6)cc5)ccc43)cc2)cc1]

### 9-([1, 1′-diphenyl]-4-yl)-6-(9-([1, 1′-diphenyl]-4-yl)-9H-carbazole-3-yl)-9H-pyridine [2, 3-b] indole

In a 250 ml three-necked flask, 3.63 g, 10 mmol of (9-([1, 1′-diphenyl]-4-yl)-9H-carbazole-3-yl)boronic acid, 3.98 g, 10 mmol of 9-([1, 1′-diphenyl]-4-yl)-6-bromo-9H-pyridine [2, 3-b]indole, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and methanol to give 5.5 g of product 9-([1, 1′-diphenyl]-4-yl)-6-(9-([1, 1′-diphenyl]-4-yl)-9H-carbazole-3-yl)-9H-pyridine [2, 3-b] indole, MS (ASAP)=637.4.

Synthesis of the Organic Compound H1

### Example 5

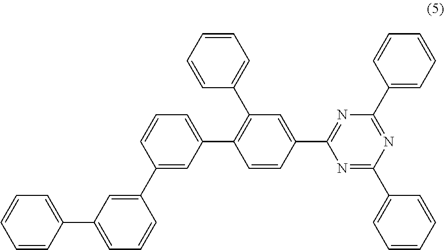 [SMILES: c1ccc(-c2cccc(-c3cccc(-c4ccc(-c5nc(-c6ccccc6)nc(-c6ccccc6)n5)cc4-c4ccccc4)c3)c2)cc1]

### 2-([1, 1′: 2′, 1″: 3″, 1′″: 3′″, 1″″-pentaphenyl]-5′-yl)-4, 6-diphenyl-1, 3, 5-triazine

In a 250 ml three-necked flask, 2.74 g, 10 mmol of [1, 1′: 3′, 1″-triphenyl]-3-yl boronic acid, 5.07 g, 11 mmol of 2-(6-bromo-[1, 1′-diphenyl]-3-yl)-4, 6-diphenyl-1, 3, 5-triazine, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and petroleum ether to give 5.0 g of product 2-([1, 1′: 2′, 1″: 3″, 1′″: 3′″, 1″″-pentaphenyl]-5′-yl)-4, 6-diphenyl-1, 3, 5-triazine, MS (ASAP)=613.2.

### Example 6

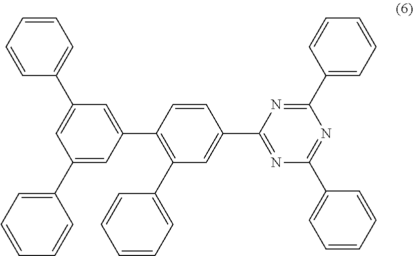 [SMILES: c1ccc(-c2cc(-c3ccccc3)cc(-c3ccc(-c4nc(-c5ccccc5)nc(-c5ccccc5)n4)cc3-c3ccccc3)c2)cc1]

### 2, 4-diphenyl-6-(5″-phenyl-[1, 1′: 2′, 1″: 3″, 1′″-pentaphenyl]-5′-yl)-1, 3, 5-triazine

In a 250 ml three-necked flask, 2.74 g 10 mmol of [1, 1′: 3′, 1″-triphenyl]-5′-yl boronic acid, 5.07 g, 11 mmol of 2-(6-bromo-[1, 1′-diphenyl]-3-yl)-4, 6-diphenyl-1, 3, 5-triazine, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from dichloromethane and petroleum ether to give 4.8 g of product 2, 4-diphenyl-6-(5″-phenyl-[1, 1′: 2′, 1″: 3″, 1″′-pentaphenyl]-5′-yl)-1, 3, 5-triazine, MS (ASAP)=613.4.

### Example 7

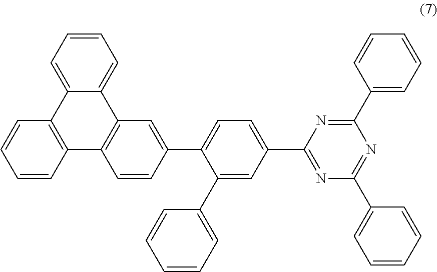 [SMILES: c1ccc(-c2nc(-c3ccccc3)nc(-c3ccc(-c4ccc5c6ccccc6c6ccccc6c5c4)c(-c4ccccc4)c3)n2)cc1]

### 2, 4-diphenyl-6-(6-(anthracene-2-yl)-[1′-diphenyl]-3-yl)-1, 3, 5-triazine

The synthesis steps were the same as the synthesis steps of Example 5. In a 250 ml three-necked flask, 2.73 g, 10 mmol of (benzophenanthryl-2-yl) boronic acid, 5.07 g, 11 mmol of 2-(6-bromo-[1, 1′-diphenyl]-3-yl)-4, 6-diphenyl-1, 3, 5-triazine, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. The crude product was recrystallized from o-dichlorobenzene by heating to give the product 5.1 g, MS (ASAP)=611.4.

### Example 8

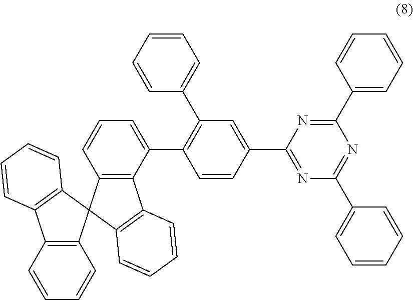 [SMILES: c1ccc(-c2nc(-c3ccccc3)nc(-c3ccc(-c4cccc5c4-c4ccccc4C54c5ccccc5-c5ccccc54)c(-c4ccccc4)c3)n2)cc1]

### 2-(6-(9, 9′-spirobi [fluorene]-4-yl)-[1, 1′-diphenyl]-3-yl-4, 6-diphenyl-1, 3, 5-triazine

The synthesis steps were similar to the synthesis steps of Example 5. In a 250 ml three-necked flask, 3.60 g, 10 mmol of 9, 9′-spirobi [fluorene]-4-yl boronic acid, 5.07 g, 11 mmol of 2-(6-bromo-[1, 1′-diphenyl]-3-yl)-4, 6-diphenyl-1, 3, 5-triazine, 6.9 g, 50 mmol of potassium carbonate, 0.58 g, 0.5 mmol of Pd (PPh₃)₄, 100 ml of toluene, 25 ml of water, and 25 ml of ethanol were added, and were reacted in an atmosphere of N₂ at a temperature of 110° C. The reaction progress was tracked by TLC. After the reaction was completed, the reaction solution was cooled to room temperature. The reaction solution was poured into water, washed to remove K₂CO₃, and then was suction-filtered to obtain a solid product, which was then washed with dichloromethane. Recrystallization was performed by using dioxane to give 4.5 g of 2-(6-(9, 9′-spirobi [fluorene]-4-yl)-[1, 1′-diphenyl]-3-yl-4, 6-diphenyl-1, 3, 5-triazine as a solid powder, MS (ASAP)=699.2.

A preparation process of the organic mixture is as follows. The organic compound H1 and the organic compound H2 having a mass ratio of 1:1 are mixed uniformly, and then the mixture is disposed in a vacuum environment of less than or equal to 10⁻³ Torr. The temperature in the vacuum environment is raised to completely melt the two host materials. After being mixed uniformly, the mixture is solidified by cooling to room temperature, and then ground into a powder by a ball mill for use.

The energy levels of the organic compound materials can be calculated by quantum, for example, using TD-DFT (time-dependent density functional theory) by Gaussian09W (Gaussian Inc.), and specific simulation methods can be referred to WO2011141110. Firstly, the molecular geometry is optimized by semi-empirical method “Ground State/Semi-empirical/Default Spin/AM1” (Charge 0/Spin Singlet). Then, the energy structure of organic molecules is calculated by TD-DFT (time-dependent density functional theory) method for “TD-SCF/DFT/Default Spin/B3PW91” and the base group “6-31G (d)” (Charge 0/Spin Singlet). The HOMO and LUMO energy levels are calculated according to the following calibration equations: S₁, T₁, and the resonance factor f (S₁) are used directly.

HOMO(eV)=((HOMO(G)×27.212)−0.9899)/1.1206

LUMO(eV)=((LUMO(G)×27.212)−2.0041)/1.385

HOMO (G) and LUMO (G) are direct calculation results of Gaussian 09W, in units of Hartree. The results are shown in Table 1.

TABLE 1

| | | | | | ΔHOMO |
|---|---|---|---|---|---|
| Materials | HOMO [eV] | LUMO [eV] | T₁ [eV] | S₁ [eV] | [eV] |
| (1) | −5.48 | −2.33 | 2.90 | 3.03 | 0.43 |
| (2) | −5.51 | −2.33 | 2.91 | 3.34 | 0.43 |
| (3) | −5.43 | −2.24 | 2.90 | 3.11 | 0.41 |
| (4) | −5.54 | −2.31 | 2.90 | 3.10 | 0.40 |
| (5) | −6.28 | −2.79 | 2.98 | 3.39 | 0.12 |
| (6) | −6.32 | −2.78 | 3.01 | 3.39 | 0.06 |
| (7) | −6.08 | −2.82 | 2.70 | 3.41 | 0.16 |
| (8) | −5.98 | −2.79 | 2.95 | 3.28 | 0.26 |

The materials (1) to (4) are the organic compounds H2 obtained in Examples 1 to 4, and the materials (5) to (8) are the organic compounds H1 obtained in Examples 5 to 8. The numbering and composition of the mixture refer to Table 2. A mass ratio of the organic compound H1 to the organic compound H2 in the mixture is 1:1.

TABLE 2

| | (1) | (2) | (3) | (4) | (5) | (6) | (7) | (8) |
|---|---|---|---|---|---|---|---|---|
| A-1 | ◯ | | | | ◯ | | | |
| A-2 | | ◯ | | | | ◯ | | |
| A-3 | | ◯ | | | | | ◯ | |
| A-4 | | | ◯ | | | | | ◯ |
| A-5 | | | | ◯ | | | | ◯ |
| A-6 | ◯ | | | | | | | ◯ |
| A-7 | | | ◯ | | | | ◯ | |
| A-8 | | ◯ | | | | | | ◯ |

In comparison with the aforementioned mixed phosphorescent host material, the host material of the currently used carbazole material system structure is marked with Ref 1:

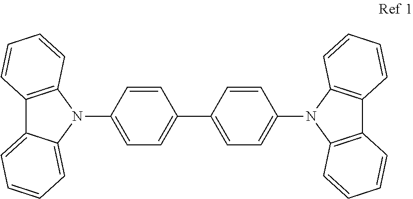 [SMILES: c1ccc2c(c1)c1ccccc1n2-c1ccc(-c2ccc(-n3c4ccccc4c4ccccc43)cc2)cc1]

Fabrication of OLED Devices:

Fabrication steps for an OLED device having ITO/HATCN (10 nm)/NPB (35 nm)/TCTA (5 nm)/(A-1)-(A-8): 5% Ir(ppy)₃/B3P YMPM (40 nm)/LiF (1 nm)/Al (150 nm) are as follows:

a. cleaning of conductive glass substrate: when the conductive glass substrate is used for the first time, a variety of solvents, such as chloroform, ketone, or isopropanol can be used for cleaning, and then treating with UV and ozone plasma;

b. HTL (35 nm), EML (15 nm), and ETL (65 nm): it is obtained by thermal evaporation in a high vacuum (1×10⁻⁶ mbar);

c. cathode: LiF/Al (1 nm/150 nm) is deposited by thermal evaporation in the high vacuum (1×10⁻⁶ mbar);

d. encapsulation: the device is encapsulated in the nitrogen glove box with UV curable resin.

The current-voltage (J-V) characteristics of each OLED device are characterized by characterization equipment while recording important parameters such as efficiency, lifetime, and external quantum efficiency. It has been tested that the luminous efficiency and lifetime of OLED 4 (corresponding to material (A-4)) are more than three times that of OLED Ref1 (corresponding to material (Ref1)). The luminous efficiency of OLED 7 (corresponding to material (A-7)) is five times that of the OLED Ref1, and the lifetime of the OLED 7 is more than eight times that of the OLED Ref1. In particular, the maximum external quantum efficiency of the OLED 7 is more than 19%. It can be seen that the OLED device manufactured by using the organic mixture of the present disclosure has greatly improved the luminous efficiency and lifetime, and the external quantum efficiency thereof is also significantly improved.

In addition, mixtures A-5, A-6, A-7, and A-8 were also used as a single host for the manufacturing of OLEDs, and the efficiency and lifetime were both more than 1.5 times than those of Ref1. However, in this device structure, a co-host can achieve better performance.

1-24. (canceled)

25. An organic compound for an electronic device having a general formula (1) as following: 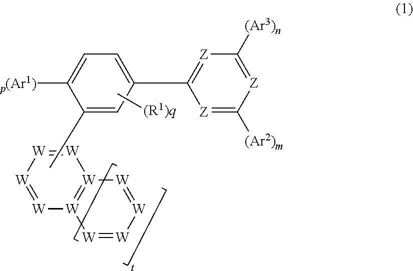
wherein,
Z is selected from N or CR⁷, and at least one Z is N;
W is selected from N or CR⁷, and two linked Ws are not N simultaneously;
Ar¹ to Ar³ are independently selected from the group consisting of an aromatic ring system containing 5 to 30 ring atoms, a heteroaromatic ring system containing 5 to 30 ring atoms, and a non-aromatic ring system containing 5 to 30 ring atoms; Ar¹ to Ar³ have a group R⁸ on rings thereof;
R¹ represents H, D, F, CN, alkenyl, alkynyl, nitrile, amino, nitro, acyl, alkoxy, carbonyl, sulfonyl, an alkyl containing 1 to 30 carbon atoms, a cycloalkyl containing 3 to 30 carbon atoms, an aromatic hydrocarbyl containing 5 to 60 ring atoms, or an aromatic heterocyclic group containing 5 to 60 ring atoms;
R⁷ and R⁸ are independently selected from the group consisting of hydrogen, deuterium, a substituted or unsubstituted alkyl containing 1 to 10 carbon atoms, a substituted or unsubstituted aromatic ring system containing 5 to 12 ring atoms, and a substituted or unsubstituted heteroaromatic ring system containing 5 to 12 ring atoms;
n, m, p, and q are independently selected from 1, 2, or 3; t is 0 or 1.

26. The organic compound of claim 25, wherein Ar¹ to Ar³ are independently selected from the group consisting of: 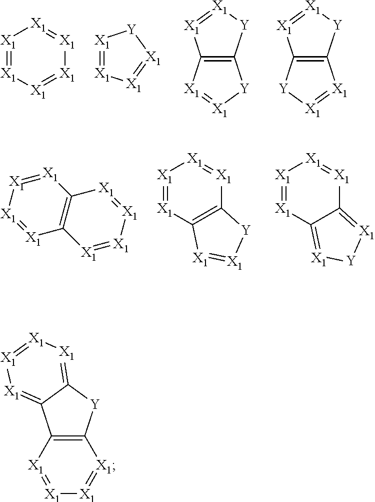
wherein X₁ is selected from CR¹⁰ or N;
Y is selected from the group consisting of CR¹¹R¹², SiR¹³R¹⁴, NR¹⁵, C(═O), S, and O; R¹⁰, R¹¹, R¹², R¹³, R¹⁴, and R¹⁵ are one or more selected from the group consisting of H, D, a linear alkyl containing 1 to 20 carbon atoms, an linear alkoxy containing 1 to 20 carbon atoms, a linear thioalkoxy group containing 1 to 20 carbon atoms, a branched or a cyclic alkyl containing 3 to 20 carbon atoms, a branched or a cyclic alkoxy containing 3 to 20 carbon atoms, a branched or a cyclic thioalkoxy group containing 3 to 20 carbon atoms, a branched or a cyclic silyl group containing 3 to 20 carbon atoms, a substituted keto group containing 1 to 20 carbon atoms, an alkoxy carbonyl group containing 2 to 20 carbon atoms, an aryloxy carbonyl group containing 7 to 20 carbon atoms, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a crosslinkable group, a substituted or unsubstituted aromatic ring system containing 5 to 40 ring atoms or a substituted or unsubstituted heteroaromatic ring system containing 5 to 40 ring atoms, and an aryloxy group containing 5 to 40 ring atoms or a heteroaryloxy group containing 5 to 40 ring atoms; wherein at least one of R¹⁰, R¹¹, R¹², R¹³, R¹⁴, and R¹⁵ forms a monocyclic or polycyclic aliphatic or aromatic ring system with a ring bonded to the group, or at least two of R¹⁰, R¹¹, R¹², R¹³, R¹⁴, and R¹⁵ form a monocyclic or polycyclic aliphatic or aromatic ring with each other.

27. The organic compound of claim 25, wherein the organic compound is a compound represented by one of general formulas (2) to (3):

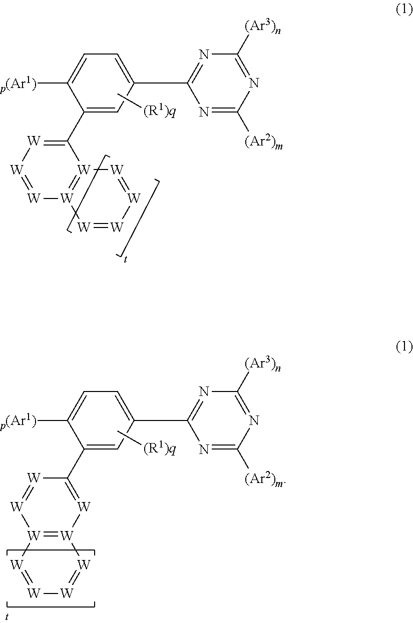

28. The organic compound of claim 25, wherein the organic compound has a general formula (1b) as following:

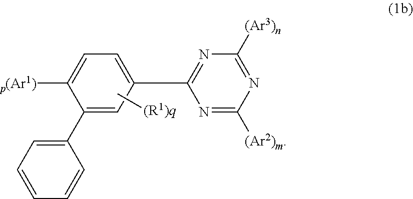

29. The organic compound of claim 25, wherein the organic compound has a general formula (1a) as following:

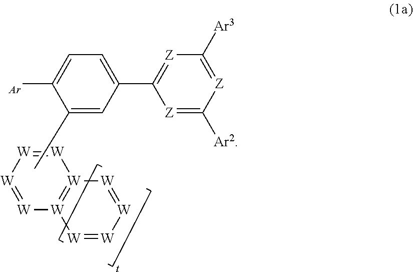

30. The organic compound of claim 26, wherein Ar¹ to Ar³ are independently selected from the group consisting of: 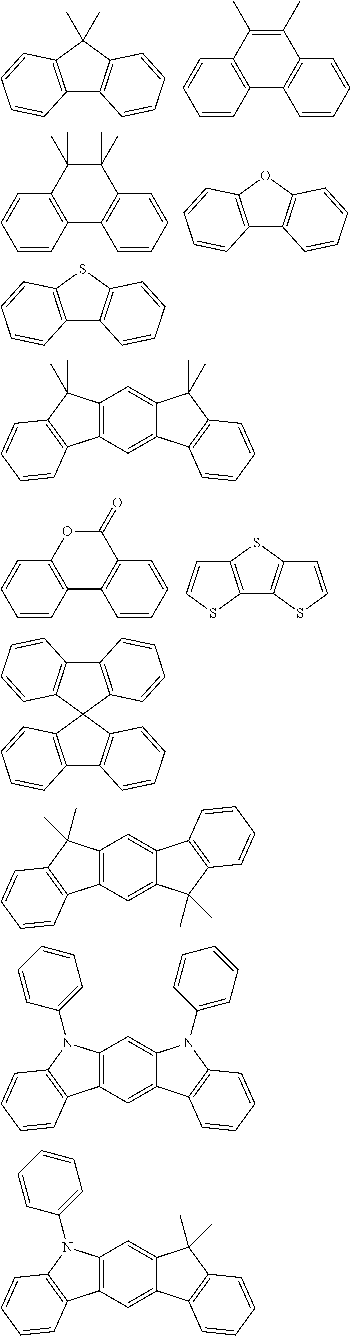 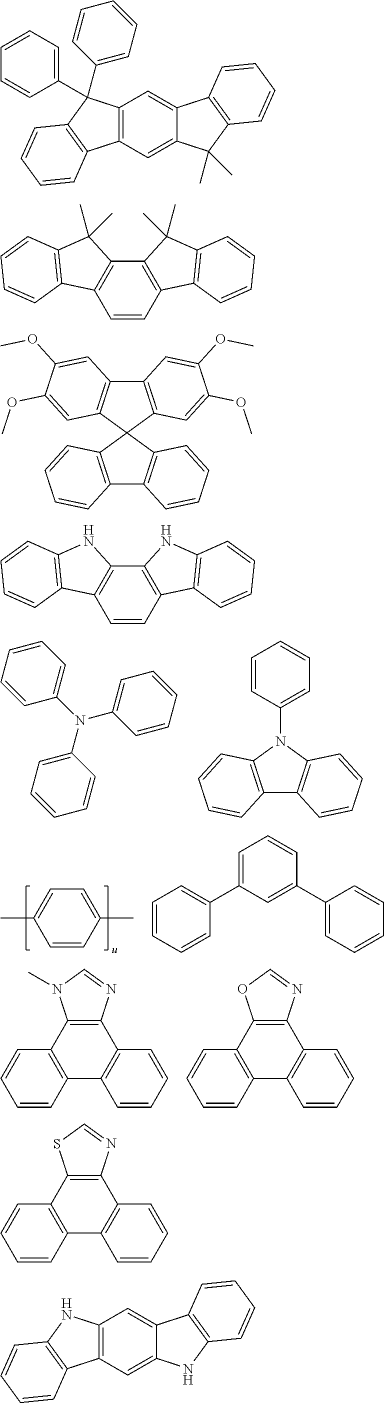 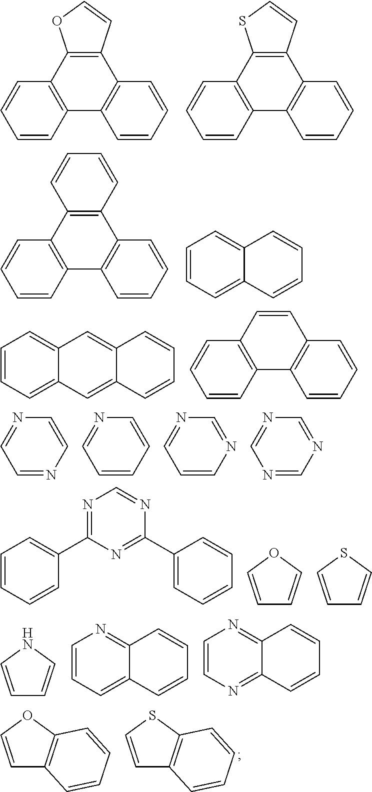
wherein u is selected from 1, 2, 3, or 4.

31. The organic compound of claim 25, wherein Ar¹, Ar², Ar³ are independently selected from the group consisting of the aromatic ring system containing 5 to 30 ring atoms.

32. The organic compound of claim 31, wherein Ar¹ is independently selected from the group consisting of: 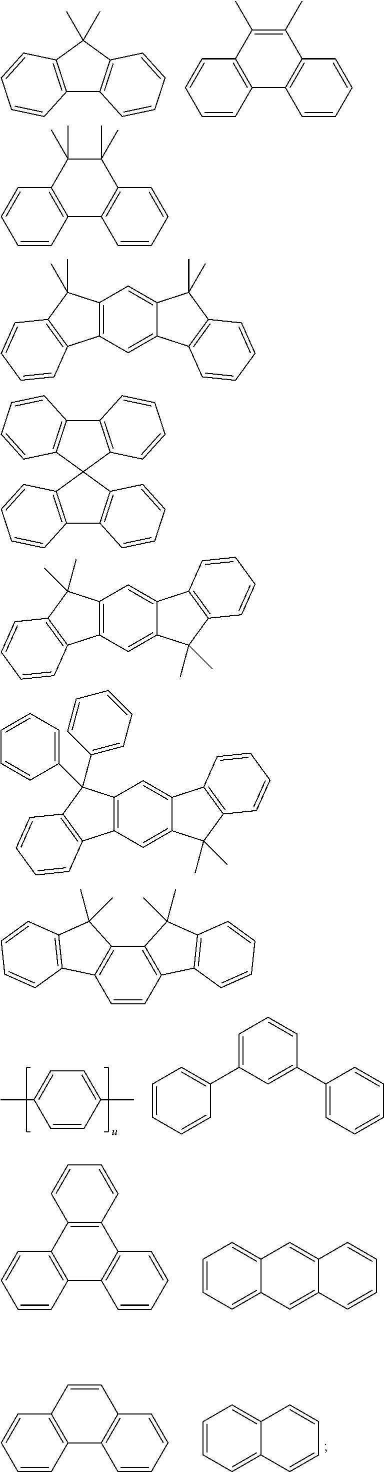
wherein u is selected from 1, 2, 3, or 4.

33. The organic compound of claim 31, wherein Ar¹ is selected from the group consisting of: 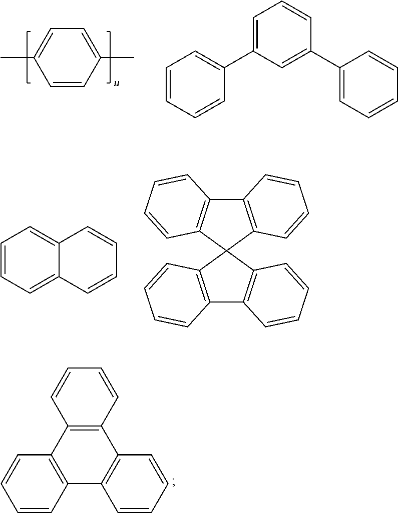
Ar² and Ar³ are selected from the group consisting of: 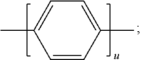
wherein u is selected from 1, 2, 3, or 4.

34. The organic compound of claim 25, wherein at least one Ar comprises an electron-donating group and/or at least one Ar comprises an electron-accepting group in multiple occurrences; wherein Ar is Ar¹, Ar², or Ar³.

35. The organic compound of claim 34, wherein the electron-donating group is selected from the group consisting of:

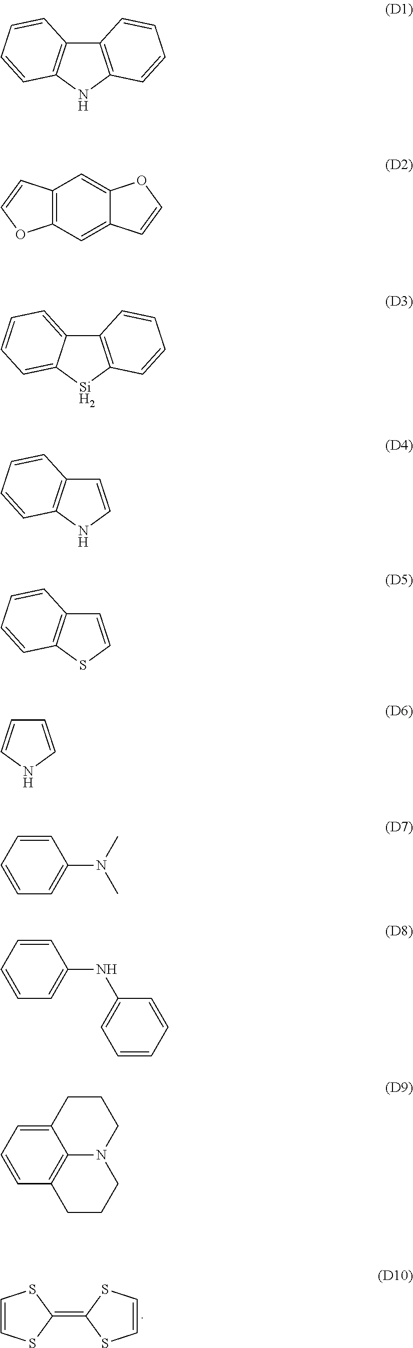

36. The organic compound of claim 34, wherein the electron-accepting group is selected from the group consisting of F, a cyano group, and any one of the following groups: 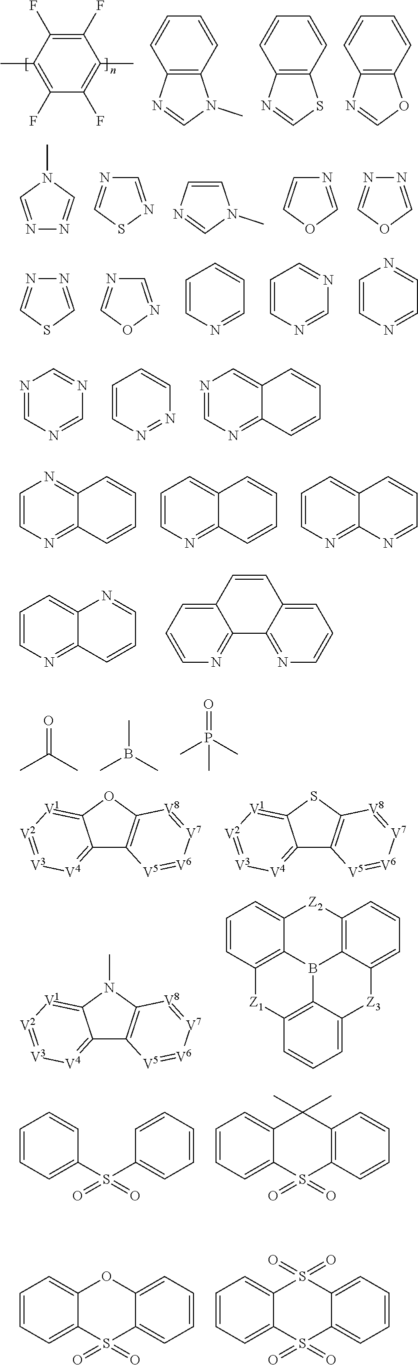
wherein n1 is 1, 2, or 3; V¹ to V⁸ are independently selected from CR¹⁶ or N, and at least one of V¹ to V⁸ is N; R¹⁶ is selected from the group consisting of hydrogen, alkyl, alkoxy, amino, alkene, alkyne, aralkyl, heteroalkyl, aryl, and heteroaryl; Z₁ to Z₃ are selected from the group consisting of a single bond, C(R¹⁶)₂, O, and S.

37. The organic compound of claim 25, wherein the organic compound is selected from the group consisting of:

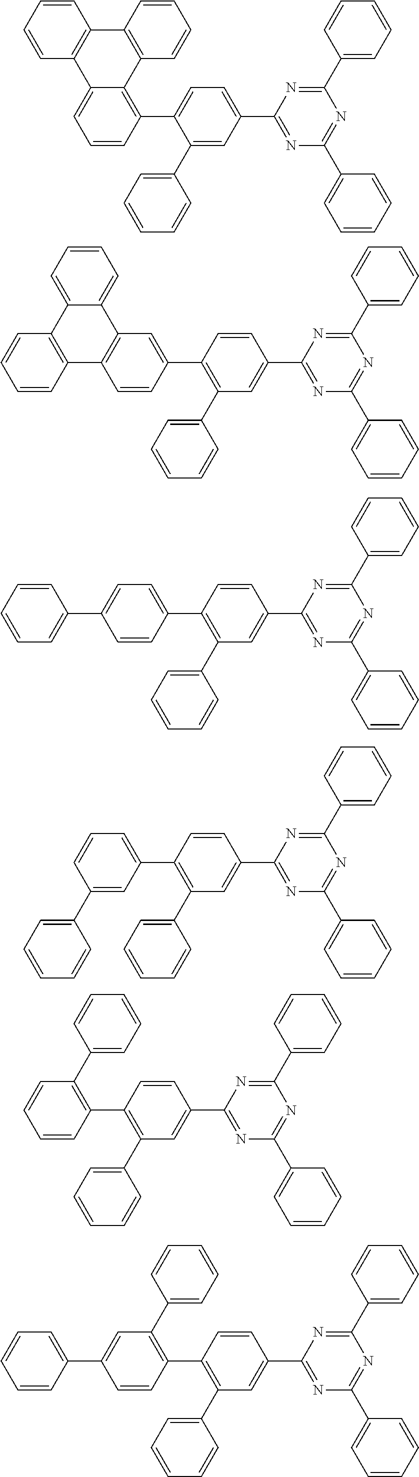

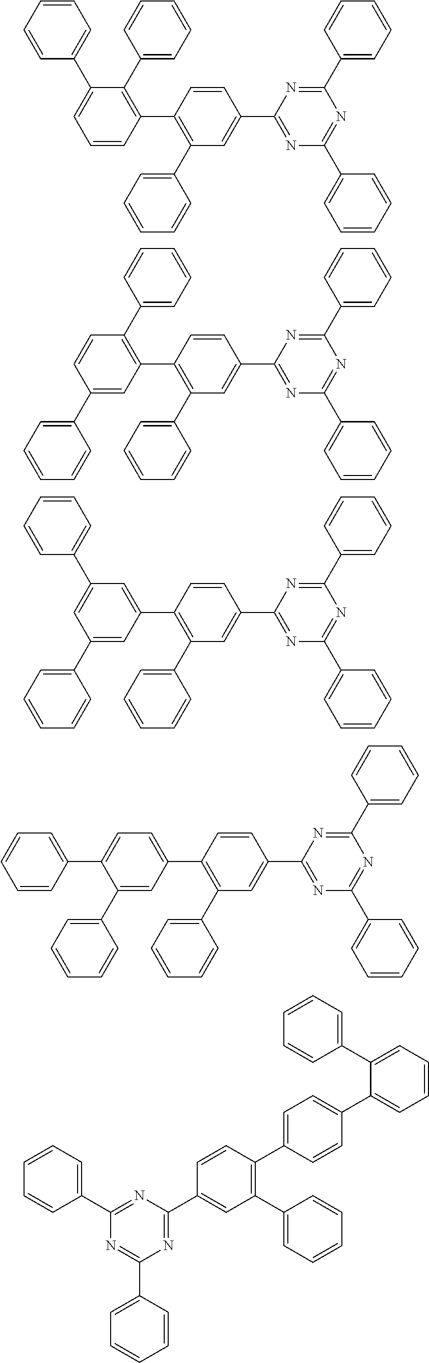

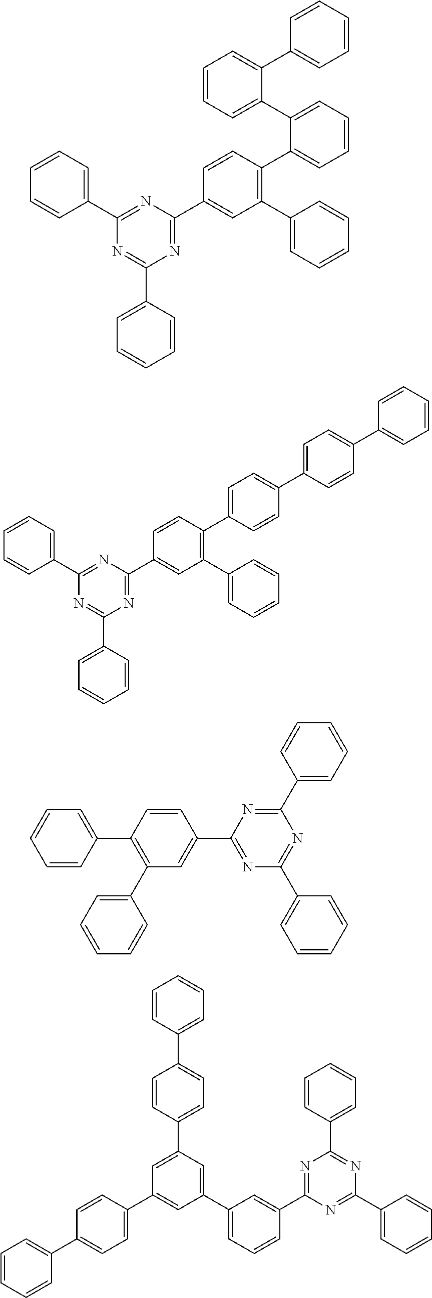 [SMILES: c1ccc(-c2nc(-c3ccccc3)nc(-c3ccc(-c4ccccc4-c4ccccc4-c4ccccc4)c(-c4ccccc4)c3)n2)cc1 ; c1ccc(-c2ccc(-c3ccc(-c4ccc(-c5nc(-c6ccccc6)nc(-c6ccccc6)n5)cc4-c4ccccc4)cc3)cc2)cc1 ; c1ccc(-c2nc(-c3ccccc3)nc(-c3ccc(-c4ccccc4)c(-c4ccccc4)c3)n2)cc1 ; c1ccc(-c2ccc(-c3cc(-c4ccc(-c5ccccc5)cc4)cc(-c4cccc(-c5nc(-c6ccccc6)nc(-c6ccccc6)n5)c4)c3)cc2)cc1]

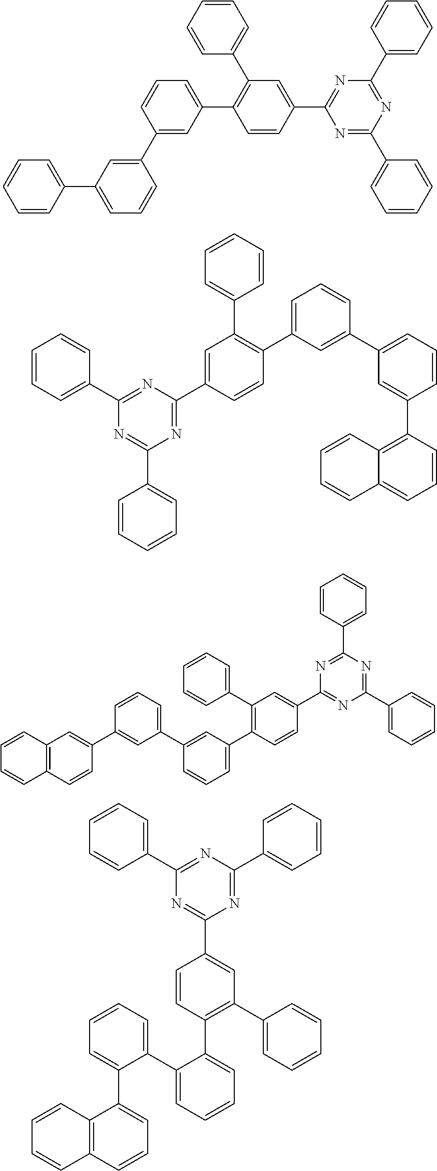

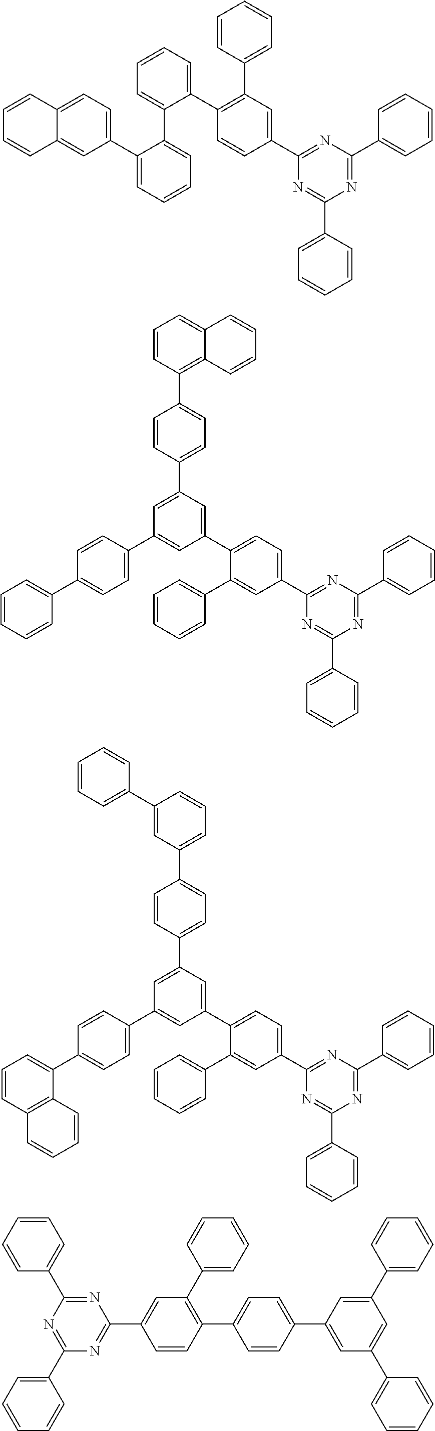 [SMILES: c1ccc(-c2nc(-c3ccccc3)nc(-c3ccc(-c4ccccc4-c4ccccc4-c4ccc5ccccc5c4)c(-c4ccccc4)c3)n2)cc1 ; c1ccc(-c2ccc(-c3cc(-c4ccc(-c5cccc6ccccc56)cc4)cc(-c4ccc(-c5nc(-c6ccccc6)nc(-c6ccccc6)n5)cc4-c4ccccc4)c3)cc2)cc1 ; c1ccc(-c2cccc(-c3ccc(-c4cc(-c5ccc(-c6cccc7ccccc67)cc5)cc(-c5ccc(-c6nc(-c7ccccc7)nc(-c7ccccc7)n6)cc5-c5ccccc5)c4)cc3)c2)cc1 ; c1ccc(-c2cc(-c3ccccc3)cc(-c3ccc(-c4ccc(-c5nc(-c6ccccc6)nc(-c6ccccc6)n5)cc4-c4ccccc4)cc3)c2)cc1]

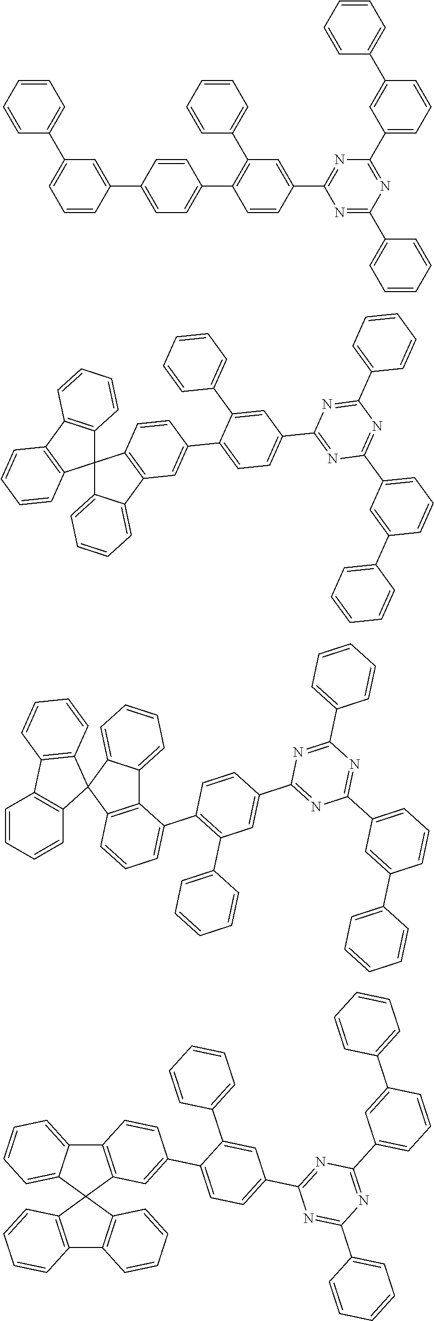

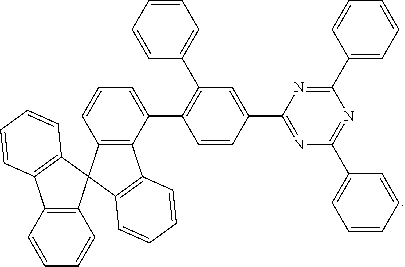 [SMILES: c1ccc(-c2nc(-c3ccccc3)nc(-c3ccc(-c4cccc5c4-c4ccccc4C54c5ccccc5-c5ccccc54)c(-c4ccccc4)c3)n2)cc1]

38. An organic mixture comprising an organic compound H2 and an organic compound H1 of claim 25, min((LUMO(H1)-HOMO(H2)), (LUMO(H2)-HOMO(H1)))≤min(E_{T}(H1), E_{T}(H2))+0.1 eV; wherein LUMO(H1), HOMO(H1), and E_{T}(H1) are energy levels of a lowest unoccupied molecular orbital, a highest occupied molecular orbital, and a triplet excited state of the organic compound H1, respectively; LUMO(H2), HOMO(H2), and E_{T}(H2) are energy levels of a lowest unoccupied molecular orbital, a highest occupied molecular orbital, and a triplet excited state of the organic compound H2, respectively.

39. The organic mixture of claim 38, wherein the organic compound H2 is a compound represented by one of the following general formulas (4) to (7): 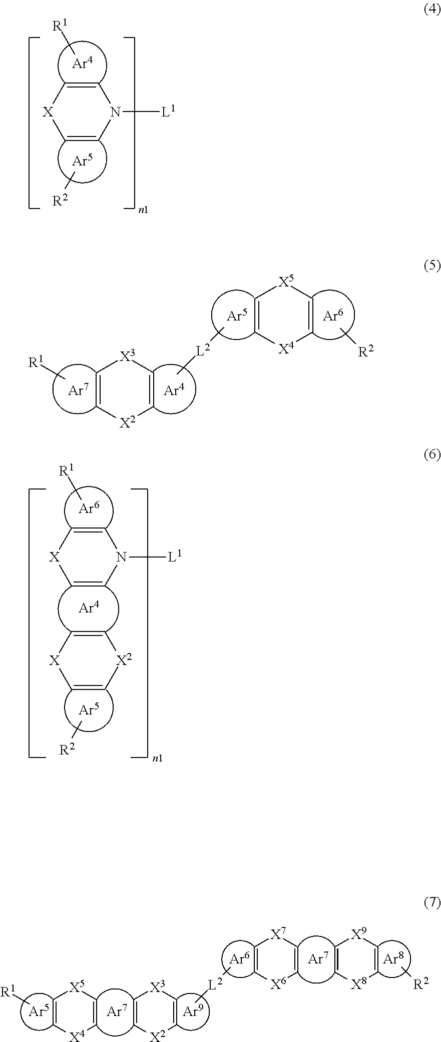
wherein,
L¹ is selected from the group consisting of an aromatic group containing 5 to 60 ring atoms and a heteroaromatic group containing 5 to 60 ring atoms;
L² is selected from the group consisting of a single bond, an aromatic group containing 5 to 30 ring atoms, and a heteroaromatic group containing 5 to 30 ring atoms, and a linking position of L² is any one of carbon atoms on a ring;
Ar⁴, Ar⁵, Ar⁶, Ar⁷, Ar⁸, and Ar⁹ are independently selected from the group consisting of an aromatic group containing 5 to 30 ring atoms and a heteroaromatic group containing 5 to 30 ring atoms;
X is selected from the group consisting of a signal bond, N(R), C(R)₂, Si(R)₂, O, C═N(R), C═C(R)₂, P(R), P(═O)R, S, S═O, and SO₂;
X², X³, X⁴, X⁵, X⁶, X⁷, X⁸, and X⁹ are independently selected from the group consisting of a single bond, N(R), C(R)₂, Si(R)₂, O, C═N(R), C═C(R)₂, P (R), P(═O)R, S, S═O, and SO₂, X² and X³ are not both single bonds simultaneously, X⁴ and X⁵ are not both single bonds simultaneously, X⁶ and X⁷ are not both single bonds simultaneously, and X⁸ and X⁹ are not both single bonds simultaneously;
R¹, R², and R are independently selected from the group consisting of H, D, F, CN, alkenyl, alkynyl, nitrile, amino, nitro, acyl, alkoxy, carbonyl, sulfonyl, an alkyl containing 1 to 30 carbon atoms, a cycloalkyl containing 3 to 30 carbon atoms, an aromatic hydrocarbyl containing 5 to 60 ring atoms, or an aromatic heterocyclic group containing 5 to 60 ring atoms; linking positions of R¹ and R² are any one or more carbon atoms on a fused ring;
n1 is 1, 2, 3, or 4.

40. The organic mixture of claim 39, wherein the organic compound H2 is a compound represented by one of the following general formulas (8) to (11): 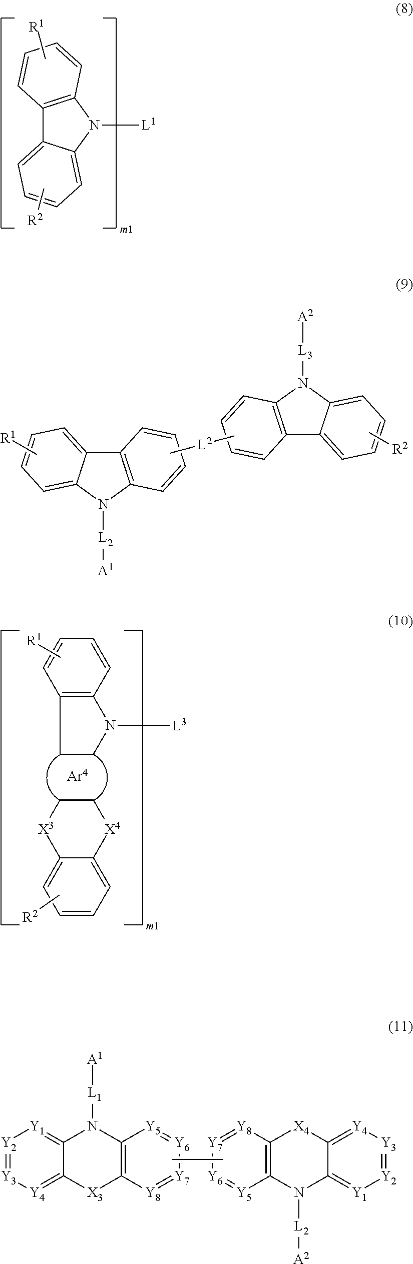
wherein L³ is selected from the group consisting of an aromatic group containing 5 to 60 ring atoms and a heteroaromatic group containing 5 to 60 ring atoms; A¹ and A² are independently selected from the group consisting of an aromatic group containing 5 to 30 ring atoms and a heteroaromatic group containing 5 to 30 ring atoms; Y¹ to Y⁸ are independently selected from N or CR, and adjacent Y¹ to Y⁸ are not both N simultaneously; ml is 1, 2, 3, or 4.

41. The organic mixture of claim 38, wherein a difference between a molecular weight of the organic compound H1 and a molecular weight of the organic compound H2 is less than or equal to 100 g/mol.

42. The organic mixture of claim 38, wherein the organic compound H2 is selected from the group consisting of:

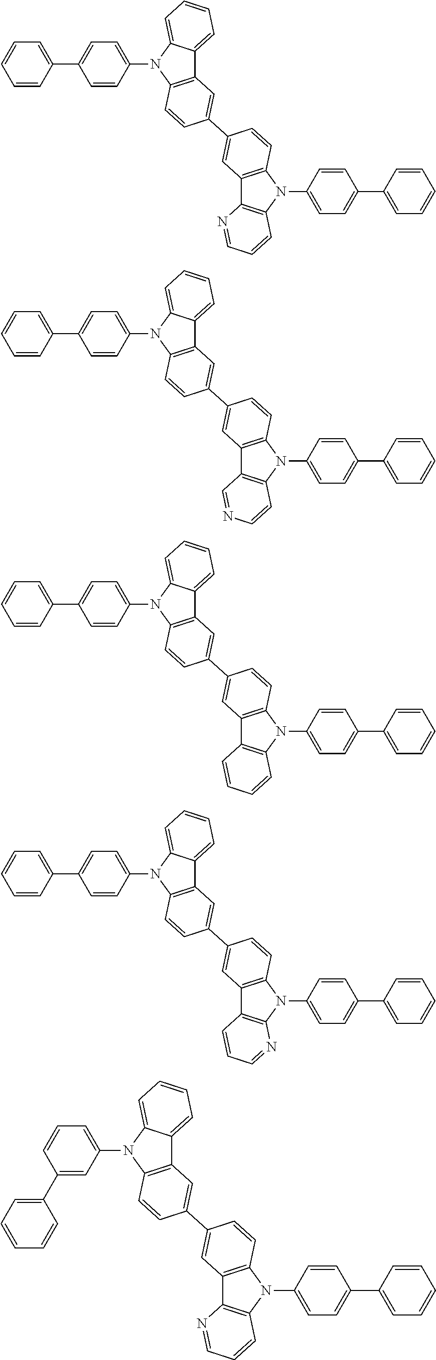 [SMILES: c1ccc(-c2ccc(-n3c4ccccc4c4cc(-c5ccc6c(c5)c5ncccc5n6-c5ccc(-c6ccccc6)cc5)ccc43)cc2)cc1 ; c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5cccnc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ncccc43)cc2)cc1]

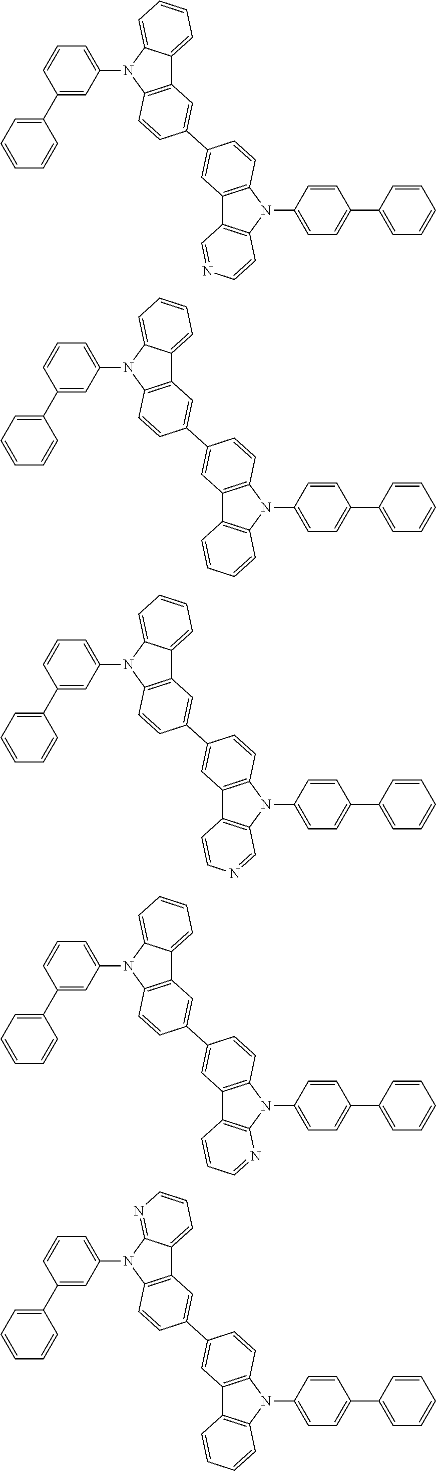 [SMILES: c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1 ; c1ccc(-c2ccc(-n3c4ccc(-c5ccc6c(c5)c5ccccc5n6-c5cccc(-c6ccccc6)c5)cc4c4cccnc43)cc2)cc1 ; c1ccc(-c2ccc(-n3c4ccccc4c4cc(-c5ccc6c(c5)c5cccnc5n6-c5cccc(-c6ccccc6)c5)ccc43)cc2)cc1]

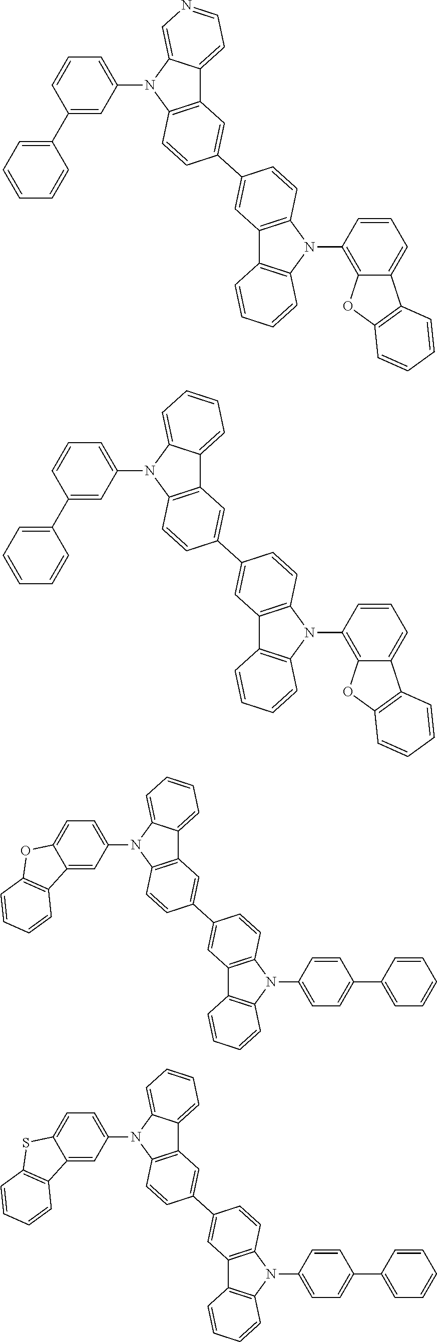

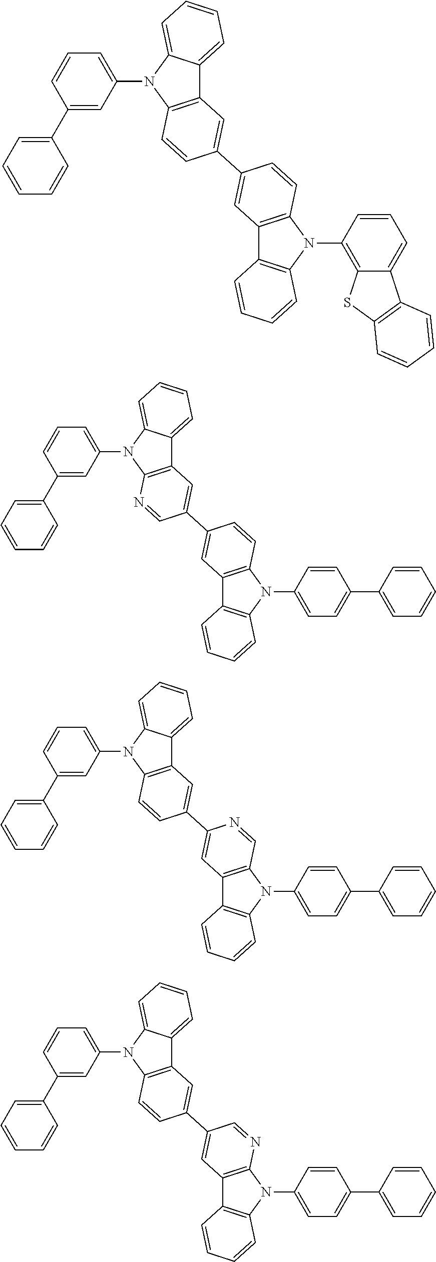 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5cnc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ncc43)cc2)cc1 ; c1ccc(-c2ccc(-n3c4ccccc4c4cc(-c5ccc6c(c5)c5ccccc5n6-c5cccc(-c6ccccc6)c5)cnc43)cc2)cc1]

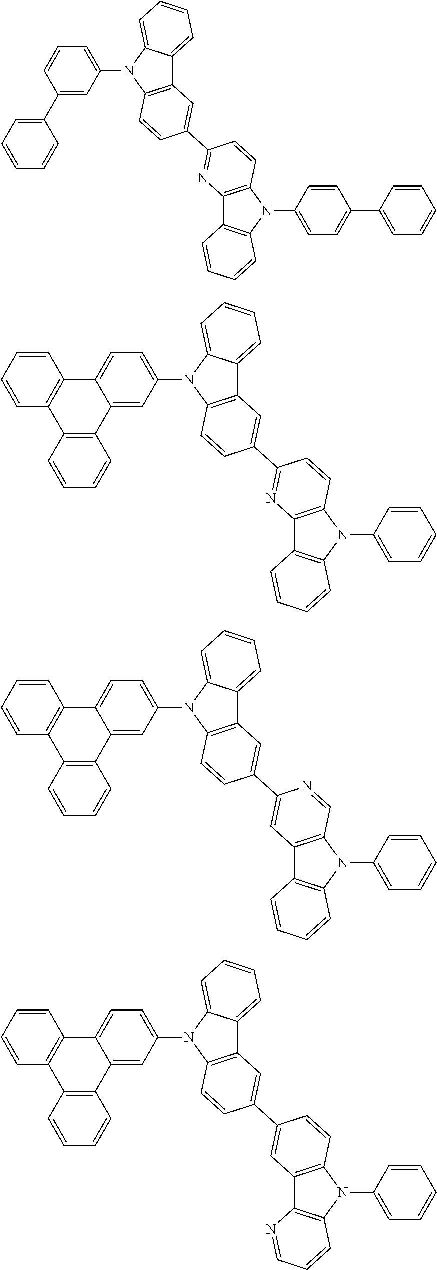

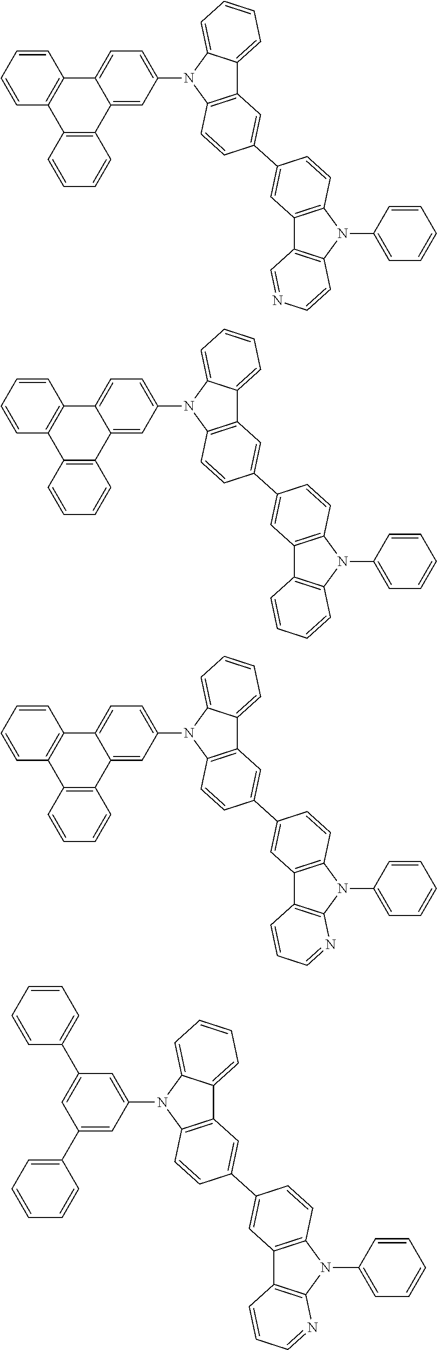 [SMILES: c1ccc(-n2c3ccncc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccc(-c4ccc5c(c4)c4ccccc4n5-c4ccc5c6ccccc6c6ccccc6c5c4)cc3c3cccnc32)cc1 ; c1ccc(-c2cc(-c3ccccc3)cc(-n3c4ccccc4c4cc(-c5ccc6c(c5)c5cccnc5n6-c5ccccc5)ccc43)c2)cc1]

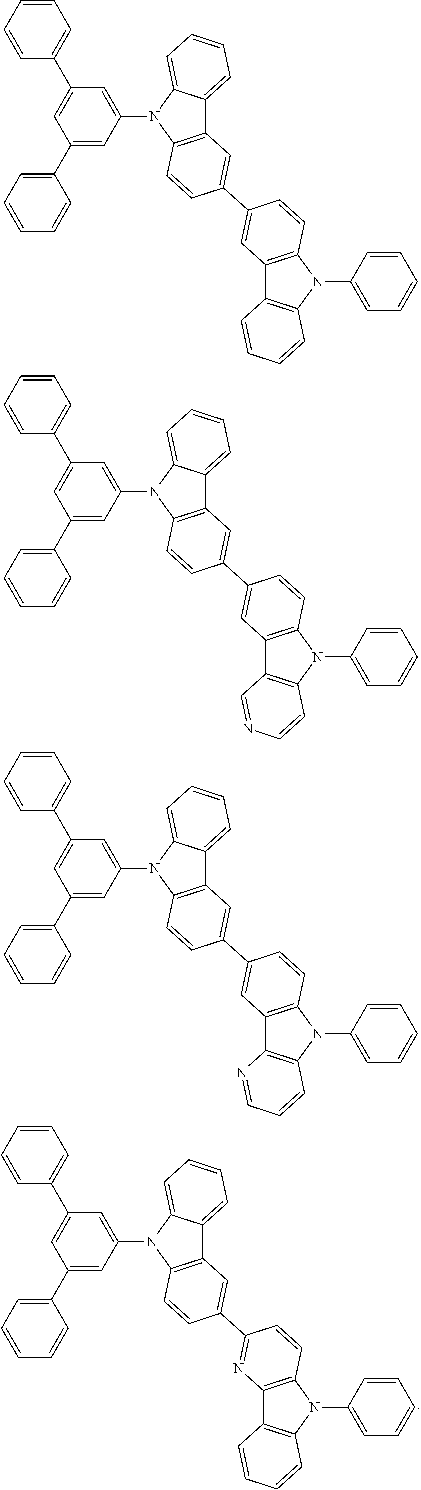 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cc(-c3ccccc3)cc(-n3c4ccccc4c4cc(-c5ccc6c(c5)c5cnccc5n6-c5ccccc5)ccc43)c2)cc1 ; c1ccc(-c2cc(-c3ccccc3)cc(-n3c4ccccc4c4cc(-c5ccc6c(c5)c5ncccc5n6-c5ccccc5)ccc43)c2)cc1 ; c1ccc(-c2cc(-c3ccccc3)cc(-n3c4ccccc4c4cc(-c5ccc6c(n5)c5ccccc5n6-c5ccccc5)ccc43)c2)cc1]

43. The organic mixture of claim 38, further comprising an organic solvent or an organic functional material selected from the group consisting of a hole injection material, a hole transport material, an electron transport material, an electron injection material, an electron blocking material, a hole blocking material, an emitter, a host material, and a thermally activated delayed fluorescent material.

44. An organic electronic device, comprising a functional layer, the functional layer comprising an organic compound of claim 25.
